(19) United States
(12) Patent Application Publication
(10) Pub. No.: US 2024/0150298 A1
(43) Pub. Date: May 9, 2024

(54) ANTIBACTERIAL COMPOUNDS

(71) Applicant: Blacksmith Medicines, Inc., San Diego, CA (US)

(72) Inventors: Min TENG, San Diego, CA (US); Baskar NAMMALWAR, San Diego, CA (US); Xiaoming LI, San Diego, CA (US); Christian PEREZ, San Diego, CA (US); David T. PUERTA, San Diego, CA (US)

(21) Appl. No.: 18/276,588

(22) PCT Filed: Feb. 8, 2022
(86) PCT No.: PCT/US2022/015679

§ 371 Date: Aug. 9, 2023

Related U.S. Application Data
(60) Provisional application No. 63/148,259, filed on Feb. 11, 2021.

(51) Int. Cl. C07D 233/64; A61P 31/04

(52) U.S. Cl. CPC C07D 233/64; A61P 31/04

(57) ABSTRACT

Provided herein are heterocyclic compounds and pharmaceutical compositions comprising said compounds that are useful for inhibiting the growth of gram-negative bacteria. The subject compounds and compositions are useful for the treatment of bacterial infections, such as pneumonia.

### CROSS-REFERENCE TO RELATED APPLICATIONS

This application claims the benefit of U.S. Provisional Patent Application No. 63/148,259 filed on Feb. 11, 2021, which is herein incorporated by reference in its entirety.

### STATEMENT AS TO FEDERALLY SPONSORED RESEARCH

This invention was made with government support under IDSEP160030 awarded by the U.S. Department of Health & Human Services. The government has certain rights in the invention.

### BACKGROUND OF THE INVENTION

A need exists in the medicinal arts for the effective treatment of illness caused by bacterial infection.

### SUMMARY OF THE INVENTION

Provided herein are heterocyclic compounds and pharmaceutical compositions comprising said compounds that are useful for inhibiting the growth of gram-negative bacteria.

The subject compounds and compositions are useful for the treatment of bacterial infection, such as pneumonia and the like. In some embodiments, compounds described herein are UDP-{3—O—[(R)—3-hydroxymyristoyl]}-N-acetylglucosamine deacetylase (LpxC) modulator compounds. In some embodiments, the compounds described herein are UDP-{3—O—[(R)—3-hydroxymyristoyl]}-N-acetylglucosamine deacetylase (LpxC) antagonists. In some embodiments, the compounds described herein are UDP-{3—O—[(R)—3-hydroxymyristoyl]}-N-acetylglucosamine deacetylase (LpxC) inhibitors.

In one aspect, described herein is a compound of Formula (I):

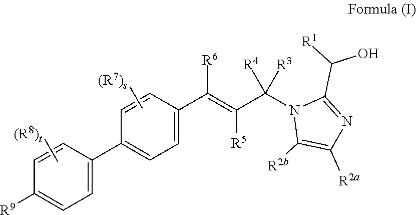

or a pharmaceutically acceptable salt, or solvate thereof, wherein:
R¹ is C₁-C₄ alkyl;
R^{2a} and R^{2b} are each independently hydrogen, halogen, or C₁-C₄ alkyl;
R³ is hydrogen or —(C₁-C₄ alkylene)-OH;
R⁴ is hydrogen or C₁-C₄ alkyl;
R⁵ is hydrogen or halogen;
R⁶ is hydrogen or halogen;
each R⁷ and R⁸ is independently hydrogen, halogen, or C₁-C₄ alkyl;
R⁹ is C₁-C₆ alkoxy, C₃-C₆ cycloalkyl, 4- to 6-membered heterocycloalkyl, —O—(C₃-C₆ cycloalkyl), —O—(4- to 6-membered heterocycloalkyl), —(C₁-C₄ alkylene)-(C₃-C₆ cycloalkyl), —(C₁-C₄ alkylene)-(4- to 6-membered heterocycloalkyl), —O—(C₁-C₄ alkylene)-(C₃-C₆ cycloalkyl), —O—(C₁-C₄ alkylene)-(4- to 6-membered heterocycloalkyl), —(C₁-C₄ alkylene)-O—(C₃-C₆ cycloalkyl), or —(C₁-C₄ alkylene)-O—(4- to 6-membered heterocycloalkyl); wherein the alkoxy, cycloalkyl, or heterocycloalkyl is unsubstituted or substituted by 1, 2, or 3 groups independently selected from —OR¹⁰, —N(R¹⁰)₂, —CO₂R¹⁰, —CON(R¹⁰)₂, —CH₂N(R¹⁰)₂, —NHCOR¹⁰, —NHSO₂R¹⁰, —OCOR¹⁰, phenyl, monocyclic heteroaryl, C₁-C₄ alkyl, C₁-C₄ hydroxyalkyl, C₁-C₄ aminoalkyl, C₃-C₆ cycloalkyl, and 4-to 6-membered heterocycloalkyl which is unsubstituted or substituted by 1, 2, or 3 —OH groups;
each R¹⁰ is independently hydrogen, 4- to 6-membered heterocycloalkyl, or C₁-C₄ alkyl which is unsubstituted or substituted by 1, 2, or 3 groups independently selected from —F, —CN, —OH, —NH₂, —OMe, —N(CH₃)₂, —CO₂H, —CONH₂, —SO₂CH₃, phenyl, oxazolidinone, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; or two R¹⁰ attached to the same nitrogen are taken together to form a 4- to 6-membered heterocycloalkyl which is unsubstituted or substituted by 1, 2, or 3 groups independently selected from —F, —CN, —OH, —NH₂, —OMe, —CO₂H, —CONH₂, and —SO₂CH₃;
s is 1 or 2; and
t is 1 or 2.

In some embodiments, the compound of Formula (I) is a compound of Formula (IIa):

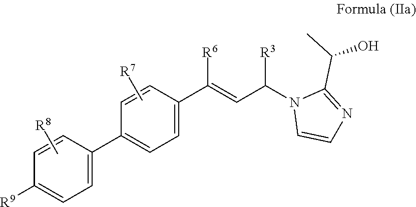

or a pharmaceutically acceptable salt, or solvate thereof, wherein:
R³ is hydrogen or —(C₁-C₄ alkylene)-OH;
R⁶ is hydrogen or fluoro;
R⁷ is hydrogen or fluoro;
R⁸ is hydrogen or fluoro;
R⁹ is C₁-C₆ alkoxy, C₃-C₆ cycloalkyl, 4- to 6-membered heterocycloalkyl, —O—(C₃-C₆ cycloalkyl), —O—(4- to 6-membered heterocycloalkyl), —(C₁-C₄ alkylene)-(C₃-C₆ cycloalkyl), —(C₁-C₄ alkylene)-(4- to 6-membered heterocycloalkyl), —O—(C₁-C₄ alkylene)-(C₃-C₆ cycloalkyl), —O—(C₁-C₄ alkylene)-(4- to 6-membered heterocycloalkyl), —(C₁-C₄ alkylene)-O—(C₃-C₆ cycloalkyl), or —(C₁-C₄ alkylene)-O—(4- to 6-membered heterocycloalkyl); wherein the alkoxy, cycloalkyl, or heterocycloalkyl is unsubstituted or substituted by 1, 2, or 3 groups independently selected from —OR¹⁰, —N(R¹⁰)₂, —CO₂R¹⁰, —CON(R¹⁰)₂, —CH₂N(R¹⁰)₂, —NHCOR¹⁰, —NHSO₂R¹⁰, —OCOR¹⁰, phenyl, monocyclic heteroaryl, C₁-C₄ alkyl, C₁-C₄ hydroxyalkyl, C₁-C₄ aminoalkyl, C₃-C₆ cycloalkyl, and 4- to 6-membered heterocycloalkyl which is unsubstituted or substituted by 1, 2, or 3 —OH groups; and
each R¹⁰ is independently hydrogen, 4- to 6-membered heterocycloalkyl, or C₁-C₄ alkyl which is unsubstituted or substituted by 1, 2, or 3 groups independently selected from —F, —CN, —OH, —NH₂, —OMe, —N(CH₃)₂, —CO₂H, —CONH₂, —SO₂CH₃, phenyl, oxazolidinone, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; or two R¹⁰ attached to the same nitrogen are taken together to form a 4- to 6-membered heterocycloalkyl which is unsubstituted or substituted by 1, 2, or 3 groups independently selected from —F, —CN, —OH, —NH₂, —OMe, —CO₂H, —CONH₂, and —SO₂CH₃.

In some embodiments, the compound of Formula (I) or (IIa) is a compound of Formula (IIIa):

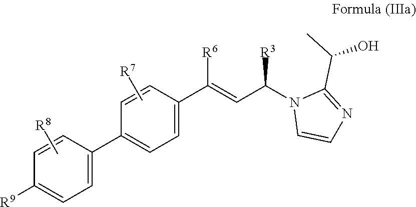

or a pharmaceutically acceptable salt, or solvate thereof, wherein:
R⁶ is hydrogen or fluoro;
R⁷ is hydrogen or fluoro;
R⁸ is hydrogen or fluoro;
R³ is —(C₁-C₄ alkylene)-OH;
R⁹ is C₁-C₆ alkoxy, C₃-C₆ cycloalkyl, 4- to 6-membered heterocycloalkyl, —O—(C₃-C₆ cycloalkyl), —O—(4- to 6-membered heterocycloalkyl), —(C₁-C₄ alkylene)-(C₃-C₆ cycloalkyl), —(C₁-C₄ alkylene)-(4- to 6-membered heterocycloalkyl), —O—(C₁-C₄ alkylene)-(C₃-C₆ cycloalkyl), —O—(C₁-C₄ alkylene)-(4- to 6-membered heterocycloalkyl), —(C₁-C₄ alkylene)-O—(C₃-C₆ cycloalkyl), or —(C₁-C₄ alkylene)-O—(4- to 6-membered heterocycloalkyl); wherein the alkoxy, cycloalkyl, or heterocycloalkyl is unsubstituted or substituted by 1, 2, or 3 groups independently selected from —OR¹⁰, —N(R¹⁰)₂, —CO₂R¹⁰, —CON(R¹⁰)₂, —CH₂N(R¹⁰)₂, —NHCOR¹⁰, —NHSO₂R¹⁰, —OCOR¹⁰, phenyl, monocyclic heteroaryl, C₁-C₄ alkyl, C₁-C₄ hydroxyalkyl, C₁-C₄ aminoalkyl, C₃-C₆ cycloalkyl, and 4- to 6-membered heterocycloalkyl which is unsubstituted or substituted by 1, 2, or 3 —OH groups; and
each R¹⁰ is independently hydrogen, 4- to 6-membered heterocycloalkyl, or C₁-C₄ alkyl which is unsubstituted or substituted by 1, 2, or 3 groups independently selected from —F, —CN, —OH, —NH₂, —OMe, —N(CH₃)₂, —CO₂H, —CONH₂, —SO₂CH₃, phenyl, oxazolidinone, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; or two R¹⁰ attached to the same nitrogen are taken together to form a 4- to 6-membered heterocycloalkyl which is unsubstituted or substituted by 1, 2, or 3 groups independently selected from —F, —CN, —OH, —NH₂, —OMe, —CO₂H, —CONH₂, and —SO₂CH₃.

In some embodiments, the compound of Formula (I), (IIa), or (IIIa) is a compound of Formula (IVa):

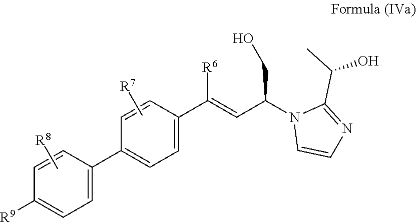

or a pharmaceutically acceptable salt, or solvate thereof, wherein:
R⁶ is hydrogen or fluoro;
R⁷ is hydrogen or fluoro;
R⁸ is hydrogen or fluoro;
R⁹ is C₁-C₆ alkoxy, C₃-C₆ cycloalkyl, 4- to 6-membered heterocycloalkyl, —O—(C₃-C₆ cycloalkyl), —O—(4- to 6-membered heterocycloalkyl), —(C₁-C₄ alkylene)-(C₃-C₆ cycloalkyl), —(C₁-C₄ alkylene)-(4- to 6-membered heterocycloalkyl), —O—(C₁-C₄ alkylene)-(C₃-C₆ cycloalkyl), —O—(C₁-C₄ alkylene)-(4- to 6-membered heterocycloalkyl), —(C₁-C₄ alkylene)-O—(C₃-C₆ cycloalkyl), or —(C₁-C₄ alkylene)-O—(4- to 6-membered heterocycloalkyl); wherein the alkoxy, cycloalkyl, or heterocycloalkyl is unsubstituted or substituted by 1, 2, or 3 groups independently selected from —OR¹⁰, —N(R¹⁰)₂, —CO₂R¹⁰, —CON(R¹⁰)₂, —CH₂N(R¹⁰)₂, —NHCOR¹⁰, —NHSO₂R¹⁰, —OCOR¹⁰, phenyl, monocyclic heteroaryl, C₁-C₄ alkyl, C₁-C₄ hydroxyalkyl, C₁-C₄ aminoalkyl, C₃-C₆ cycloalkyl, and 4- to 6-membered heterocycloalkyl which is unsubstituted or substituted by 1, 2, or 3 —OH groups; and
each R¹⁰ is independently hydrogen, 4- to 6-membered heterocycloalkyl, or C₁-C₄ alkyl which is unsubstituted or substituted by 1, 2, or 3 groups independently selected from —F, —CN, —OH, —NH₂, —OMe, —N(CH₃)₂, —CO₂H, —CONH₂, —SO₂CH₃, phenyl, oxazolidinone, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; or two R¹⁰ attached to the same nitrogen are taken together to form a 4- to 6-membered heterocycloalkyl which is unsubstituted or substituted by 1, 2, or 3 groups independently selected from —F, —CN, —OH, —NH₂, —OMe, —CO₂H, —CONH₂, and —SO₂CH₃.

In some embodiments, the compound of Formula (I) is a compound of Formula (V):

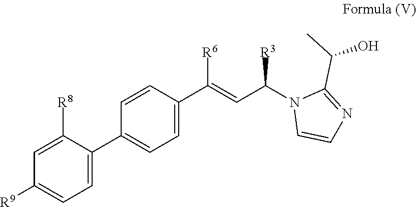

or a pharmaceutically acceptable salt, or solvate thereof, wherein:
R³ is hydrogen or —(C₁-C₄ alkylene)-OH;
R⁶ is hydrogen or fluoro;
R⁸ is hydrogen or fluoro;
R⁹ is C₁-C₆ alkoxy, C₃-C₆ cycloalkyl, —O—(C₃-C₆ cycloalkyl), —O—(4- to 6-membered heterocycloalkyl), —O—(C₁-C₄ alkylene)-(C₃-C₆ cycloalkyl), —O—(C₁-C₄ alkylene)-(4- to 6-membered heterocycloalkyl), or —(C₁-C₄ alkylene)-O—(C₃-C₆ cycloalkyl); wherein the alkoxy, cycloalkyl, or heterocycloalkyl is unsubstituted or substituted by 1 or 2 groups independently selected from —OR¹⁰, —N(R¹⁰)₂, —CO₂R¹⁰, —CON(R¹⁰)₂, —CH₂N(R¹⁰)₂, —NHCOR¹⁰, —NHSO₂R¹⁰, —OCOR¹⁰, C₁-C₄ hydroxyalkyl, C₁-C₄ aminoalkyl, and 4- to 6-membered heterocycloalkyl which is unsubstituted or substituted by 1 —OH group; and
each R¹⁰ is independently hydrogen, 4- to 6-membered heterocycloalkyl, or C₁-C₄ alkyl which is unsubstituted or substituted by 1, 2, or 3 groups independently selected from —F, —CN, —OH, —N(CH₃)₂, —CO₂H, —SO₂CH₃, oxazolidinone, and monocyclic heteroaryl which is unsubstituted or substituted by 1 —CONH₂ group;
or two R¹⁰ attached to the same nitrogen are taken together to form an azetidinyl which is unsubstituted or substituted by 1 —SO₂CH₃ group.

In some embodiments, the compound of Formula (I) is a compound of Formula (VI):

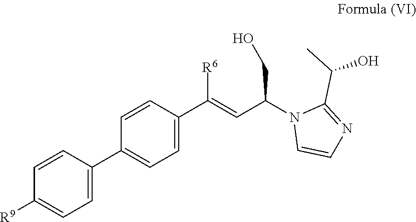

or a pharmaceutically acceptable salt, or solvate thereof, wherein:
R⁶ is hydrogen or fluoro;
R⁹ is C₁-C₆ alkoxy, C₃-C₆ cycloalkyl, —O—(C₃-C₆ cycloalkyl), —O—(4- to 6-membered heterocycloalkyl), —O—(C₁-C₄ alkylene)-(C₃-C₆ cycloalkyl), —O—(C₁-C₄ alkylene)-(4- to 6-membered heterocycloalkyl), or —(C₁-C₄ alkylene)-O—(C₃-C₆ cycloalkyl); wherein the alkoxy, cycloalkyl, or heterocycloalkyl is unsubstituted or substituted by 1 or 2 groups independently selected from —OR¹⁰, —N(R¹⁰)₂, —CO₂R¹⁰, —CON(R¹⁰)₂, —CH₂N(R¹⁰)₂, —NHCOR¹⁰, —NHSO₂R¹⁰, —OCOR¹⁰, C₁-C₄ hydroxyalkyl, C₁-C₄ aminoalkyl, and 4- to 6-membered heterocycloalkyl which is unsubstituted or substituted by 1 —OH group; and
each R¹⁰ is independently hydrogen, 4- to 6-membered heterocycloalkyl, or C₁-C₄ alkyl which is unsubstituted or substituted by 1, 2, or 3 groups independently selected from —F, —CN, —OH, —N(CH₃)₂, —CO₂H, —SO₂CH₃, oxazolidinone, and monocyclic heteroaryl which is unsubstituted or substituted by 1 —CONH₂ group;
or two R¹⁰ attached to the same nitrogen are taken together to form an azetidinyl which is unsubstituted or substituted by 1 —SO₂CH₃ group.

Any combination of the groups described above for the various variables is contemplated herein. Throughout the specification, groups and substituents thereof are chosen by one skilled in the field to provide stable moieties and compounds.

Also described herein is a pharmaceutical composition comprising a compound described herein, or a pharmaceutically acceptable salt, or solvate thereof, and at least one pharmaceutically acceptable excipient. In some embodiments, the pharmaceutical composition is formulated for administration to a mammal by intravenous administration, subcutaneous administration, oral administration, inhalation, nasal administration, dermal administration, or ophthalmic administration. In some embodiments, the pharmaceutical composition is formulated for administration to a mammal by oral administration. In some embodiments, the pharmaceutical composition is in the form of a tablet, a pill, a capsule, a liquid, a suspension, a gel, a dispersion, a solution, an emulsion, an ointment, or a lotion. In some embodiments, the pharmaceutical composition is in the form of a tablet, a pill, or a capsule.

In another aspect provided herein is a method of treating or preventing a gram-negative bacterial infection in a patient in need thereof comprising administering to the patient a pharmaceutical composition comprising a compound described herein, or a pharmaceutically acceptable salt thereof, and a pharmaceutically acceptable excipient. In some embodiments, the gram-negative bacterial infection is associated with Pseudomonas aeruginosa. In some embodiments, the gram-negative bacterial infection is a respiratory infection. In some embodiments, the gram-negative bacterial infection is pneumonia. In some embodiments, provided herein, the gram-negative bacterial infection is community-acquired pneumonia (CAP), health care-associated pneumonia (HCAP), hospital-acquired pneumonia (HAP), ventilator-associate pneumonia (VAP), or a combination thereof. In some embodiments, the patient has been identified as having a lung disease. In some embodiments, the lung disease is a structural lung disease. In some embodiments, the lung disease is cystic fibrosis, bronchiectasis, emphysema, chronic obstructive pulmonary disease (COPD), chronic destroyed lung disease, or a combination thereof. In some embodiments, the administration is to treat an existing infection.

In some embodiments, the administration is provided as prophylaxis. In some embodiments, the compound or a pharmaceutically acceptable salt, or solvate thereof, or the pharmaceutical composition described herein is administered in a solution by inhalation, intravenous injection, or intraperitoneal injection.

In another aspect provided herein is a method of inhibiting UDP-{3—O—[(R)—3-hydroxymyristoyl]}-N-acetylglucosamine deacetylase enzyme comprising contacting the enzyme with a compound described herein.

In another aspect provided herein is a method for treating bacterial infection in a patient in need thereof comprising administering to the patient a composition comprising a compound described herein, or a pharmaceutically acceptable salt thereof, and a pharmaceutically acceptable excipient.

In any of the aforementioned aspects are further embodiments in which the effective amount of the compound of Formula (I), or a pharmaceutically acceptable salt, or solvate thereof, is: (a) systemically administered to the mammal; and/or (b) administered orally to the mammal; and/or (c) intravenously administered to the mammal; and/or (d) administered by inhalation; and/or (e) administered by nasal administration; or and/or (f) administered by injection to the mammal; and/or (g) administered topically to the mammal; and/or (h) administered by ophthalmic administration; and/or (i) administered rectally to the mammal; and/or (j) administered non-systemically or locally to the mammal.

In any of the aforementioned aspects are further embodiments comprising single administrations of the effective amount of the compound, including further embodiments in which the compound is administered once a day to the mammal or the compound is administered to the mammal multiple times over the span of one day. In some embodiments, the compound is administered on a continuous dosing schedule. In some embodiments, the compound is administered on a continuous daily dosing schedule.

In any of the embodiments disclosed herein, the mammal is a human.

Articles of manufacture, which include packaging material, a compound of Formula (I), or a pharmaceutically acceptable salt thereof, within the packaging material, and a label that indicates that the compound or composition, or pharmaceutically acceptable salt, tautomers, pharmaceutically acceptable N-oxide, pharmaceutically active metabolite, pharmaceutically acceptable prodrug, or pharmaceutically acceptable solvate thereof, is used for modulating UDP-{3—O—[(R)—3-hydroxymyristoyl]}-N-acetylglucosamine deacetylase (LpxC), or for the treatment, prevention or amelioration of one or more symptoms of a disease or condition that would benefit from modulating UDP-{3—O—[(R)—3-hydroxymyristoyl]}-N-acetylglucosamine deacetylase (LpxC), are provided.

Other objects, features, and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### INCORPORATION BY REFERENCE

All publications, patents, and patent applications mentioned in this specification are herein incorporated by reference for the specific purposes identified herein.

### DETAILED DESCRIPTION OF THE INVENTION

### LpxC, Lipid A and Gram-Negative Bacteria

Metalloproteins influence a vast diversity of biological systems, biological processes, and diseases. For example, UDP-{3—O—[(R)—3-hydroxymyristoyl]}-N-acetylglucosamine deacetylase (LpxC) is an essential enzyme involved in the first committed step in lipid A biosynthesis for gram-negative bacteria. Lipid A is an essential component of the outer membrane of gram-negative bacteria. LpxC is a zinc(II)-dependent metalloenzyme, with two histidines and an aspartic acid residue bound to the zinc(II) ion. Structures of LpxC show the zinc(II) ion is bound to two water molecules, both of which have been implicated in the mechanism of the enzyme. LpxC is highly conserved across strains of gram-negative bacteria, making LpxC an attractive target to treat gram-negative infections. To the contrary, LpxC is not a component of Gram-positive bacteria, such as Staphylococcus aureus.

In recent years, there has been an increase in resistant and multi-drug resistant strains of bacteria. Thus, there is a need for new antibiotics, especially with new mechanisms of action. There remains a need for metalloprotein modulators of LpxC useful in the field of therapeutics, diagnostics, and research.

Some embodiments provide a method of inhibiting UDP-{3—O—[(R)—3-hydroxymyristoyl]}-N-acetylglucosamine deacetylase enzyme comprising contacting the enzyme with a compound of Formula (I).

In some embodiments provided herein is a pharmaceutical composition comprising a compound of Formula (I) or a pharmaceutically acceptable salt thereof, and a pharmaceutically acceptable excipient.

### Methods of Use

Disclosed herein are methods of treating disease wherein the inhibition of bacterial growth is indicated. Such disease includes gram-negative bacterial infection. In some embodiments, the gram-negative bacterial infection is associated with Pseudomonas aeruginosa. In some embodiments, the method of treating a gram-negative bacterial infection in a patient in need thereof comprises administering to the patient a compound of Formula (I), a pharmaceutical composition comprising a compound of Formula (I), or a pharmaceutically acceptable salt thereof, and a pharmaceutically acceptable excipient. In some embodiments, the method of treating a Pseudomonas aeruginosa infection in a patient in need thereof comprises administering to the patient the compound of Formula (I), a pharmaceutical composition comprising a compound of Formula (I), or a pharmaceutically acceptable salt thereof, and a pharmaceutically acceptable excipient.

In some embodiments, the gram-negative bacterial infection is associated with Pseudomonas aeruginosa. In some embodiments, the gram-negative bacterial infection is a respiratory infection. In some embodiments, the gram-negative bacterial infection is pneumonia. In some embodiments, the gram-negative bacterial infection is community-acquired pneumonia (CAP), health care-associated pneumonia (HCAP), hospital-acquired pneumonia (HAP), ventilator-associate pneumonia (VAP), or a combination thereof. In some embodiments, the gram-negative bacterial infection is community-acquired pneumonia (CAP). In some embodiments, the gram-negative bacterial infection is health care-associated pneumonia (HCAP). In some embodiments, the gram-negative bacterial infection is hospital-acquired pneumonia (HAP). In some embodiments, the gram-negative bacterial infection is ventilator-associate pneumonia (VAP).

In some embodiments, the patient has been identified as having a lung disease. In some embodiments, the lung disease is a structural lung disease. In some embodiments, the lung disease is cystic fibrosis, bronchiectasis, emphysema, chronic obstructive pulmonary disease (COPD), chronic destroyed lung disease, or a combination thereof. In some embodiments, the patient has cystic fibrosis. In some embodiments, the patient has bronchiectasis. In some embodiments, the patient has emphysema. In some embodiments, the patient has chronic obstructive pulmonary disease (COPD). In some embodiments, the patient has chronic destroyed lung disease.

In some embodiments the administration is to treat an existing infection.

In some embodiments the administration is provided as prophylaxis.

In some embodiments, the LpxC inhibitory compound as described herein is used for treating or preventing conditions caused by the bacterial production of endotoxin and, in particular, by gram-negative bacteria and bacteria that use LpxC in the biosynthesis of lipopolysaccharide (LPS) or endotoxin. In some embodiments, the method of treating or preventing a condition caused by endotoxin or LPS in a patient in need thereof comprises administering to the patient a pharmaceutical composition comprising a compound of Formula (I), or a pharmaceutically acceptable salt thereof, and a pharmaceutically acceptable excipient. In another embodiment, the heterocyclic LpxC inhibitory compounds as described herein are useful in the treatment of conditions that are caused or exacerbated by the bacterial production of lipid A and LPS or endotoxin, such as chronic obstructive pulmonary disease (COPD) and acute exacerbations of chronic bronchitis (AECB). In some embodiments, the method of treating or preventing a condition caused by endotoxin or LPS in a patient in need thereof comprises administering to the patient a pharmaceutical composition comprising a compound of Formula (I), or a pharmaceutically acceptable salt thereof, and a pharmaceutically acceptable excipient, wherein the condition caused by endotoxin or LPS is selected from chronic obstructive pulmonary disease (COPD) and acute exacerbations of chronic bronchitis (AECB).

In other embodiments, the compounds of the disclosure can be used for the treatment of a serious or chronic respiratory tract infection including serious lung and nosocomial infections such as those caused by Enterobacter aerogenes, Enterobacter cloacae, Escherichia coli, Klebsiella pneumoniae, Klebsiella oxytoca, Kuyvera ascorbata, Kuyvera cryocrescense, Shigella sonnei, Proteus mirabilis, Serratia marcescens, Stenotrophomonas maltophilia, Pseudomonas aeruginosa, Burkholderia cepacia, Acinetobacter baumannii, Alcaligenes xylosoxidans, Flavobacterium meningosepticum, and Citrobacter freundi, Haemophilus influenzae, Kluyvera species, Legionella species, Moraxella catarrhalis, Enterobacter species, Acinetobacter species, Klebsiella species, Burkholderia species and Proteus species, and infections caused by other bacterial species such as Neisseria species, Shigella species, Salmonella species, Helicobacler pylori, Vibrionaceae and Bordetella species as well as the infections caused by a Brucella species, Francisella tularensis and/or Yersiniapestis. In some embodiments, the infection is associated with a Pseudomonas species. In some embodiments, the infection is associated with Pseudomonas aeruginosa. In some embodiments, the compounds of the disclosure do not inhibit the growth of Gram-positive bacteria, such as Staphylococcus aureus.

In some embodiments, the LpxC inhibitory compound as described herein is used in a method of preventing growth of a Pseudomonas species. In some embodiments, the Pseudomonas species is Pseudomonas aeruginosa.

In some instances, antibiotics have suboptimal concentrations in the lung leading to therapeutic failures for lung infections. In some embodiments, the heterocyclic LpxC inhibitory compound of Formula (I) have optimal concentrations in the lung for treating or preventing a gram-negative bacterial infection in the lung. In some embodiments, the compounds are present in the lung in a therapeutically effective amount after administration.

In some embodiments, disclosed herein is a compound described herein, or a pharmaceutically acceptable salt thereof, for use as therapeutically active substance.

In some embodiments, disclosed herein is a compound described herein, or a pharmaceutically acceptable salt thereof, for use in treating or preventing a gram-negative bacterial infection. In some embodiments, the gram-negative bacterial infection is associated with Pseudomonas aeruginosa. In some embodiments, the gram-negative bacterial infection is a respiratory infection. In some embodiments, the respiratory infection is pneumonia.

In some embodiments, disclosed herein is the use of a compound described herein, or a pharmaceutically acceptable salt thereof, for the preparation of a medicament for treating or preventing a gram-negative bacterial infection. In some embodiments, the gram-negative bacterial infection is associated with Pseudomonas aeruginosa. In some embodiments, the gram-negative bacterial infection is a respiratory infection. In some embodiments, the respiratory infection is pneumonia.

### LpxC Inhibitory Compounds

Provided herein, in some embodiments, are heterocyclic LpxC inhibitory compounds and pharmaceutical compositions comprising said compounds. The subject compounds and compositions are useful for inhibiting UDP-{3—O—[(R)—3-hydroxymyristoyl]}-N-acetylglucosamine deacetylase (LpxC) and for the treatment of bacterial infection.

In some embodiments, compounds of Formula (I), including pharmaceutically acceptable salts, prodrugs, active metabolites, and pharmaceutically acceptable solvates thereof, are UDP-{3—O—[(R)—3-hydroxymyristoyl]}-N-acetylglucosamine deacetylase (LpxC) modulators. In some embodiments, the compounds of Formula (I), including pharmaceutically acceptable salts, prodrugs, active metabolites, and pharmaceutically acceptable solvates thereof, are UDP-{3—O—[(R)—3-hydroxymyristoyl]}-N-acetylglucosamine deacetylase (LpxC) antagonists. In some embodiments, the compounds of Formula (I), including pharmaceutically acceptable salts, prodrugs, active metabolites, and pharmaceutically acceptable solvates thereof, are UDP-{3—O—[(R)—3-hydroxymyristoyl]}-N-acetylglucosamine deacetylase (LpxC) inhibitors.

One aspect of the disclosure provides a compound having the structure of Formula (I):

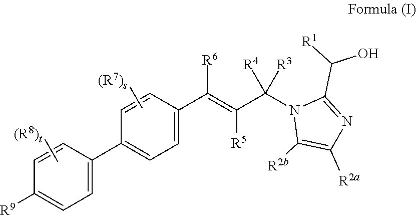

or a pharmaceutically acceptable salt, or solvate thereof, wherein:
R¹ is C₁-C₄ alkyl;
R^{2a} and R^{2b} are each independently hydrogen, halogen, or C₁-C₄ alkyl;
R³ is hydrogen or —(C₁-C₄ alkylene)-OH;
R⁴ is hydrogen or C₁-C₄ alkyl;
R⁵ is hydrogen or halogen;
R⁶ is hydrogen or halogen;
each R⁷ and R⁸ is independently hydrogen, halogen, or C₁-C₄ alkyl;
R⁹ is C₁-C₆ alkoxy, C₃-C₆ cycloalkyl, 4- to 6-membered heterocycloalkyl, —O—(C₃-C₆ cycloalkyl), —O—(4- to 6-membered heterocycloalkyl), —(C₁-C₄ alkylene)-(C₃-C₆ cycloalkyl), —(C₁-C₄ alkylene)-(4- to 6-membered heterocycloalkyl), —O—(C₁-C₄ alkylene)-(C₃-C₆ cycloalkyl), —O—(C₁-C₄ alkylene)-(4- to 6-membered heterocycloalkyl), —(C₁-C₄ alkylene)-O—(C₃-C₆ cycloalkyl), or —(C₁-C₄ alkylene)-O—(4- to 6-membered heterocycloalkyl); wherein the alkoxy, cycloalkyl, or heterocycloalkyl is unsubstituted or substituted by 1, 2, or 3 groups independently selected from —OR¹⁰, —N(R¹⁰)₂, —CO₂R¹⁰, —CON(R¹⁰)₂, —CH₂N(R¹⁰)₂, —NHCOR¹⁰, —NHSO₂R¹⁰, —OCOR¹⁰, phenyl, monocyclic heteroaryl, C₁-C₄ alkyl, C₁-C₄ hydroxyalkyl, C₁-C₄ aminoalkyl, C₃-C₆ cycloalkyl, and 4- to 6-membered heterocycloalkyl which is unsubstituted or substituted by 1, 2, or 3 —OH groups;
each R¹⁰ is independently hydrogen, 4- to 6-membered heterocycloalkyl, or C₁-C₄ alkyl which is unsubstituted or substituted by 1, 2, or 3 groups independently selected from —F, —CN, —OH, —NH₂, —OMe, —N(CH₃)₂, —CO₂H, —CONH₂, —SO₂CH₃, phenyl, oxazolidinone, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃;
or two R¹⁰ attached to the same nitrogen are taken together to form a 4- to 6-membered heterocycloalkyl which is unsubstituted or substituted by 1, 2, or 3 groups independently selected from —F, —CN, —OH, —NH₂, —OMe, —CO₂H, —CONH₂, and —SO₂CH₃;
s is 1 or 2; and
t is 1 or 2.

In some embodiments,
R¹ is C₁-C₄ alkyl;
R^{2a} and R^{2b} are each independently hydrogen, halogen, or C₁-C₄ alkyl;
R³ is hydrogen or —(C₁-C₄ alkylene)-OH;
R⁴ is hydrogen or C₁-C₄ alkyl;
R⁵ is hydrogen or halogen;
R⁶ is hydrogen or halogen;
each R⁷ and R⁸ is independently hydrogen, halogen, or C₁-C₄ alkyl;
R⁹ is C₁-C₆ alkoxy, C₃-C₆ cycloalkyl, 4- to 6-membered heterocycloalkyl, —O—(C₃-C₆ cycloalkyl), —O—(4- to 6-membered heterocycloalkyl), —(C₁-C₄ alkylene)-(C₃-C₆ cycloalkyl), —(C₁-C₄ alkylene)-(4- to 6-membered heterocycloalkyl), —O—(C₁-C₄ alkylene)-(C₃-C₆ cycloalkyl), —O—(C₁-C₄ alkylene)-(4- to 6-membered heterocycloalkyl), —(C₁-C₄ alkylene)-O—(C₃-C₆ cycloalkyl), or —(C₁-C₄ alkylene)-O—(4- to 6-membered heterocycloalkyl); wherein the alkoxy, cycloalkyl, or heterocycloalkyl is unsubstituted or substituted by 1, 2, or 3 groups independently selected from —OR¹⁰, —N(R¹⁰)₂, —CO₂R¹⁰, —CON(R¹⁰)₂, —CH₂N(R¹⁰)₂, —NHCOR¹⁰, —NHSO₂R¹⁰, phenyl, monocyclic heteroaryl, C₁-C₄ alkyl, C₁-C₄ hydroxyalkyl, C₁-C₄ aminoalkyl, C₃-C₆ cycloalkyl, and 4- to 6-membered heterocycloalkyl which is unsubstituted or substituted by 1, 2, or 3 —OH groups; each R¹⁰ is independently hydrogen, 4- to 6-membered heterocycloalkyl, or C₁-C₄ alkyl which is unsubstituted or substituted by 1, 2, or 3 groups independently selected from —F, —CN, —OH, —NH₂, —OMe, —N(CH₃)₂, —CO₂H, —CONH₂, —SO₂CH₃, phenyl, oxazolidinone, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; or two R¹⁰ attached to the same nitrogen are taken together to form a 4- to 6-membered heterocycloalkyl which is unsubstituted or substituted by 1, 2, or 3 groups independently selected from —F, —CN, —OH, —NH₂, —OMe, —CO₂H, —CONH₂, and —SO₂CH₃;
s is 1 or 2; and
t is 1 or 2.

In some embodiments, the compound of Formula (I) is a compound of Formula (Ia) or Formula (Ib):

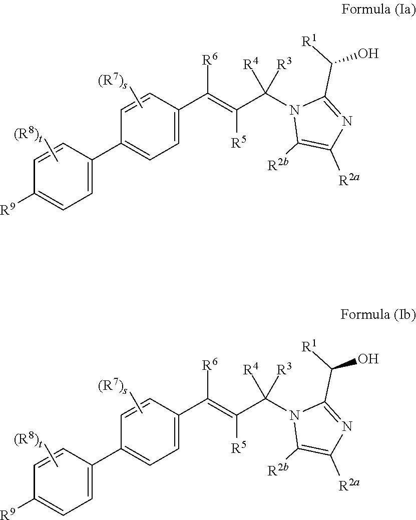

or a pharmaceutically acceptable salt, or solvate thereof.

In some embodiments, the compound is a compound of Formula (Ia), or a pharmaceutically acceptable salt, or solvate thereof. In some embodiments, the compound is a compound of Formula (Ib), or a pharmaceutically acceptable salt, or solvate thereof.

For any and all of the embodiments, substituents are selected from among a subset of the listed alternatives. For example, in some embodiments of a compound of Formula (I), (Ia), or (Ib), R¹ is unsubstituted C₁-C₄ alkyl. In some embodiments, R¹ is C₁-C₂ alkyl. In some embodiments, R¹ is —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), —C(CH₃)₃. In some embodiments, R¹ is —CH₃ or —CH₂CH₃. In some embodiments, R¹ is —CH₃.

In some embodiments of a compound of Formula (I), (Ia), or (Ib), R^{2a} and R^{2b} are each independently R^{2a} and R^{2b} are each independently hydrogen, halogen, or unsubstituted C₁-C₄ alkyl. In some embodiments, R^{2a} and R^{2b} are each independently hydrogen, —F, —Cl, —Br, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), or —C(CH₃)₃. In some embodiments, R^{2a} and R^{2b} are each independently hydrogen, —F, —Cl, —CH₃, —CH₂CH₃, or —CH(CH₃)₂.

In some embodiments of a compound of Formula (I), (Ia), or (Ib), R^{2a} is hydrogen. In some embodiments, R^{2b} is hydrogen. In some embodiments, R^{2a} and R^{2b} are each hydrogen.

In some embodiments of a compound of Formula (I), (Ia), or (Ib), R¹ is —CH₃; R^{2a} is hydrogen; and R^{2b} is hydrogen.

In some embodiments of a compound of Formula (I), (Ia), or (Ib), R⁴ is hydrogen or unsubstituted C₁-C₄ alkyl. In some embodiments, R⁴ is hydrogen or C₁-C₂ alkyl. In some embodiments, R⁴ is hydrogen, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), —C(CH₃)₃. In some embodiments, R⁴ is hydrogen, —CH₃ or —CH₂CH₃. In some embodiments, R⁴ is hydrogen or —CH₃. In some embodiments, R⁴ is hydrogen.

In some embodiments, R⁴ is —CH₃.

In some embodiments of a compound of Formula (I), (Ia), or (Ib), R⁵ is hydrogen, fluoro, chloro, or bromo. In some embodiments, R⁵ is hydrogen, fluoro, or chloro. In some embodiments, R⁵ is hydrogen or fluoro. In some embodiments, R⁵ is hydrogen. In some embodiments, R⁵ is halogen. In some embodiments, R⁵ is fluoro, chloro, or bromo. In some embodiments, R⁵ is fluoro or chloro. In some embodiments, R⁵ is fluoro.

In some embodiments of a compound of Formula (I), (Ia), or (Ib), R⁶ is hydrogen, fluoro, chloro, or bromo. In some embodiments, R⁶ is hydrogen, fluoro, or chloro. In some embodiments, R⁶ is hydrogen or fluoro. In some embodiments, R⁶ is hydrogen. In some embodiments, R⁶ is halogen. In some embodiments, R⁶ is fluoro, chloro, or bromo. In some embodiments, R⁶ is fluoro or chloro. In some embodiments, R⁶ is fluoro.

In some embodiments of a compound of Formula (I), (Ia), or (Ib), R⁴ is hydrogen; R⁵ is hydrogen; and R⁶ is hydrogen or fluoro. In some embodiments, R⁴ is hydrogen; R⁵ is hydrogen; and R⁶ is hydrogen. In some embodiments, R⁴ is hydrogen; R⁵ is hydrogen; and R⁶ is fluoro.

In some embodiments of a compound of Formula (I), (Ia), or (Ib), each R⁷ and R⁸ is independently hydrogen, halogen, or unsubstituted C₁-C₄ alkyl. In some embodiments, each R⁷ and R⁸ is independently hydrogen, —F, —Cl, —Br, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), or —C(CH₃)₃. In some embodiments, each R⁷ and R⁸ is independently hydrogen, —F, —Cl, —CH₃, —CH₂CH₃, or —CH(CH₃)₂. In some embodiments, each R⁷ and R⁸ is independently hydrogen, fluoro, chloro, or —CH₃.

In some embodiments of a compound of Formula (I), (Ia), or (Ib), each R⁷ is independently hydrogen, halogen, or unsubstituted C₁-C₄ alkyl. In some embodiments, each R⁷ is independently hydrogen, —F, —Cl, —Br, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), or —C(CH₃)₃. In some embodiments, each R⁷ is independently hydrogen, —F, —Cl, —CH₃, —CH₂CH₃, or —CH(CH₃)₂. In some embodiments, each R⁷ is independently hydrogen, fluoro, chloro, or —CH₃.

In some embodiments of a compound of Formula (I), (Ia), or (Ib), each R⁸ is independently hydrogen, halogen, or unsubstituted C₁-C₄ alkyl. In some embodiments, each R⁸ is independently hydrogen, —F, —Cl, —Br, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), or —C(CH₃)₃. In some embodiments, each R⁸ is independently hydrogen, —F, —Cl, —CH₃, —CH₂CH₃, or —CH(CH₃)₂. In some embodiments, each R⁸ is independently hydrogen, fluoro, chloro, or —CH₃.

In some embodiments of a compound of Formula (I), (Ia), or (Ib), each R⁷ is independently hydrogen, —F, —Cl, —Br, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), or —C(CH₃)₃; and each R⁸ is hydrogen. In some embodiments, each R⁷ is independently hydrogen, —F, —Cl, —CH₃, —CH₂CH₃, or —CH(CH₃)₂; and each R⁸ is hydrogen. In some embodiments, each R⁷ is independently hydrogen, fluoro, chloro, or —CH₃; and each R⁸ is hydrogen.

In some embodiments of a compound of Formula (I), (Ia), or (Ib), each R⁷ is hydrogen; and each R⁸ is independently hydrogen, —F, —Cl, —Br, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), or —C(CH₃)₃. In some embodiments, each R⁷ is hydrogen; and each R⁸ is independently hydrogen, —F, —Cl, —CH₃, —CH₂CH₃, or —CH(CH₃)₂. In some embodiments, each R⁷ is hydrogen; and each R⁸ is independently hydrogen, fluoro, chloro, or —CH₃.

In some embodiments of a compound of Formula (I), (Ia), or (Ib), each R⁷ and R⁸ is hydrogen.

In some embodiments of a compound of Formula (I), (Ia), or (Ib), s is 1. In some embodiments, s is 2.

In some embodiments of a compound of Formula (I), (Ia), or (Ib), t is 1. In some embodiments, t is 2.

In some embodiments of a compound of Formula (I), (Ia), or (Ib), s is 1 or 2; and t is 1.

In some embodiments, s is 1 or 2; and t is 2. In some embodiments, s is 1 and t is 1. In some embodiments, s is 2 and t is 1.

In some embodiments of a compound of Formula (I), (Ia), or (Ib), t is 1 or 2; and s is 1.

In some embodiments, t is 1 or 2; and s is 2. In some embodiments, t is 1 and s is 1. In some embodiments, t is 2 and s is 1.

In some embodiments of a compound of Formula (I), (Ia), or (Ib), each R⁷ and R⁸ is independently hydrogen, fluoro, chloro, or —CH₃; s is 1; and t is 1.

In some embodiments, the compound of Formula (I) is a compound of Formula (II):

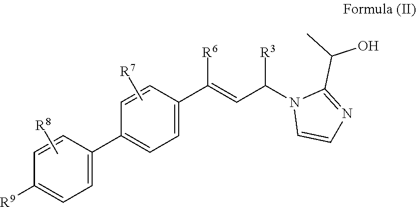

or a pharmaceutically acceptable salt, or solvate thereof.

In some embodiments, R⁶ is hydrogen or fluoro; R⁷ is hydrogen or fluoro; and R⁸ is hydrogen or fluoro.

In some embodiments, R³ is hydrogen or —(C₁-C₄ alkylene)-OH; R⁶ is hydrogen or fluoro; R⁷ is hydrogen or fluoro; R⁸ is hydrogen or fluoro; R⁹ is C₁-C₆ alkoxy, C₃-C₆ cycloalkyl, 4- to 6-membered heterocycloalkyl, —O—(C₃-C₆ cycloalkyl), —O—(4- to 6-membered heterocycloalkyl), —(C₁-C₄ alkylene)-(C₃-C₆ cycloalkyl), —(C₁-C₄ alkylene)-(4- to 6-membered heterocycloalkyl), —O—(C₁-C₄ alkylene)-(C₃-C₆ cycloalkyl), —O—(C₁-C₄ alkylene)-(4- to 6-membered heterocycloalkyl), —(C₁-C₄ alkylene)-O—(C₃-C₆ cycloalkyl), or —(C₁-C₄ alkylene)-O—(4- to 6-membered heterocycloalkyl); wherein the alkoxy, cycloalkyl, or heterocycloalkyl is unsubstituted or substituted by 1, 2, or 3 groups independently selected from —OR¹⁰, —N(R¹⁰)₂, —CO₂R¹⁰, —CON(R¹⁰)₂, —CH₂N(R¹⁰)₂, —NHCOR¹⁰, —NHSO₂R¹⁰, —OCOR¹⁰, phenyl, monocyclic heteroaryl, C₁-C₄ alkyl, C₁-C₄ hydroxyalkyl, C₁-C₄ aminoalkyl, C₃-C₆ cycloalkyl, and 4- to 6-membered heterocycloalkyl which is unsubstituted or substituted by 1, 2, or 3 —OH groups; and each R¹⁰ is independently hydrogen, 4- to 6-membered heterocycloalkyl, or C₁-C₄ alkyl which is unsubstituted or substituted by 1, 2, or 3 groups independently selected from —F, —CN, —OH, —NH₂, —OMe, —N(CH₃)₂, —CO₂H, —CONH₂, —SO₂CH₃, phenyl, oxazolidinone, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; or two R¹⁰ attached to the same nitrogen are taken together to form a 4- to 6-membered heterocycloalkyl which is unsubstituted or substituted by 1, 2, or 3 groups independently selected from —F, —CN, —OH, —NH₂, —OMe, —CO₂H, —CONH₂, and —SO₂CH₃.

In some embodiments, R³ is hydrogen or —(C₁-C₄ alkylene)-OH; R⁶ is hydrogen or fluoro; R⁷ is hydrogen or fluoro; R⁸ is hydrogen or fluoro; R⁹ is C₁-C₆ alkoxy, C₃-C₆ cycloalkyl, 4- to 6-membered heterocycloalkyl, —O—(C₃-C₆ cycloalkyl), —O—(4- to 6-membered heterocycloalkyl), —(C₁-C₄ alkylene)-(C₃-C₆ cycloalkyl), —(C₁-C₄ alkylene)-(4- to 6-membered heterocycloalkyl), —O—(C₁-C₄ alkylene)-(C₃-C₆ cycloalkyl), —O—(C₁-C₄ alkylene)-(4- to 6-membered heterocycloalkyl), —(C₁-C₄ alkylene)-O—(C₃-C₆ cycloalkyl), or —(C₁-C₄ alkylene)-O—(4- to 6-membered heterocycloalkyl); wherein the alkoxy, cycloalkyl, or heterocycloalkyl is unsubstituted or substituted by 1, 2, or 3 groups independently selected from —OR¹⁰, —N(R¹⁰)₂—CO₂R¹⁰, —CON(R¹⁰)₂, —CH₂N(R¹⁰)₂, —NHCOR¹⁰, —NHSO₂R¹⁰, phenyl, monocyclic heteroaryl, C₁-C₄ alkyl, C₁-C₄ hydroxyalkyl, C₁-C₄ aminoalkyl, C₃-C₆ cycloalkyl, and 4- to 6-membered heterocycloalkyl which is unsubstituted or substituted by 1, 2, or 3 —OH groups; and each R¹⁰ is independently hydrogen, 4- to 6-membered heterocycloalkyl, or C₁-C₄ alkyl which is unsubstituted or substituted by 1, 2, or 3 groups independently selected from —F, —CN, —OH, —NH₂, —OMe, —N(CH₃)₂, —CO₂H, —CONH₂, —SO₂CH₃, phenyl, oxazolidinone, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; or two R¹⁰ attached to the same nitrogen are taken together to form a 4- to 6-membered heterocycloalkyl which is unsubstituted or substituted by 1, 2, or 3 groups independently selected from —F, —CN, —OH, —NH₂, —OMe, —CO₂H, —CONH₂, and —SO₂CH₃.

In some embodiments, the compound of Formula (I) or (II) is a compound of Formula (IIa) or Formula (IIb):

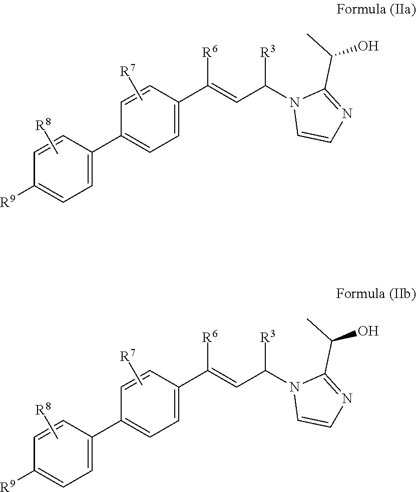

or a pharmaceutically acceptable salt, or solvate thereof.

In some embodiments, R⁶ is hydrogen or fluoro; R⁷ is hydrogen or fluoro; and R⁸ is hydrogen or fluoro.

In some embodiments, R³ is hydrogen or —(C₁-C₄ alkylene)-OH; R⁶ is hydrogen or fluoro; R⁷ is hydrogen or fluoro; R⁸ is hydrogen or fluoro; R⁹ is C₁-C₆ alkoxy, C₃-C₆ cycloalkyl, 4- to 6-membered heterocycloalkyl, —O—(C₃-C₆ cycloalkyl), —O—(4- to 6-membered heterocycloalkyl), —(C₁-C₄ alkylene)-(C₃-C₆ cycloalkyl), —(C₁-C₄ alkylene)-(4- to 6-membered heterocycloalkyl), —O—(C₁-C₄ alkylene)-(C₃-C₆ cycloalkyl), —O—(C₁-C₄ alkylene)-(4- to 6-membered heterocycloalkyl), —(C₁-C₄ alkylene)-O—(C₃-C₆ cycloalkyl), or —(C₁-C₄ alkylene)-O—(4- to 6-membered heterocycloalkyl); wherein the alkoxy, cycloalkyl, or heterocycloalkyl is unsubstituted or substituted by 1, 2, or 3 groups independently selected from —OR¹⁰, —N(R¹⁰)₂, —CO₂R¹⁰, —CON(R¹⁰)₂, —CH₂N(R¹⁰)₂, —NHCOR¹⁰, —NHSO₂R¹⁰, —OCOR¹⁰, phenyl, monocyclic heteroaryl, C₁-C₄ alkyl, C₁-C₄ hydroxyalkyl, C₁-C₄ aminoalkyl, C₃-C₆ cycloalkyl, and 4- to 6-membered heterocycloalkyl which is unsubstituted or substituted by 1, 2, or 3 —OH groups; and each R¹⁰ is independently hydrogen, 4- to 6-membered heterocycloalkyl, or C₁-C₄ alkyl which is unsubstituted or substituted by 1, 2, or 3 groups independently selected from —F, —CN, —OH, —NH₂, —OMe, —N(CH₃)₂, —CO₂H, —CONH₂, —SO₂CH₃, phenyl, oxazolidinone, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; or two R¹⁰ attached to the same nitrogen are taken together to form a 4- to 6-membered heterocycloalkyl which is unsubstituted or substituted by 1, 2, or 3 groups independently selected from —F, —CN, —OH, —NH₂, —OMe, —CO₂H, —CONH₂, and —SO₂CH₃.

In some embodiments, R³ is hydrogen or —(C₁-C₄ alkylene)-OH; R⁶ is hydrogen or fluoro; R⁷ is hydrogen or fluoro; R⁸ is hydrogen or fluoro; R⁹ is C₁-C₆ alkoxy, C₃-C₆ cycloalkyl, 4- to 6-membered heterocycloalkyl, —O—(C₃-C₆ cycloalkyl), —O—(4- to 6-membered heterocycloalkyl), —(C₁-C₄ alkylene)-(C₃-C₆ cycloalkyl), —(C₁-C₄ alkylene)-(4- to 6-membered heterocycloalkyl), —O—(C₁-C₄ alkylene)-(C₃-C₆ cycloalkyl), —O—(C₁-C₄ alkylene)-(4- to 6-membered heterocycloalkyl), —(C₁-C₄ alkylene)-O—(C₃-C₆ cycloalkyl), or —(C₁-C₄ alkylene)-O—(4- to 6-membered heterocycloalkyl); wherein the alkoxy, cycloalkyl, or heterocycloalkyl is unsubstituted or substituted by 1, 2, or 3 groups independently selected from —OR¹⁰, —N(R¹⁰)₂, —CO₂R¹⁰, —CON(R¹⁰)₂, —CH₂N(R¹⁰)₂, —NHCOR¹⁰, —NHSO₂R¹⁰, phenyl, monocyclic heteroaryl, C₁-C₄ alkyl, C₁-C₄ hydroxyalkyl, C₁-C₄ aminoalkyl, C₃-C₆ cycloalkyl, and 4- to 6-membered heterocycloalkyl which is unsubstituted or substituted by 1, 2, or 3 —OH groups; and each R¹⁰ is independently hydrogen, 4- to 6-membered heterocycloalkyl, or C₁-C₄ alkyl which is unsubstituted or substituted by 1, 2, or 3 groups independently selected from —F, —CN, —OH, —NH₂, —OMe, —N(CH₃)₂, —CO₂H, —CONH₂, —SO₂CH₃, phenyl, oxazolidinone, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; or two R¹⁰ attached to the same nitrogen are taken together to form a 4- to 6-membered heterocycloalkyl which is unsubstituted or substituted by 1, 2, or 3 groups independently selected from —F, —CN, —OH, —NH₂, —OMe, —CO₂H, —CONH₂, and —SO₂CH₃.

In some embodiments, the compound is a compound of Formula (IIa), or a pharmaceutically acceptable salt, or solvate thereof. In some embodiments, the compound is a compound of Formula (IIb), or a pharmaceutically acceptable salt, or solvate thereof.

In some embodiments of a compound of Formula (II), (IIa), or (IIb), R⁶ is hydrogen. In some embodiments, R⁶ is fluoro.

In some embodiments of a compound of Formula (II), (IIa), or (IIb), R⁷ is hydrogen. In some embodiments, R⁷ is fluoro.

In some embodiments of a compound of Formula (II), (IIa), or (IIb), R⁸ is hydrogen. In some embodiments, R⁸ is fluoro.

In some embodiments of a compound of Formula (II), (IIa), or (IIb), R⁶ is hydrogen; R⁷ is hydrogen; and R⁸ is hydrogen. In some embodiments, R⁶ is fluoro; R⁷ is hydrogen; and R⁸ is hydrogen. In some embodiments, R⁶ is hydrogen; R⁷ is fluoro; and R⁸ is hydrogen. In some embodiments, R⁶ is hydrogen; R⁷ is hydrogen; and R⁸ is fluoro. In some embodiments, R⁶ is fluoro; R⁷ is fluoro; and R⁸ is hydrogen. In some embodiments, R⁶ is fluoro; R⁷ is hydrogen; and R⁸ is fluoro. In some embodiments, R⁶ is hydrogen; R⁷ is fluoro; and R⁸ is fluoro. In some embodiments, R⁶ is fluoro; R⁷ is fluoro; and R⁸ is fluoro.

In some embodiments of a compound of Formula (I), (Ia), (Ib), (II), (IIa), or (IIb), R³ is hydrogen or —(C₁-C₂ alkylene)-OH. In some embodiments, R³ is hydrogen, —CH₂OH, —CH₂CH₂OH, —CH₂CH₂CH₂OH, —CH₂CH₂CH₂CH₂OH, —CH(CH₃)OH, —CH₂CH(CH₃)OH, —CH(CH₃)CH₂OH, —CH₂CH(CH₂CH₃)OH, or —CH(CH₂CH₃)CH₂OH. In some embodiments, R³ is hydrogen, —CH₂OH, —CH₂CH₂OH, or —CH(CH₃)OH. In some embodiments, R³ is hydrogen, —CH₂OH or —CH₂CH₂OH. In some embodiments, R³ is hydrogen or —CH₂OH.

In some embodiments of a compound of Formula (I), (Ia), (Ib), (II), (IIa), or (IIb), R³ is hydrogen.

In some embodiments of a compound of Formula (I), (Ia), (Ib), (II), (IIa), or (IIb), R³ is —(C₁-C₄ alkylene)-OH. In some embodiments, R³ is —(C₁-C₂ alkylene)-OH. In some embodiments, R³ is —CH₂OH, —CH₂CH₂OH, —CH₂CH₂CH₂OH, —CH₂CH₂CH₂CH₂OH, —CH(CH₃)OH, —CH₂CH(CH₃)OH, —CH(CH₃)CH₂OH, —CH₂CH(CH₂CH₃)OH, or —CH(CH₂CH₃)CH₂OH. In some embodiments, R³ is —CH₂OH, —CH₂CH₂OH, or —CH(CH₃)OH. In some embodiments, R³ is —CH₂OH or —CH₂CH₂OH. In some embodiments, R³ is —CH₂OH. In some embodiments, R³ is —CH₂CH₂OH.

In some embodiments of a compound of Formula (I) or (II), the compound is a compound of Formula (IIIa), Formula (IIIb), Formula (IIIc), or Formula (IIId):

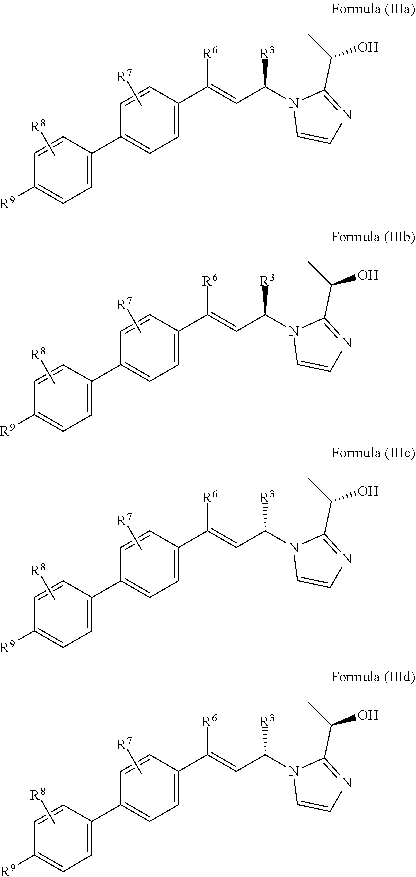

or a pharmaceutically acceptable salt, or solvate thereof.

In some embodiments, R³ is —(C₁-C₄ alkylene)-OH; R⁶ is hydrogen or fluoro; R⁷ is hydrogen or fluoro; and R⁸ is hydrogen or fluoro.

In some embodiments, R⁶ is hydrogen or fluoro; R⁷ is hydrogen or fluoro; R⁸ is hydrogen or fluoro; R³ is —(C₁-C₄ alkylene)-OH; R⁹ is C₁-C₆ alkoxy, C₃-C₆ cycloalkyl, 4- to 6-membered heterocycloalkyl, —O—(C₃-C₆ cycloalkyl), —O—(4- to 6-membered heterocycloalkyl), —(C₁-C₄ alkylene)-(C₃-C₆ cycloalkyl), —(C₁-C₄ alkylene)-(4- to 6-membered heterocycloalkyl), —O—(C₁-C₄ alkylene)-(C₃-C₆ cycloalkyl), —O—(C₁-C₄ alkylene)-(4- to 6-membered heterocycloalkyl), —(C₁-C₄ alkylene)-O—(C₃-C₆ cycloalkyl), or —(C₁-C₄ alkylene)-O—(4- to 6-membered heterocycloalkyl); wherein the alkoxy, cycloalkyl, or heterocycloalkyl is unsubstituted or substituted by 1, 2, or 3 groups independently selected from —OR¹⁰, —N(R¹⁰)₂, —CO₂R¹⁰, —CON(R¹⁰)₂, —CH₂N(R¹⁰)₂, —NHCOR¹⁰, —NHSO₂R¹⁰, —OCOR¹⁰, phenyl, monocyclic heteroaryl, C₁-C₄ alkyl, C₁-C₄ hydroxyalkyl, C₁-C₄ aminoalkyl, C₃-C₆ cycloalkyl, and 4- to 6-membered heterocycloalkyl which is unsubstituted or substituted by 1, 2, or 3 —OH groups; and each R¹⁰ is independently hydrogen, 4- to 6-membered heterocycloalkyl, or C₁-C₄ alkyl which is unsubstituted or substituted by 1, 2, or 3 groups independently selected from —F, —CN, —OH, —NH₂, —OMe, —N(CH₃)₂, —CO₂H, —CONH₂, —SO₂CH₃, phenyl, oxazolidinone, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; or two R¹⁰ attached to the same nitrogen are taken together to form a 4- to 6-membered heterocycloalkyl which is unsubstituted or substituted by 1, 2, or 3 groups independently selected from —F, —CN, —OH, —NH₂, —OMe, —CO₂H, —CONH₂, and —SO₂CH₃.

In some embodiments, R⁶ is hydrogen or fluoro; R⁷ is hydrogen or fluoro; R⁸ is hydrogen or fluoro; R³ is —(C₁-C₄ alkylene)-OH; R⁹ is C₁-C₆ alkoxy, C₃-C₆ cycloalkyl, 4- to 6-membered heterocycloalkyl, —O—(C₃-C₆ cycloalkyl), —O—(4- to 6-membered heterocycloalkyl), —(C₁-C₄ alkylene)-(C₃-C₆ cycloalkyl), —(C₁-C₄ alkylene)-(4- to 6-membered heterocycloalkyl), —O—(C₁-C₄ alkylene)-(C₃-C₆ cycloalkyl), —O—(C₁-C₄ alkylene)-(4- to 6-membered heterocycloalkyl), —(C₁-C₄ alkylene)-O—(C₃-C₆ cycloalkyl), or —(C₁-C₄ alkylene)-O—(4- to 6-membered heterocycloalkyl); wherein the alkoxy, cycloalkyl, or heterocycloalkyl is unsubstituted or substituted by 1, 2, or 3 groups independently selected from —OR¹⁰, —N(R¹⁰)₂, —CO₂R¹⁰, —CON(R¹⁰)₂, —CH₂N(R¹⁰)₂, —NHCOR¹⁰, —NHSO₂R¹⁰, phenyl, monocyclic heteroaryl, C₁-C₄ alkyl, C₁-C₄ hydroxyalkyl, C₁-C₄ aminoalkyl, C₃-C₆ cycloalkyl, and 4- to 6-membered heterocycloalkyl which is unsubstituted or substituted by 1, 2, or 3 —OH groups; and each R¹⁰ is independently hydrogen, 4- to 6-membered heterocycloalkyl, or C₁-C₄ alkyl which is unsubstituted or substituted by 1, 2, or 3 groups independently selected from —F, —CN, —OH, —NH₂, —OMe, —N(CH₃)₂, —CO₂H, —CONH₂, —SO₂CH₃, phenyl, oxazolidinone, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; or two R¹⁰ attached to the same nitrogen are taken together to form a 4- to 6-membered heterocycloalkyl which is unsubstituted or substituted by 1, 2, or 3 groups independently selected from —F, —CN, —OH, —NH₂, —OMe, —CO₂H, —CONH₂, and —SO₂CH₃.

In some embodiments, the compound is a compound of Formula (IIIa), or a pharmaceutically acceptable salt, or solvate thereof. In some embodiments, the compound is a compound of Formula (IIIb), or a pharmaceutically acceptable salt, or solvate thereof. In some embodiments, the compound is a compound of Formula (IIIc), or a pharmaceutically acceptable salt, or solvate thereof. In some embodiments, the compound is a compound of Formula (IIId), or a pharmaceutically acceptable salt, or solvate thereof.

In some embodiments of a compound of Formula (IIIa), (IIIb), (IIIc), or (IIId), R⁶ is hydrogen. In some embodiments, R⁶ is fluoro.

In some embodiments of a compound of Formula (IIIa), (IIIb), (IIIc), or (IIId), R⁷ is hydrogen. In some embodiments, R⁷ is fluoro.

In some embodiments of a compound of Formula (IIIa), (IIIb), (IIIc), or (IIId), R⁸ is hydrogen. In some embodiments, R⁸ is fluoro.

In some embodiments of a compound of Formula (IIIa), (IIIb), (IIIc), or (IIId), R⁶ is hydrogen; R⁷ is hydrogen; and R⁸ is hydrogen. In some embodiments, R⁶ is fluoro; R⁷ is hydrogen; and R⁸ is hydrogen. In some embodiments, R⁶ is hydrogen; R⁷ is fluoro; and R⁸ is hydrogen. In some embodiments, R⁶ is hydrogen; R⁷ is hydrogen; and R⁸ is fluoro. In some embodiments, R⁶ is fluoro; R⁷ is fluoro; and R⁸ is hydrogen. In some embodiments, R⁶ is fluoro; R⁷ is hydrogen; and R⁸ is fluoro. In some embodiments, R⁶ is hydrogen; R⁷ is fluoro; and R⁸ is fluoro. In some embodiments, R⁶ is fluoro; R⁷ is fluoro; and R⁸ is fluoro.

In some embodiments of a compound of Formula (IIIa), (IIIb), (IIIc), or (IIId), R³ is —(C₁-C₂ alkylene)-OH. In some embodiments, R³ is —CH₂OH, —CH₂CH₂OH, —CH₂CH₂CH₂OH, —CH₂CH₂CH₂CH₂OH, —CH(CH₃)OH, —CH₂CH(CH₃)OH, —CH(CH₃)CH₂OH, —CH₂CH(CH₂CH₃)OH, or —CH(CH₂CH₃)CH₂OH. In some embodiments, R³ is —CH₂OH, —CH₂CH₂OH, or —CH(CH₃)OH. In some embodiments, R³ is —CH₂OH or —CH₂CH₂OH. In some embodiments, R³ is —CH₂OH. In some embodiments, R³ is —CH₂CH₂OH.

In some embodiments, the compound of Formula (I) or (II) is a compound of Formula (IV):

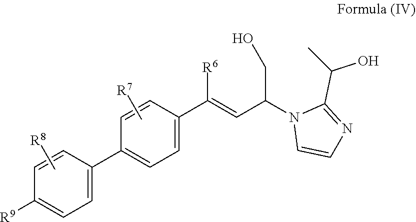

or a pharmaceutically acceptable salt, or solvate thereof.

In some embodiments, R⁶ is hydrogen or fluoro; R⁷ is hydrogen or fluoro; and R⁸ is hydrogen or fluoro.

In some embodiments, R⁶ is hydrogen or fluoro; R⁷ is hydrogen or fluoro; R⁸ is hydrogen or fluoro; R⁹ is C₁-C₆ alkoxy, C₃-C₆ cycloalkyl, 4- to 6-membered heterocycloalkyl, —O—(C₃-C₆ cycloalkyl), —O—(4- to 6-membered heterocycloalkyl), —(C₁-C₄ alkylene)-(C₃-C₆ cycloalkyl), —(C₁-C₄ alkylene)-(4- to 6-membered heterocycloalkyl), —O—(C₁-C₄ alkylene)-(C₃-C₆ cycloalkyl), —O—(C₁-C₄ alkylene)-(4- to 6-membered heterocycloalkyl), —(C₁-C₄ alkylene)-O—(C₃-C₆ cycloalkyl), or —(C₁-C₄ alkylene)-O—(4- to 6-membered heterocycloalkyl); wherein the alkoxy, cycloalkyl, or heterocycloalkyl is unsubstituted or substituted by 1, 2, or 3 groups independently selected from —OR¹⁰, —N(R¹⁰)₂, —CO₂R¹⁰, —CON(R¹⁰)₂, —CH₂N(R¹⁰)₂, —NHCOR¹⁰, —NHSO₂R¹⁰, —OCOR¹⁰, phenyl, monocyclic heteroaryl, C₁-C₄ alkyl, C₁-C₄ hydroxyalkyl, C₁-C₄ aminoalkyl, C₃-C₆ cycloalkyl, and 4- to 6-membered heterocycloalkyl which is unsubstituted or substituted by 1, 2, or 3 —OH groups; and each R¹⁰ is independently hydrogen, 4- to 6-membered heterocycloalkyl, or C₁-C₄ alkyl which is unsubstituted or substituted by 1, 2, or 3 groups independently selected from —CN, —OH, —NH₂, —OMe, —N(CH₃)₂, —CO₂H, —CONH₂, —SO₂CH₃, phenyl, oxazolidinone, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; or two R¹⁰ attached to the same nitrogen are taken together to form a 4- to 6-membered heterocycloalkyl which is unsubstituted or substituted by 1, 2, or 3 groups independently selected from —F, —CN, —OH, —NH₂, —OMe, —CO₂H, —CONH₂, and —SO₂CH₃.

In some embodiments, R⁶ is hydrogen or fluoro; R⁷ is hydrogen or fluoro; R⁸ is hydrogen or fluoro; R⁹ is C₁-C₆ alkoxy, C₃-C₆ cycloalkyl, 4- to 6-membered heterocycloalkyl, —O—(C₃-C₆ cycloalkyl), —O—(4- to 6-membered heterocycloalkyl), —(C₁-C₄ alkylene)-(C₃-C₆ cycloalkyl), —(C₁-C₄ alkylene)-(4- to 6-membered heterocycloalkyl), —O—(C₁-C₄ alkylene)-(C₃-C₆ cycloalkyl), —O—(C₁-C₄ alkylene)-(4- to 6-membered heterocycloalkyl), —(C₁-C₄ alkylene)-O—(C₃-C₆ cycloalkyl), or —(C₁-C₄ alkylene)-O—(4- to 6-membered heterocycloalkyl); wherein the alkoxy, cycloalkyl, or heterocycloalkyl is unsubstituted or substituted by 1, 2, or 3 groups independently selected from —OR¹⁰, —N(R¹⁰)₂, —CO₂R¹⁰, —CON(R¹⁰)₂, —CH₂N(R¹⁰)₂, —NHCOR¹⁰, —NHSO₂R¹⁰, phenyl, monocyclic heteroaryl, C₁-C₄ alkyl, C₁-C₄ hydroxyalkyl, C₁-C₄ aminoalkyl, C₃-C₆ cycloalkyl, and 4- to 6-membered heterocycloalkyl which is unsubstituted or substituted by 1, 2, or 3 —OH groups; and each R¹⁰ is independently hydrogen, 4- to 6-membered heterocycloalkyl, or C₁-C₄ alkyl which is unsubstituted or substituted by 1, 2, or 3 groups independently selected from —CN, —OH, —NH₂, —OMe, —N(CH₃)₂, —CO₂H, —CONH₂, —SO₂CH₃, phenyl, oxazolidinone, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; or two R¹⁰ attached to the same nitrogen are taken together to form a 4- to 6-membered heterocycloalkyl which is unsubstituted or substituted by 1, 2, or 3 groups independently selected from —F, —CN, —OH, —NH₂, —OMe, —CO₂H, —CONH₂, and —SO₂CH₃.

In some embodiments, the compound of a compound of Formula (I) or (II) is a compound of Formula (IVa), Formula (IVb), Formula (IVc), or Formula (IVd):

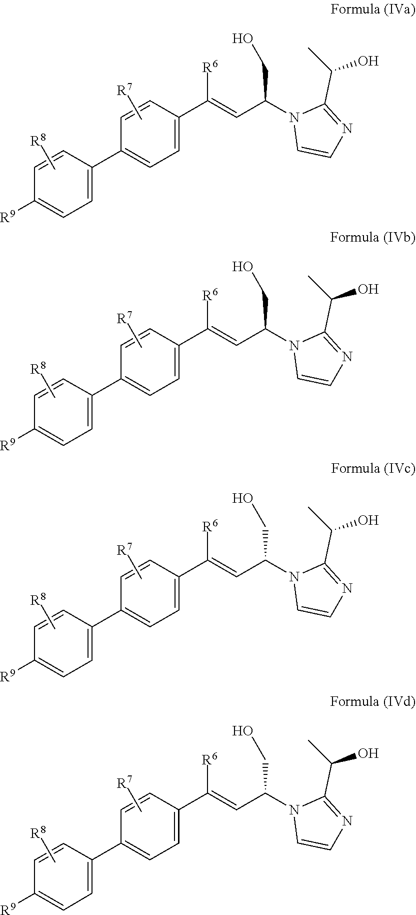

or a pharmaceutically acceptable salt, or solvate thereof.

In some embodiments, R⁶ is hydrogen or fluoro; R⁷ is hydrogen or fluoro; and R⁸ is hydrogen or fluoro.

In some embodiments, R⁶ is hydrogen or fluoro; R⁷ is hydrogen or fluoro; R⁸ is hydrogen or fluoro; R⁹ is C₁-C₆ alkoxy, C₃-C₆ cycloalkyl, 4- to 6-membered heterocycloalkyl, —O—(C₃-C₆ cycloalkyl), —O—(4- to 6-membered heterocycloalkyl), —(C₁-C₄ alkylene)-(C₃-C₆ cycloalkyl), —(C₁-C₄ alkylene)-(4- to 6-membered heterocycloalkyl), —O—(C₁-C₄ alkylene)-(C₃-C₆ cycloalkyl), —O—(C₁-C₄ alkylene)-(4- to 6-membered heterocycloalkyl), —(C₁-C₄ alkylene)-O—(C₃-C₆ cycloalkyl), or —(C₁-C₄ alkylene)-O—(4- to 6-membered heterocycloalkyl); wherein the alkoxy, cycloalkyl, or heterocycloalkyl is unsubstituted or substituted by 1, 2, or 3 groups independently selected from —OR¹⁰, —N(R¹⁰)₂, —CO₂R¹⁰, —CON(R¹⁰)₂, —CH₂N(R¹⁰)₂, —NHCOR¹⁰, —NHSO₂R¹⁰, —OCOR¹⁰, phenyl, monocyclic heteroaryl, C₁-C₄ alkyl, C₁-C₄ hydroxyalkyl, C₁-C₄ aminoalkyl, C₃-C₆ cycloalkyl, and 4- to 6-membered heterocycloalkyl which is unsubstituted or substituted by 1, 2, or 3 —OH groups; and each R¹⁰ is independently hydrogen, 4- to 6-membered heterocycloalkyl, or C₁-C₄ alkyl which is unsubstituted or substituted by 1, 2, or 3 groups independently selected from —F, —CN, —OH, —NH₂, —OMe, —N(CH₃)₂, —CO₂H, —CONH₂, —SO₂CH₃, phenyl, oxazolidinone, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; or two R¹⁰ attached to the same nitrogen are taken together to form a 4- to 6-membered heterocycloalkyl which is unsubstituted or substituted by 1, 2, or 3 groups independently selected from —F, —CN, —OH, —NH₂, —OMe, —CO₂H, —CONH₂, and —SO₂CH₃.

In some embodiments, R⁶ is hydrogen or fluoro; R⁷ is hydrogen or fluoro; R⁸ is hydrogen or fluoro; R⁹ is C₁-C₆ alkoxy, C₃-C₆ cycloalkyl, 4- to 6-membered heterocycloalkyl, —O—(C₃-C₆ cycloalkyl), —O—(4- to 6-membered heterocycloalkyl), —(C₁-C₄ alkylene)-(C₃-C₆ cycloalkyl), —(C₁-C₄ alkylene)-(4- to 6-membered heterocycloalkyl), —O—(C₁-C₄ alkylene)-(C₃-C₆ cycloalkyl), —O—(C₁-C₄ alkylene)-(4- to 6-membered heterocycloalkyl), —(C₁-C₄ alkylene)-O—(C₃-C₆ cycloalkyl), or —(C₁-C₄ alkylene)-O—(4- to 6-membered heterocycloalkyl); wherein the alkoxy, cycloalkyl, or heterocycloalkyl is unsubstituted or substituted by 1, 2, or 3 groups independently selected from —OR¹⁰, —N(R¹⁰)₂, —CO₂R¹⁰, —CON(R¹⁰)₂, —CH₂N(R¹⁰)₂, —NHCOR¹⁰, —NHSO₂R¹⁰, phenyl, monocyclic heteroaryl, C₁-C₄ alkyl, C₁-C₄ hydroxyalkyl, C₁-C₄ aminoalkyl, C₃-C₆ cycloalkyl, and 4- to 6-membered heterocycloalkyl which is unsubstituted or substituted by 1, 2, or 3 —OH groups; and each R¹⁰ is independently hydrogen, 4- to 6-membered heterocycloalkyl, or C₁-C₄ alkyl which is unsubstituted or substituted by 1, 2, or 3 groups independently selected from —F, —CN, —OH, —NH₂, —OMe, —N(CH₃)₂, —CO₂H, —CONH₂, —SO₂CH₃, phenyl, oxazolidinone, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; or two R¹⁰ attached to the same nitrogen are taken together to form a 4- to 6-membered heterocycloalkyl which is unsubstituted or substituted by 1, 2, or 3 groups independently selected from —F, —CN, —OH, —NH₂, —OMe, —CO₂H, —CONH₂, and —SO₂CH₃.

In some embodiments, the compound is a compound of Formula (IVa), or a pharmaceutically acceptable salt, or solvate thereof. In some embodiments, the compound is a compound of Formula (IVb), or a pharmaceutically acceptable salt, or solvate thereof. In some embodiments, the compound is a compound of Formula (IVc), or a pharmaceutically acceptable salt, or solvate thereof. In some embodiments, the compound is a compound of Formula (IVd), or a pharmaceutically acceptable salt, or solvate thereof.

In some embodiments of a compound of Formula (IVa), (IVb), (IVc), or (IVd), R⁶ is hydrogen. In some embodiments, R⁶ is fluoro.

In some embodiments of a compound of Formula (IVa), (IVb), (IVc), or (IVd), R⁷ is hydrogen. In some embodiments, R⁷ is fluoro.

In some embodiments of a compound of Formula (IVa), (IVb), (IVc), or (IVd), R⁸ is hydrogen. In some embodiments, R⁸ is fluoro.

In some embodiments of a compound of Formula (IVa), (IVb), (IVc), or (IVd), R⁶ is hydrogen; R⁷ is hydrogen; and R⁸ is hydrogen. In some embodiments, R⁶ is fluoro; R⁷ is hydrogen; and R⁸ is hydrogen. In some embodiments, R⁶ is hydrogen; R⁷ is fluoro; and R⁸ is hydrogen. In some embodiments, R⁶ is hydrogen; R⁷ is hydrogen; and R⁸ is fluoro. In some embodiments, R⁶ is fluoro; R⁷ is fluoro; and R⁸ is hydrogen. In some embodiments, R⁶ is fluoro; R⁷ is hydrogen; and R⁸ is fluoro. In some embodiments, R⁶ is hydrogen; R⁷ is fluoro; and R⁸ is fluoro. In some embodiments, R⁶ is fluoro; R⁷ is fluoro; and R⁸ is fluoro.

In some embodiments of a compound of Formula (I), (Ia), (Ib), (II), (IIa), (IIb), (IIIa), (IIIb), (IIIc), (IIId), (IV), (IVa), (IVb), (IVc), or (IVd), R⁹ is C₁-C₆ alkoxy, C₃-C₆ cycloalkyl, —O—(C₃-C₆ cycloalkyl), —O—(4- to 6-membered heterocycloalkyl), —O—(C₁-C₄ alkylene)-(C₃-C₆ cycloalkyl), —O—(C₁-C₄ alkylene)-(4- to 6-membered heterocycloalkyl), or —(C₁-C₄ alkylene)-O—(C₃-C₆ cycloalkyl). In some embodiments, R⁹ is C₁-C₆ alkoxy, C₃-C₆ cycloalkyl, —O—(C₃-C₆ cycloalkyl), —O—(4- to 6-membered heterocycloalkyl), —O—(C₁-C₄ alkylene)-(C₃-C₆ cycloalkyl), —O—(C₁-C₄ alkylene)-(4- to 6-membered heterocycloalkyl), or —(C₁-C₄ alkylene)-O—(C₃-C₆ cycloalkyl); wherein the alkoxy, cycloalkyl, or heterocycloalkyl is unsubstituted or substituted by 1 or 2 groups independently selected from —OR¹⁰, —N(R¹⁰)₂, —CO₂R¹⁰, —CON(R¹⁰)₂, —CH₂N(R¹⁰)₂, —NHCOR¹⁰, —NHSO₂R¹⁰, C₁-C₄ hydroxyalkyl, C₁-C₄ aminoalkyl, and 4- to 6-membered heterocycloalkyl which is unsubstituted or substituted by 1 —OH group. In some embodiments, R⁹ is C₁-C₆ alkoxy, C₃-C₆ cycloalkyl, —O—(C₃-C₆ cycloalkyl), —O—(4- to 6-membered heterocycloalkyl), —O—(C₁-C₄ alkylene)-(C₃-C₆ cycloalkyl), —O—(C₁-C₄ alkylene)-(4- to 6-membered heterocycloalkyl), or —(C₁-C₄ alkylene)-O—(C₃-C₆ cycloalkyl); wherein the alkoxy, cycloalkyl, or heterocycloalkyl is unsubstituted or substituted by 1 or 2 groups independently selected from —OR¹⁰, —N(R¹⁰)₂, —CO₂R¹⁰, —CON(R¹⁰)₂, —CH₂N(R¹⁰)₂, —NHCOR¹⁰, —NHSO₂R¹⁰—OCOR¹⁰, C₁-C₄ hydroxyalkyl, C₁-C₄ aminoalkyl, and 4- to 6-membered heterocycloalkyl which is unsubstituted or substituted by 1 —OH group. some embodiments, R⁹ is C₁-C₆ alkoxy, C₃-C₆ cycloalkyl, —O—(C₃-C₆ cycloalkyl), —O—(4- to 6-membered heterocycloalkyl), —O—(C₁-C₄ alkylene)-(C₃-C₆ cycloalkyl), —O—(C₁-C₄ alkylene)-(4- to 6-membered heterocycloalkyl), or —(C₁-C₄ alkylene)-O—(C₃-C₆ cycloalkyl); wherein the alkoxy, cycloalkyl, or heterocycloalkyl is unsubstituted or substituted by 1 or 2 groups independently selected from —OR¹⁰, —N(R¹⁰)₂—CO₂R¹⁰, —CON(R¹⁰)₂, —CH₂N(R¹⁰)₂, —NHCOR¹⁰, —NHSO₂R¹⁰, C₁-C₄ hydroxyalkyl, C₁-C₄ aminoalkyl, and 4- to 6-membered heterocycloalkyl which is unsubstituted or substituted by 1 —OH group. In some embodiments, R⁹ is C₁-C₄ alkoxy, C₃-C₄ cycloalkyl, —O—(C₃-C₄ cycloalkyl), —O-(4- to 6-membered heterocycloalkyl), —O—(C₁-C₃ alkylene)-(C₃-C₄ cycloalkyl), —O—(C₁-C₃ alkylene)-(4- to 6-membered heterocycloalkyl), or —(C₁-C₃ alkylene)-O—(C₃-C₄ cycloalkyl); wherein the alkoxy, cycloalkyl, or heterocycloalkyl is substituted by 1 or 2 groups independently selected from —OR¹⁰, —N(R¹⁰)₂, —CO₂R¹⁰, —CON(R¹⁰)₂, —CH₂N(R¹⁰)₂, —NHCOR¹⁰, —NHSO₂R¹⁰—OCOR¹⁰, C₁-C₃ hydroxyalkyl, C₁-C₃ aminoalkyl, and 4- to 6-membered heterocycloalkyl which is unsubstituted or substituted by 1 —OH group. In some embodiments, R⁹ is C₁-C₄ alkoxy, C₃-C₄ cycloalkyl, —O—(C₃-C₄ cycloalkyl), —O—(4- to 6-membered heterocycloalkyl), —O—(C₁-C₃ alkylene)-(C₃-C₄ cycloalkyl), —O—(C₁-C₃ alkylene)-(4- to 6-membered heterocycloalkyl), or —(C₁-C₃ alkylene)-O—(C₃-C₄ cycloalkyl); wherein the alkoxy, cycloalkyl, or heterocycloalkyl is substituted by 1 or 2 groups independently selected from —OR¹⁰, —N(R¹⁰)₂, —CO₂R¹⁰, —CON(R¹⁰)₂, —CH₂N(R¹⁰)₂, —NHCOR¹⁰, —NHSO₂R¹⁰, C₁-C₃ hydroxyalkyl, C₁-C₃ aminoalkyl, and 4- to 6-membered heterocycloalkyl which is unsubstituted or substituted by 1 —OH group. In some embodiments, R⁹ is C₁-C₄ alkoxy, C₃-C₄ cycloalkyl, —O—(C₃-C₄ cycloalkyl), or —O—(4- to 6-membered heterocycloalkyl); wherein the alkoxy, cycloalkyl, or heterocycloalkyl is substituted by 1 or 2 groups independently selected from —OH, —N(R¹⁰)₂, —CON(R¹⁰)₂, —NHCOR¹⁰, —NHSO₂R¹⁰, —CH₂OH, —CH₂CH₂OH, and azetidinyl which is substituted by 1 —OH group. In some embodiments, R⁹ is C₁-C₄ alkoxy, C₃-C₄ cycloalkyl, —O—(C₃-C₄ cycloalkyl), or —O—(4- to 6-membered heterocycloalkyl); wherein the alkoxy, cycloalkyl, or heterocycloalkyl is substituted by 1 or 2 groups independently selected from —OH, —N(R¹⁰)₂, —CON(R¹⁰)₂, —NHCOR¹⁰, —NHSO₂R¹⁰, —CH₂CH₂OH, and azetidinyl which is substituted by 1 —OH group.

In some embodiments of a compound of Formula (I), (Ia), (Ib), (II), (IIa), (IIb), (IIIa), (IIIb), (IIIc), (IIId), (IV), (IVa), (IVb), (IVc), or (IVd), each R¹⁰ is independently hydrogen, 4- to 6-membered heterocycloalkyl, or C₁-C₄ alkyl which is unsubstituted or substituted by 1, 2, or 3 groups independently selected from —F, —CN, —OH, —N(CH₃)₂, —CO₂H, —SO₂CH₃, oxazolidinone, and monocyclic heteroaryl which is unsubstituted or substituted by 1 —CONH₂ group; or two R¹⁰ attached to the same nitrogen are taken together to form an azetidinyl which is unsubstituted or substituted by 1 —SO₂CH₃ group. In some embodiments, each R¹⁰ is independently hydrogen or C₁-C₂ alkyl which is unsubstituted or substituted by 1, 2, or 3 groups independently selected from —F —CN, —OH, —N(CH₃)₂, —CO₂H, —SO₂CH₃, oxazolidinone, and monocyclic 5-membered heteroaryl which is unsubstituted or substituted by 1 —CONH₂ group. In some embodiments, each R¹⁰ is independently hydrogen or C₁-C₂ alkyl which is unsubstituted or substituted by a —CN, —OH, oxazolyl, or imidazolyl group.

In some embodiments of a compound of Formula (I), (Ia), (Ib), (II), (IIa), (IIb), (IIIa), (IIIb), (IIIc), (IIId), (IV), (IVa), (IVb), (IVc), or (IVd), R⁹ is C₁-C₆ alkoxy, C₃-C₆ cycloalkyl, —O—(C₃-C₆ cycloalkyl), —O—(4- to 6-membered heterocycloalkyl), —O—(C₁-C₄ alkylene)-(C₃-C₆ cycloalkyl), —O—(C₁-C₄ alkylene)-(4- to 6-membered heterocycloalkyl), or —(C₁-C₄ alkylene)-O—(C₃-C₆ cycloalkyl); wherein the alkoxy, cycloalkyl, or heterocycloalkyl is unsubstituted or substituted by 1 or 2 groups independently selected from —OR¹⁰, —N(R¹⁰)₂, —CO₂R¹⁰, —CON(R¹⁰)₂, —CH₂N(R¹⁰)₂, —NHCOR¹⁰, —NHSO₂R¹⁰, —OCOR¹⁰, C₁-C₄ hydroxyalkyl, C₁-C₄ aminoalkyl, and 4- to 6-membered heterocycloalkyl which is unsubstituted or substituted by 1 —OH group; and each R¹⁰ is independently hydrogen, 4- to 6-membered heterocycloalkyl, or C₁-C₄ alkyl which is unsubstituted or substituted by 1, 2, or 3 groups independently selected from —F, —CN, —OH, —N(CH₃)₂, —CO₂H, —SO₂CH₃, oxazolidinone, and monocyclic heteroaryl which is unsubstituted or substituted by 1 —CONH₂ group; or two R¹⁰ attached to the same nitrogen are taken together to form an azetidinyl which is unsubstituted or substituted by 1 —SO₂CH₃ group.

In some embodiments of a compound of Formula (I), (Ia), (Ib), (II), (IIa), (IIb), (IIIa), (IIIb), (IIIc), (IIId), (IV), (IVa), (IVb), (IVc), or (IVd), R⁹ is C₁-C₆ alkoxy, C₃-C₆ cycloalkyl, —O—(C₃-C₆ cycloalkyl), —O—(4- to 6-membered heterocycloalkyl), —O—(C₁-C₄ alkylene)-(C₃-C₆ cycloalkyl), —O—(C₁-C₄ alkylene)-(4- to 6-membered heterocycloalkyl), or —(C₁-C₄ alkylene)-O—(C₃-C₆ cycloalkyl); wherein the alkoxy, cycloalkyl, or heterocycloalkyl is unsubstituted or substituted by 1 or 2 groups independently selected from —OR¹⁰, —N(R¹⁰)₂, —CO₂R¹⁰, —CON(R¹⁰)₂, —CH₂N(R¹⁰)₂, —NHCOR¹⁰, —NHSO₂R¹⁰, C₁-C₄ hydroxyalkyl, C₁-C₄ aminoalkyl, and 4- to 6-membered heterocycloalkyl which is unsubstituted or substituted by 1 —OH group; and each R¹⁰ is independently hydrogen, 4- to 6-membered heterocycloalkyl, or C₁-C₄ alkyl which is unsubstituted or substituted by 1, 2, or 3 groups independently selected from —F, —CN, —OH, —N(CH₃)₂, —CO₂H, —SO₂CH₃, oxazolidinone, and monocyclic heteroaryl which is unsubstituted or substituted by 1 —CONH₂ group; or two R¹⁰ attached to the same nitrogen are taken together to form an azetidinyl which is unsubstituted or substituted by 1 —SO₂CH₃ group.

In some embodiments of a compound of Formula (I), (Ia), (Ib), (II), (IIa), (IIb), (IIIa), (IIIb), (IIIc), (IIId), (IV), (IVa), (IVb), (IVc), or (IVd), R⁹ is C₁-C₄ alkoxy, C₃-C₄ cycloalkyl, —O—(C₃-C₄ cycloalkyl), —O—(4- to 6-membered heterocycloalkyl), —O—(C₁-C₃ alkylene)-(C₃-C₄ cycloalkyl), —O—(C₁-C₃ alkylene)-(4- to 6-membered heterocycloalkyl), or —(C₁-C₃ alkylene)-O—(C₃-C₄ cycloalkyl); wherein the alkoxy, cycloalkyl, or heterocycloalkyl is substituted by 1 or 2 groups independently selected from —OR¹⁰, —N(R¹⁰)₂, —CO₂R¹⁰, —CON(R¹⁰)₂, —CH₂N(R¹⁰)₂, —NHCOR¹⁰, —NHSO₂R¹⁰, —OCOR¹⁰, C₁-C₃ hydroxyalkyl, C₁-C₃ aminoalkyl, and 4- to 6-membered heterocycloalkyl which is unsubstituted or substituted by 1 —OH group; and each R¹⁰ is independently hydrogen or C₁-C₂ alkyl which is unsubstituted or substituted by 1, 2, or 3 groups independently selected from —F, —CN, —OH, —N(CH₃)₂, —CO₂H, —SO₂CH₃, oxazolidinone, and monocyclic 5-membered heteroaryl which is unsubstituted or substituted by 1 —CONH₂ group.

In some embodiments of a compound of Formula (I), (Ia), (Ib), (II), (IIa), (IIb), (IIIa), (III b), (IIIc), (IIId), (IV), (IVa), (IVb), (IVc), or (IVd), R⁹ is C₁-C₄ alkoxy, C₃-C₄ cycloalkyl, —O—(C₃-C₄ cycloalkyl), —O—(4- to 6-membered heterocycloalkyl), —O—(C₁-C₃ alkylene)-(C₃-C₄ cycloalkyl), —O—(C₁-C₃ alkylene)-(4- to 6-membered heterocycloalkyl), or —(C₁-C₃ alkylene)-O—(C₃-C₄ cycloalkyl); wherein the alkoxy, cycloalkyl, or heterocycloalkyl is substituted by 1 or 2 groups independently selected from —OR¹⁰, —N(R¹⁰)₂, —CO₂R¹⁰, —CON(R¹⁰)₂, —CH₂N(R¹⁰)₂, —NHCOR¹⁰, —NHSO₂R¹⁰, C₁-C₃ hydroxyalkyl, C₁-C₃ aminoalkyl, and 4- to 6-membered heterocycloalkyl which is unsubstituted or substituted by 1 —OH group; and each R¹⁰ is independently hydrogen or C₁-C₂ alkyl which is unsubstituted or substituted by 1, 2, or 3 groups independently selected from —F, —CN, —OH, —N(CH₃)₂, —CO₂H, —SO₂CH₃, oxazolidinone, and monocyclic 5-membered heteroaryl which is unsubstituted or substituted by 1 —CONH₂ group.

In some embodiments of a compound of Formula (I), (Ia), (Ib), (II), (IIa), (IIb), (IIIa), (IIIb), (IIIc), (IIId), (IV), (IVa), (IVb), (IVc), or (IVd), R⁹ is C₁-C₄ alkoxy, C₃-C₄ cycloalkyl, —O—(C₃-C₄ cycloalkyl), or —O—(4- to 6-membered heterocycloalkyl); wherein the alkoxy, cycloalkyl, or heterocycloalkyl is substituted by 1 or 2 groups independently selected from —OH, —N(R¹⁰)₂, —CON(R¹⁰)₂, —NHCOR¹⁰, —NHSO₂R¹⁰, —CH₂OH, —CH₂CH₂OH, and azetidinyl which is substituted by 1 —OH group; and each R¹⁰ is independently hydrogen or C₁-C₂ alkyl which is unsubstituted or substituted by a —CN, —OH, oxazolyl, or imidazolyl group.

In some embodiments of a compound of Formula (I), (Ia), (Ib), (II), (IIa), (IIb), (IIIa), (IIIb), (IIIc), (IIId), (IV), (IVa), (IVb), (IVc), or (IVd), R⁹ is C₁-C₄ alkoxy, C₃-C₄ cycloalkyl, —O—(C₃-C₄ cycloalkyl), or —O—(4- to 6-membered heterocycloalkyl); wherein the alkoxy, cycloalkyl, or heterocycloalkyl is substituted by 1 or 2 groups independently selected from —OH, —N(R¹⁰)₂, —CON(R¹⁰)₂, —NHCOR¹⁰, —NHSO₂R¹⁰, —CH₂CH₂OH, and azetidinyl which is substituted by 1 —OH group; and each R¹⁰ is independently hydrogen or C₁-C₂ alkyl which is unsubstituted or substituted by a —CN, —OH, oxazolyl, or imidazolyl group.

In some embodiments of a compound of Formula (I), (Ia), (Ib), (II), (IIa), (IIb), (IIIa), (IIIb), (IIIc), (IIId), (IV), (IVa), (IVb), (IVc), or (IVd), R⁹ is C₁-C₆ alkoxy. In some embodiments, R⁹ is C₁-C₄ alkoxy. In some embodiments, R⁹ is C₁-C₃ alkoxy. In some embodiments, R⁹ is C₂-C₄ alkoxy. In some embodiments, R⁹ is C₃-C₄ alkoxy.

In some embodiments of a compound of Formula (I), (Ia), (Ib), (II), (IIa), (IIb), (IIIa), (IIIb), (IIIc), (IIId), (IV), (IVa), (IVb), (IVc), or (IVd), R⁹ is C₁-C₆ alkoxy; wherein the alkoxy is unsubstituted or substituted by 1, 2, or 3 groups independently selected from —OR¹⁰ and —N(R¹⁰)₂; and each R¹⁰ is independently hydrogen or C₁-C₄ alkyl. In some embodiments, R⁹ is C₁-C₆ alkoxy; wherein the alkoxy is substituted by 1, 2, or 3 groups independently selected from —OR¹⁰ and —N(R¹⁰)₂; and each R¹⁰ is independently hydrogen or C₁-C₄ alkyl. In some embodiments, R⁹ is C₁-C₆ alkoxy; wherein the alkoxy is unsubstituted or substituted by 1, 2, or 3 groups independently selected from —OR¹⁰ and —N(R¹⁰)₂; and each R¹⁰ is independently hydrogen, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), or —C(CH₃)₃. In some embodiments, R⁹ is C₁-C₆ alkoxy; wherein the alkoxy is substituted by 1, 2, or 3 groups independently selected from —OR¹⁰ and —N(R¹⁰)₂; and each R¹⁰ is independently hydrogen, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), or —C(CH₃)₃. In some embodiments, R⁹ is C₁-C₆ alkoxy; wherein the alkoxy is unsubstituted or substituted by 1, 2, or 3 groups independently selected from —OR¹⁰ and —N(R¹⁰)₂; and each R¹⁰ is independently hydrogen, —CH₃, —CH₂CH₃, or —CH(CH₃)₂. In some embodiments, R⁹ is C₁-C₆ alkoxy; wherein the alkoxy is substituted by 1, 2, or 3 groups independently selected from —OR¹⁰ and —N(R¹⁰)₂; and each R¹⁰ is independently hydrogen, —CH₃, —CH₂CH₃, or —CH(CH₃)₂. In some embodiments, R⁹ is C₁-C₆ alkoxy; wherein the alkoxy is unsubstituted or substituted by 1, 2, or 3 groups independently selected from —OH, —OCH₃, —NH₂, —N(CH₃)₂, and —CH₂OH. In some embodiments, R⁹ is C₁-C₆ alkoxy; wherein the alkoxy is unsubstituted or substituted by 1, 2, or 3 groups independently selected from —OH, —OCH₃, —NH₂, and —N(CH₃)₂. In some embodiments, R⁹ is C₁-C₆ alkoxy; wherein the alkoxy is substituted by 1, 2, or 3 groups independently selected from —OH, —OCH₃, —NH₂, —N(CH₃)₂, and —CH₂OH. In some embodiments, R⁹ is C₁-C₆ alkoxy; wherein the alkoxy is substituted by 1, 2, or 3 groups independently selected from —OH, —OCH₃, —NH₂, and —N(CH₃)₂. In some embodiments, R⁹ is C₁-C₆ alkoxy; wherein the alkoxy is unsubstituted or substituted by 1 or 2 groups independently selected from —OH, —OCH₃, —NH₂, —N(CH₃)₂, and —CH₂OH. In some embodiments, R⁹ is C₁-C₆ alkoxy; wherein the alkoxy is unsubstituted or substituted by 1 or 2 groups independently selected from —OH, —OCH₃, —NH₂, and —N(CH₃)₂. In some embodiments, R⁹ is C₁-C₆ alkoxy; wherein the alkoxy is substituted by 1 or 2 groups independently selected from —OH, —OCH₃, —NH₂, —N(CH₃)₂, and —CH₂OH. In some embodiments, R⁹ is C₁-C₆ alkoxy; wherein the alkoxy is substituted by 1 or 2 groups independently selected from —OH, —OCH₃, —NH₂, and —N(CH₃)₂.

In some embodiments of a compound of Formula (I), (Ia), (Ib), (II), (IIa), (IIb), (IIIa), (IIIb), (IIIc), (IIId), (IV), (IVa), (IVb), (IVc), or (IVd), R⁹ is C₁-C₄ alkoxy; wherein the alkoxy is unsubstituted or substituted by 1, 2, or 3 groups independently selected from —OR¹⁰ and —N(R¹⁰)₂; and each R¹⁰ is independently hydrogen, —CH₃, —CH₂CH₃, or —CH(CH₃)₂. In some embodiments, R⁹ is C₁-C₄ alkoxy; wherein the alkoxy is substituted by 1, 2, or 3 groups independently selected from —OR¹⁰ and —N(R¹⁰)₂; and each R¹⁰ is independently hydrogen, —CH₃, —CH₂CH₃, or —CH(CH₃)₂. In some embodiments, R⁹ is C₁-C₄ alkoxy; wherein the alkoxy is unsubstituted or substituted by 1, 2, or 3 groups independently selected from —OH, —OCH₃, —NH₂, and —N(CH₃)₂. In some embodiments, R⁹ is C₁-C₄ alkoxy; wherein the alkoxy is substituted by 1, 2, or 3 groups independently selected from —OH, —OCH₃, —NH₂, and —N(CH₃)₂. In some embodiments, R⁹ is C₁-C₄ alkoxy; wherein the alkoxy is unsubstituted or substituted by 1 or 2 groups independently selected from —OH, —OCH₃, —NH₂, and —N(CH₃)₂. In some embodiments, R⁹ is C₁-C₄ alkoxy; wherein the alkoxy is substituted by 1 or 2 groups independently selected from —OH, —OCH₃, —NH₂, and —N(CH₃)₂. In some embodiments, R⁹ is C₁-C₄ alkoxy; wherein the alkoxy is substituted by 2 —OH groups.

In some embodiments of a compound of Formula (I), (Ia), (Ib), (II), (IIa), (IIb), (IIIa), (IIIb), (IIIc), (IIId), (IV), (IVa), (IVb), (IVc), or (IVd), R⁹ is C₃-C₄ alkoxy; wherein the alkoxy is unsubstituted or substituted by 1, 2, or 3 groups independently selected from —OR¹⁰ and —N(R¹⁰)₂; and each R¹⁰ is independently hydrogen, —CH₃, —CH₂CH₃, or —CH(CH₃)₂. In some embodiments, R⁹ is C₃-C₄ alkoxy; wherein the alkoxy is substituted by 1, 2, or 3 groups independently selected from —OR¹⁰ and —N(R¹⁰)₂; and each R¹⁰ is independently hydrogen, —CH₃, —CH₂CH₃, or —CH(CH₃)₂. In some embodiments, R⁹ is C₃-C₄ alkoxy; wherein the alkoxy is unsubstituted or substituted by 1, 2, or 3 groups independently selected from —OH, —OCH₃, —NH₂, and —N(CH₃)₂. In some embodiments, R⁹ is C₃-C₄ alkoxy; wherein the alkoxy is substituted by 1, 2, or 3 groups independently selected from —OH, —OCH₃, —NH₂, and —N(CH₃)₂. In some embodiments, R⁹ is C₃-C₄ alkoxy; wherein the alkoxy is unsubstituted or substituted by 1 or 2 groups independently selected from —OH, —OCH₃, —NH₂, and —N(CH₃)₂. In some embodiments, R⁹ is C₃-C₄ alkoxy; wherein the alkoxy is substituted by 1 or 2 groups independently selected from —OH, —OCH₃, —NH₂, and —N(CH₃)₂. In some embodiments, R⁹ is C₃-C₄ alkoxy; wherein the alkoxy is substituted by 2 —OH groups.

In some embodiments of a compound of Formula (I), (Ia), (Ib), (II), (IIa), (IIb), (IIIa), (IIIb), (IIIc), (IIId), (IV), (IVa), (IVb), (IVc), or (IVd), R⁹ is C₃-C₄ cycloalkyl, —O—(C₃-C₄ cycloalkyl), —O—(4- to 6-membered heterocycloalkyl), —O—(C₁-C₃ alkylene)-(C₃-C₄ cycloalkyl), —O—(C₁-C₃ alkylene)-(4- to 6-membered heterocycloalkyl), or —(C₁-C₃ alkylene)-O—(C₃-C₄ cycloalkyl). In some embodiments, R⁹ is C₃-C₄ cycloalkyl, —O—(C₃-C₄ cycloalkyl), —O—(4- to 6-membered heterocycloalkyl), —O—(C₁-C₃ alkylene)-(C₃-C₄ cycloalkyl), —O—(C₁-C₃ alkylene)-(4- to 6-membered heterocycloalkyl), or —(C₁-C₃ alkylene)-O—(C₃-C₄ cycloalkyl); wherein the cycloalkyl or heterocycloalkyl is substituted by 1 or 2 groups independently selected from —OR¹⁰, —N(R¹⁰)₂, —CO₂R¹⁰, —CON(R¹⁰)₂, —CH₂N(R¹⁰)₂, —NHCOR¹⁰, —NHSO₂R¹⁰, —OCOR¹⁰, C₁-C₃ hydroxyalkyl, C₁-C₃ aminoalkyl, and 4- to 6-membered heterocycloalkyl which is unsubstituted or substituted by 1 —OH group; and each R¹⁰ is independently hydrogen or C₁-C₂ alkyl which is unsubstituted or substituted by 1, 2, or 3 groups independently selected from —F, —CN, —OH, —N(CH₃)₂, —CO₂H, —SO₂CH₃, oxazolidinone, and monocyclic 5-membered heteroaryl which is unsubstituted or substituted by 1 —CONH₂ group; or two R¹⁰ attached to the same nitrogen are taken together to form an azetidinyl which is unsubstituted or substituted by 1 —SO₂CH₃ group.

In some embodiments, R⁹ is C₃-C₄ cycloalkyl, —O—(C₃-C₄ cycloalkyl), —O—(4- to 6-membered heterocycloalkyl), —O—(C₁-C₃ alkylene)-(C₃-C₄ cycloalkyl), —O—(C₁-C₃ alkylene)-(4- to 6-membered heterocycloalkyl), or —(C₁-C₃ alkylene)-O—(C₃-C₄ cycloalkyl); wherein the cycloalkyl or heterocycloalkyl is substituted by 1 or 2 groups independently selected from —OR¹⁰, —N(R¹⁰)₂, —CO₂R¹⁰, —CON(R¹⁰)₂, —CH₂N(R¹⁰)₂, —NHCOR¹⁰, —NHSO₂R¹⁰, C₁-C₃ hydroxyalkyl, C₁-C₃ aminoalkyl, and 4- to 6-membered heterocycloalkyl which is unsubstituted or substituted by 1 —OH group; and each R¹⁰ is independently hydrogen or C₁-C₂ alkyl which is unsubstituted or substituted by 1, 2, or 3 groups independently selected from —F, —CN, —OH, —N(CH₃)₂, —CO₂H, —SO₂CH₃, oxazolidinone, and monocyclic 5-membered heteroaryl which is unsubstituted or substituted by 1 —CONH₂ group; or two R¹⁰ attached to the same nitrogen are taken together to form an azetidinyl which is unsubstituted or substituted by 1 —SO₂CH₃ group. In some embodiments, R⁹ is C₃-C₄ cycloalkyl, —O—(C₃-C₄ cycloalkyl), or —O—(4- to 6-membered heterocycloalkyl); wherein the cycloalkyl or heterocycloalkyl is substituted by 1 or 2 groups independently selected from —OR¹⁰, —N(R¹⁰)₂, —CO₂R¹⁰, —CON(R¹⁰)₂, —CH₂N(R¹⁰)₂, —NHCOR¹⁰, —NHSO₂R¹⁰, C₁-C₃ hydroxyalkyl, C₁-C₃ aminoalkyl, and 4- to 6-membered heterocycloalkyl which is unsubstituted or substituted by 1 —OH group; and each R¹⁰ is independently hydrogen or C₁-C₂ alkyl which is unsubstituted or substituted by 1, 2, or 3 groups independently selected from —F, —CN, —OH, —N(CH₃)₂, —CO₂H, —SO₂CH₃, oxazolidinone, and monocyclic 5-membered heteroaryl which is unsubstituted or substituted by 1 —CONH₂ group; or two R¹⁰ attached to the same nitrogen are taken together to form an azetidinyl which is unsubstituted or substituted by 1 —SO₂CH₃ group.

In some embodiments of a compound of Formula (I), (Ia), (Ib), (II), (IIa), (IIb), (IIIa), (IIIb), (IIIc), (IIId), (IV), (IVa), (IVb), (IVc), or (IVd), R⁹ is —O—(C₃-C₆ cycloalkyl) or —O—(3- to 6-membered heterocycloalkyl); wherein the cycloalkyl or heterocycloalkyl is unsubstituted or substituted by 1, 2, or 3 groups independently selected from —OR¹⁰, —N(R¹⁰)₂, C₁-C₄ alkyl, and C₁-C₄ hydroxyalkyl. In some embodiments, R⁹ is —O—(C₃-C₆ cycloalkyl) or —O—(3- to 6-membered heterocycloalkyl); wherein the cycloalkyl or heterocycloalkyl is unsubstituted or substituted by 1, 2, or 3 groups independently selected from —OH, —OCH₃, —NH₂, —N(CH₃)₂, —CH₃, —CH₂OH, and —CH₂CH₂OH. In some embodiments, R⁹ is —O—(C₃-C₆ cycloalkyl) or —O—(3- to 6-membered heterocycloalkyl); wherein the cycloalkyl or heterocycloalkyl is unsubstituted or substituted by 1, 2, or 3 groups independently selected from —OH, —OCH₃, —NH₂, —N(CH₃)₂, —CH₃, and —CH₂CH₂OH. In some embodiments, R⁹ is —O—(C₃-C₆ cycloalkyl) or —O—(3- to 6-membered heterocycloalkyl); wherein the cycloalkyl or heterocycloalkyl is unsubstituted or substituted by 1 or 2 groups independently selected from —OH, —OCH₃, —NH₂, —N(CH₃)₂, —CH₃, and —CH₂CH₂OH.

In some embodiments of a compound of Formula (I), (Ia), (Ib), (II), (IIa), (IIb), (IIIa), (IIIb), (IIIc), (IIId), (IV), (IVa), (IVb), (IVc), or (IVd), R⁹ is —O—(C₃-C₆ cycloalkyl). In some embodiments, R⁹ is —O—(C₃-C₆ cycloalkyl); wherein the cycloalkyl is unsubstituted or substituted by 1, 2, or 3 groups independently selected from —OR¹⁰, —N(R¹⁰)₂, C₁-C₄ alkyl, and C₁-C₄ hydroxyalkyl. In some embodiments, R⁹ is —O—(C₃-C₆ cycloalkyl); wherein the cycloalkyl is unsubstituted or substituted by 1, 2, or 3 groups independently selected from —OR¹⁰ and —N(R¹⁰)₂. In some embodiments, R⁹ is —O—(C₃-C₆ cycloalkyl); wherein the cycloalkyl is unsubstituted or substituted by 1, 2, or 3 groups independently selected from —OH, —OCH₃, —NH₂, and —N(CH₃)₂. In some embodiments, R⁹ is —O—(C₃-C₆ cycloalkyl); wherein the cycloalkyl is unsubstituted or substituted by 1 or 2 groups independently selected from —OH, —OCH₃, —NH₂, and —N(CH₃)₂. In some embodiments, R⁹ is —O—(C₃-C₆ cycloalkyl); wherein the cycloalkyl is unsubstituted or substituted by 1 or 2 groups independently selected from —OH and —NH₂.

In some embodiments of a compound of Formula (I), (Ia), (Ib), (II), (IIa), (IIb), (IIIa), (IIIb), (IIIc), (IIId), (IV), (IVa), (IVb), (IVc), or (IVd), R⁹ is —O—(C₃-C₄ cycloalkyl); wherein the cycloalkyl is unsubstituted or substituted by 1, 2, or 3 groups independently selected from —OR¹⁰, —N(R¹⁰)₂, C₁-C₄ alkyl, and C₁-C₄ hydroxyalkyl. In some embodiments, R⁹ is —O—(C₃-C₄ cycloalkyl); wherein the cycloalkyl is unsubstituted or substituted by 1, 2, or 3 groups independently selected from —OR¹⁰ and —N(R¹⁰)₂. In some embodiments, R⁹ is —O—(C₃-C₄ cycloalkyl); wherein the cycloalkyl is unsubstituted or substituted by 1, 2, or 3 groups independently selected from —OH, —OCH₃, —NH₂, and —N(CH₃)₂. In some embodiments, R⁹ is —O—(C₃-C₄ cycloalkyl); wherein the cycloalkyl is unsubstituted or substituted by 1 or 2 groups independently selected from —OH, —OCH₃, —NH₂, and —N(CH₃)₂. In some embodiments, R⁹ is —O—(C₃-C₄ cycloalkyl); wherein the cycloalkyl is unsubstituted or substituted by 1 or 2 groups independently selected from —OH and —NH₂.

In some embodiments of a compound of Formula (I), (Ia), (Ib), (II), (IIa), (IIb), (IIIa), (IIIb), (IIIc), (IIId), (IV), (IVa), (IVb), (IVc), or (IVd), R⁹ is —O-(cyclopropyl); wherein the cyclopropyl is unsubstituted or substituted by 1 or 2 groups independently selected from —OH and —NH₂. In some embodiments, R⁹ is —O-(cyclopropyl); wherein the cyclopropyl is substituted by 1 group selected from —OH and —NH₂. In some embodiments, R⁹ is —O-(cyclopropyl); wherein the cyclopropyl is substituted by 1 —OH group. In some embodiments, R⁹ is —O-(cyclopropyl); wherein the cyclopropyl is substituted by 1 —NH₂ group.

In some embodiments of a compound of Formula (I), (Ia), (Ib), (II), (IIa), (IIb), (IIIa), (IIIb), (IIIc), (IIId), (IV), (IVa), (IVb), (IVc), or (IVd), R⁹ is —O—(4- to 6-membered heterocycloalkyl). In some embodiments, R⁹ is —O—(4- to 6-membered heterocycloalkyl); wherein the heterocycloalkyl is unsubstituted or substituted by 1, 2, or 3 groups independently selected from —OR¹⁰, —N(R¹⁰)₂, C₁-C₄ alkyl, and C₁-C₄ hydroxyalkyl. In some embodiments, R⁹ is —O—(4- to 6-membered heterocycloalkyl); wherein the heterocycloalkyl is unsubstituted or substituted by 1, 2, or 3 groups independently selected from —OH, —OCH₃, —NH₂, —N(CH₃)₂, —CH₂OH, and —CH₂CH₂OH. In some embodiments, R⁹ is —O—(4- to 6-membered heterocycloalkyl); wherein the heterocycloalkyl is unsubstituted or substituted by 1, 2, or 3 groups independently selected from —OH, —OCH₃, —NH₂, —N(CH₃)₂, and —CH₂CH₂OH. In some embodiments, R⁹ is —O—(4- to 6-membered heterocycloalkyl); wherein the heterocycloalkyl is unsubstituted or substituted by 1 or 2 groups independently selected from —OH, —OCH₃, —NH₂, —N(CH₃)₂, —CH₂OH, and —CH₂CH₂OH. In some embodiments, R⁹ is —O—(4- to 6-membered heterocycloalkyl); wherein the heterocycloalkyl is unsubstituted or substituted by 1 or 2 groups independently selected from —OH, —OCH₃, —NH₂, —N(CH₃)₂, and —CH₂CH₂OH.

In some embodiments of a compound of Formula (I), (Ia), (Ib), (II), (IIa), (IIb), (IIIa), (IIIb), (IIIc), (IIId), (IV), (IVa), (IVb), (IVc), or (IVd), R⁹ is —O-(azetidinyl); wherein the azetidinyl is unsubstituted or substituted by 1 or 2 groups independently selected from —OH, —OCH₃, —NH₂, —N(CH₃)₂, and —CH₂CH₂OH. In some embodiments, R⁹ is —O-(azetidinyl); wherein the azetidinyl is substituted by 1 group selected from —OH and —NH₂. In some embodiments, R⁹ is —O-(azetidinyl); wherein the azetidinyl is substituted by 1 —CH₂CH₂OH group.

In some embodiments of a compound of Formula (I), (Ia), (Ib), (II), (IIa), (IIb), (IIIa), (IIIb), (IIIc), (IIId), (IV), (IVa), (IVb), (IVc), or (IVd), R⁹ is C₃-C₄ cycloalkyl. In some embodiments, R⁹ is C₃-C₄ cycloalkyl; wherein the cycloalkyl is substituted by 1 or 2 groups independently selected from —N(R¹⁰)₂, —CO₂R¹⁰, —CON(R¹⁰)₂, —CH₂N(R¹⁰)₂, —NHCOR¹⁰, —NHSO₂R¹⁰, —OCOR¹⁰, and 4- to 6-membered heterocycloalkyl which is unsubstituted or substituted by 1 —OH group. In some embodiments, R⁹ is C₃-C₄ cycloalkyl; wherein the cycloalkyl is substituted by 1 or 2 groups independently selected from —N(R¹⁰)₂, —CO₂R¹⁰, —CON(R¹⁰)₂, —CH₂N(R¹⁰)₂, —NHCOR¹⁰, —NHSO₂R¹⁰, and 4- to 6-membered heterocycloalkyl which is unsubstituted or substituted by 1 —OH group. In some embodiments, R⁹ is C₃-C₄ cycloalkyl; wherein the cycloalkyl is substituted by 1 group selected from —N(R¹⁰)₂, —CO₂R¹⁰, —CON(R¹⁰)₂, —CH₂N(R¹⁰)₂, —NHCOR¹⁰, —NHSO₂R¹⁰, —OCOR¹⁰, and 4- to 6-membered heterocycloalkyl which is unsubstituted or substituted by 1 —OH group. In some embodiments, R⁹ is C₃-C₄ cycloalkyl; wherein the cycloalkyl is substituted by 1 group selected from —N(R¹⁰)₂, —CO₂R¹⁰, —CON(R¹⁰)₂, —CH₂N(R¹⁰)₂, —NHCOR¹⁰, —NHSO₂R¹⁰, and 4- to 6-membered heterocycloalkyl which is unsubstituted or substituted by 1 —OH group. In some embodiments, R⁹ is C₃-C₄ cycloalkyl; wherein the cycloalkyl is substituted by 1 —N(R¹⁰)₂ group. In some embodiments, R⁹ is C₃-C₄ cycloalkyl; wherein the cycloalkyl is substituted by 1 —CO₂R¹⁰ group.

In some embodiments, R⁹ is C₃-C₄ cycloalkyl; wherein the cycloalkyl is substituted by 1 —CON(R¹⁰)₂ group. In some embodiments, R⁹ is C₃-C₄ cycloalkyl; wherein the cycloalkyl is substituted by 1 —CH₂N(R¹⁰)₂ group. In some embodiments, R⁹ is C₃-C₄ cycloalkyl; wherein the cycloalkyl is substituted by 1 —NHCOR¹⁰ group. In some embodiments, R⁹ is C₃-C₄ cycloalkyl; wherein the cycloalkyl is substituted by 1 —NHSO₂R¹⁰ group. In some embodiments, R⁹ is C₃-C₄ cycloalkyl; wherein the cycloalkyl is substituted by 1 —OCOR¹⁰ group. In some embodiments, R⁹ is C₃-C₄ cycloalkyl; wherein the cycloalkyl is substituted by a 4- to 6-membered heterocycloalkyl which is unsubstituted or substituted by 1 —OH group. In some embodiments, R⁹ is cyclobutyl; wherein the cyclobutyl is substituted by 1 group selected from —N(R¹⁰)₂, —CO₂R¹⁰, —CON(R¹⁰)₂, —CH₂N(R¹⁰)₂, —NHCOR¹⁰, —NHSO₂R¹⁰, and azetidinyl which is substituted by 1 —OH group. In some embodiments, R⁹ is cyclobutyl; wherein the cyclobutyl is substituted by 1 group selected from —N(R¹⁰)₂, —CO₂R¹⁰, —CON(R¹⁰)₂, —CH₂N(R¹⁰)₂, —NHCOR¹⁰, and —NHSO₂R¹⁰. In some embodiments, each R¹⁰ is independently hydrogen, 4- to 6-membered heterocycloalkyl, or C₁-C₄ alkyl which is unsubstituted or substituted by 1, 2, or 3 groups independently selected from —F, —CN, —OH, —NH₂, —OMe, —N(CH₃)₂, —CO₂H, —SO₂CH₃, oxazolidinone, phenyl, and monocyclic heteroaryl which is unsubstituted or substituted by 1 —CONH₂ group; or two R¹⁰ attached to the same nitrogen are taken together to form an azetidinyl which is unsubstituted or substituted by 1 —SO₂CH₃ group. In some embodiments, each R¹⁰ is independently hydrogen or C₁-C₄ alkyl which is unsubstituted or substituted by 1 or 2 groups independently selected from —F, —CN, —OH, —N(CH₃)₂, —CO₂H, —SO₂CH₃, oxazolidinone, and monocyclic heteroaryl which is unsubstituted or substituted by 1 —CONH₂ group; or two R¹⁰ attached to the same nitrogen are taken together to form an azetidinyl which is unsubstituted or substituted by 1 —SO₂CH₃ group. In some embodiments, each R¹⁰ is independently hydrogen or C₁-C₂ alkyl which is unsubstituted or substituted by 1, 2, or 3 groups independently selected from —F —CN, —OH, —N(CH₃)₂, —CO₂H, —SO₂CH₃, oxazolidinone, and monocyclic 5-membered heteroaryl which is unsubstituted or substituted by 1 —CONH₂ group. In some embodiments, each R¹⁰ is independently hydrogen or C₁-C₂ alkyl which is unsubstituted or substituted by a —CN, —OH, oxazolyl, or imidazolyl group.

In some embodiments of a compound of Formula (I), (Ia), (Ib), (II), (IIa), (IIb), (IIIa), (IIIb), (IIIc), (IIId), (IV), (IVa), (IVb), (IVc), or (IVd), R⁹ is

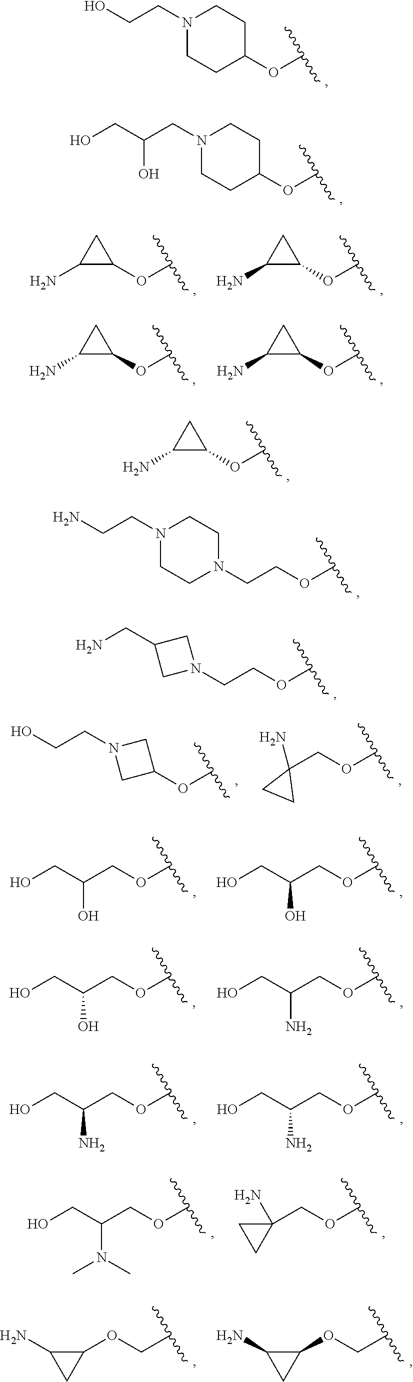

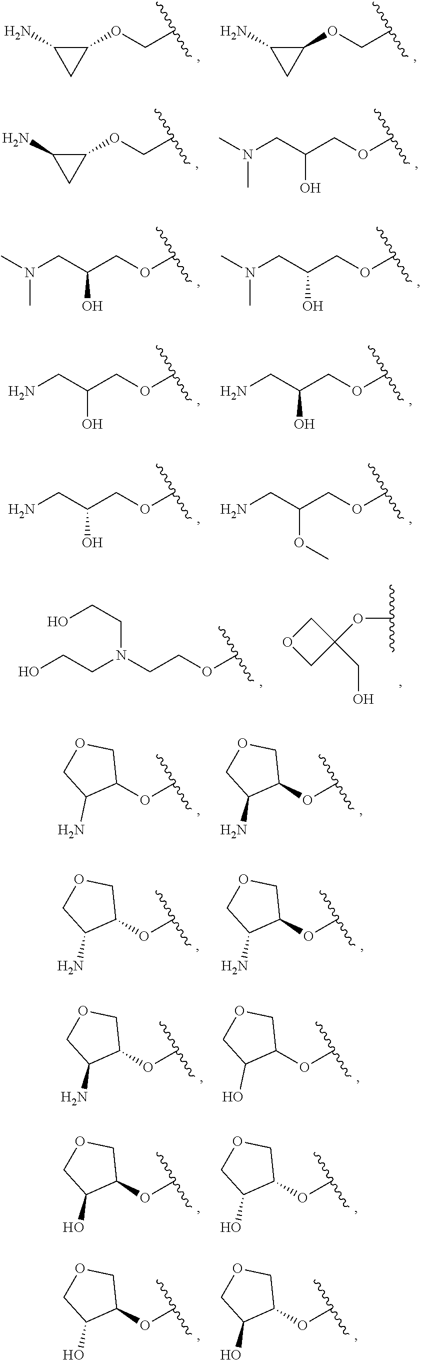

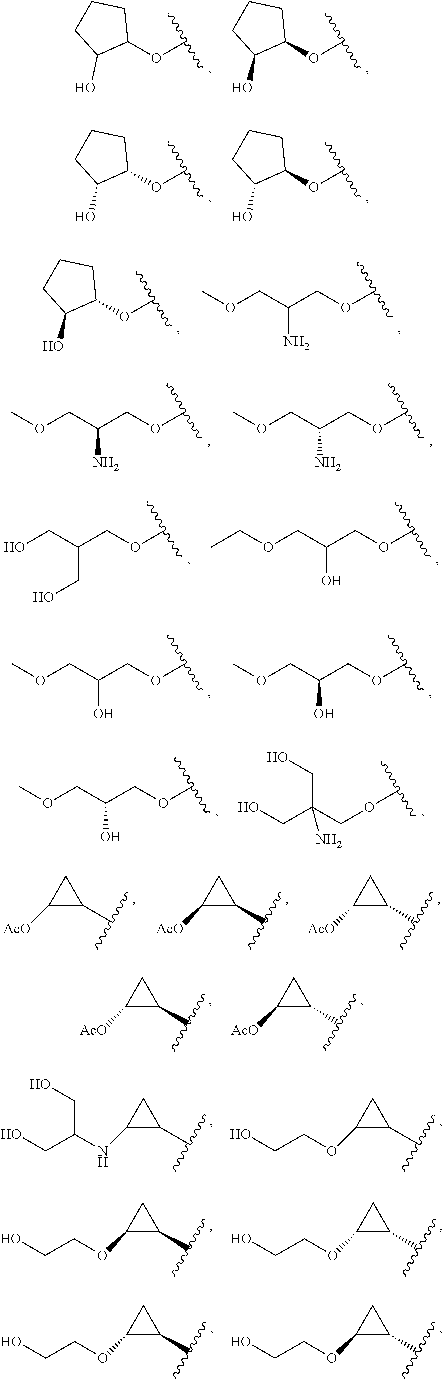

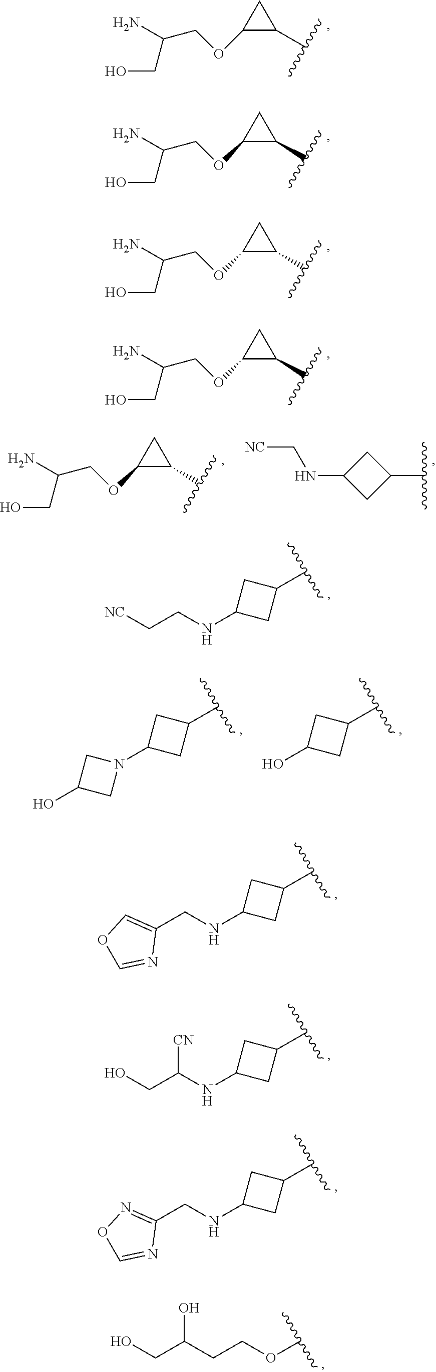

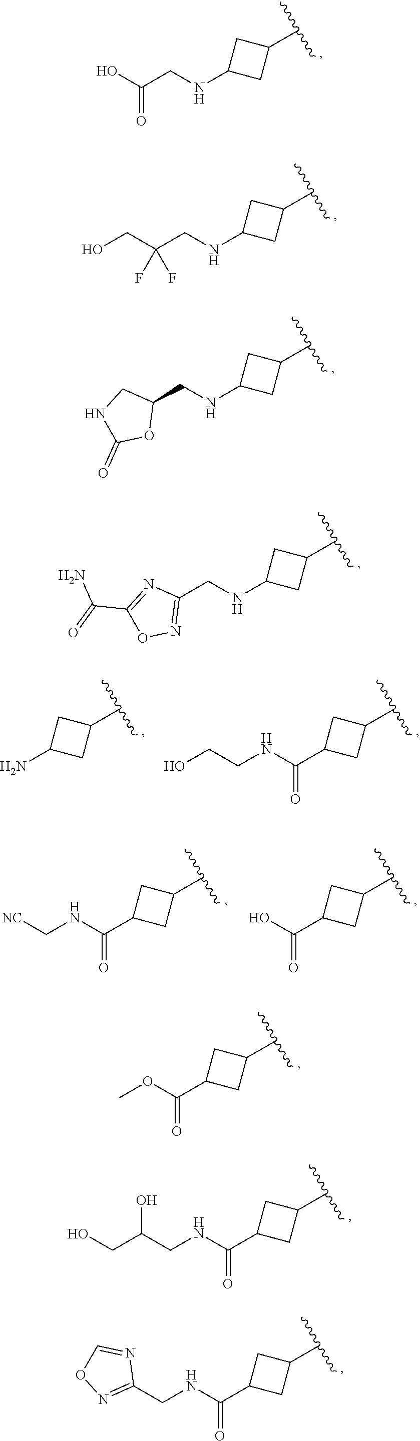

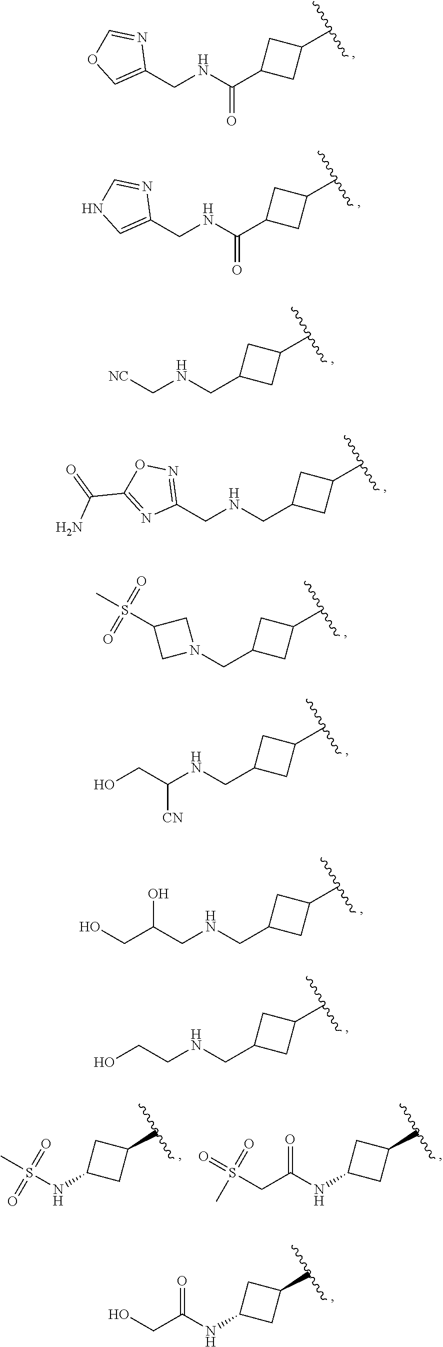

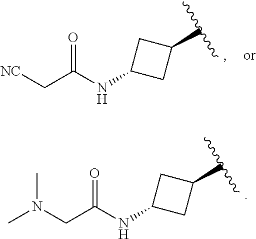 [SMILES: *[C@H]1C[C@H](NC(=O)CC#N)C1 ; *[C@H]1C[C@H](NC(=O)CN(C)C)C1]

In some embodiments of a compound of Formula (I), (Ia), (Ib), (II), (IIa), (IIb), (IIIa), (IIIb), (IIIc), (IIId), (IV), (IVa), (IVb), (IVc), or (IVd), R⁹ is

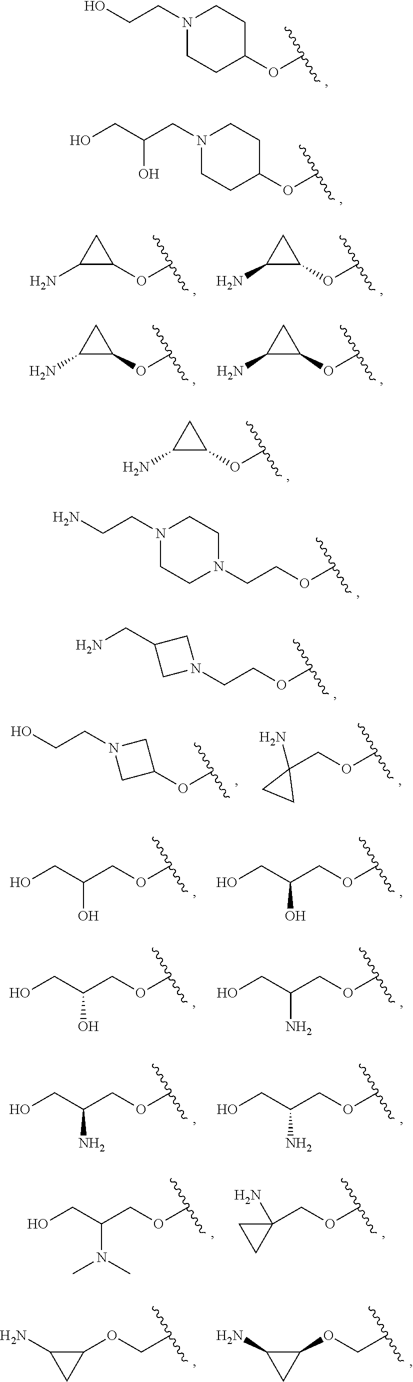

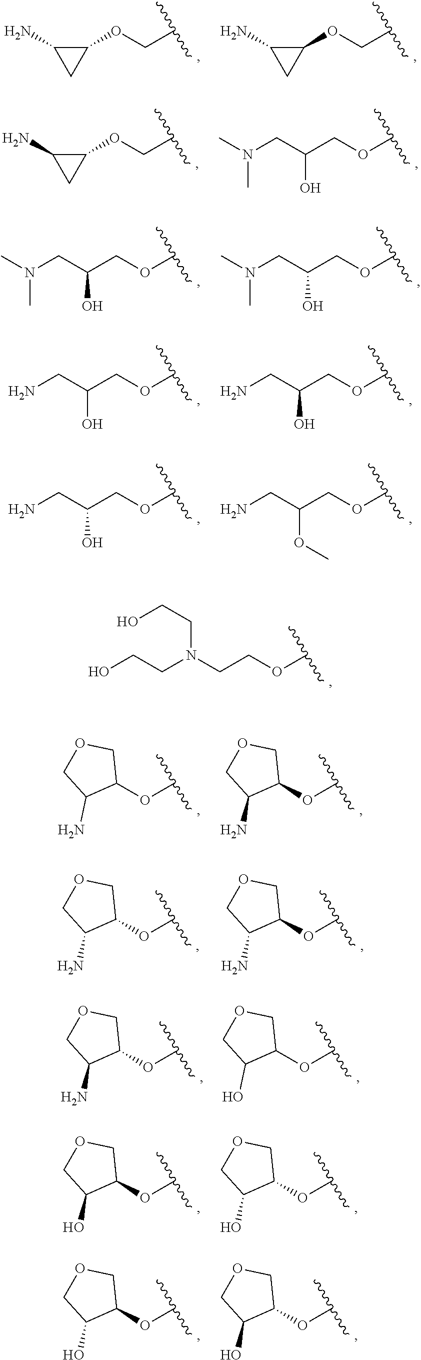

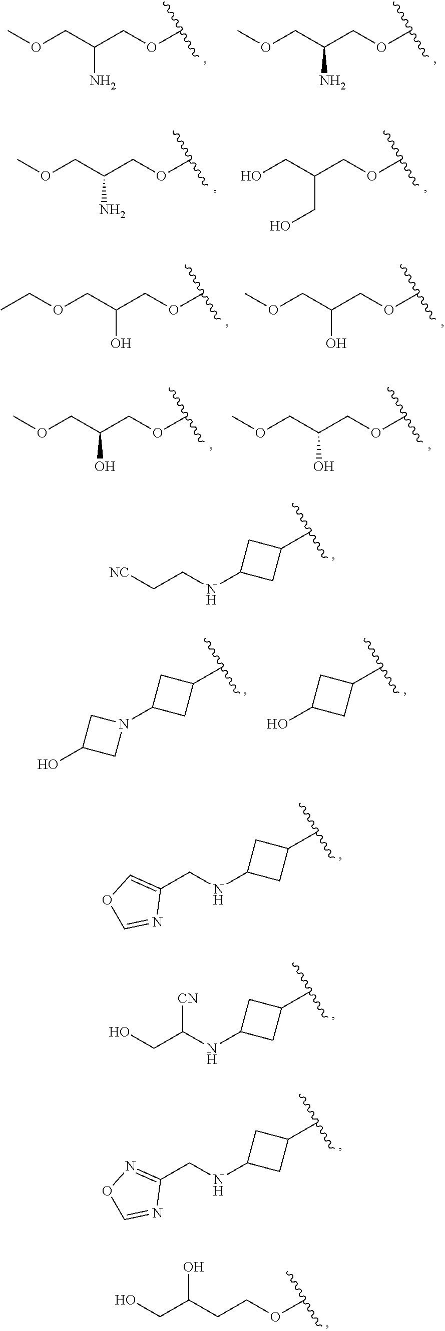

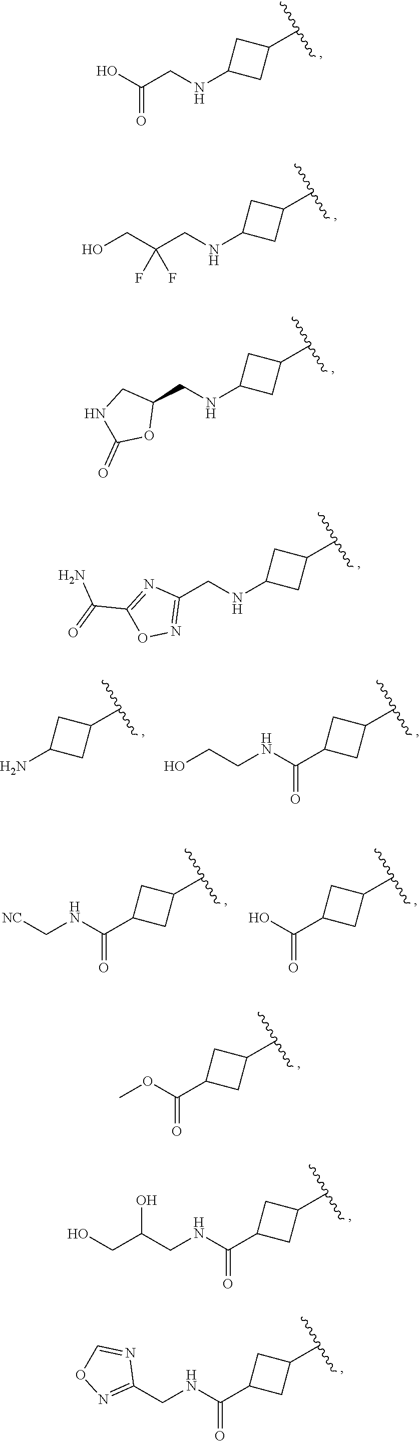

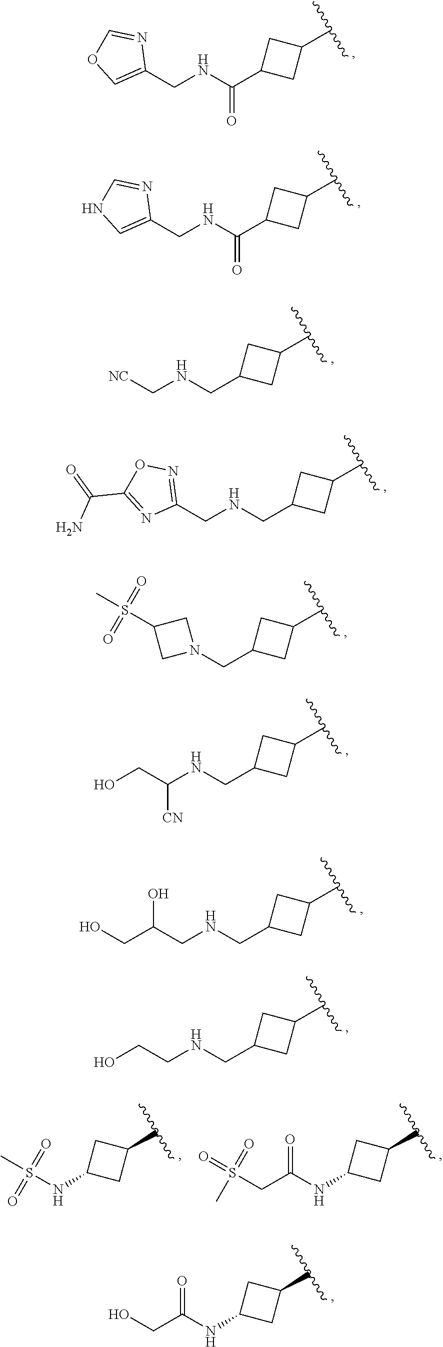

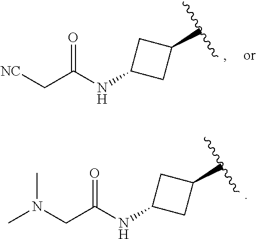 [SMILES: *[C@H]1C[C@H](NC(=O)CC#N)C1 ; *[C@H]1C[C@H](NC(=O)CN(C)C)C1]

In some embodiments of a compound of Formula (I), (Ia), (Ib), (II), (IIa), (IIb), (IIIa), (IIIb), (IIIc), (IIId), (IV), (IVa), (IVb), (IVc), or (IVd), R⁹ is

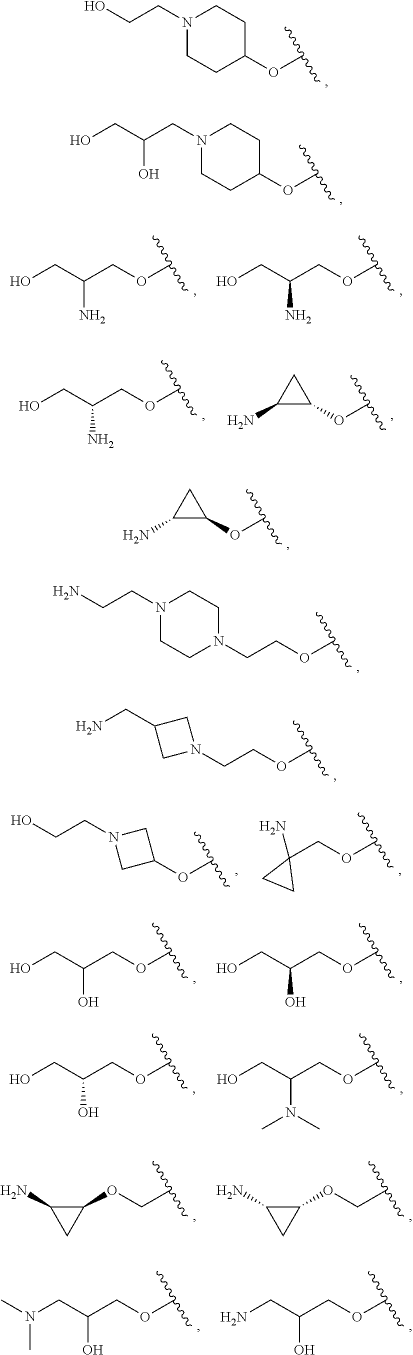

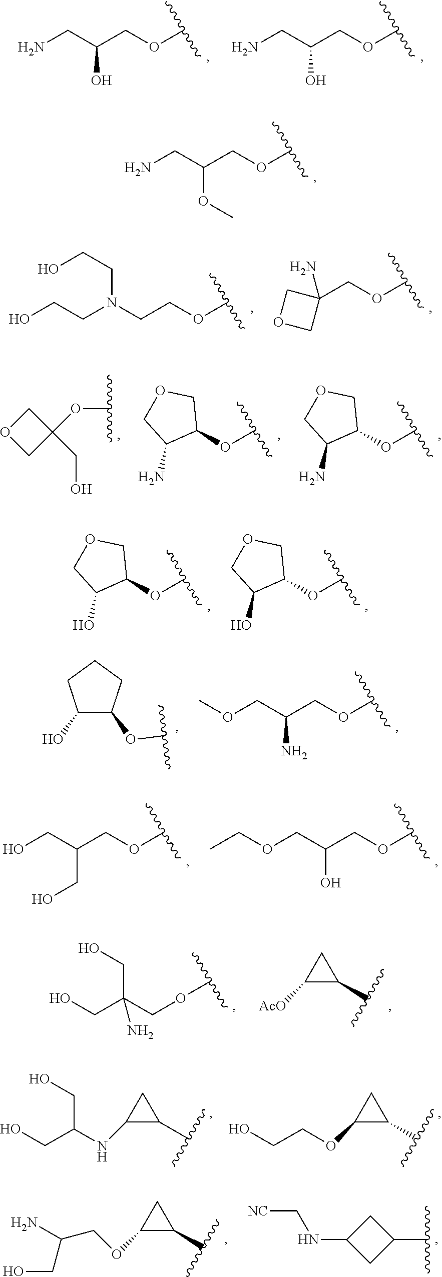

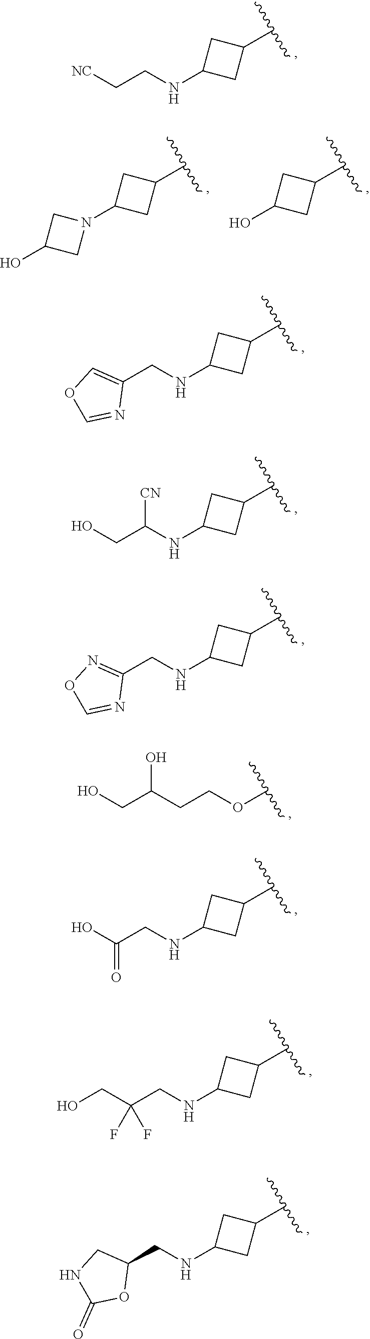

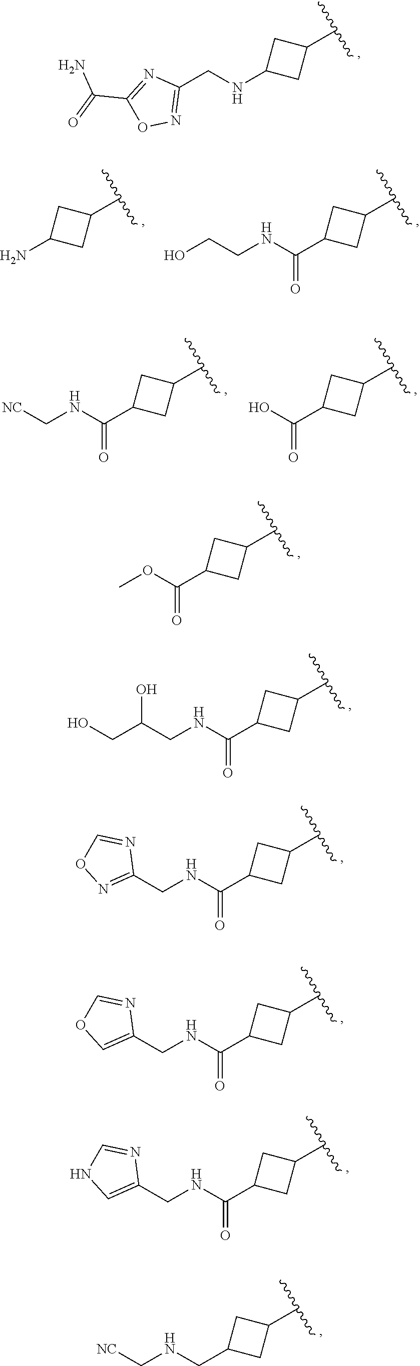

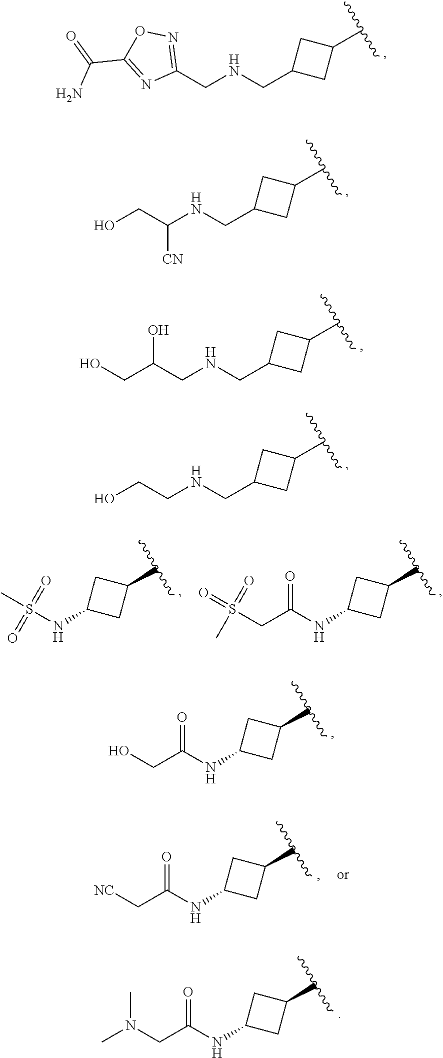

In some embodiments of a compound of Formula (I), (Ia), (Ib), (II), (IIa), (IIb), (IIIa), (IIIb), (IIIc), (IIId), (IV), (IVa), (IVb), (IVc), or (IVd), R⁹ is

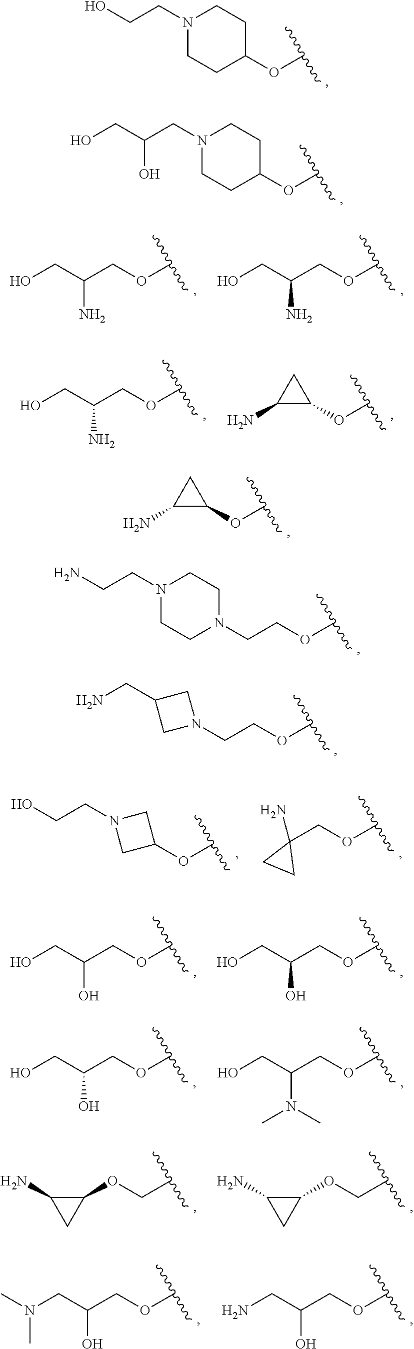

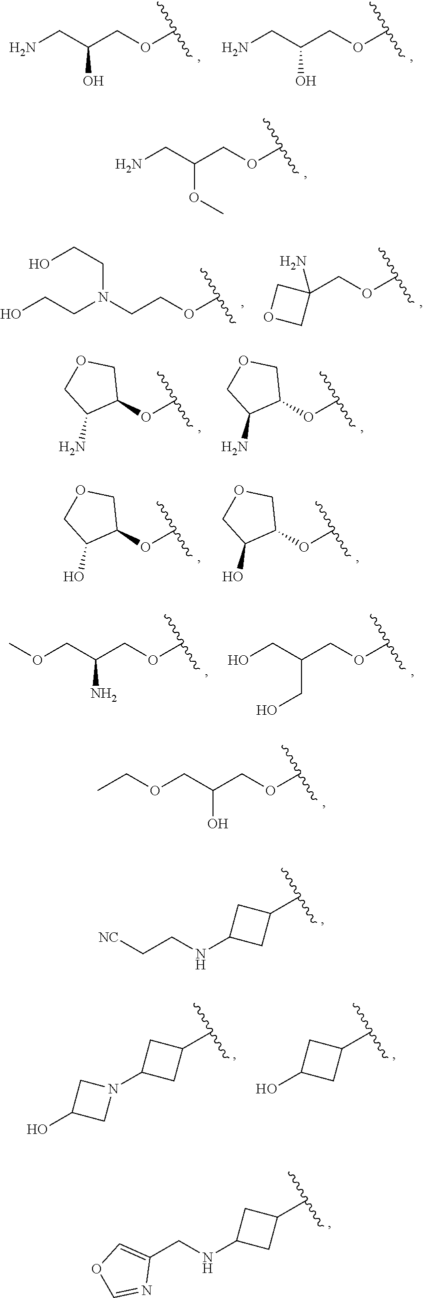

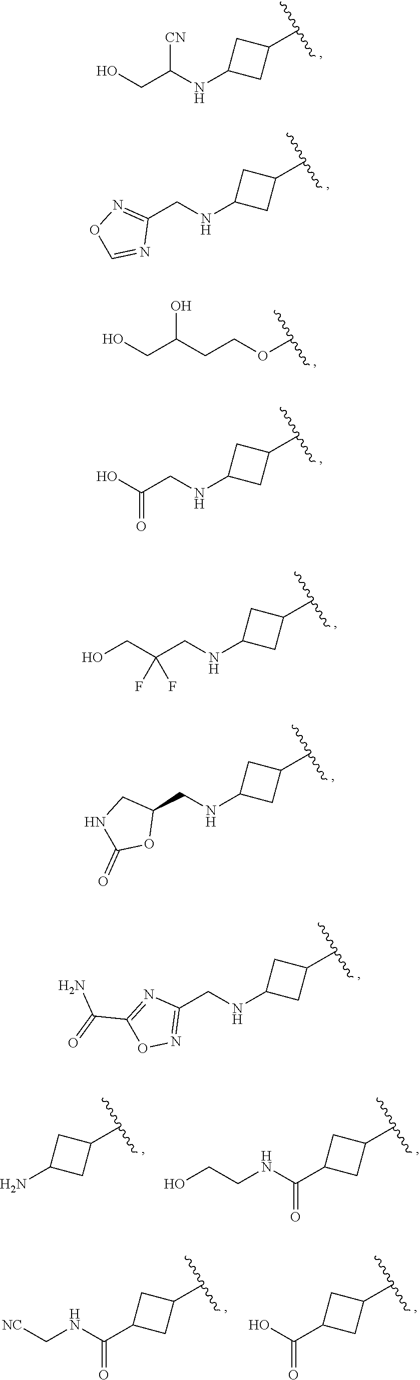

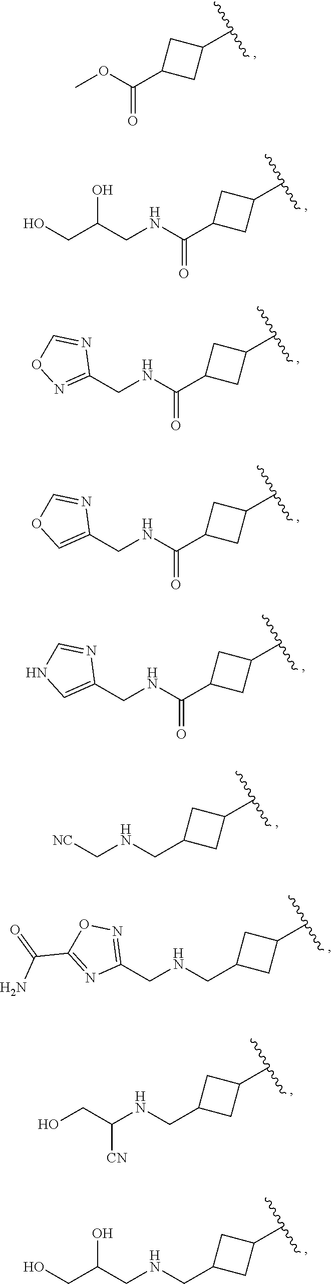

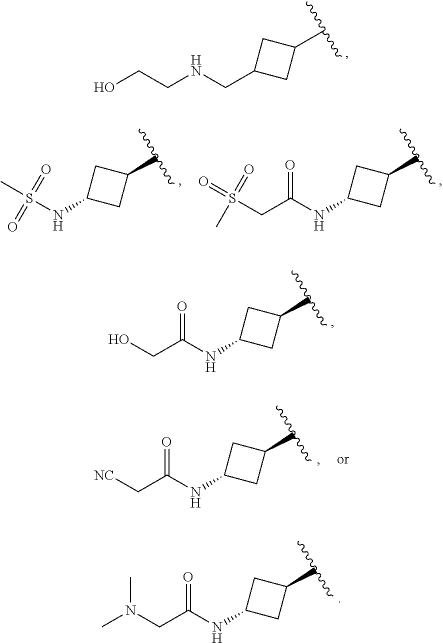

In some embodiments of a compound of Formula (I), (Ia), (Ib), (II), (IIa), (IIb), (IIIa), (IIIb), (IIIc), (IIId), (IV), (IVa), (IVb), (IVc), or (IVd), R⁹ is

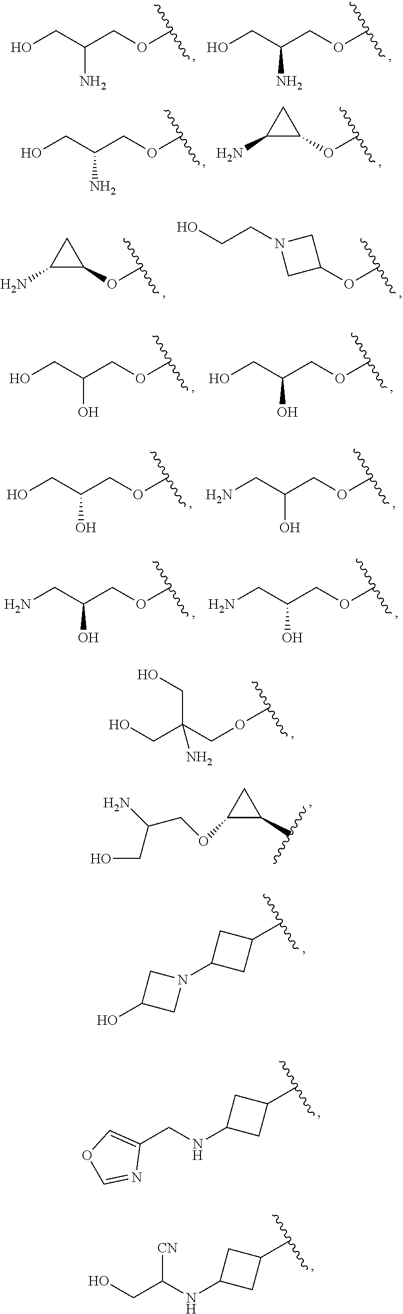

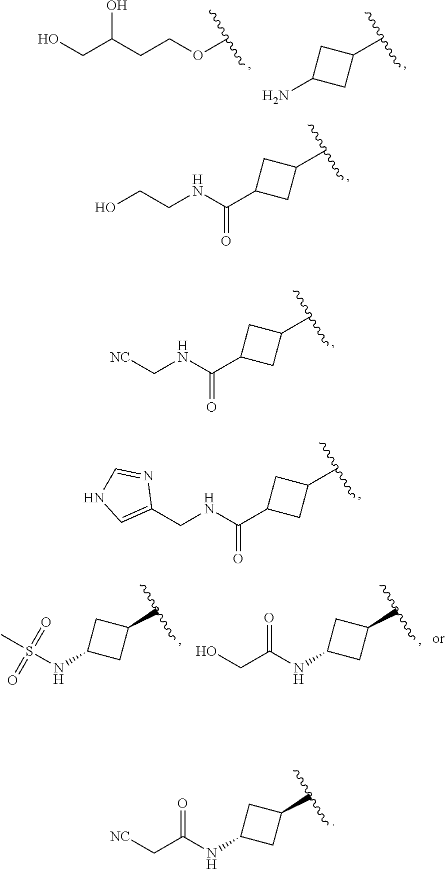

In some embodiments of a compound of Formula (I), (Ia), (Ib), (II), (IIa), (IIb), (IIIa), (IIIb), (IIIc), (IIId), (IV), (IVa), (IVb), (IVc), or (IVd), R⁹ is

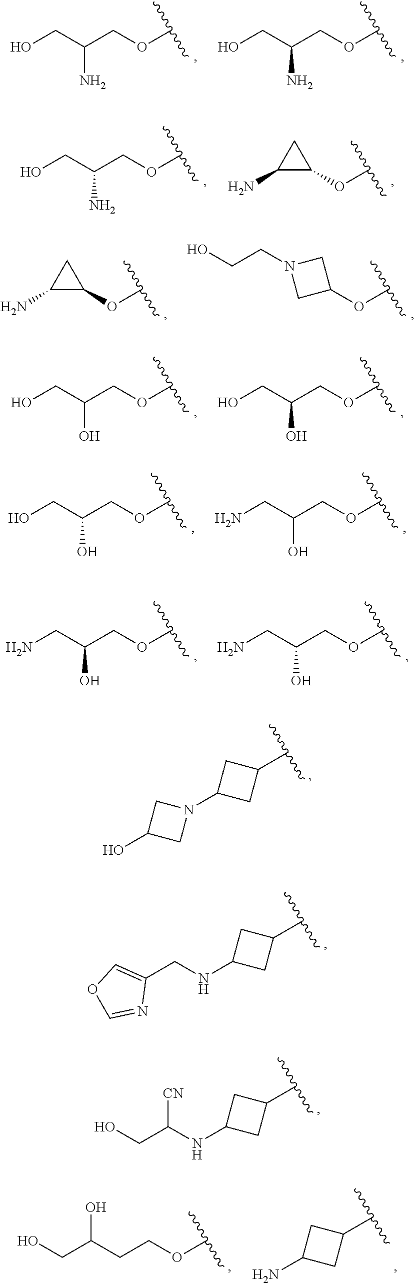

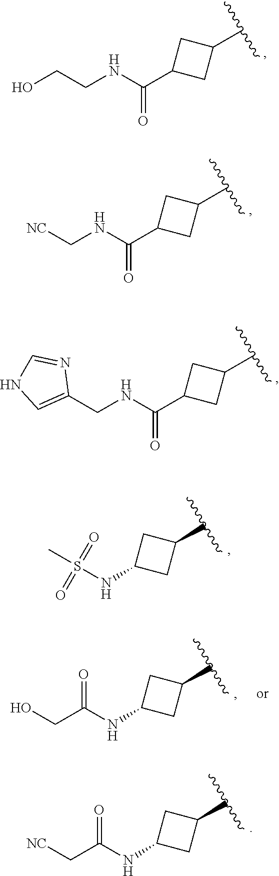

In some embodiments of a compound of Formula (I),
R¹ is —CH₃;
R^{2a} and R^{2b} are each hydrogen;
R³ is hydrogen or —(C₁-C₄ alkylene)-OH;
R⁴ is hydrogen;
R⁵ is hydrogen;
R⁶ is hydrogen or fluoro;
each R⁷ and R⁸ is independently hydrogen or fluoro; R⁹ is C₁-C₆ alkoxy, C₃-C₆ cycloalkyl, —O—(C₃-C₆ cycloalkyl), —O—(4- to 6-membered heterocycloalkyl), —O—(C₁-C₄ alkylene)-(C₃-C₆ cycloalkyl), —O—(C₁-C₄ alkylene)-(4- to 6-membered heterocycloalkyl), or —(C₁-C₄ alkylene)-O—(C₃-C₆ cycloalkyl); wherein the alkoxy, cycloalkyl, or heterocycloalkyl is unsubstituted or substituted by 1 or 2 groups independently selected from —OR¹⁰, —N(R¹⁰)₂, —CO₂R¹⁰, —CON(R¹⁰)₂, —CH₂N(R¹⁰)₂, —NHCOR¹⁰, —NHSO₂R¹⁰, —OCOR¹⁰, C₁-C₄ hydroxyalkyl, C₁-C₄ aminoalkyl, and 4- to 6-membered heterocycloalkyl which is unsubstituted or substituted by 1 —OH group;
each R¹⁰ is independently hydrogen, 4- to 6-membered heterocycloalkyl, or C₁-C₄ alkyl which is unsubstituted or substituted by 1, 2, or 3 groups independently selected from —F, —CN, —OH, —N(CH₃)₂, —CO₂H, —SO₂CH₃, oxazolidinone, and monocyclic heteroaryl which is unsubstituted or substituted by 1 —CONH₂ group;
or two R¹⁰ attached to the same nitrogen are taken together to form an azetidinyl which is unsubstituted or substituted by 1 —SO₂CH₃ group;
s is 1; and
t is 1.

In some embodiments of a compound of Formula (I),
R¹ is —CH₃;
R^{2a} and R^{2b} are each hydrogen;
R³ is hydrogen or —(C₁-C₄ alkylene)-OH;
R⁴ is hydrogen;
R⁵ is hydrogen;
R⁶ is hydrogen or fluoro;
each R⁷ and R⁸ is independently hydrogen or fluoro;
R⁹ is C₁-C₆ alkoxy, C₃-C₆ cycloalkyl, —O—(C₃-C₆ cycloalkyl), —O—(4- to 6-membered heterocycloalkyl), —O—(C₁-C₄ alkylene)-(C₃-C₆ cycloalkyl), —O—(C₁-C₄ alkylene)-(4- to 6-membered heterocycloalkyl), or —(C₁-C₄ alkylene)-O—(C₃-C₆ cycloalkyl); wherein the alkoxy, cycloalkyl, or heterocycloalkyl is unsubstituted or substituted by 1 or 2 groups independently selected from —OR¹⁰, —N(R¹⁰)₂, —CO₂R¹⁰, —CON(R¹⁰)₂, —CH₂N(R¹⁰)₂, —NHCOR¹⁰, —NHSO₂R¹⁰, C₁-C₄ hydroxyalkyl, C₁-C₄ aminoalkyl, and 4- to 6-membered heterocycloalkyl which is unsubstituted or substituted by 1 —OH group;
each R¹⁰ is independently hydrogen, 4- to 6-membered heterocycloalkyl, or C₁-C₄ alkyl which is unsubstituted or substituted by 1, 2, or 3 groups independently selected from —F, —CN, —OH, —N(CH₃)₂, —CO₂H, —SO₂CH₃, oxazolidinone, and monocyclic heteroaryl which is unsubstituted or substituted by 1 —CONH₂ group;
or two R¹⁰ attached to the same nitrogen are taken together to form an azetidinyl which is unsubstituted or substituted by 1 —SO₂CH₃ group;
s is 1; and
t is 1.

In some embodiments of a compound of Formula (I),
R¹ is —CH₃;
R^{2a} and R^{2b} are each hydrogen;
R³ is hydrogen or —(C₁-C₄ alkylene)-OH;
R⁴ is hydrogen;
R⁵ is hydrogen;
R⁶ is hydrogen or fluoro;
each R⁷ and R⁸ is independently hydrogen or fluoro;
R⁹ is C₁-C₄ alkoxy, C₃-C₄ cycloalkyl, —O—(C₃-C₄ cycloalkyl), —O—(4- to 6-membered heterocycloalkyl), —O—(C₁-C₃ alkylene)-(C₃-C₄ cycloalkyl), —O—(C₁-C₃ alkylene)-(4- to 6-membered heterocycloalkyl), or —(C₁-C₃ alkylene)-O—(C₃-C₄ cycloalkyl); wherein the alkoxy, cycloalkyl, or heterocycloalkyl is substituted by 1 or 2 groups independently selected from —OR¹⁰, —N(R¹⁰)₂, —CO₂R¹⁰, —CON(R¹⁰)₂, —CH₂N(R¹⁰)₂, —NHCOR¹⁰, —NHSO₂R¹⁰, —OCOR¹⁰, C₁-C₃ hydroxyalkyl, C₁-C₃ aminoalkyl, and 4- to 6-membered heterocycloalkyl which is unsubstituted or substituted by 1 —OH group;
each R¹⁰ is independently hydrogen or C₁-C₂ alkyl which is unsubstituted or substituted by 1, 2, or 3 groups independently selected from —F, —CN, —OH, —N(CH₃)₂, —CO₂H, —SO₂CH₃, oxazolidinone, and monocyclic 5-membered heteroaryl which is unsubstituted or substituted by 1 —CONH₂ group;
s is 1; and
t is 1.

In some embodiments of a compound of Formula (I),
R¹ is —CH₃;
R^{2a} and R^{2b} are each hydrogen;
R³ is hydrogen or —(C₁-C₄ alkylene)-OH;
R⁴ is hydrogen;
R⁵ is hydrogen;
R⁶ is hydrogen or fluoro;
each R⁷ and R⁸ is independently hydrogen or fluoro;
R⁹ is C₁-C₄ alkoxy, C₃-C₄ cycloalkyl, —O—(C₃-C₄ cycloalkyl), —O—(4- to 6-membered heterocycloalkyl), —O—(C₁-C₃ alkylene)-(C₃-C₄ cycloalkyl), —O—(C₁-C₃ alkylene)-(4- to 6-membered heterocycloalkyl), or —(C₁-C₃ alkylene)-O—(C₃-C₄ cycloalkyl); wherein the alkoxy, cycloalkyl, or heterocycloalkyl is substituted by 1 or 2 groups independently selected from —OR¹⁰, —N(R¹⁰)₂, —CO₂R¹⁰, —CON(R¹⁰)₂, —CH₂N(R¹⁰)₂, —NHCOR¹⁰, —NHSO₂R¹⁰, C₁-C₃ hydroxyalkyl, C₁-C₃ aminoalkyl, and 4- to 6-membered heterocycloalkyl which is unsubstituted or substituted by 1 —OH group;
each R¹⁰ is independently hydrogen or C₁-C₂ alkyl which is unsubstituted or substituted by 1, 2, or 3 groups independently selected from —F, —CN, —OH, —N(CH₃)₂, —CO₂H, —SO₂CH₃, oxazolidinone, and monocyclic 5-membered heteroaryl which is unsubstituted or substituted by 1 —CONH₂ group;
s is 1; and
t is 1.

In some embodiments of a compound of Formula (I),
R¹ is —CH₃;
R^{2a} and R^{2b} are each hydrogen;
R³ is —CH₂OH;
R⁴ is hydrogen;
R⁵ is hydrogen;
R⁶ is hydrogen or fluoro;
each R⁷ and R⁸ is independently hydrogen or fluoro;
R⁹ is C₁-C₄ alkoxy, C₃-C₄ cycloalkyl, —O—(C₃-C₄ cycloalkyl), or —O—(4- to 6-membered heterocycloalkyl); wherein the alkoxy, cycloalkyl, or heterocycloalkyl is substituted by 1 or 2 groups independently selected from —OH, —N(R¹⁰)₂, —CON(R¹⁰)₂, —NHCOR¹⁰, —NHSO₂R¹⁰, —CH₂OH, —CH₂CH₂OH, and azetidinyl which is substituted by 1 —OH group;
each R¹⁰ is independently hydrogen or C₁-C₂ alkyl which is unsubstituted or substituted by a —CN, —OH, oxazolyl, or imidazolyl group;
s is 1; and
t is 1.

In some embodiments of a compound of Formula (I),
R¹ is —CH₃;
R^{2a} and R^{2b} are each hydrogen;
R³ is —CH₂OH;
R⁴ is hydrogen;
R⁵ is hydrogen;
R⁶ is hydrogen or fluoro;
each R⁷ and R⁸ is independently hydrogen or fluoro;
R⁹ is C₁-C₄ alkoxy, C₃-C₄ cycloalkyl, —O—(C₃-C₄ cycloalkyl), or —O—(4- to 6-membered heterocycloalkyl); wherein the alkoxy, cycloalkyl, or heterocycloalkyl is substituted by 1 or 2 groups independently selected from —OH, —N(R¹⁰)₂, —CON(R¹⁰)₂, —NHCOR¹⁰, —NHSO₂R¹⁰, —CH₂CH₂OH, and azetidinyl which is substituted by 1 —OH group;
each R¹⁰ is independently hydrogen or C₁-C₂ alkyl which is unsubstituted or substituted by a —CN, —OH, oxazolyl, or imidazolyl group;
s is 1; and
t is 1.

In some embodiments, the compound of Formula (I) is a compound of Formula (V):

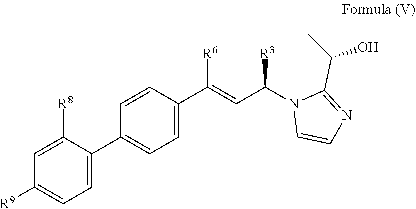

or a pharmaceutically acceptable salt, or solvate thereof. In some embodiments,
R³ is hydrogen or —(C₁-C₄ alkylene)-OH;
R⁶ is hydrogen or fluoro;
R⁸ is hydrogen or fluoro;
R⁹ is C₁-C₆ alkoxy, C₃-C₆ cycloalkyl, —O—(C₃-C₆ cycloalkyl), —O—(4- to 6-membered heterocycloalkyl), —O—(C₁-C₄ alkylene)-(C₃-C₆ cycloalkyl), —O—(C₁-C₄ alkylene)-(4- to 6-membered heterocycloalkyl), or —(C₁-C₄ alkylene)-O—(C₃-C₆ cycloalkyl); wherein the alkoxy, cycloalkyl, or heterocycloalkyl is unsubstituted or substituted by 1 or 2 groups independently selected from —OR¹⁰, —N(R¹⁰)₂, —CO₂R¹⁰, —CON(R¹⁰)₂, —CH₂N(R¹⁰)₂, —NHCOR¹⁰, —NHSO₂R¹⁰, —OCOR¹⁰, C₁-C₄ hydroxyalkyl, C₁-C₄ aminoalkyl, and 4- to 6-membered heterocycloalkyl which is unsubstituted or substituted by 1 —OH group; and
each R¹⁰ is independently hydrogen, 4- to 6-membered heterocycloalkyl, or C₁-C₄ alkyl which is unsubstituted or substituted by 1, 2, or 3 groups independently selected from —F, —CN, —OH, —N(CH₃)₂, —CO₂H, —SO₂CH₃, oxazolidinone, and monocyclic heteroaryl which is unsubstituted or substituted by 1 —CONH₂ group;
or two R¹⁰ attached to the same nitrogen are taken together to form an azetidinyl which is unsubstituted or substituted by 1 —SO₂CH₃ group.

In some embodiments, the compound of Formula (I) is a compound of Formula (V):

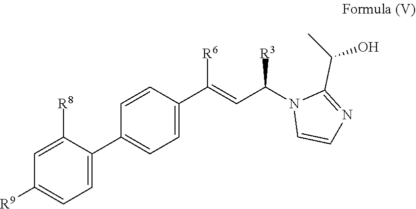

or a pharmaceutically acceptable salt, or solvate thereof. In some embodiments,
R³ is hydrogen or —(C₁-C₄ alkylene)-OH;
R⁶ is hydrogen or fluoro;
R⁸ is hydrogen or fluoro;
R⁹ is C₁-C₆ alkoxy, C₃-C₆ cycloalkyl, —O—(C₃-C₆ cycloalkyl), —O—(4- to 6-membered heterocycloalkyl), —O—(C₁-C₄ alkylene)-(C₃-C₆ cycloalkyl), —O—(C₁-C₄ alkylene)-(4- to 6-membered heterocycloalkyl), or —(C₁-C₄ alkylene)-O—(C₃-C₆ cycloalkyl); wherein the alkoxy, cycloalkyl, or heterocycloalkyl is unsubstituted or substituted by 1 or 2 groups independently selected from —OR¹⁰, —N(R¹⁰)₂, —CO₂R¹⁰, —CON(R¹⁰)₂, —CH₂N(R¹⁰)₂, —NHCOR¹⁰, —NHSO₂R¹⁰, C₁-C₄ hydroxyalkyl, C₁-C₄ aminoalkyl, and 4- to 6-membered heterocycloalkyl which is unsubstituted or substituted by 1 —OH group; and
each R¹⁰ is independently hydrogen, 4- to 6-membered heterocycloalkyl, or C₁-C₄ alkyl which is unsubstituted or substituted by 1, 2, or 3 groups independently selected from —F, —CN, —OH, —N(CH₃)₂, —CO₂H, —SO₂CH₃, oxazolidinone, and monocyclic heteroaryl which is unsubstituted or substituted by 1 —CONH₂ group;
or two R¹⁰ attached to the same nitrogen are taken together to form an azetidinyl which is unsubstituted or substituted by 1 —SO₂CH₃ group.

In some embodiments of a compound of Formula (V),
R⁶ is hydrogen or fluoro;
R⁸ is hydrogen or fluoro;
R⁹ is C₁-C₄ alkoxy, C₃-C₄ cycloalkyl, —O—(C₃-C₄ cycloalkyl), —O—(4- to 6-membered heterocycloalkyl), —O—(C₁-C₃ alkylene)-(C₃-C₄ cycloalkyl), —O—(C₁-C₃ alkylene)-(4- to 6-membered heterocycloalkyl), or —(C₁-C₃ alkylene)-O—(C₃-C₄ cycloalkyl); wherein the alkoxy, cycloalkyl, or heterocycloalkyl is substituted by 1 or 2 groups independently selected from —OR¹⁰, —N(R¹⁰)₂, —CO₂R¹⁰, —CON(R¹⁰)₂, —CH₂N(R¹⁰)₂, —NHCOR¹⁰, —NHSO₂R¹⁰, —OCOR¹⁰, C₁-C₃ hydroxyalkyl, C₁-C₃ aminoalkyl, and 4- to 6-membered heterocycloalkyl which is unsubstituted or substituted by 1 —OH group; and
each R¹⁰ is independently hydrogen or C₁-C₂ alkyl which is unsubstituted or substituted by 1, 2, or 3 groups independently selected from —F, —CN, —OH, —N(CH₃)₂, —CO₂H, —SO₂CH₃, oxazolidinone, and monocyclic 5-membered heteroaryl which is unsubstituted or substituted by 1 —CONH₂ group.

In some embodiments of a compound of Formula (V),
R⁶ is hydrogen or fluoro;
R⁸ is hydrogen or fluoro;
R⁹ is C₁-C₄ alkoxy, C₃-C₄ cycloalkyl, —O—(C₃-C₄ cycloalkyl), —O—(4- to 6-membered heterocycloalkyl), —O—(C₁-C₃ alkylene)-(C₃-C₄ cycloalkyl), —O—(C₁-C₃ alkylene)-(4- to 6-membered heterocycloalkyl), or —(C₁-C₃ alkylene)-O—(C₃-C₄ cycloalkyl); wherein the alkoxy, cycloalkyl, or heterocycloalkyl is substituted by 1 or 2 groups independently selected from —OR¹⁰, —N(R¹⁰)₂, —CO₂R¹⁰, —CON(R¹⁰)₂, —CH₂N(R¹⁰)₂, —NHCOR¹⁰, —NHSO₂R¹⁰, C₁-C₃ hydroxyalkyl, C₁-C₃ aminoalkyl, and 4- to 6-membered heterocycloalkyl which is unsubstituted or substituted by 1 —OH group; and
each R¹⁰ is independently hydrogen or C₁-C₂ alkyl which is unsubstituted or substituted by 1, 2, or 3 groups independently selected from —F, —CN, —OH, —N(CH₃)₂, —CO₂H, —SO₂CH₃, oxazolidinone, and monocyclic 5-membered heteroaryl which is unsubstituted or substituted by 1 —CONH₂ group.

In some embodiments of a compound of Formula (V),
R³ is —CH₂OH;
R⁶ is hydrogen or fluoro;
R⁸ is hydrogen;
R⁹ is C₁-C₄ alkoxy, C₃-C₄ cycloalkyl, —O—(C₃-C₄ cycloalkyl), or —O—(4- to 6-membered heterocycloalkyl); wherein the alkoxy, cycloalkyl, or heterocycloalkyl is substituted by 1 or 2 groups independently selected from —OH, —N(R¹⁰)₂, —CON(R¹⁰)₂, —NHCOR¹⁰, —NHSO₂R¹⁰, —CH₂OH, —CH₂CH₂OH, and azetidinyl which is substituted by 1 —OH group; and
each R¹⁰ is independently hydrogen or C₁-C₂ alkyl which is unsubstituted or substituted by a —CN, —OH, oxazolyl, or imidazolyl group.

In some embodiments of a compound of Formula (V),
R³ is —CH₂OH;
R⁶ is hydrogen or fluoro;
R⁸ is hydrogen;
R⁹ is C₁-C₄ alkoxy, C₃-C₄ cycloalkyl, —O—(C₃-C₄ cycloalkyl), or —O—(4- to 6-membered heterocycloalkyl); wherein the alkoxy, cycloalkyl, or heterocycloalkyl is substituted by 1 or 2 groups independently selected from —OH, —N(R¹⁰)₂, —CON(R¹⁰)₂, —NHCOR¹⁰, —NHSO₂R¹⁰, —CH₂CH₂OH, and azetidinyl which is substituted by 1 —OH group; and
each R¹⁰ is independently hydrogen or C₁-C₂ alkyl which is unsubstituted or substituted by a —CN, —OH, oxazolyl, or imidazolyl group.

In some embodiments, the compound of Formula (I) is a compound of Formula (VI):

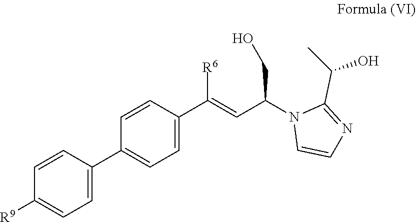

or a pharmaceutically acceptable salt, or solvate thereof. In some embodiments,
R⁶ is hydrogen or fluoro;
R⁹ is C₁-C₆ alkoxy, C₃-C₆ cycloalkyl, —O—(C₃-C₆ cycloalkyl), —O—(4- to 6-membered heterocycloalkyl), —O—(C₁-C₄ alkylene)-(C₃-C₆ cycloalkyl), —O—(C₁-C₄ alkylene)-(4- to 6-membered heterocycloalkyl), or —(C₁-C₄ alkylene)-O—(C₃-C₆ cycloalkyl); wherein the alkoxy, cycloalkyl, or heterocycloalkyl is unsubstituted or substituted by 1 or 2 groups independently selected from —OR¹⁰, —N(R¹⁰)₂, —CO₂R¹⁰, —CON(R¹⁰)₂, —CH₂N(R¹⁰)₂, —NHCOR¹⁰, —NHSO₂R¹⁰, —OCOR¹⁰, C₁-C₄ hydroxyalkyl, C₁-C₄ aminoalkyl, and 4- to 6-membered heterocycloalkyl which is unsubstituted or substituted by 1 —OH group; and
each R¹⁰ is independently hydrogen, 4- to 6-membered heterocycloalkyl, or C₁-C₄ alkyl which is unsubstituted or substituted by 1, 2, or 3 groups independently selected from —F, —CN, —OH, —N(CH₃)₂, —CO₂H, —SO₂CH₃, oxazolidinone, and monocyclic heteroaryl which is unsubstituted or substituted by 1 —CONH₂ group;
or two R¹⁰ attached to the same nitrogen are taken together to form an azetidinyl which is unsubstituted or substituted by 1 —SO₂CH₃ group.

In some embodiments, the compound of Formula (I) is a compound of Formula (VI):

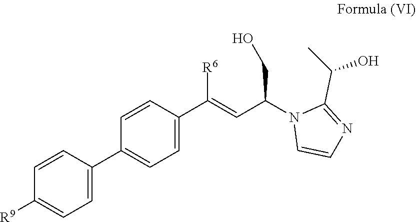

or a pharmaceutically acceptable salt, or solvate thereof. In some embodiments,
R⁶ is hydrogen or fluoro;
R⁹ is C₁-C₆ alkoxy, C₃-C₆ cycloalkyl, —O—(C₃-C₆ cycloalkyl), —O—(4- to 6-membered heterocycloalkyl), —O—(C₁-C₄ alkylene)-(C₃-C₆ cycloalkyl), —O—(C₁-C₄ alkylene)-(4- to 6-membered heterocycloalkyl), or —(C₁-C₄ alkylene)-O—(C₃-C₆ cycloalkyl); wherein the alkoxy, cycloalkyl, or heterocycloalkyl is unsubstituted or substituted by 1 or 2 groups independently selected from —OR¹⁰, —N(R¹⁰)₂, —CO₂R¹⁰, —CON(R¹⁰)₂, —CH₂N(R¹⁰)₂, —NHCOR¹⁰, —NHSO₂R¹⁰, C₁-C₄ hydroxyalkyl, C₁-C₄ aminoalkyl, and 4- to 6-membered heterocycloalkyl which is unsubstituted or substituted by 1 —OH group; and
each R¹⁰ is independently hydrogen, 4- to 6-membered heterocycloalkyl, or C₁-C₄ alkyl which is unsubstituted or substituted by 1, 2, or 3 groups independently selected from —F, —CN, —OH, —N(CH₃)₂, —CO₂H, —SO₂CH₃, oxazolidinone, and monocyclic heteroaryl which is unsubstituted or substituted by 1 —CONH₂ group;
or two R¹⁰ attached to the same nitrogen are taken together to form an azetidinyl which is unsubstituted or substituted by 1 —SO₂CH₃ group.

In some embodiments of a compound of Formula (VI),
R⁹ is C₁-C₄ alkoxy, C₃-C₄ cycloalkyl, —O—(C₃-C₄ cycloalkyl), —O—(4- to 6-membered heterocycloalkyl), —O—(C₁-C₃ alkylene)-(C₃-C₄ cycloalkyl), —O—(C₁-C₃ alkylene)-(4- to 6-membered heterocycloalkyl), or —(C₁-C₃ alkylene)-O—(C₃-C₄ cycloalkyl); wherein the alkoxy, cycloalkyl, or heterocycloalkyl is substituted by 1 or 2 groups independently selected from —OR¹⁰, —N(R¹⁰)₂, —CO₂R¹⁰, —CON(R¹⁰)₂, —CH₂N(R¹⁰)₂, —NHCOR¹⁰, —NHSO₂R¹⁰, —OCOR¹⁰, C₁-C₃ hydroxyalkyl, C₁-C₃ aminoalkyl, and 4- to 6-membered heterocycloalkyl which is unsubstituted or substituted by 1 —OH group; and
each R¹⁰ is independently hydrogen or C₁-C₂ alkyl which is unsubstituted or substituted by 1, 2, or 3 groups independently selected from —F, —CN, —OH, —N(CH₃)₂, —CO₂H, —SO₂CH₃, oxazolidinone, and monocyclic 5-membered heteroaryl which is unsubstituted or substituted by 1 —CONH₂ group.

In some embodiments of a compound of Formula (VI),
R⁹ is C₁-C₄ alkoxy, C₃-C₄ cycloalkyl, —O—(C₃-C₄ cycloalkyl), —O—(4- to 6-membered heterocycloalkyl), —O—(C₁-C₃ alkylene)-(C₃-C₄ cycloalkyl), —O—(C₁-C₃ alkylene)-(4- to 6-membered heterocycloalkyl), or —(C₁-C₃ alkylene)-O—(C₃-C₄ cycloalkyl); wherein the alkoxy, cycloalkyl, or heterocycloalkyl is substituted by 1 or 2 groups independently selected from —OR¹⁰, —N(R¹⁰)₂, —CO₂R¹⁰, —CON(R¹⁰)₂, —CH₂N(R¹⁰)₂, —NHCOR¹⁰, —NHSO₂R¹⁰, C₁-C₃ hydroxyalkyl, C₁-C₃ aminoalkyl, and 4- to 6-membered heterocycloalkyl which is unsubstituted or substituted by 1 —OH group; and
each R¹⁰ is independently hydrogen or C₁-C₂ alkyl which is unsubstituted or substituted by 1, 2, or 3 groups independently selected from —F, —CN, —OH, —N(CH₃)₂, —CO₂H, —SO₂CH₃, oxazolidinone, and monocyclic 5-membered heteroaryl which is unsubstituted or substituted by 1 —CONH₂ group.

In some embodiments of a compound of Formula (VI),
R⁹ is C₁-C₄ alkoxy, C₃-C₄ cycloalkyl, —O—(C₃-C₄ cycloalkyl), or —O—(4- to 6-membered heterocycloalkyl); wherein the alkoxy, cycloalkyl, or heterocycloalkyl is substituted by 1 or 2 groups independently selected from —OH, —N(R¹⁰)₂, —CON(R¹⁰)₂, —NHCOR¹⁰, —NHSO₂R¹⁰, —CH₂OH, —CH₂CH₂OH, and azetidinyl which is substituted by 1 —OH group; and
each R¹⁰ is independently hydrogen or C₁-C₂ alkyl which is unsubstituted or substituted by a —CN, —OH, oxazolyl, or imidazolyl group.

In some embodiments of a compound of Formula (VI),
R⁹ is C₁-C₄ alkoxy, C₃-C₄ cycloalkyl, —O—(C₃-C₄ cycloalkyl), or —O—(4- to 6-membered heterocycloalkyl); wherein the alkoxy, cycloalkyl, or heterocycloalkyl is substituted by 1 or 2 groups independently selected from —OH, —N(R¹⁰)₂, —CON(R¹⁰)₂, —NHCOR¹⁰, —NHSO₂R¹⁰, —CH₂CH₂OH, and azetidinyl which is substituted by 1 —OH group; and
each R¹⁰ is independently hydrogen or C₁-C₂ alkyl which is unsubstituted or substituted by a —CN, —OH, oxazolyl, or imidazolyl group.

In some embodiments of a compound of Formula (V) or (VI), R⁹ is

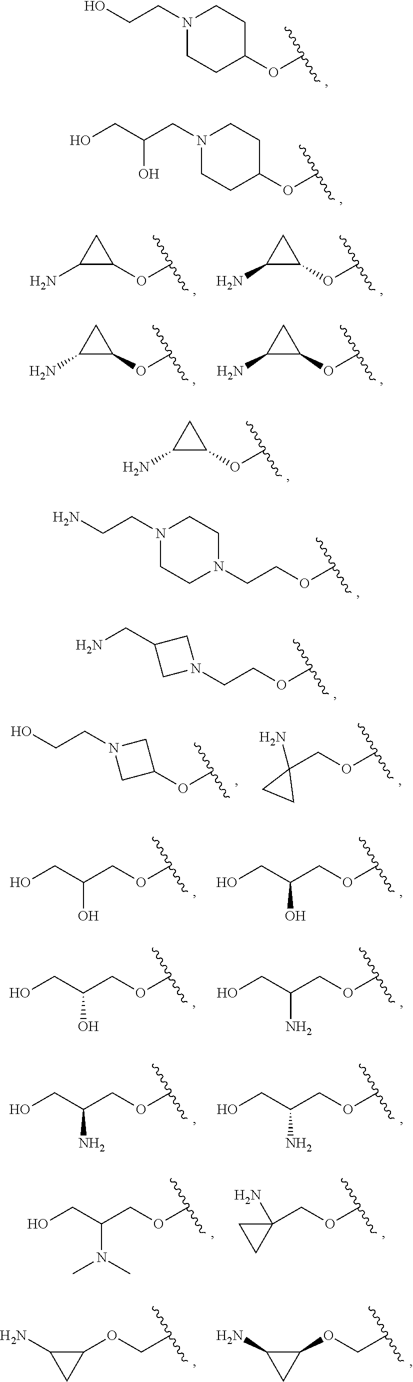

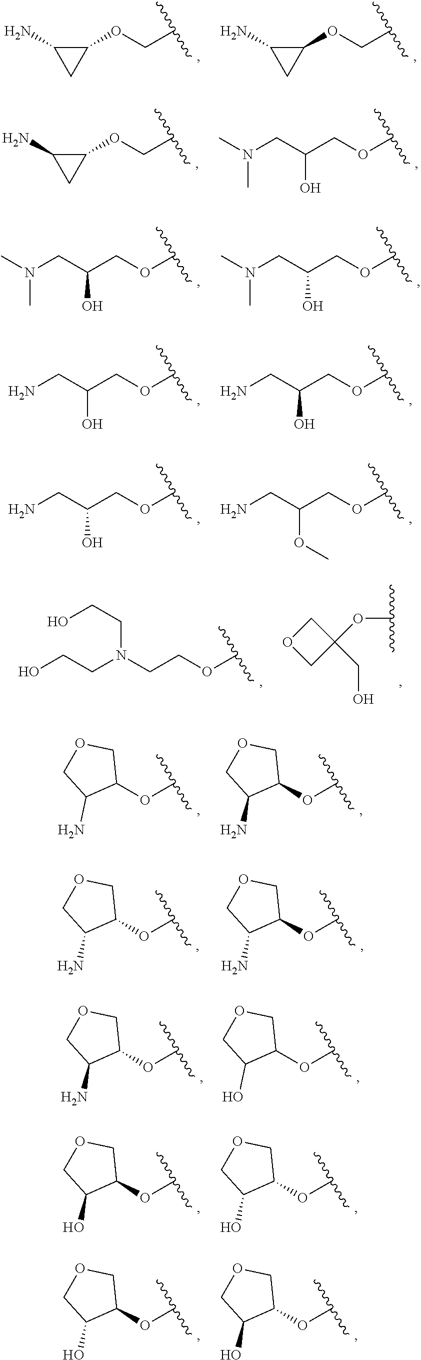

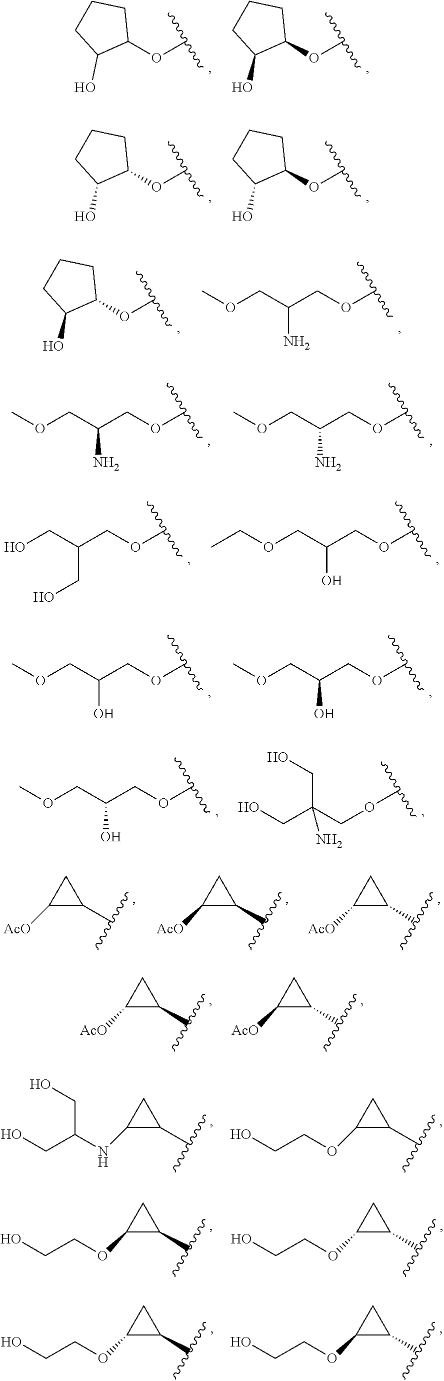

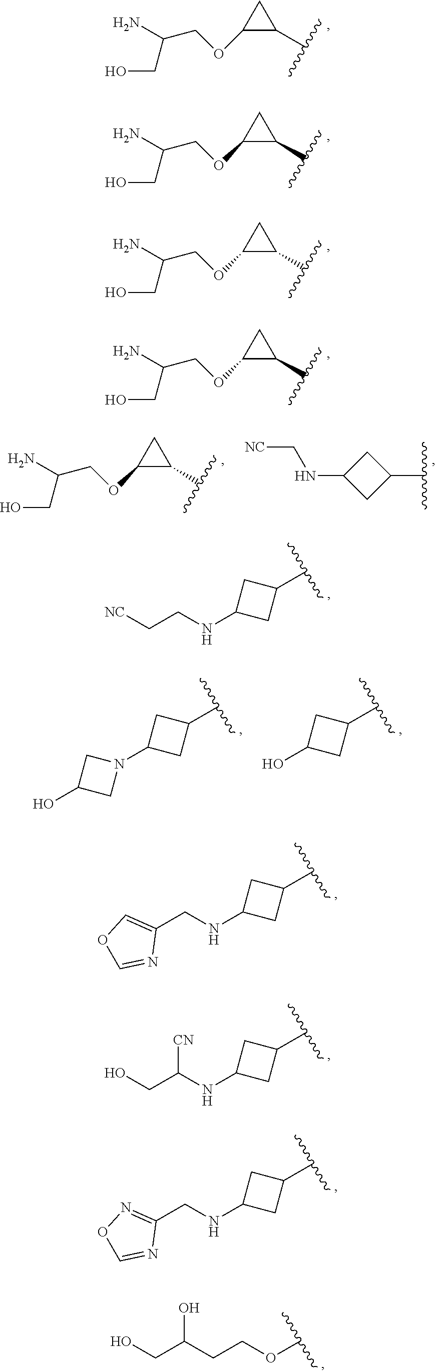

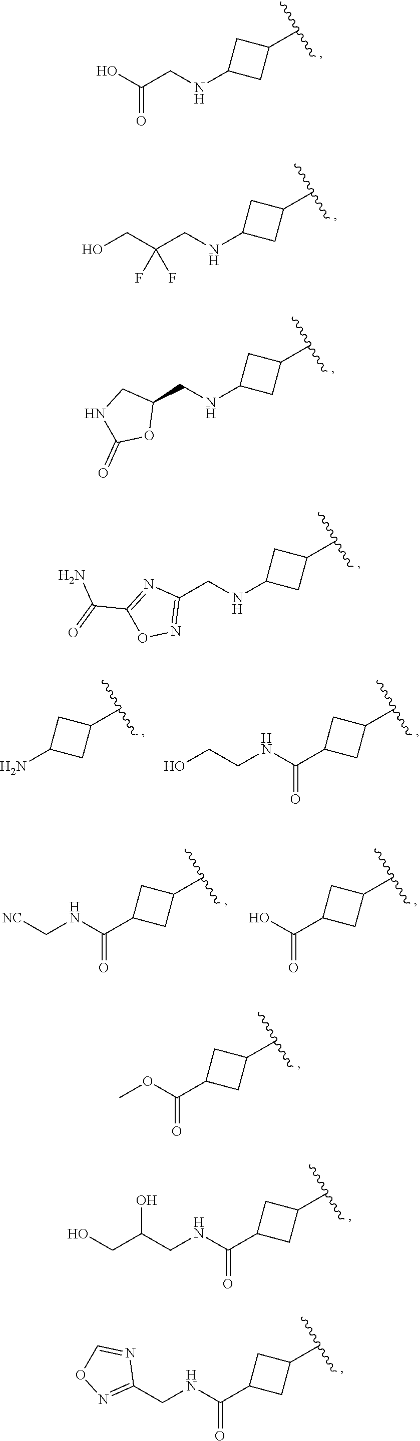

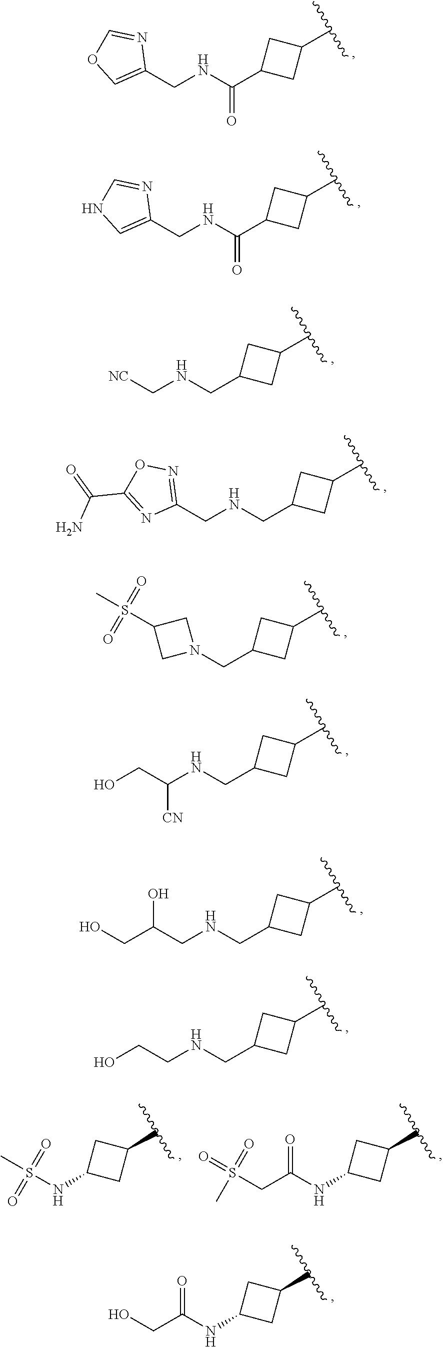

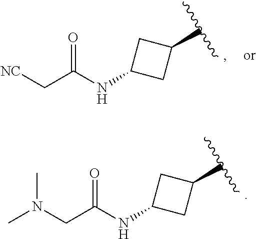 [SMILES: *[C@H]1C[C@H](NC(=O)CC#N)C1 ; *[C@H]1C[C@H](NC(=O)CN(C)C)C1]

In some embodiments of a compound of Formula (V) or (VI), R⁹ is

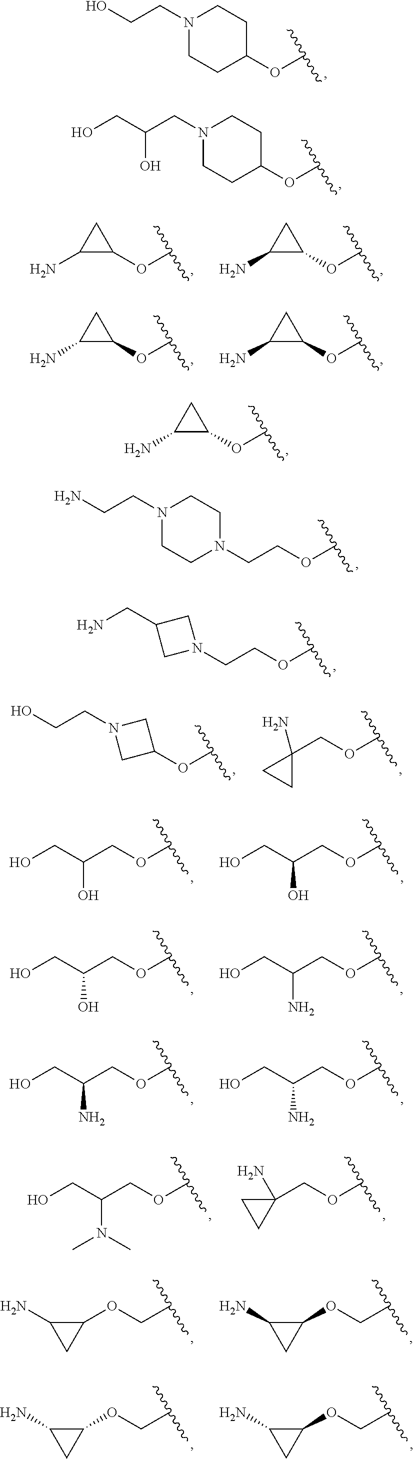

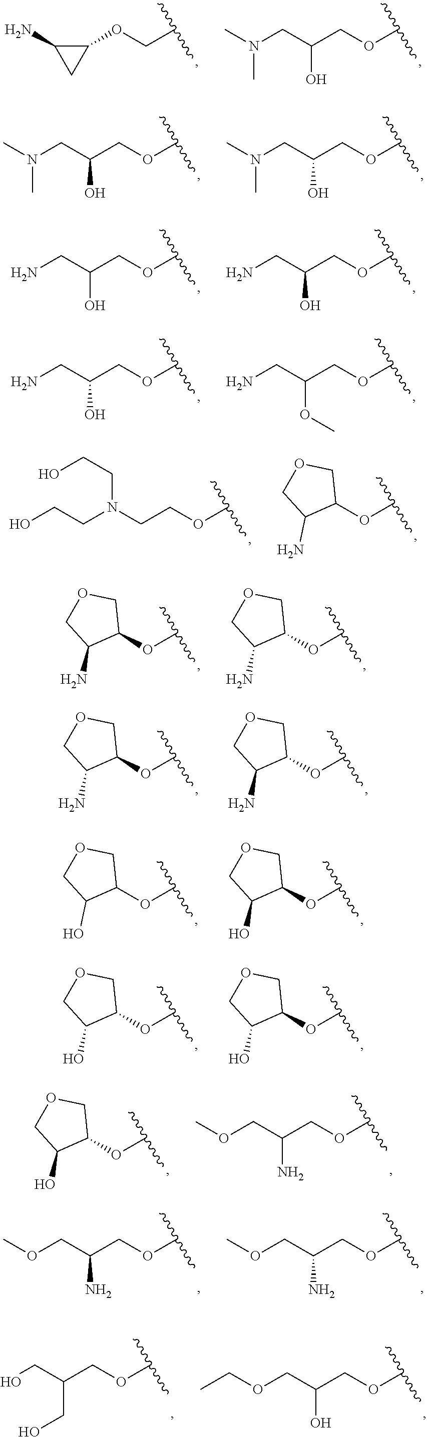

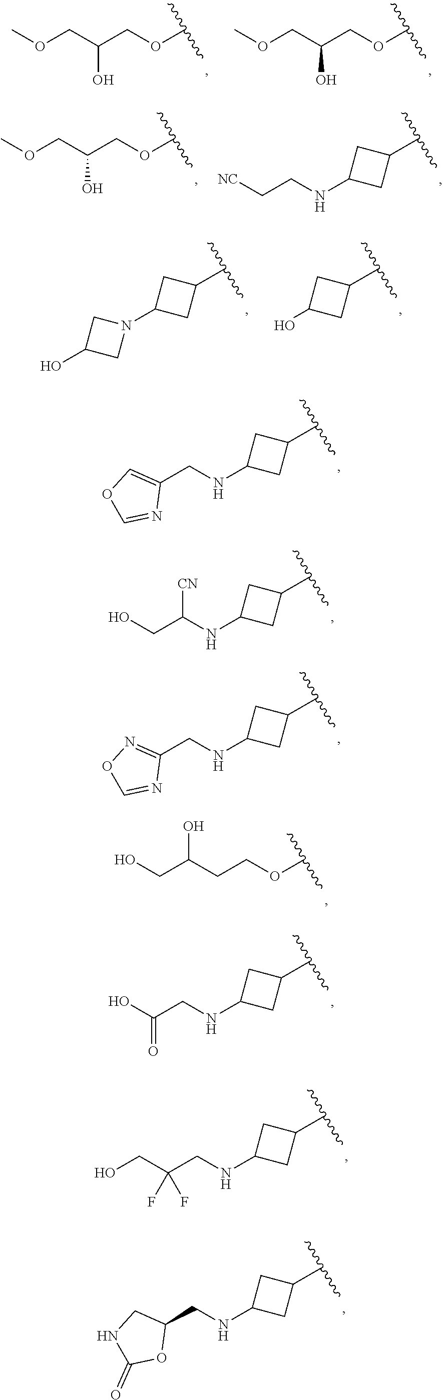

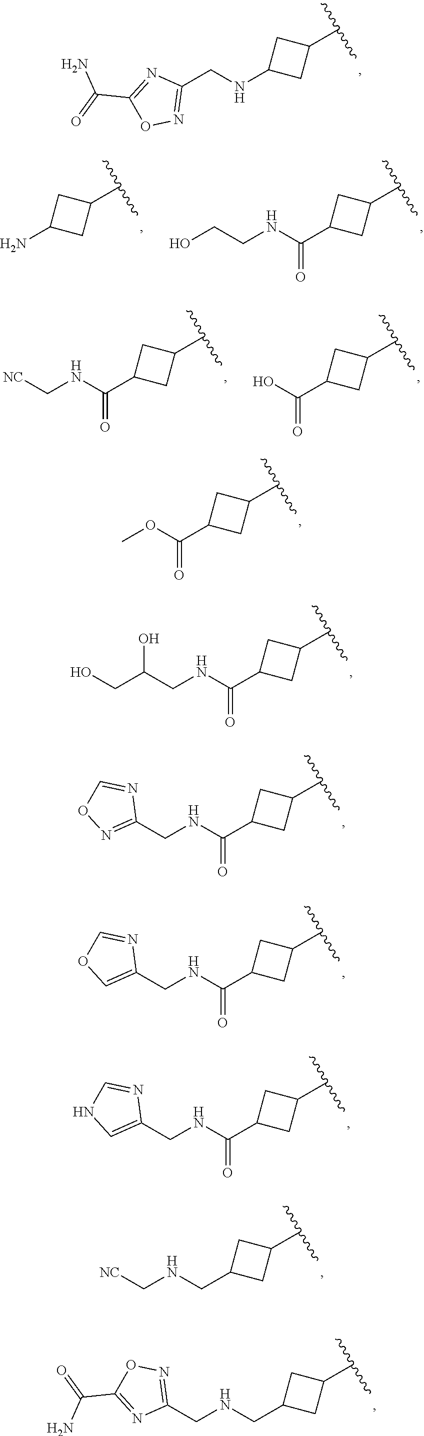

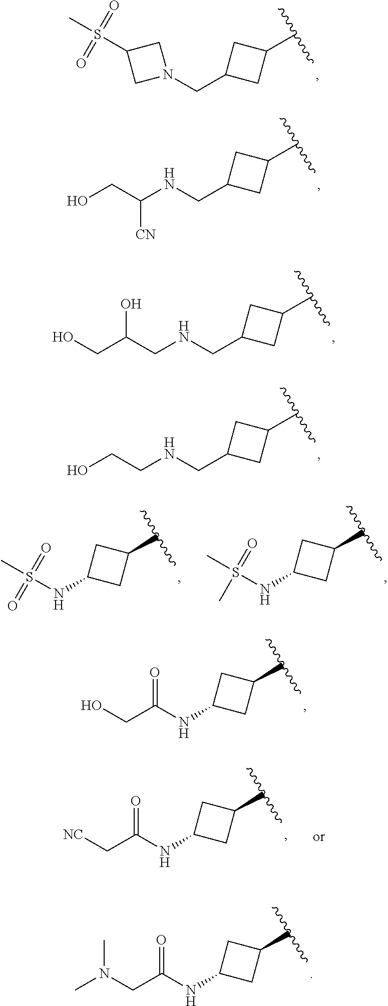

In some embodiments of a compound of Formula (V) or (VI), R⁹ is

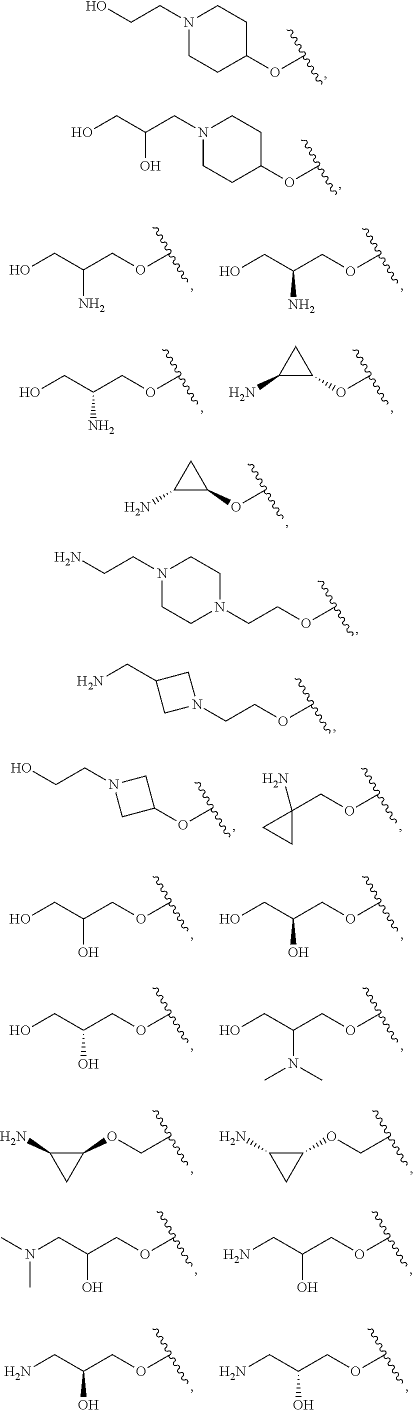

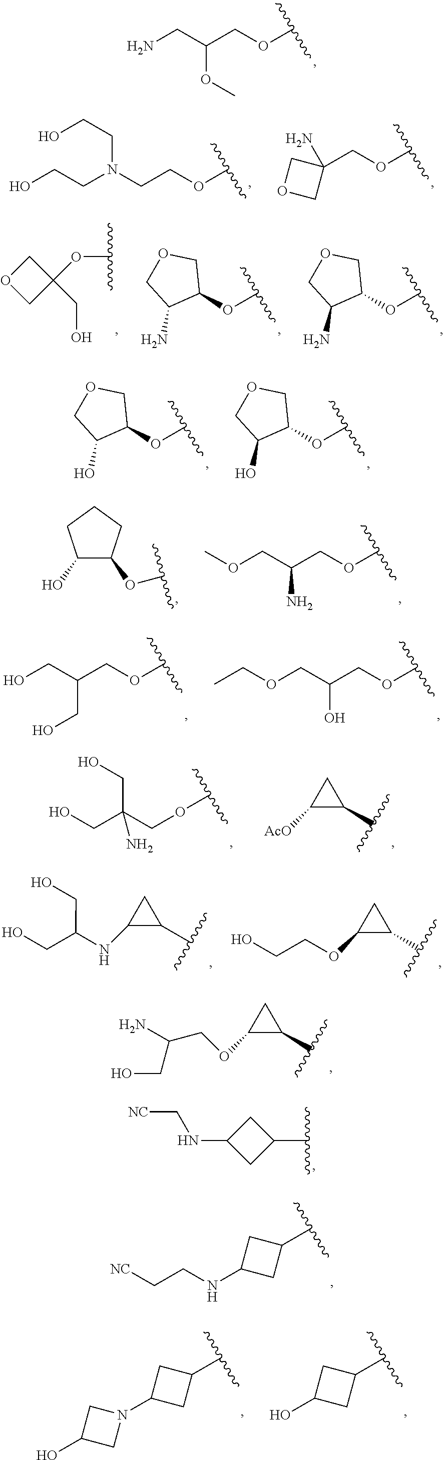

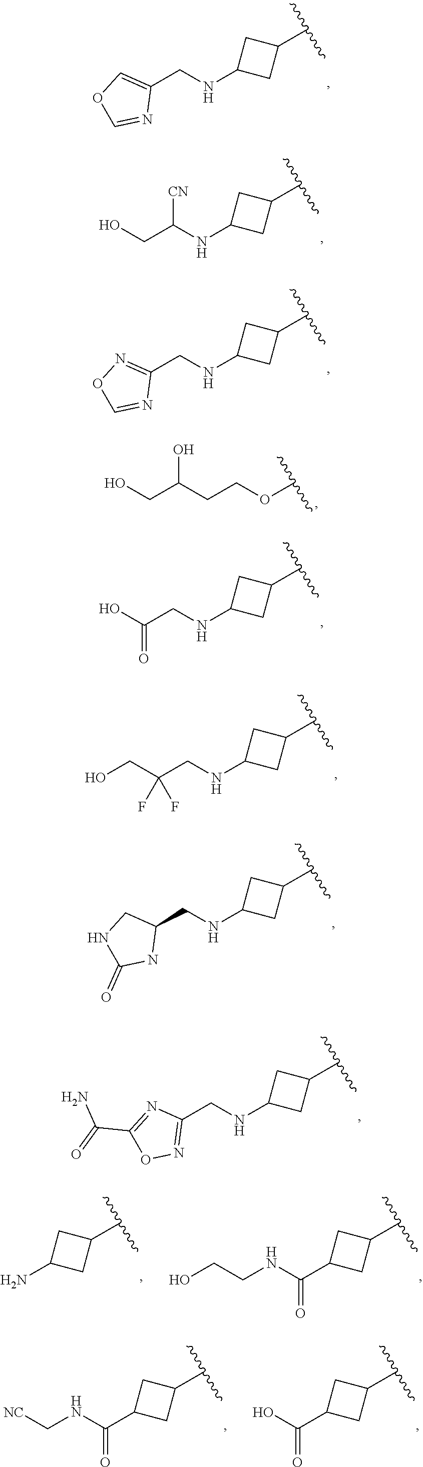

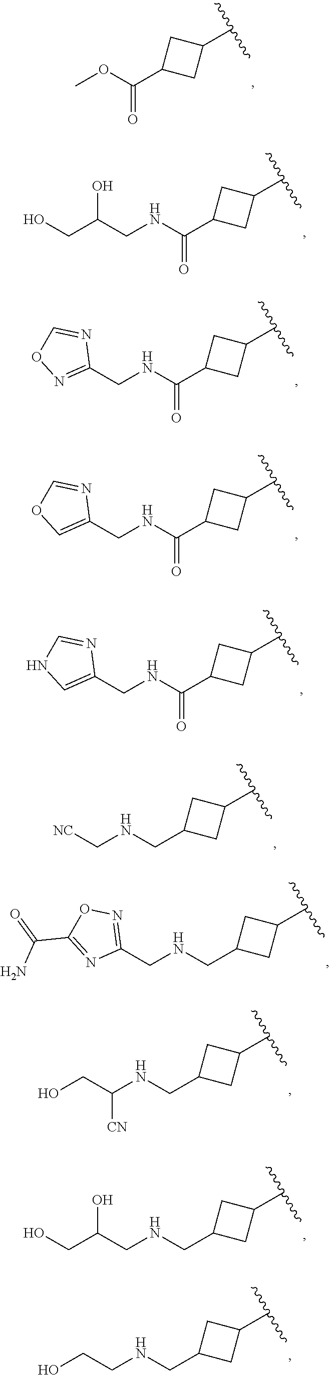

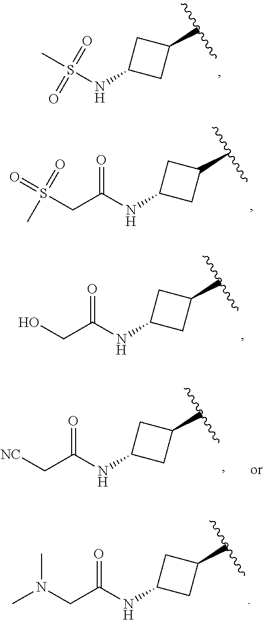

In some embodiments of a compound of Formula V or VI R⁹ is

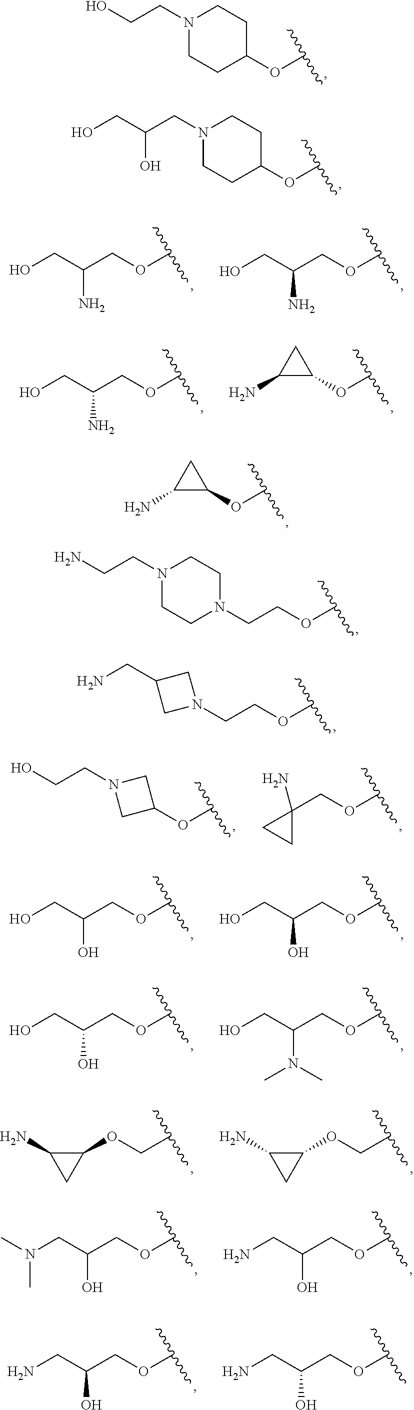

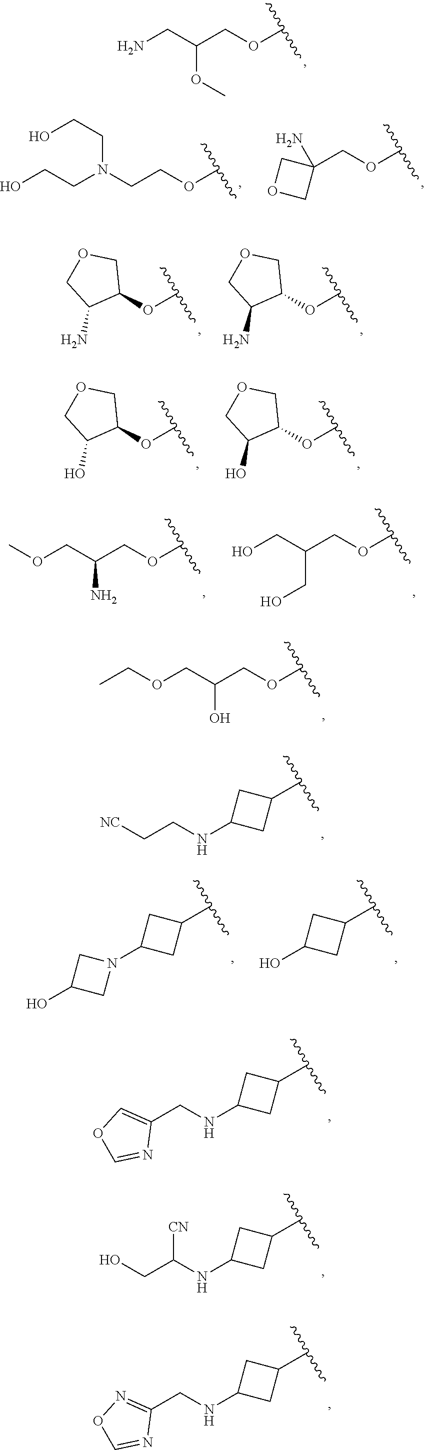

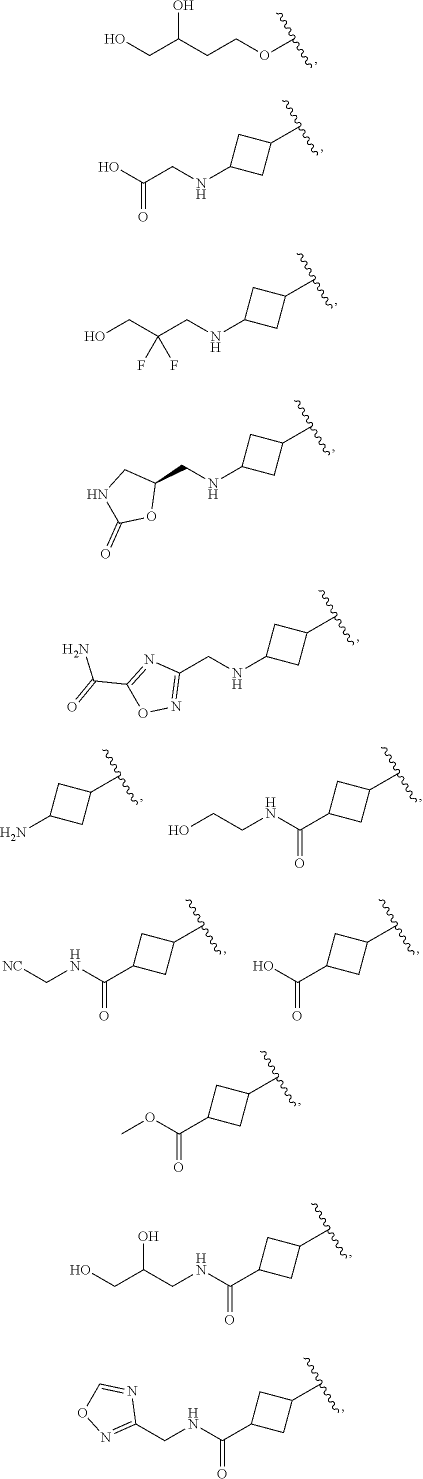

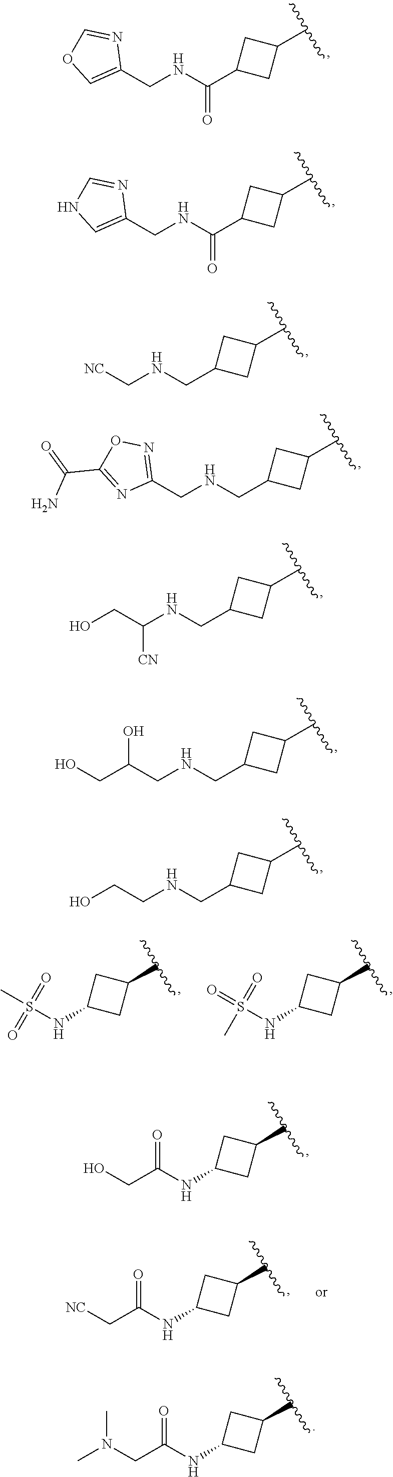

In some embodiments of a compound Formula (V) or (VI), R⁹ is

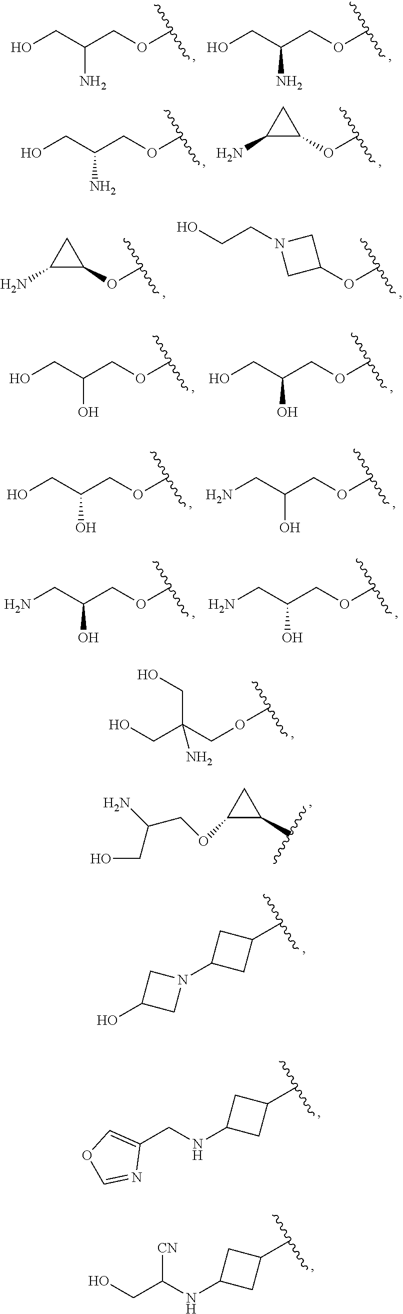

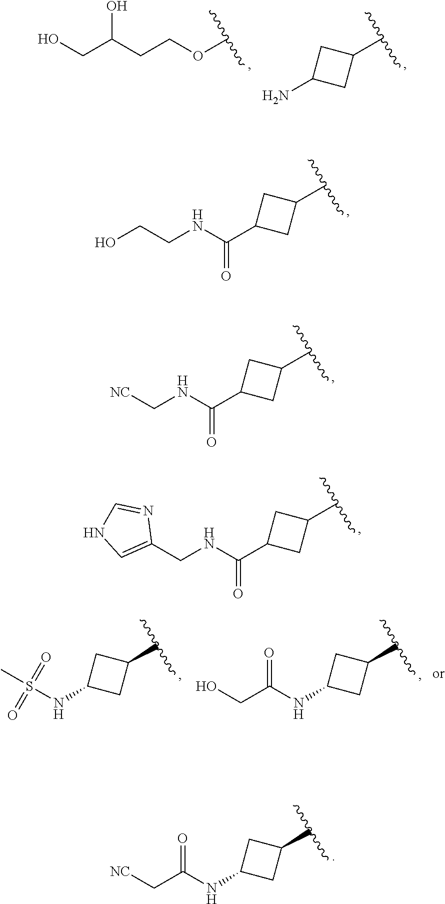

In some embodiments of a compound Formula (V) or (VI), R⁹ is

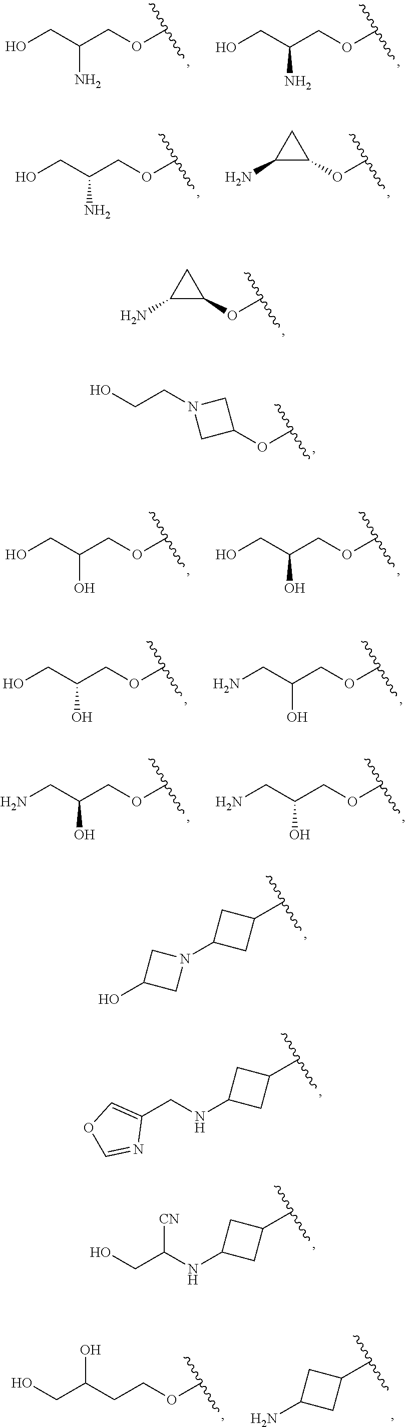

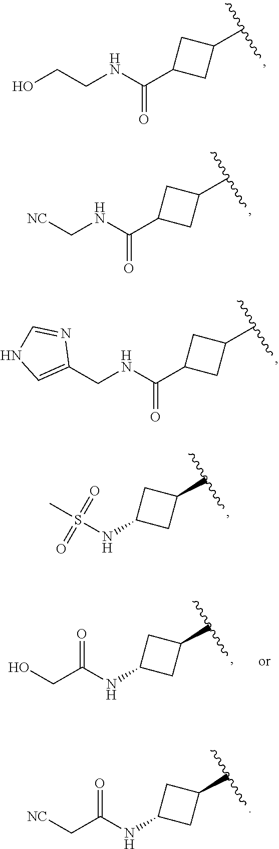

In some embodiments, compounds described herein have the following structure:

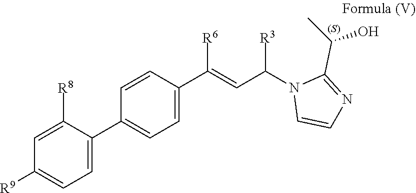

wherein R³, R⁶, R⁸, and R⁹ are as described in Table 1.

In some embodiments, R³ is hydrogen, CH₂OH, or CH₂CH₂OH.

In some embodiments, R⁶ is hydrogen or F.

In some embodiments, R⁸ is hydrogen or F.

In some embodiments, R⁹ is

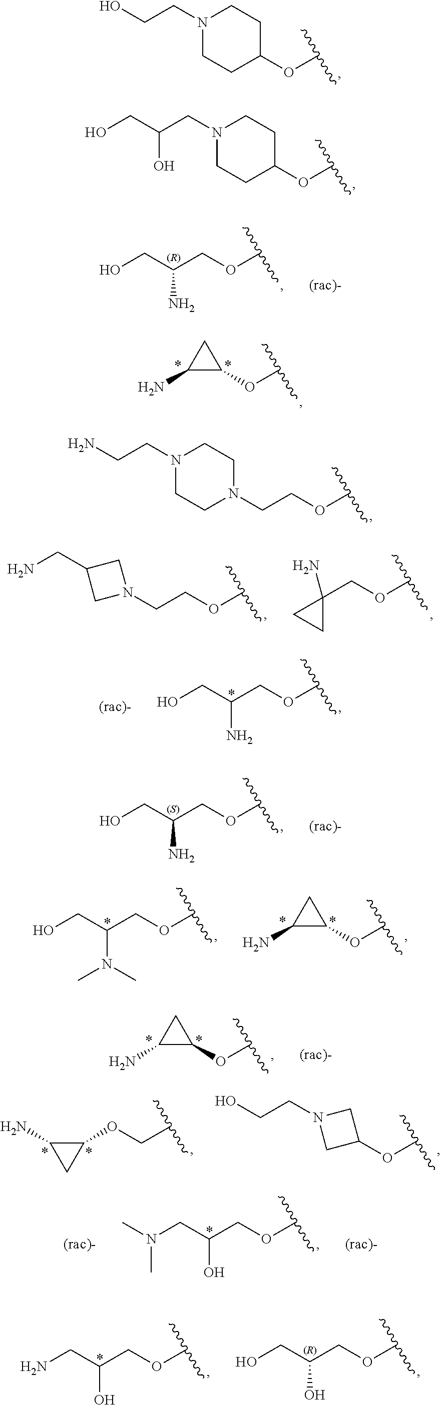

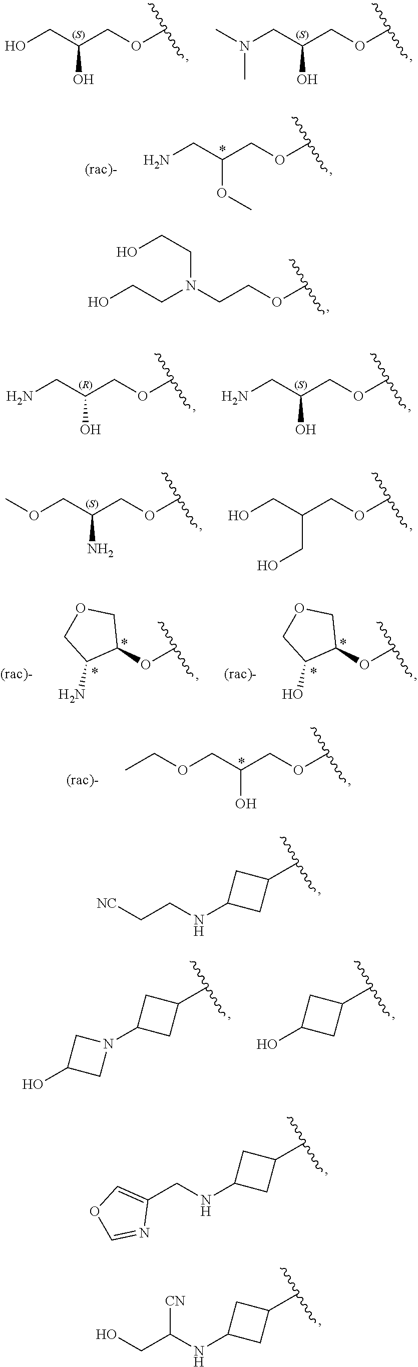

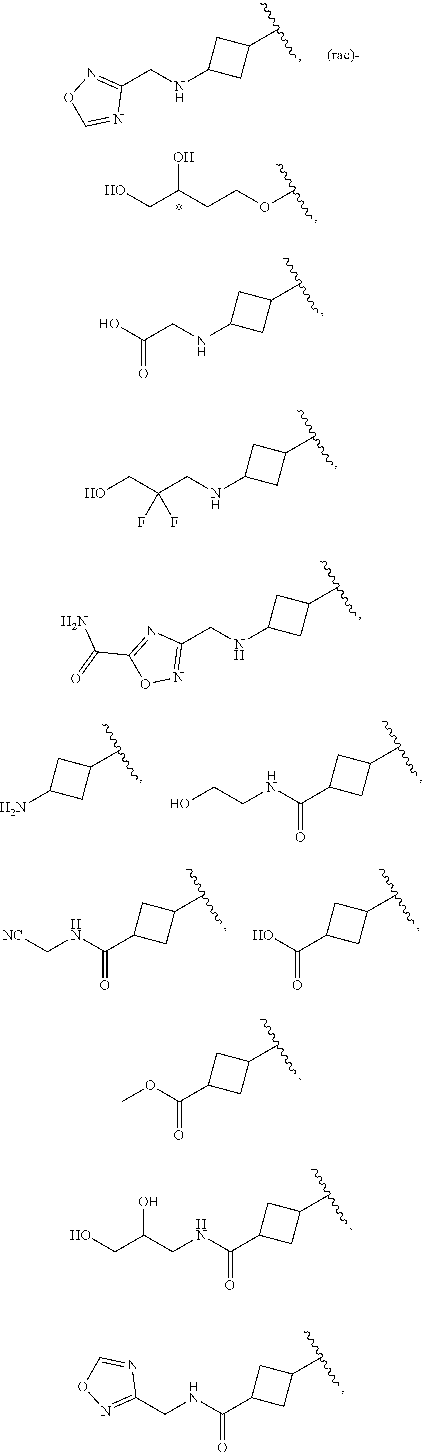

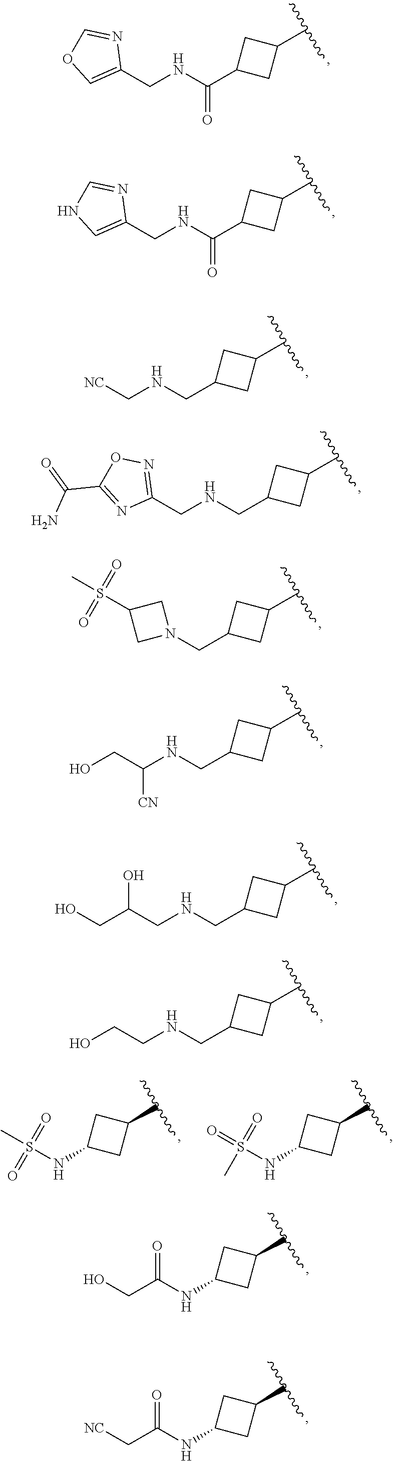

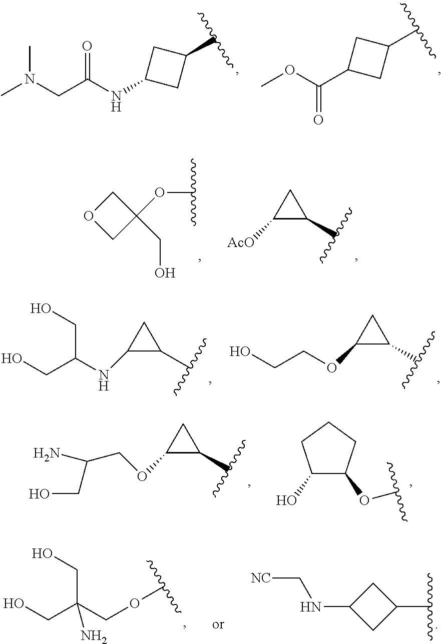

In some embodiments R⁹ is

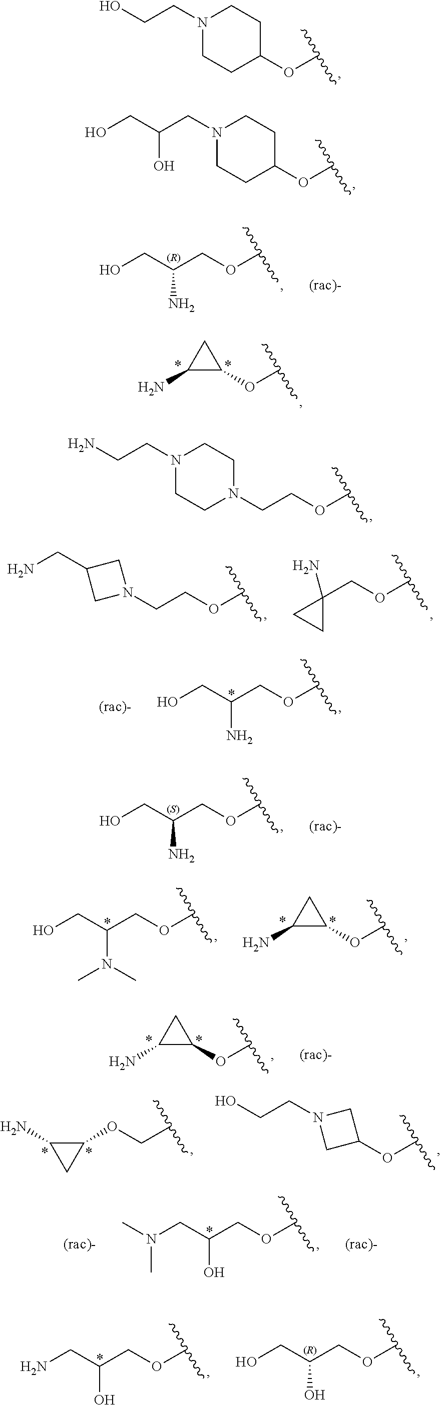

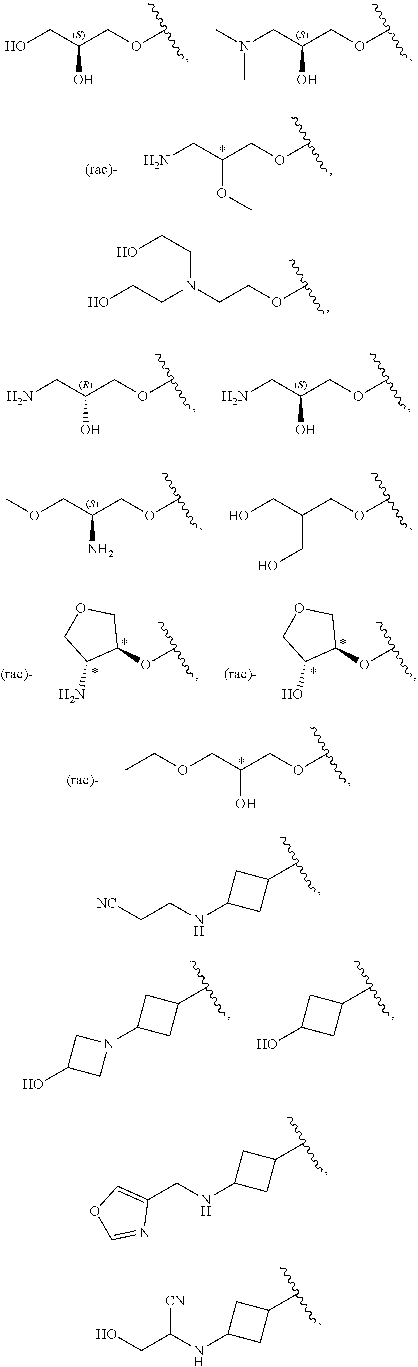

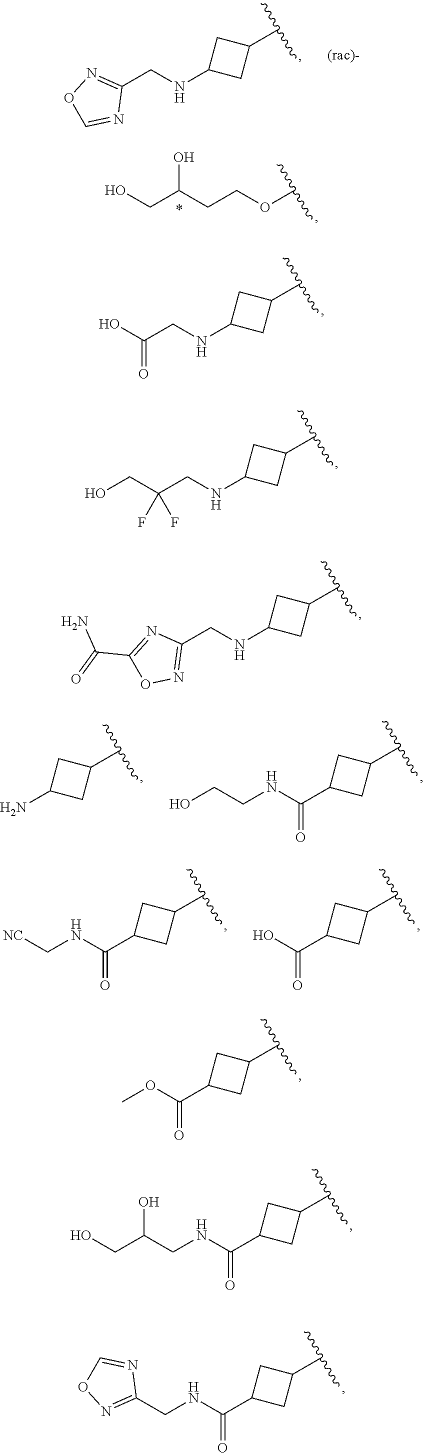

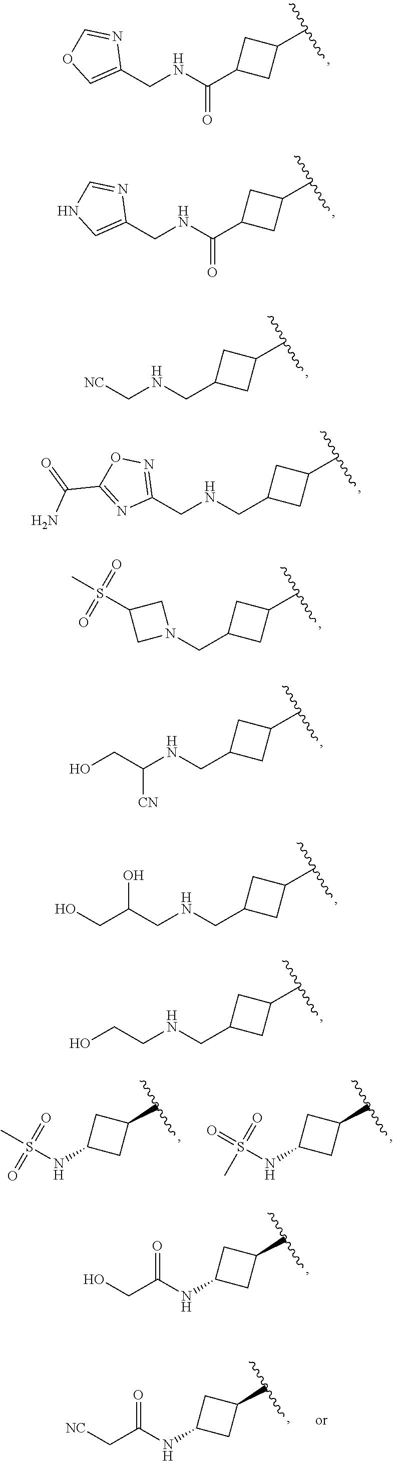

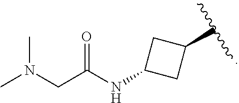 [SMILES: *[C@H]1C[C@H](NC(=O)CN(C)C)C1]

Any combination of the groups described above for the various variables is contemplated herein. Throughout the specification, groups and substituents thereof are chosen by one skilled in the field to provide stable moieties and compounds.

Exemplary compounds described herein include the compounds described in the following Tables:

TABLE 1

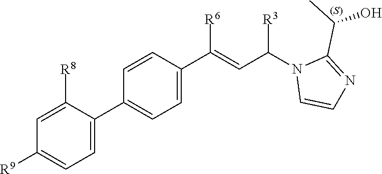

| Compound No. | R⁹ | R⁸ | R⁶ | 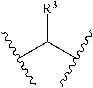 |
|---|---|---|---|---|
| 1 | 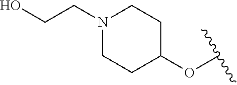 [SMILES: *OC1CCN(CCO)CC1] | H | H | 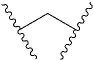 [SMILES: *C*] |
| 2 | 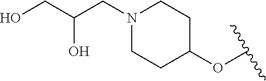 [SMILES: *OC1CCN(CC(O)CO)CC1] | H | H | 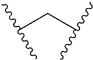 [SMILES: *C*] |
| 3 | 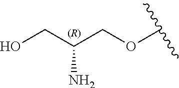 | H | H | 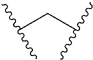 [SMILES: *C*] |
| 4 | 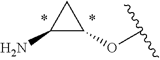 [SMILES: *O[C@H]1C[C@@H]1N] | H | H | 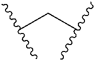 [SMILES: *C*] |
| | *racemic-trans | | | |
| 5 | 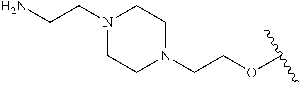 [SMILES: *OCCN1CCN(CCN)CC1] | H | H | 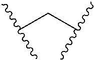 [SMILES: *C*] |
| 6 | 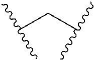 [SMILES: *C*] | H | H | 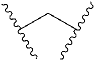 [SMILES: *C*] |
| 7 | 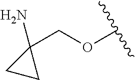 [SMILES: *OCC1(N)CC1] | H | H | 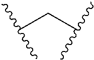 [SMILES: *C*] |
| 8 | 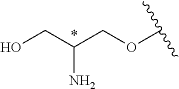 [SMILES: *OCC(N)CO] | H | H | 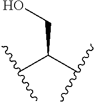 |
| | * racemic | | | |
| 9 | 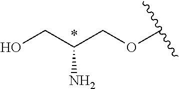 [SMILES: *OC[C@H](N)CO] | H | H | 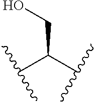 |
| | * single isomer, absolute | | | |
| | stereochemistry unknown | | | |
| 10 | 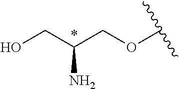 [SMILES: *OC[C@@H](N)CO] | H | H | 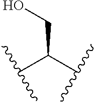 |
| | * single isomer, absolute | | | |
| | stereochemistry unknown | | | |
| 11 | 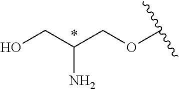 [SMILES: *OCC(N)CO] | H | F | 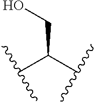 |
| | * racemic | | | |
| 12 | 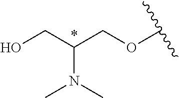 [SMILES: *OCC(CO)N(C)C] | H | H | 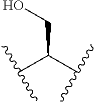 |
| | * racemic | | | |
| 13 | 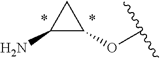 [SMILES: *O[C@H]1C[C@@H]1N] * racemic-trans | H | H | 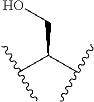 |
| 14 | 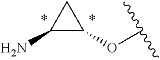 [SMILES: *O[C@H]1C[C@@H]1N] * single isomer, absolute stereochemistry unknown | H | H | 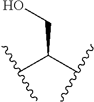 |
| 15 | 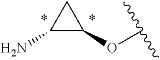 [SMILES: *O[C@@H]1C[C@H]1N] * single isomer, absolute stereochemistry unknown | H | H | 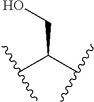 |
| 16 | 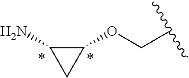 [SMILES: *CO[C@@H]1C[C@@H]1N] *racemic-cis | H | H | 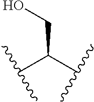 |
| 17 | 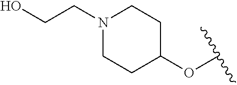 [SMILES: *OC1CCN(CCO)CC1] | H | H | 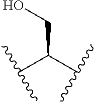 |
| 18 | 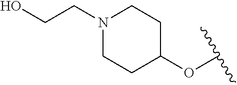 [SMILES: *OC1CCN(CCO)CC1] | H | H | 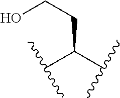 |
| 19 | 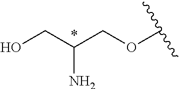 [SMILES: *OCC(N)CO] * racemic | H | H | 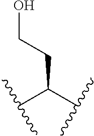 |
| 20 | 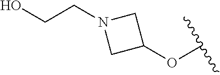 [SMILES: *OC1CN(CCO)C1] | H | H | 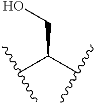 |
| 21 | 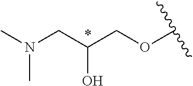 [SMILES: *OCC(O)CN(C)C] | H | H | 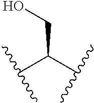 |
| | * racemic | | | |
| 22 | 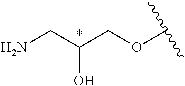 [SMILES: *OCC(O)CN] | H | H | 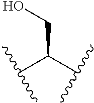 |
| | * racemic | | | |
| 23 | 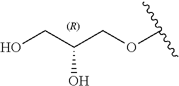 | H | H | 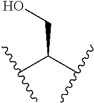 |
| 24 | 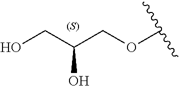 | H | H | 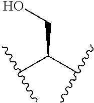 |
| 25 | 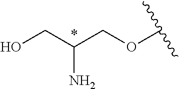 [SMILES: *OCC(N)CO] | H | H | 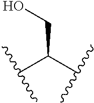 |
| | racemic | | | |
| 26 | 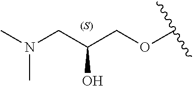 | H | H | 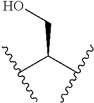 |
| 27 | 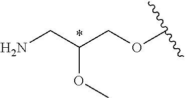 [SMILES: *OCC(CN)OC] | H | H | 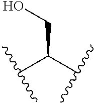 |
| | racemic | | | |
| 28 | 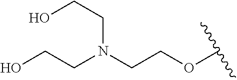 [SMILES: *OCCN(CCO)CCO] | H | H | 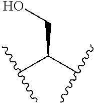 |
| 29 | 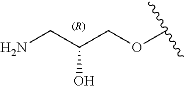 | H | H | 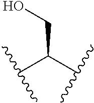 |
| 30 | 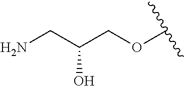 [SMILES: *OC[C@H](O)CN] | H | F | 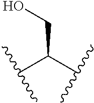 |
| | * single isomer, absolute | | | |
| | stereochemistry unknown | | | |
| 31 | 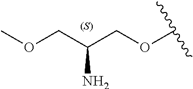 | H | H | 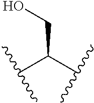 |
| 32 | 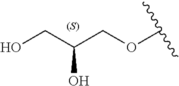 | F | H | 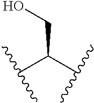 |
| 33 | 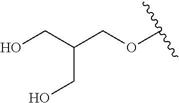 [SMILES: *OCC(CO)CO] | H | H | 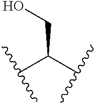 |
| 34 | 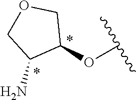 [SMILES: *O[C@@H]1COC[C@H]1N] | H | H | 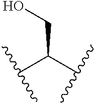 |
| | *racemic-trans | | | |
| 35 | 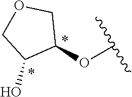 [SMILES: *O[C@@H]1COC[C@H]1O] | H | H | 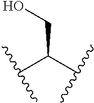 |
| | *racemic-trans | | | |
| 36 | 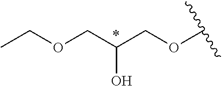 [SMILES: *OCC(O)COCC] | H | H | 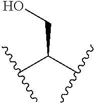 |
| | * racemic | | | |
| 37 | 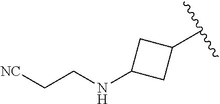 [SMILES: *C1CC(NCCC#N)C1] | H | H | 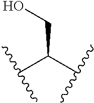 |
| 38 | 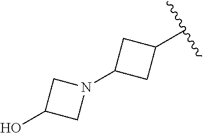 [SMILES: *C1CC(N2CC(O)C2)C1] | H | H | 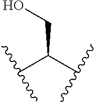 |
| 39 | 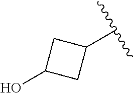 [SMILES: *C1CC(O)C1] | H | H | 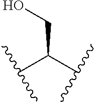 |
| 40 | 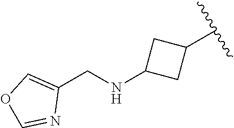 [SMILES: *C1CC(NCc2cocn2)C1] | H | H | 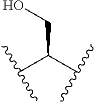 |
| 41 | 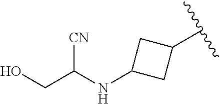 [SMILES: *C1CC(NC(C#N)CO)C1] | H | H | 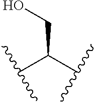 |
| 42 | 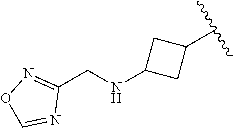 [SMILES: *C1CC(NCc2ncon2)C1] | H | H | 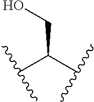 |
| 43 | 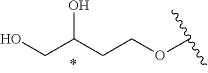 [SMILES: *OCCC(O)CO] * racemic | H | H | 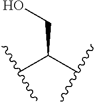 |
| 44 | 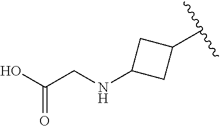 [SMILES: *C1CC(NCC(=O)O)C1] | H | H | 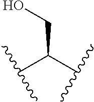 |
| 45 | 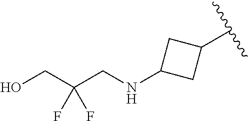 [SMILES: *C1CC(NCC(F)(F)CO)C1] | H | H | 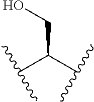 |
| 46 | 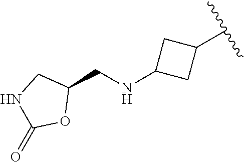 [SMILES: *C1CC(NC[C@@H]2CNC(=O)O2)C1] | H | H | 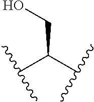 |
| 47 | 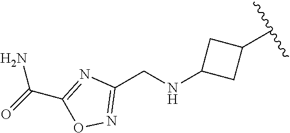 [SMILES: *C1CC(NCc2noc(C(N)=O)n2)C1] | H | H | 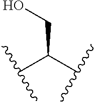 |
| 48 | 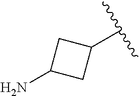 [SMILES: *C1CC(N)C1] | H | H | 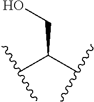 |
| 49 | 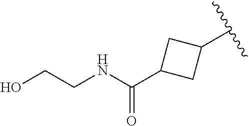 [SMILES: *C1CC(C(=O)NCCO)C1] | H | H | 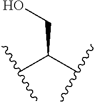 |
| 50 | 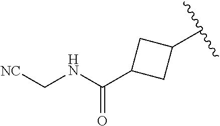 [SMILES: *C1CC(C(=O)NCC#N)C1] | H | H | 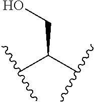 |
| 51 | 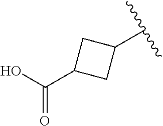 [SMILES: *C1CC(C(=O)O)C1] | H | H | 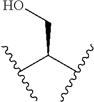 |
| 52 | 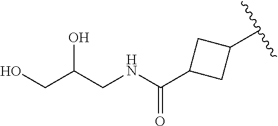 [SMILES: *C1CC(C(=O)NCC(O)CO)C1] | H | H | 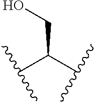 |
| 53 | 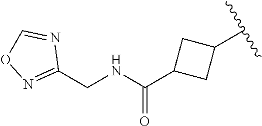 [SMILES: *C1CC(C(=O)NCc2ncon2)C1] | H | H | 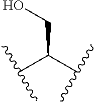 |
| 54 | 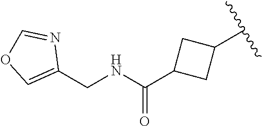 [SMILES: *C1CC(C(=O)NCc2cocn2)C1] | H | H | 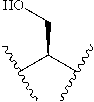 |
| 55 | 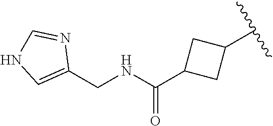 [SMILES: *C1CC(C(=O)NCc2c[nH]cn2)C1] | H | H | 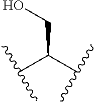 |
| 56 | 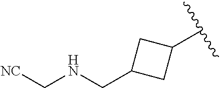 [SMILES: *C1CC(CNCC#N)C1] | H | H | 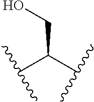 |
| 57 | 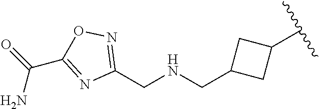 [SMILES: *C1CC(CNCc2noc(C(N)=O)n2)C1] | H | H | 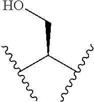 |
| 58 | 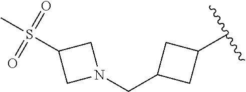 [SMILES: *C1CC(CN2CC(S(C)(=O)=O)C2)C1] | H | H | 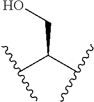 |
| 59 | 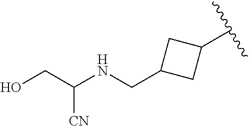 [SMILES: *C1CC(CNC(C#N)CO)C1] | H | H | 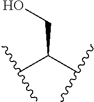 |
| 60 | 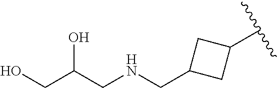 [SMILES: *C1CC(CNCC(O)CO)C1] | H | H | 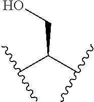 |
| 61 | 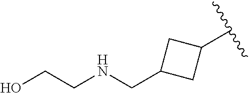 [SMILES: *C1CC(CNCCO)C1] | H | H | 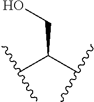 |
| 62 | 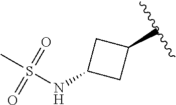 [SMILES: *[C@H]1C[C@H](NS(C)(=O)=O)C1] | H | H | 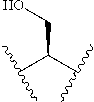 |
| 63 | 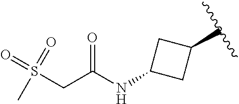 [SMILES: *[C@H]1C[C@H](NC(=O)CS(C)(=O)=O)C1] | H | H | 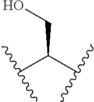 |
| 64 | 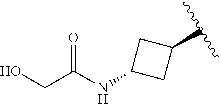 [SMILES: *[C@H]1C[C@H](NC(=O)CO)C1] | H | H | 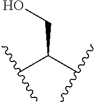 |
| 65 | 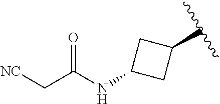 [SMILES: *[C@H]1C[C@H](NC(=O)CC#N)C1] | H | H | 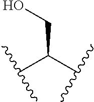 |
| 66 | 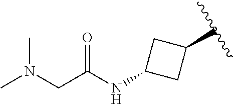 [SMILES: *[C@H]1C[C@H](NC(=O)CN(C)C)C1] | H | H | 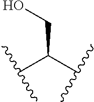 |
| 67 | 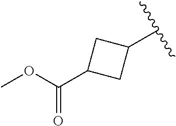 [SMILES: *C1CC(C(=O)OC)C1] | H | H | 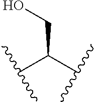 |
| 68 | 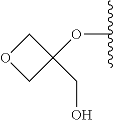 [SMILES: *OC1(CO)COC1] | H | H | 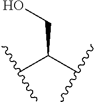 |
| 69 | 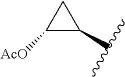 [SMILES: *[C@@H]1C[C@H]1OC(C)=O] | H | H | 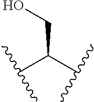 |
| 70 | 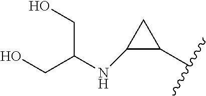 [SMILES: *C1CC1NC(CO)CO] | H | H | 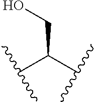 |
| 71 | 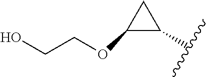 [SMILES: *[C@H]1C[C@@H]1OCCO] | H | H | 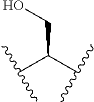 |
| 72 | 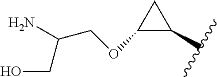 [SMILES: *[C@@H]1C[C@H]1OCC(N)CO] | H | H | 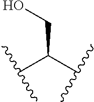 |
| 73 | 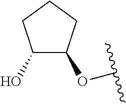 [SMILES: *O[C@@H]1CCC[C@H]1O] | H | H | 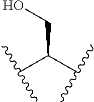 |
| 74 | 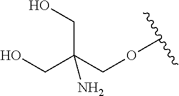 [SMILES: *OCC(N)(CO)CO] | H | H | 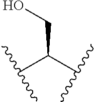 |
| 75 | 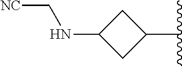 [SMILES: *C1CC(NCC#N)C1] | F | H | 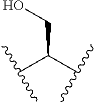 |

In some embodiments, the compound is a compound of Table 1, or a pharmaceutically acceptable salt, or solvate thereof. In some embodiments, the compound is a diastereomer of a compound of Table 1, or a pharmaceutically acceptable salt, or solvate thereof.

Structures and names of the compounds of Table 1 are found in Table 1a:

TABLE 1a

| Cmpd | | |
|---|---|---|
| No. | Structure | Name |
| 1 | 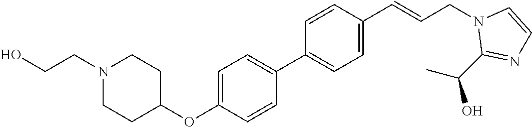 [SMILES: C[C@H](O)c1nccn1C/C=C/c1ccc(-c2ccc(OC3CCN(CCO)CC3)cc2)cc1] | (S,E)-2-(4-((4′-(3-(2-(1- hydroxyethyl)-1H-imidazol-1- yl)prop-1-en-1-yl)-[1,1′-biphenyl]-4- yl)oxy)piperidin-1-yl)ethan-1-ol |
| 2 | 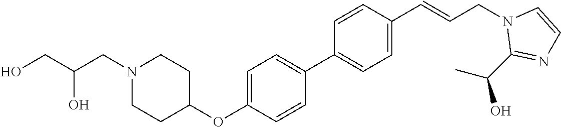 [SMILES: C[C@H](O)c1nccn1C/C=C/c1ccc(-c2ccc(OC3CCN(CC(O)CO)CC3)cc2)cc1] | 3-(4-((4′-((E)-3-(2-((S)-1- hydroxyethyl)-1H-imidazol-1- yl)prop-1-en-1-yl)-[1,1′-biphenyl]-4- yl)oxy)piperidin-1-yl)propane-1,2- diol |
| 3 | 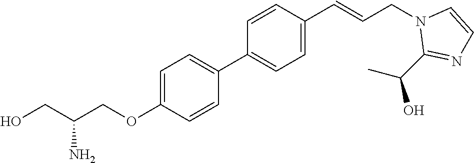 [SMILES: C[C@H](O)c1nccn1C/C=C/c1ccc(-c2ccc(OC[C@H](N)CO)cc2)cc1] | (R)-2-amino-3-((4′-((E)-3-(2-((S)-1- hydroxyethyl)-1H-imidazol-1- yl)prop-1-en-1-yl)-[1,1′-biphenyl]-4- yl)oxy)propan-1-ol |
| 4 | 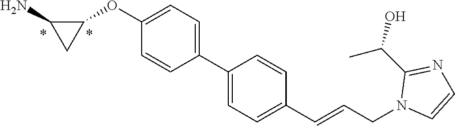 [SMILES: C[C@H](O)c1nccn1C/C=C/c1ccc(-c2ccc(O[C@@H]3C[C@H]3N)cc2)cc1] | (S)-1-(1-((E)-3-(4′-((trans)-2- aminocyclopropoxy)-[1,1′- biphenyl]-4-yl)allyl)-1H-imidazol-2- yl)ethan-1-ol |
| | *racemic-trans | |
| 5 | 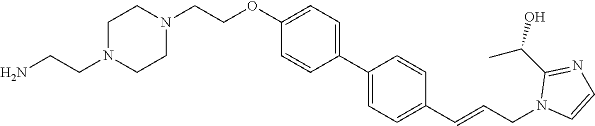 [SMILES: C[C@H](O)c1nccn1C/C=C/c1ccc(-c2ccc(OCCN3CCN(CCN)CC3)cc2)cc1] | (S,E)-1-(1-(3-(4′-(2-(4-(2- aminoethyl)piperazin-1-yl)ethoxy)- [1,1′-biphenyl]-4-yl)allyl)-1H- imidazol-2-yl)ethan-1-ol |
| 6 | 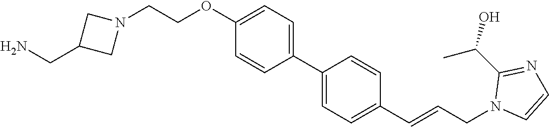 [SMILES: C[C@H](O)c1nccn1C/C=C/c1ccc(-c2ccc(OCCN3CC(CN)C3)cc2)cc1] | (S,E)-1-(1-(3-(4′-(2-(3- (aminomethyl)azetidin-1-yl)ethoxy)- [1,1′-biphenyl]-4-yl)allyl)-1H- imidazol-2-yl)ethan-1-ol |
| 7 | 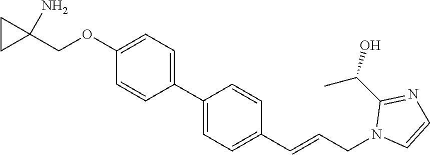 [SMILES: C[C@H](O)c1nccn1C/C=C/c1ccc(-c2ccc(OCC3(N)CC3)cc2)cc1] | (S,E)-1-(1-(3-(4′-((1- aminocyclopropyl)methoxy)-[1,1′- biphenyl]-4-yl)allyl)-1H-imidazol-2- yl)ethan-1-ol |
| 8 | 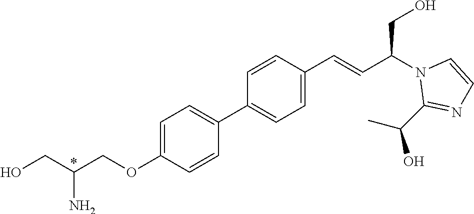 [SMILES: C[C@H](O)c1nccn1[C@@H](/C=C/c1ccc(-c2ccc(OCC(N)CO)cc2)cc1)CO] | (2S,E)-4-(4′-(2-amino-3- hydroxypropoxy)-[1,1′-biphenyl]-4- yl)-2-(2-((S)-1-hydroxyethyl)-1H- imidazol-1-yl)but-3-en-1-ol |
| | * racemic | |
| 9 | 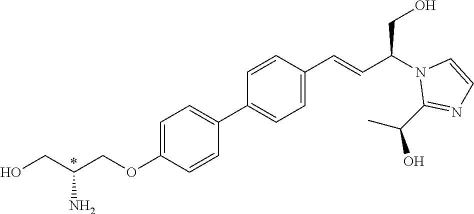 [SMILES: C[C@H](O)c1nccn1[C@@H](/C=C/c1ccc(-c2ccc(OC[C@H](N)CO)cc2)cc1)CO] | (S,E)-4-(4′-((R)-2-amino-3- hydroxypropoxy)-[1,1′-biphenyl]-4- yl)-2-(2-((S)-1-hydroxyethyl)-1H- imidazol-1-yl)but-3-en-1-ol |
| | * single isomer, absolute stereochemistry | |
| | unknown | |
| 10 | 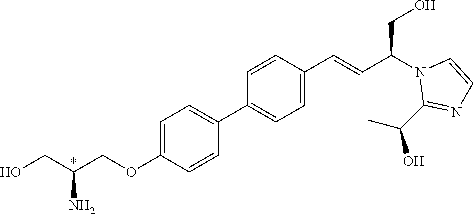 [SMILES: C[C@H](O)c1nccn1[C@@H](/C=C/c1ccc(-c2ccc(OC[C@@H](N)CO)cc2)cc1)CO] | (S,E)-4-(4′-((S)-2-amino-3- hydroxypropoxy)-[1,1′-biphenyl]-4- yl)-2-(2-((S)-1-hydroxyethyl)-1H- imidazol-1-yl)but-3-en-1-ol |
| | * single isomer, absolute stereochemistry | |
| | unknown | |
| 11 | 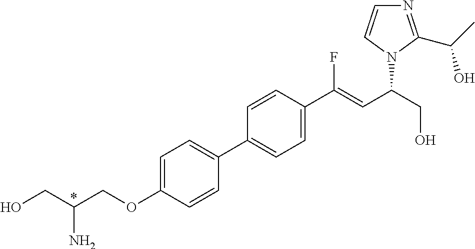 [SMILES: C[C@H](O)c1nccn1[C@@H](/C=C(\F)c1ccc(-c2ccc(OCC(N)CO)cc2)cc1)CO] | (2S,Z)-4-(4′-(2-amino-3- hydroxypropoxy)-[1,1′-biphenyl]-4- yl)-4-fluoro-2-(2-((S)-1- hydroxyethyl)-1H-imidazol-1- yl)but-3-en-1-ol |
| | * racemic | |
| 12 | 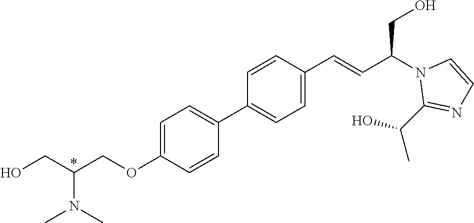 [SMILES: C[C@H](O)c1nccn1[C@@H](/C=C/c1ccc(-c2ccc(OCC(CO)N(C)C)cc2)cc1)CO] | (2S,E)-4-(4′-(2-(dimethylamino)-3- hydroxypropoxy)-[1,1′-biphenyl]-4- yl)-2-(2-((S)-1-hydroxyethyl)-1H- imidazol-1-yl)but-3-en-1-ol |
| | * racemic | |
| 13 | 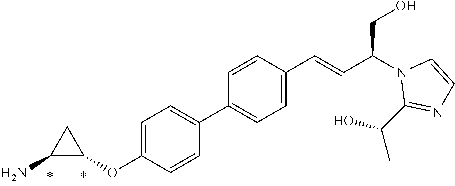 [SMILES: C[C@H](O)c1nccn1[C@@H](/C=C/c1ccc(-c2ccc(O[C@H]3C[C@@H]3N)cc2)cc1)CO] | (S,E)-4-(4′-((trans)-2- aminocyclopropoxy)-[1,1′- biphenyl]-4-yl)-2-(2-((S)-1- hydroxyethyl)-1H-imidazol-1- yl)but-3-en-1-ol |
| | * racemic-trans | |
| 14 | 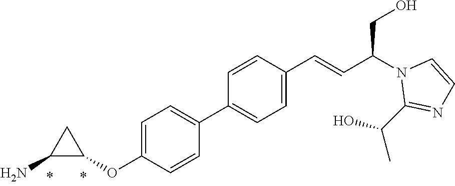 [SMILES: C[C@H](O)c1nccn1[C@@H](/C=C/c1ccc(-c2ccc(O[C@H]3C[C@@H]3N)cc2)cc1)CO] | (S,E)-4-(4′-((1S,2S)-2- aminocyclopropoxy)-[1,1′- biphenyl]-4-yl)-2-(2-((S)-1- hydroxyethyl)-1H-imidazol-1- yl)but-3-en-1-ol |
| | * single isomer, absolute stereochemistry | |
| | unknown | |
| 15 | 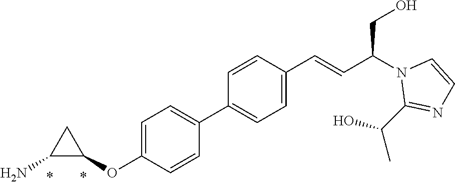 [SMILES: C[C@H](O)c1nccn1[C@@H](/C=C/c1ccc(-c2ccc(O[C@@H]3C[C@H]3N)cc2)cc1)CO] | (S,E)-4-(4′-((1R,2R)-2- aminocyclopropoxy)-[1,1′- biphenyl]-4-yl)-2-(2-((S)-1- hydroxyethyl)-1H-imidazol-1- yl)but-3-en-1-ol |
| | * single isomer, absolute stereochemistry | |
| | unknown | |
| 16 | 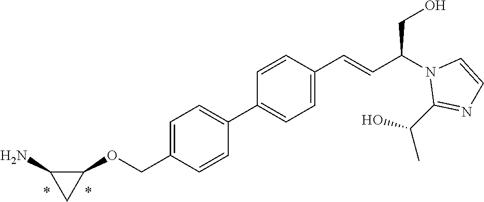 [SMILES: C[C@H](O)c1nccn1[C@@H](/C=C/c1ccc(-c2ccc(CO[C@H]3C[C@H]3N)cc2)cc1)CO] | (S,E)-4-(4′-(((cis)-2- aminocyclopropoxy)methyl)-[1,1′- biphenyl]-4-yl)-2-(2-((S)-1- hydroxyethyl)-1H-imidazol-1- yl)but-3-en-1-ol |
| | *racemic-cis | |
| 17 | 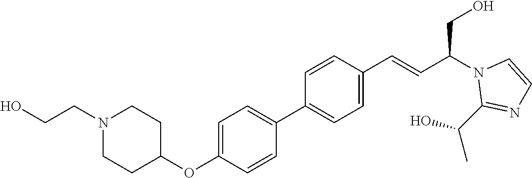 [SMILES: C[C@H](O)c1nccn1[C@@H](/C=C/c1ccc(-c2ccc(OC3CCN(CCO)CC3)cc2)cc1)CO] | (S,E)-2-(2-((S)-1-hydroxyethyl)-1H- imidazol-1-yl)-4-(4′-((1-(2- hydroxyethyl)piperidin-4-yl)oxy)- [1,1′-biphenyl]-4-yl)but-3-en-1-ol |
| 18 | 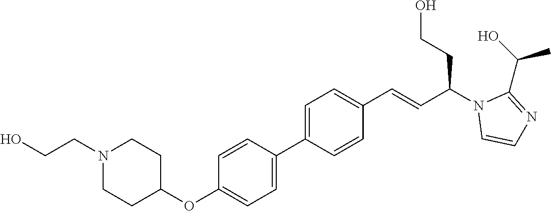 [SMILES: C[C@H](O)c1nccn1[C@@H](/C=C/c1ccc(-c2ccc(OC3CCN(CCO)CC3)cc2)cc1)CCO] | (R,E)-3-(2-((S)-1-hydroxyethyl)-1H- imidazol-1-yl)-5-(4′-((1-(2- hydroxyethyl)piperidin-4-yl)oxy)- [1,1′-biphenyl]-4-yl)pent-4-en-1-ol |
| 19 | 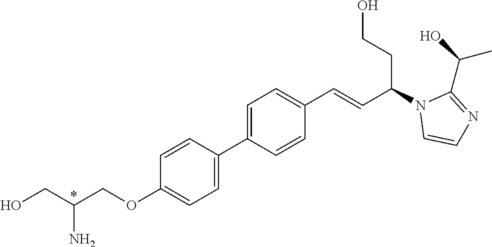 [SMILES: C[C@H](O)c1nccn1[C@@H](/C=C/c1ccc(-c2ccc(OCC(N)CO)cc2)cc1)CCO] | (3R,E)-5-(4′-(2-amino-3- hydroxypropoxy)-[1,1′-biphenyl]-4- yl)-3-(2-((S)-1-hydroxyethyl)-1H- imidazol-1-yl)pent-4-en-1-ol |
| | * racemic | |
| 20 | 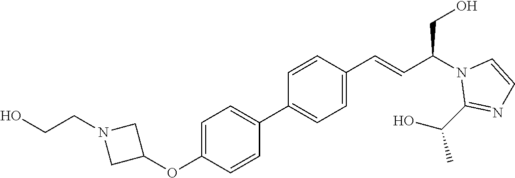 [SMILES: C[C@H](O)c1nccn1[C@@H](/C=C/c1ccc(-c2ccc(OC3CN(CCO)C3)cc2)cc1)CO] | (S,E)-2-(2-((S)-1-hydroxyethyl)-1H- imidazol-1-yl)-4-(4′-((1-(2- hydroxyethyl)azetidin-3-yl)oxy)- [1,1′-biphenyl]-4-yl)but-3-en-1-ol |
| 21 | 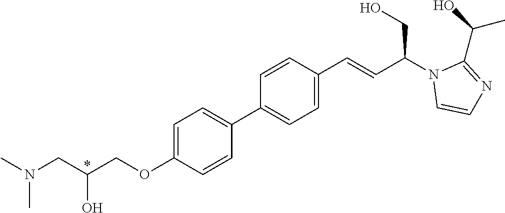 [SMILES: C[C@H](O)c1nccn1[C@@H](/C=C/c1ccc(-c2ccc(OCC(O)CN(C)C)cc2)cc1)CO] | (2S,E)-4-(4′-(3-(dimethylamino)-2- hydroxypropoxy)-[1,1′-biphenyl]-4- yl)-2-(2-((S)-1-hydroxyethyl)-1H- imidazol-1-yl)but-3-en-1-ol |
| | * racemic | |
| 22 | 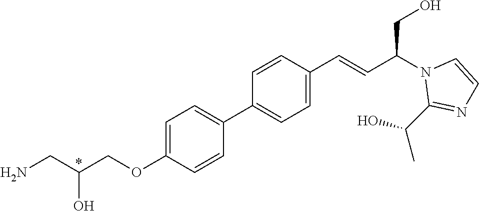 [SMILES: C[C@H](O)c1nccn1[C@@H](/C=C/c1ccc(-c2ccc(OCC(O)CN)cc2)cc1)CO] | (2S,E)-4-(4′-(3-amino-2- hydroxypropoxy)-[1,1′-biphenyl]-4- yl)-2-(2-((S)-1-hydroxyethyl)-1H- imidazol-1-yl)but-3-en-1-ol |
| | * racemic | |
| 23 | 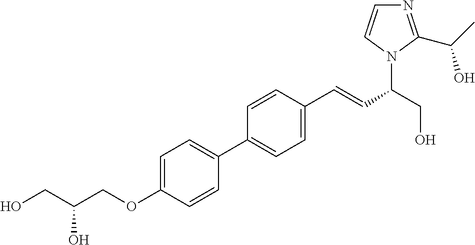 [SMILES: C[C@H](O)c1nccn1[C@@H](/C=C/c1ccc(-c2ccc(OC[C@H](O)CO)cc2)cc1)CO] | (R)-3-((4′-((S,E)-4-hydroxy-3-(2- ((S)-1-hydroxyethyl)-1H-imidazol- 1-yl)but-1-en-1-yl)-[1,1′-biphenyl]- 4-yl)oxy)propane-1,2-diol |
| 24 | 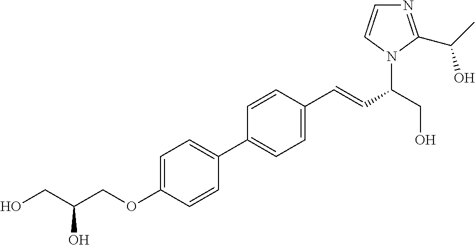 [SMILES: C[C@H](O)c1nccn1[C@@H](/C=C/c1ccc(-c2ccc(OC[C@@H](O)CO)cc2)cc1)CO] | (S)-3-((4′-((S,E)-4-hydroxy-3-(2- ((S)-1-hydroxyethyl)-1H-imidazol- 1-yl)but-1-en-1-yl)-[1,1′-biphenyl]- 4-yl)oxy)propane-1,2-diol |
| 25 | 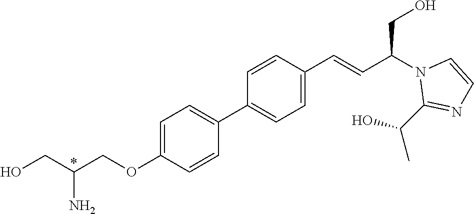 [SMILES: C[C@H](O)c1nccn1[C@@H](/C=C/c1ccc(-c2ccc(OCC(N)CO)cc2)cc1)CO] | (2S,E)-4-(4′-(2-amino-3- hydroxypropoxy)-[1,1′-biphenyl]-4- yl)-2-(2-((S)-1-hydroxyethyl)-1H- imidazol-1-yl)but-3-en-1-ol |
| | * racemic | |
| 26 | 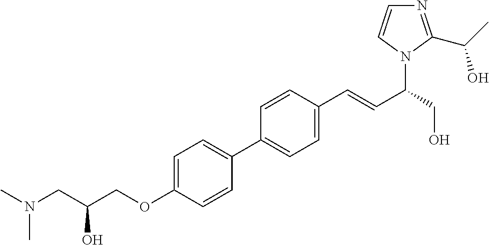 [SMILES: C[C@H](O)c1nccn1[C@@H](/C=C/c1ccc(-c2ccc(OC[C@@H](O)CN(C)C)cc2)cc1)CO] | (S,E)-4-(4′-((S)-3-(dimethylamino)- 2-hydroxypropoxy)-[1,1′-biphenyl]- 4-yl)-2-(2-((S)-1-hydroxyethyl)-1H- imidazol-1-yl)but-3-en-1-ol |
| 27 | 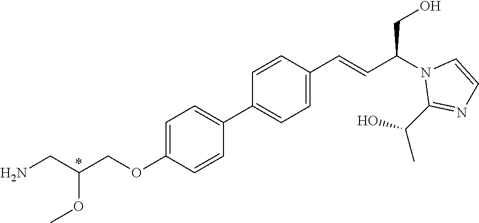 [SMILES: COC(CN)COc1ccc(-c2ccc(/C=C/[C@@H](CO)n3ccnc3[C@H](C)O)cc2)cc1] | (2S,E)-4-(4′-(3-amino-2- methoxypropoxy)-[1,1′-biphenyl]-4- yl)-2-(2-((S)-1-hydroxyethyl)-1H- imidazol-1-yl)but-3-en-1-ol |
| | * racemic | |
| 28 | 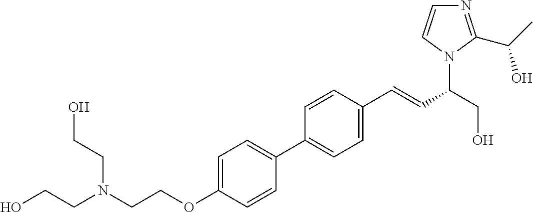 [SMILES: C[C@H](O)c1nccn1[C@@H](/C=C/c1ccc(-c2ccc(OCCN(CCO)CCO)cc2)cc1)CO] | 2,2′-((2-((4′-((S,E)-4-hydroxy-3-(2- ((S)-1-hydroxyethyl)-1H-imidazol- 1-yl)but-1-en-1-yl)-[1,1′-biphenyl]- 4-yl)oxy)ethyl)azanediyl)bis(ethan- 1-ol) |
| 29 | 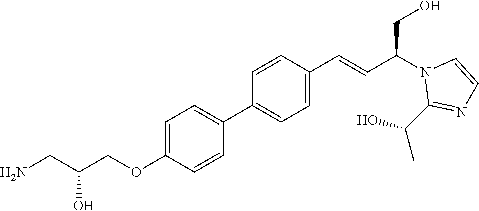 [SMILES: C[C@H](O)c1nccn1[C@@H](/C=C/c1ccc(-c2ccc(OC[C@H](O)CN)cc2)cc1)CO] | (S,E)-4-(4′-((R)-3-amino-2- hydroxypropoxy)-[1,1′-biphenyl]-4- yl)-2-(2-((S)-1-hydroxyethyl)-1H- imidazol-1-yl)but-3-en-1-ol |
| 30 | 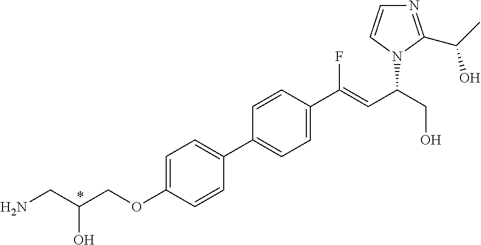 [SMILES: C[C@H](O)c1nccn1[C@@H](/C=C(\F)c1ccc(-c2ccc(OCC(O)CN)cc2)cc1)CO] | (S,Z)-4-(4′-((R)-3-amino-2- hydroxypropoxy)-[1,1′-biphenyl]-4- yl)-4-fluoro-2-(2-((S)-1- hydroxyethyl)-1H-imidazol-1- yl)but-3-en-1-ol; or (S,Z)-4-(4′-((S)- 3-amino-2-hydroxypropoxy)-[1,1′- biphenyl]-4-yl)-4-fluoro-2-(2-((S)-1- hydroxyethyl)-1H-imidazol-1- yl)but-3-en-1-ol |
| | * single isomer, absolute stereochemistry | |
| | unknown | |
| 31 | 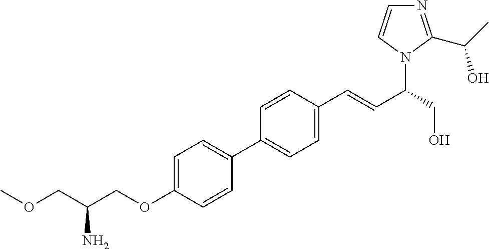 [SMILES: COC[C@H](N)COc1ccc(-c2ccc(/C=C/[C@@H](CO)n3ccnc3[C@H](C)O)cc2)cc1] | (S,E)-4-(4′-((S)-2-amino-3- methoxypropoxy)-[1,1′-biphenyl]-4- yl)-2-(2-((S)-1-hydroxyethyl)-1H- imidazol-1-yl)but-3-en-1-ol |
| 32 | 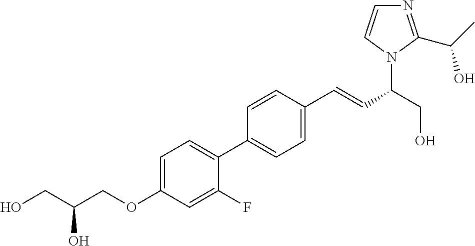 [SMILES: C[C@H](O)c1nccn1[C@@H](/C=C/c1ccc(-c2ccc(OC[C@@H](O)CO)cc2F)cc1)CO] | (S)-3-((2-fluoro-4′-((S,E)-4- hydroxy-3-(2-((S)-1-hydroxyethyl)- 1H-imidazol-1-yl)but-1-en-1-yl)- [1,1′-biphenyl]-4-yl)oxy)propane- 1,2-diol |
| 33 | 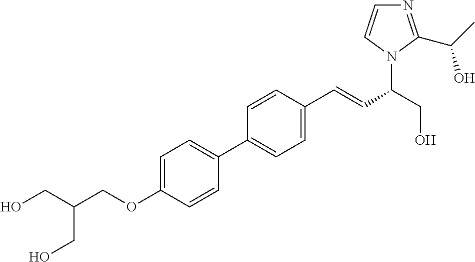 [SMILES: C[C@H](O)c1nccn1[C@@H](/C=C/c1ccc(-c2ccc(OCC(CO)CO)cc2)cc1)CO] | 2-(((4′-((S,E)-4-hydroxy-3-(2-((S)-1- hydroxyethyl)-1H-imidazol-1- yl)but-1-en-1-yl)-[1,1′-biphenyl]-4- yl)oxy)methyl)propane-1,3-diol |
| 34 | 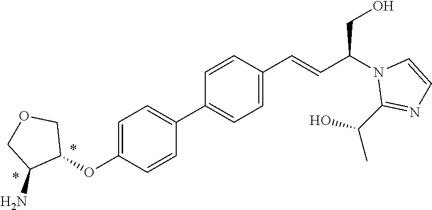 [SMILES: C[C@H](O)c1nccn1[C@@H](/C=C/c1ccc(-c2ccc(O[C@H]3COC[C@@H]3N)cc2)cc1)CO] | (S,E)-4-(4′-(((trans)-4- aminotetrahydrofuran-3-yl)oxy)- [1,1′-biphenyl]-4-yl)-2-(2-((S)-1- hydroxyethyl)-1H-imidazol-1- yl)but-3-en-1-ol |
| | *racemic-trans | |
| 35 | 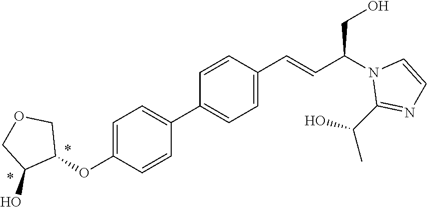 [SMILES: C[C@H](O)c1nccn1[C@@H](/C=C/c1ccc(-c2ccc(O[C@H]3COC[C@@H]3O)cc2)cc1)CO] | (trans)-4-((4′-((S,E)-4-hydroxy-3-(2- ((S)-1-hydroxyethyl)-1H-imidazol- 1-yl)but-1-en-1-yl)-[1,1′-biphenyl]- 4-yl)oxy)tetrahydrofuran-3-ol |
| | *racemic-trans | |
| 36 | 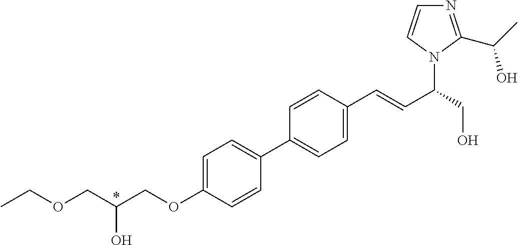 [SMILES: CCOCC(O)COc1ccc(-c2ccc(/C=C/[C@@H](CO)n3ccnc3[C@H](C)O)cc2)cc1] | (2S,E)-4-(4′-(3-ethoxy-2- hydroxypropoxy)-[1,1′-biphenyl]-4- yl)-2-(2-((S)-1-hydroxyethyl)-1H- imidazol-1-yl)but-3-en-1-ol |
| | * racemic | |
| 37 | 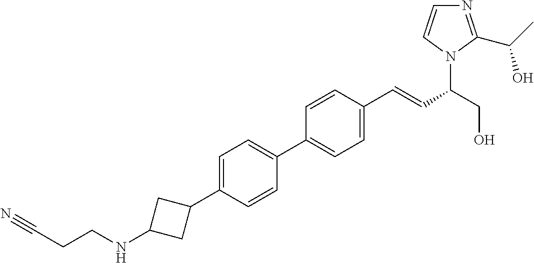 [SMILES: C[C@H](O)c1nccn1[C@@H](/C=C/c1ccc(-c2ccc(C3CC(NCCC#N)C3)cc2)cc1)CO] | 3-((3-(4′-((S,E)-4-hydroxy-3-(2-((S)- 1-hydroxyethyl)-1H-imidazol-1- yl)but-1-en-1-yl)-[1,1′-biphenyl]-4- yl)cyclobutyl)amino)propanenitrile |
| 38 | 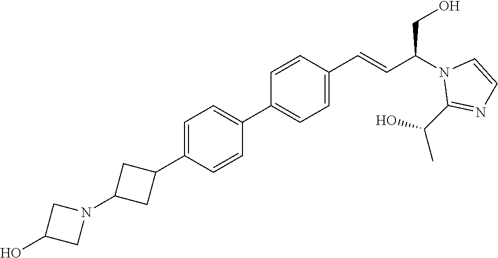 [SMILES: C[C@H](O)c1nccn1[C@@H](/C=C/c1ccc(-c2ccc(C3CC(N4CC(O)C4)C3)cc2)cc1)CO] | 1-(3-(4′-((S,E)-4-hydroxy-3-(2-((S)- 1-hydroxyethyl)-1H-imidazol-1- yl)but-1-en-1-yl)-[1,1′-biphenyl]-4- yl)cyclobutyl)azetidin-3-ol |
| 39 | 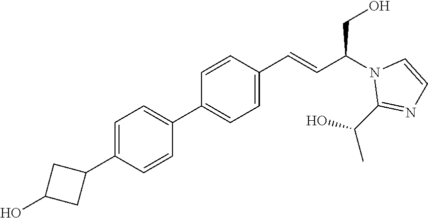 [SMILES: C[C@H](O)c1nccn1[C@@H](/C=C/c1ccc(-c2ccc(C3CC(O)C3)cc2)cc1)CO] | 3-(4′-((S,E)-4-hydroxy-3-(2-((S)-1- hydroxyethyl)-1H-imidazol-1- yl)but-1-en-1-yl)-[1,1′-biphenyl]-4- yl)cyclobutan-1-ol |
| 40 | 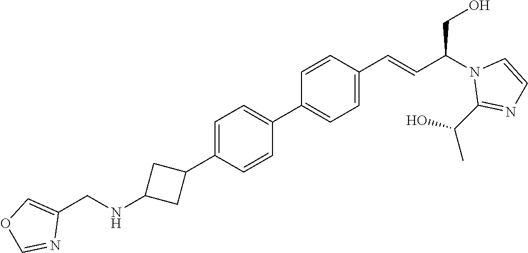 [SMILES: C[C@H](O)c1nccn1[C@@H](/C=C/c1ccc(-c2ccc(C3CC(NCc4cocn4)C3)cc2)cc1)CO] | (S,E)-2-(2-((S)-1-hydroxyethyl)-1H- imidazol-1-yl)-4-(4′-(3-((oxazol-4- ylmethyl)amino)cyclobutyl)-[1,1′- biphenyl]-4-yl)but-3-en-1-ol |
| 41 | 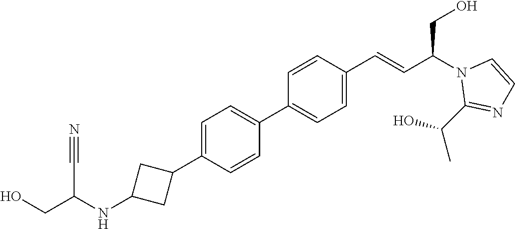 [SMILES: C[C@H](O)c1nccn1[C@@H](/C=C/c1ccc(-c2ccc(C3CC(NC(C#N)CO)C3)cc2)cc1)CO] | 3-hydroxy-2-((3-(4′-((S,E)-4- hydroxy-3-(2-((S)-1-hydroxyethyl)- 1H-imidazol-1-yl)but-1-en-1-yl)- [1,1′-biphenyl]-4- yl)cyclobutyl)amino)propanenitrile |
| 42 | 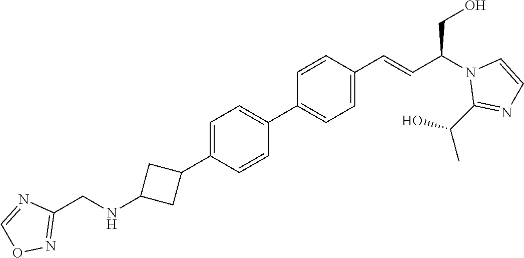 [SMILES: C[C@H](O)c1nccn1[C@@H](/C=C/c1ccc(-c2ccc(C3CC(NCc4ncon4)C3)cc2)cc1)CO] | (S,E)-4-(4′-(3-(((1,2,4-oxadiazol-3- yl)methyl)amino)cyclobutyl)-[1,1′- biphenyl]-4-yl)-2-(2-((S)-1- hydroxyethyl)-1H-imidazol-1- yl)but-3-en-1-ol |
| 43 | 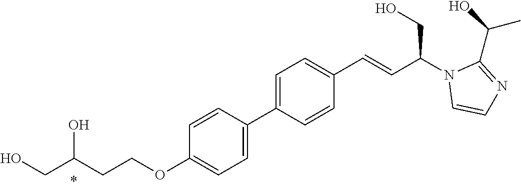 [SMILES: C[C@H](O)c1nccn1[C@@H](/C=C/c1ccc(-c2ccc(OCCC(O)CO)cc2)cc1)CO] | 4-((4′-((S,E)-4-hydroxy-3-(2-((S)-1- hydroxyethyl)-1H-imidazol-1- yl)but-1-en-1-yl)-[1,1′-biphenyl]-4- yl)oxy)butane-1,2-diol |
| | * racemic | |
| 44 | 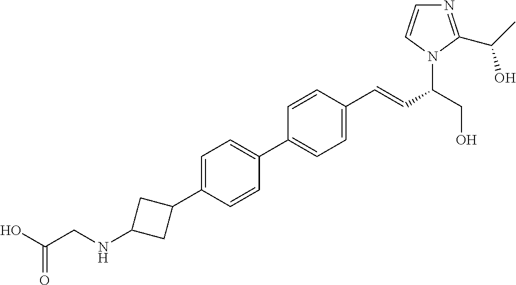 [SMILES: C[C@H](O)c1nccn1[C@@H](/C=C/c1ccc(-c2ccc(C3CC(NCC(=O)O)C3)cc2)cc1)CO] | (3-(4′-((S,E)-4-hydroxy-3-(2-((S)-1- hydroxyethyl)-1H-imidazol-1- yl)but-1-en-1-yl)-[1,1′-biphenyl]-4- yl)cyclobutyl)glycine |
| 45 | 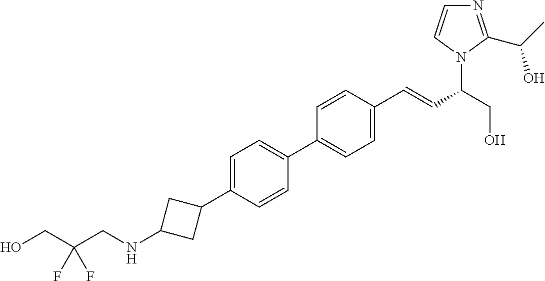 [SMILES: C[C@H](O)c1nccn1[C@@H](/C=C/c1ccc(-c2ccc(C3CC(NCC(F)(F)CO)C3)cc2)cc1)CO] | (S,E)-4-(4′-(3-((2,2-difluoro-3- hydroxypropyl)amino)cyclobutyl)- [1,1′-biphenyl]-4-yl)-2-(2-((S)-1- hydroxyethyl)-1H-imidazol-1- yl)but-3-en-1-ol |
| 46 | 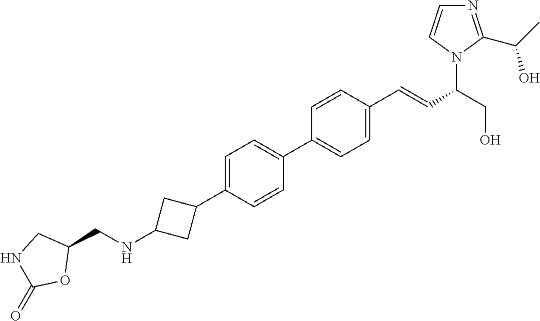 [SMILES: C[C@H](O)c1nccn1[C@@H](/C=C/c1ccc(-c2ccc(C3CC(NC[C@@H]4CNC(=O)O4)C3)cc2)cc1)CO] | (R)-5-(((3-(4′-((S,E)-4-hydroxy-3- (2-((S)-1-hydroxyethyl)-1H- imidazol-1-yl)but-1-en-1-yl)-[1,1′- biphenyl]-4- yl)cyclobutyl)amino)methyl) oxazolidin-2-one |
| 47 | 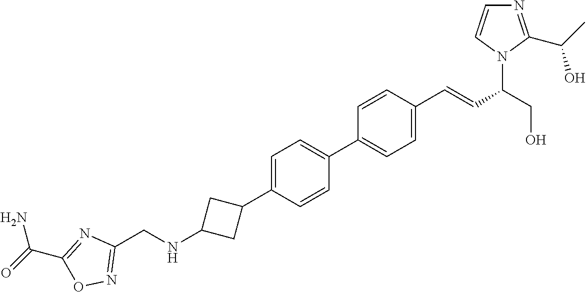 [SMILES: C[C@H](O)c1nccn1[C@@H](/C=C/c1ccc(-c2ccc(C3CC(NCc4noc(C(N)=O)n4)C3)cc2)cc1)CO] | 3-(((3-(4′-((S,E)-4-hydroxy-3-(2- ((S)-1-hydroxyethyl)-1H-imidazol- 1-yl)but-1-en-1-yl)-[1,1′-biphenyl]- 4-yl)cyclobutyl)amino)methyl)- 1,2,4-oxadiazole-5-carboxamide |
| 48 | 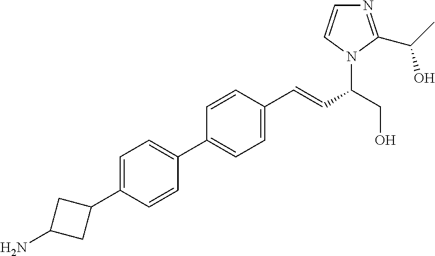 [SMILES: C[C@H](O)c1nccn1[C@@H](/C=C/c1ccc(-c2ccc(C3CC(N)C3)cc2)cc1)CO] | (S,E)-4-(4′-(3-aminocyclobutyl)- [1,1′-biphenyl]-4-yl)-2-(2-((S)-1- hydroxyethyl)-1H-imidazol-1- yl)but-3-en-1-ol |
| 49 | 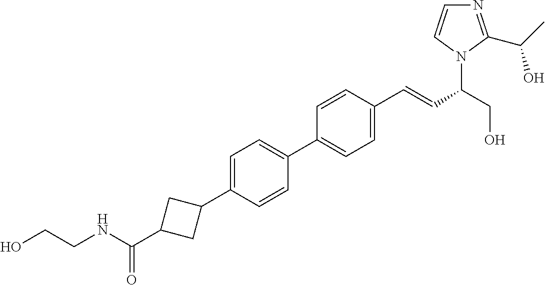 [SMILES: C[C@H](O)c1nccn1[C@@H](/C=C/c1ccc(-c2ccc(C3CC(C(=O)NCCO)C3)cc2)cc1)CO] | 3-(4′-((S,E)-4-hydroxy-3-(2-((S)-1- hydroxyethyl)-1H-imidazol-1- yl)but-1-en-1-yl)-[1,1′-biphenyl]-4- yl)-N-(2-hydroxyethyl)cyclobutane- 1-carboxamide |
| 50 | 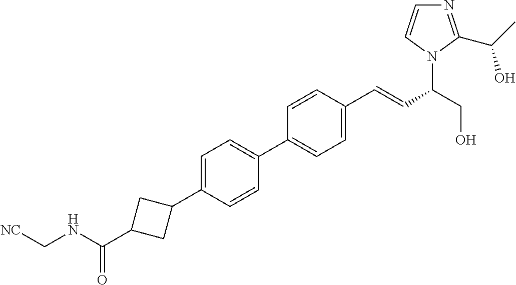 [SMILES: C[C@H](O)c1nccn1[C@@H](/C=C/c1ccc(-c2ccc(C3CC(C(=O)NCC#N)C3)cc2)cc1)CO] | N-(cyanomethyl)-3-(4′-((S,E)-4- hydroxy-3-(2-((S)-1-hydroxyethyl)- 1H-imidazol-1-yl)but-1-en-1-yl)- [1,1′-biphenyl]-4-yl)cyclobutane-1- carboxamide |
| 51 | 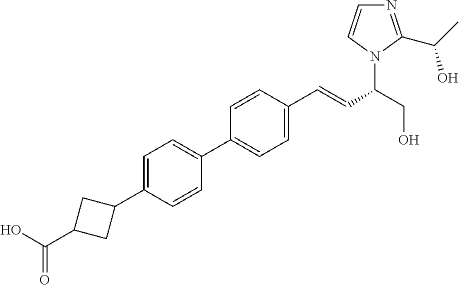 [SMILES: C[C@H](O)c1nccn1[C@@H](/C=C/c1ccc(-c2ccc(C3CC(C(=O)O)C3)cc2)cc1)CO] | 3-(4′-((S,E)-4-hydroxy-3-(2-((S)-1- hydroxyethyl)-1H-imidazol-1- yl)but-1-en-1-yl)-[1,1′-biphenyl]-4- yl)cyclobutane-1-carboxylic acid |
| 52 | 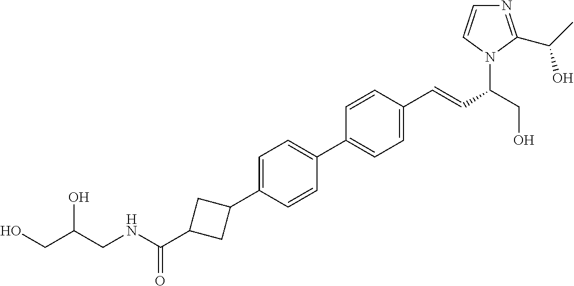 [SMILES: C[C@H](O)c1nccn1[C@@H](/C=C/c1ccc(-c2ccc(C3CC(C(=O)NCC(O)CO)C3)cc2)cc1)CO] | N-(2,3-dihydroxypropyl)-3-(4′- ((S,E)-4-hydroxy-3-(2-((S)-1- hydroxyethyl)-1H-imidazol-1- yl)but-1-en-1-yl)-[1,1′-biphenyl]-4- yl)cyclobutane-1-carboxamide |
| 53 | 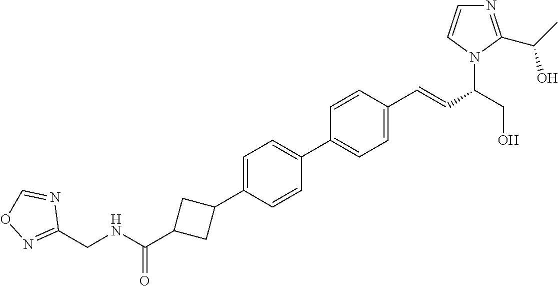 [SMILES: C[C@H](O)c1nccn1[C@@H](/C=C/c1ccc(-c2ccc(C3CC(C(=O)NCc4ncon4)C3)cc2)cc1)CO] | N-((1,2,4-oxadiazol-3-yl)methyl)-3- (4′-((S,E)-4-hydroxy-3-(2-((S)-1- hydroxyethyl)-1H-imidazol-1- yl)but-1-en-1-yl)-[1,1′-biphenyl]-4- yl)cyclobutane-1-carboxamide |
| 54 | 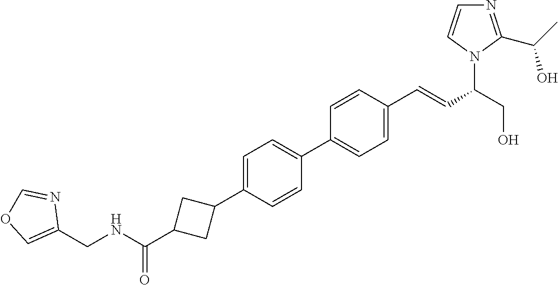 [SMILES: C[C@H](O)c1nccn1[C@@H](/C=C/c1ccc(-c2ccc(C3CC(C(=O)NCc4cocn4)C3)cc2)cc1)CO] | 3-(4′-((S,E)-4-hydroxy-3-(2-((S)-1- hydroxyethyl)-1H-imidazol-1- yl)but-1-en-1-yl)-[1,1′-biphenyl]-4- yl)-N-(oxazol-4- ylmethyl)cyclobutane-1- carboxamide |
| 55 | 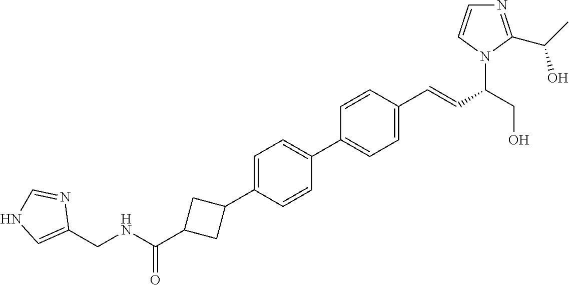 [SMILES: C[C@H](O)c1nccn1[C@@H](/C=C/c1ccc(-c2ccc(C3CC(C(=O)NCc4c[nH]cn4)C3)cc2)cc1)CO] | N-((1H-imidazol-4-yl)methyl)-3-(4′- ((S,E)-4-hydroxy-3-(2-((S)-1- hydroxyethyl)-1H-imidazol-1- yl)but-1-en-1-yl)-[1,1′-biphenyl]-4- yl)cyclobutane-1-carboxamide |
| 56 | 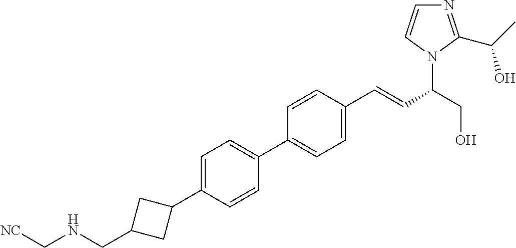 [SMILES: C[C@H](O)c1nccn1[C@@H](/C=C/c1ccc(-c2ccc(C3CC(CNCC#N)C3)cc2)cc1)CO] | 2-(((3-(4′-((S,E)-4-hydroxy-3-(2- ((S)-1-hydroxyethyl)-1H-imidazol- 1-yl)but-1-en-1-yl)-[1,1′-biphenyl]-4- yl)cyclobutyl)methyl)amino) acetonitrile |
| 57 | 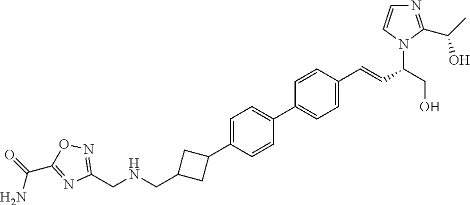 [SMILES: C[C@H](O)c1nccn1[C@@H](/C=C/c1ccc(-c2ccc(C3CC(CNCc4noc(C(N)=O)n4)C3)cc2)cc1)CO] | 3-((((3-(4′-((S,E)-4-hydroxy-3-(2- ((S)-1-hydroxyethyl)-1H-imidazol- 1-yl)but-1-en-1-yl)-[1,1′-biphenyl]-4- yl)cyclobutyl)methyl)amino)methyl)- 1,2,4-oxadiazole-5-carboxamide |
| 58 | 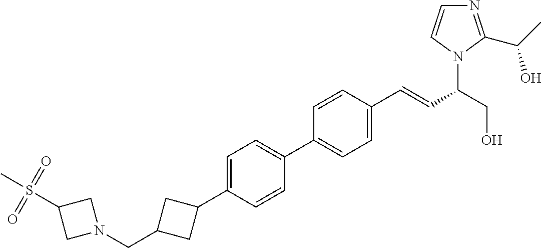 [SMILES: C[C@H](O)c1nccn1[C@@H](/C=C/c1ccc(-c2ccc(C3CC(CN4CC(S(C)(=O)=O)C4)C3)cc2)cc1)CO] | (S,E)-2-(2-((S)-1-hydroxyethyl)-1H- imidazol-1-yl)-4-(4′-(3-((3- (methylsulfonyl)azetidin-1- yl)methyl)cyclobutyl)-[1,1′- biphenyl]-4-yl)but-3-en-1-ol |
| 59 | 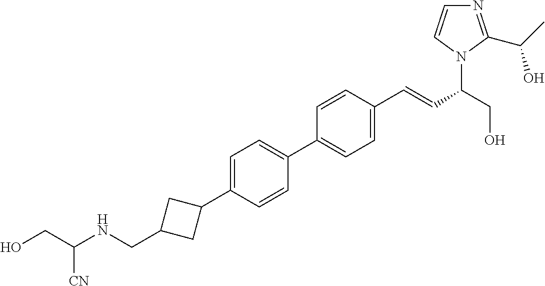 [SMILES: C[C@H](O)c1nccn1[C@@H](/C=C/c1ccc(-c2ccc(C3CC(CNC(C#N)CO)C3)cc2)cc1)CO] | 3-hydroxy-2-(((3-(4′-((S,E)-4- hydroxy-3-(2-((S)-1-hydroxyethyl)- 1H-imidazol-1-yl)but-1-en-1-yl)- [1,1′-biphenyl]-4- yl)cyclobutyl)methyl)amino)propane nitrile |
| 60 | 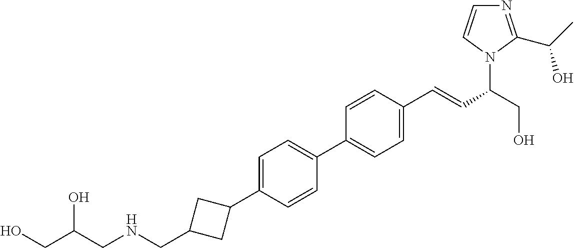 [SMILES: C[C@H](O)c1nccn1[C@@H](/C=C/c1ccc(-c2ccc(C3CC(CNCC(O)CO)C3)cc2)cc1)CO] | 3-(((3-(4′-((S,E)-4-hydroxy-3-(2- ((S)-1-hydroxyethyl)-1H-imidazol- 1-yl)but-1-en-1-yl)-[1,1′- biphenyl]-4- yl)cyclobutyl)methyl)amino) propane-1,2-diol |
| 61 | 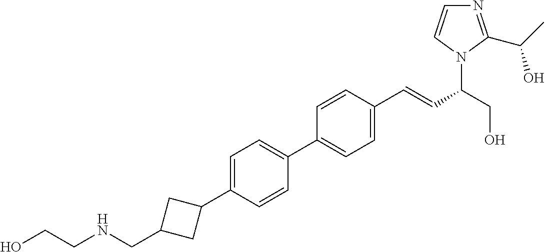 [SMILES: C[C@H](O)c1nccn1[C@@H](/C=C/c1ccc(-c2ccc(C3CC(CNCCO)C3)cc2)cc1)CO] | (S,E)-2-(2-((S)-1-hydroxyethyl)-1H- imidazol-1-yl)-4-(4′-(3-(((2- hydroxyethyl)amino)methyl) cyclobutyl)-[1,1′-biphenyl]-4-yl) but-3-en-1- ol |
| 62 | 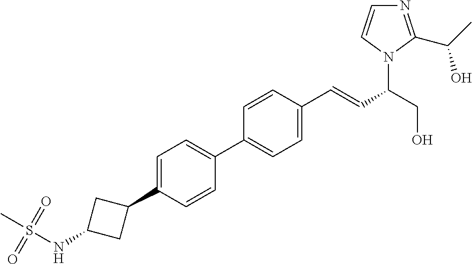 [SMILES: C[C@H](O)c1nccn1[C@@H](/C=C/c1ccc(-c2ccc([C@H]3C[C@H](NS(C)(=O)=O)C3)cc2)cc1)CO] | N-((1S,3r)-3-(4′-((S,E)-4-hydroxy-3- (2-((S)-1-hydroxyethyl)-1H- imidazol-1-yl)but-1-en-1-yl)-[1,1′- biphenyl]-4- yl)cyclobutyl)methanesulfonamide |
| 63 | 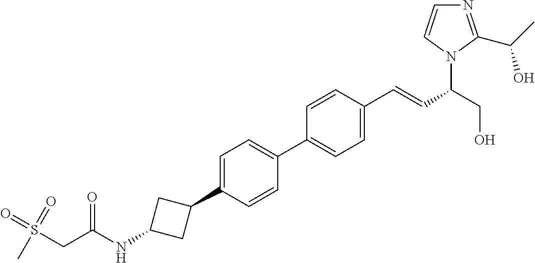 [SMILES: C[C@H](O)c1nccn1[C@@H](/C=C/c1ccc(-c2ccc([C@H]3C[C@H](NC(=O)CS(C)(=O)=O)C3)cc2)cc1)CO] | N-((1S,3r)-3-(4′-((S,E)-4-hydroxy-3- (2-((S)-1-hydroxyethyl)-1H- imidazol-1-yl)but-1-en-1-yl)-[1,1′- biphenyl]-4-yl)cyclobutyl)-2- (methylsulfonyl)acetamide |
| 64 | 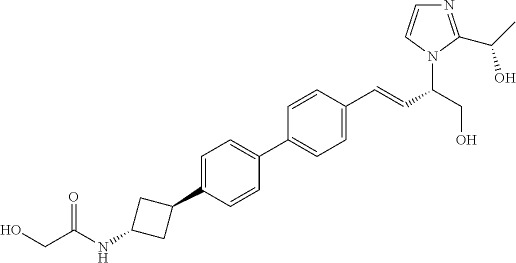 [SMILES: C[C@H](O)c1nccn1[C@@H](/C=C/c1ccc(-c2ccc([C@H]3C[C@H](NC(=O)CO)C3)cc2)cc1)CO] | 2-hydroxy-N-((1S,3r)-3-(4′-((S,E)-4- hydroxy-3-(2-((S)-1-hydroxyethyl)- 1H-imidazol-1-yl)but-1-en-1-yl)- [1,1′-biphenyl]-4- yl)cyclobutyl)acetamide |
| 65 | 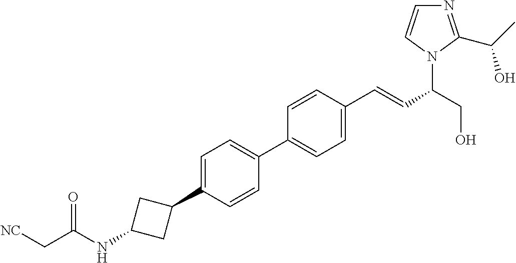 [SMILES: C[C@H](O)c1nccn1[C@@H](/C=C/c1ccc(-c2ccc([C@H]3C[C@H](NC(=O)CC#N)C3)cc2)cc1)CO] | 2-cyano-N-((1S,3r)-3-(4′-((S,E)-4- hydroxy-3-(2-((S)-1-hydroxyethyl)- 1H-imidazol-1-yl)but-1-en-1-yl)- [1,1′-biphenyl]-4- yl)cyclobutyl)acetamide |
| 66 | 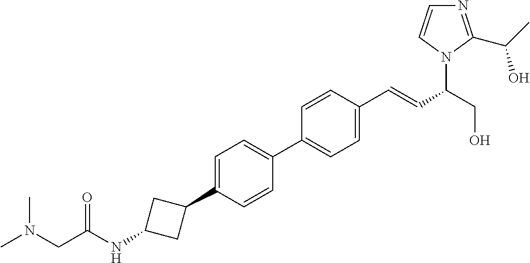 [SMILES: C[C@H](O)c1nccn1[C@@H](/C=C/c1ccc(-c2ccc([C@H]3C[C@H](NC(=O)CN(C)C)C3)cc2)cc1)CO] | 2-(dimethylamino)-N-((1S,3r)-3-(4′- ((S,E)-4-hydroxy-3-(2-((S)-1- hydroxyethyl)-1H-imidazol-1- yl)but-1-en-1-yl)-[1,1′-biphenyl]-4- yl)cyclobutyl)acetamide |
| 67 | 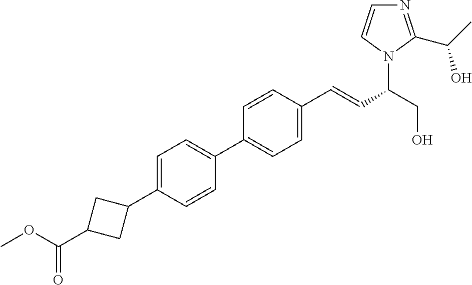 [SMILES: COC(=O)C1CC(c2ccc(-c3ccc(/C=C/[C@@H](CO)n4ccnc4[C@H](C)O)cc3)cc2)C1] | methyl 3-(4′-((S,E)-4-hydroxy-3-(2- ((S)-1-hydroxyethyl)-1H-imidazol- 1-yl)but-1-en-1-yl)-[1,1′-biphenyl]- 4-yl)cyclobutane-1-carboxylate |
| 68 | 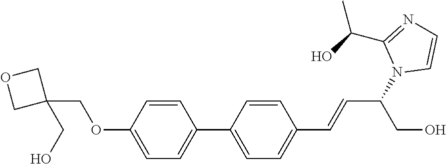 [SMILES: C[C@H](O)c1nccn1[C@@H](/C=C/c1ccc(-c2ccc(OCC3(CO)COC3)cc2)cc1)CO] | (S,E)-2-(2-((S)-1-hydroxyethyl)-1H- imidazol-1-yl)-4-(4′-((3- (hydroxymethyl)oxetan-3- yl)methoxy)-[1,1′-biphenyl]-4- yl)but-3-en-1-ol |
| 69 | 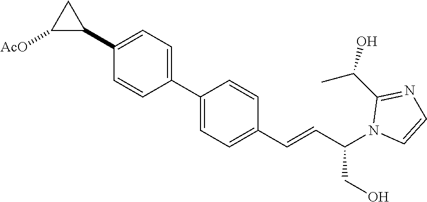 | (1R,2S)-2-(4′-((S,E)-4-hydroxy-3- (2-((S)-1-hydroxyethyl)-1H- imidazol-1-yl)but-1-en-1-yl)-[1,1′- biphenyl]-4-yl)cyclopropyl acetate |
| 70 | 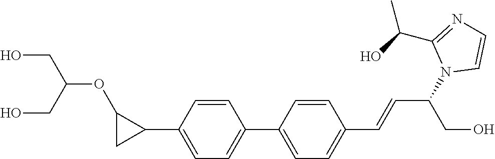 [SMILES: C[C@H](O)c1nccn1[C@@H](/C=C/c1ccc(-c2ccc(C3CC3OC(CO)CO)cc2)cc1)CO] | 2-(2-(4′-((S,E)-4-hydroxy-3-(2-((S)- 1-hydroxyethyl)-1H-imidazol-1- yl)but-1-en-1-yl)-[1,1′-biphenyl]-4- yl)cyclopropoxy)propane-1,3-diol |
| 71 | 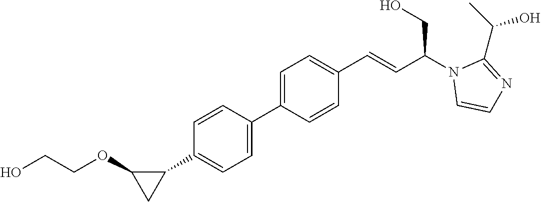 [SMILES: C[C@H](O)c1nccn1[C@@H](/C=C/c1ccc(-c2ccc([C@@H]3C[C@H]3OCCO)cc2)cc1)CO] | (S,E)-4-(4′-((1S,2R)-2-(2- hydroxyethoxy)cyclopropyl)-[1,1′- biphenyl]-4-yl)-2-(2-((S)-1- hydroxyethyl)-1H-imidazol-1- yl)but-3-en-1-ol |
| 72 | 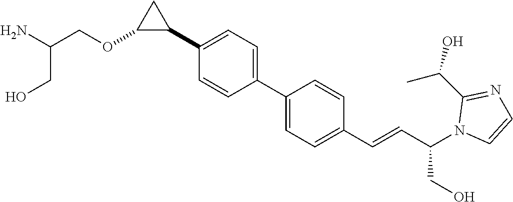 | (2S,E)-4-(4′-((1S,2R)-2-(2-amino-3- hydroxypropoxy)cyclopropyl)-[1,1′- biphenyl]-4-yl)-2-(2-((S)-1- hydroxyethyl)-1H-imidazol-1- yl)but-3-en-1-ol |
| 73 | 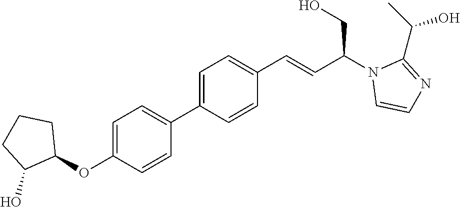 [SMILES: C[C@H](O)c1nccn1[C@@H](/C=C/c1ccc(-c2ccc(O[C@@H]3CCC[C@H]3O)cc2)cc1)CO] | (1R,2R)-2-((4′-((S,E)-4-hydroxy-3- (2-((S)-1-hydroxyethyl)-1H- imidazol-1-yl)but-1-en-1-yl)-[1,1′- biphenyl]-4-yl)oxy)cyclopentan-1-ol |
| 74 | 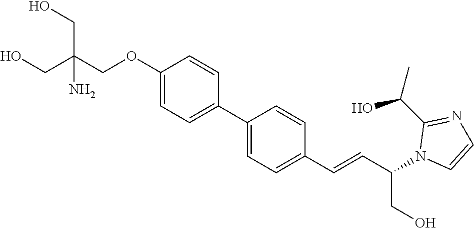 [SMILES: C[C@H](O)c1nccn1[C@@H](/C=C/c1ccc(-c2ccc(OCC(N)(CO)CO)cc2)cc1)CO] | 2-amino-2-(((4′-((S,E)-4-hydroxy-3- (2-((S)-1-hydroxyethyl)-1H- imidazol-1-yl)but-1-en-1-yl)-[1,1′- biphenyl]-4-yl)oxy)methyl)propane- 1,3-diol |
| 75 | 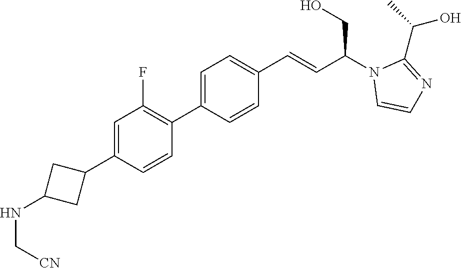 [SMILES: C[C@H](O)c1nccn1[C@@H](/C=C/c1ccc(-c2ccc(C3CC(NCC#N)C3)cc2F)cc1)CO] | 2-((3-(2-fluoro-4′-((S,E)-4-hydroxy- 3-(2-((S)-1-hydroxyethyl)-1H- imidazol-1-yl)but-1-en-1-yl)-[1,1′- biphenyl]-4- yl)cyclobutyl)amino)acetonitrile |

In one aspect, compounds described herein are in the form of pharmaceutically acceptable salts. As well, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

“Pharmaceutically acceptable,” as used herein, refers a material, such as a carrier or diluent, which does not abrogate the biological activity or properties of the compound, and is relatively nontoxic at the concentration or amount used, i.e., the material is administered to an individual without causing undesirable biological effects or interacting in a deleterious manner with any of the components of the composition in which it is contained.

The term “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. Handbook of Pharmaceutical Salts: Properties, Selection and Use. International Union of Pure and Applied Chemistry, Wiley-VCH 2002. S. M. Berge, L. D. Bighley, D.C. Monkhouse, J. Pharm. Sci. 1977, 66, 1-19. P. H. Stahl and C. G. Wermuth, editors, Handbook of Pharmaceutical Salts: Properties, Selection and Use, Weinheim/Zurich:Wiley-VCH/VHCA, 2002. Pharmaceutical salts typically are more soluble and more rapidly soluble in stomach and intestinal juices than non-ionic species and so are useful in solid dosage forms. Furthermore, because their solubility often is a function of pH, selective dissolution in one or another part of the digestive tract is possible and this capability can be manipulated as one aspect of delayed and sustained release behaviors. Also, because the salt-forming molecule can be in equilibrium with a neutral form, passage through biological membranes can be adjusted.

In some embodiments, pharmaceutically acceptable salts are obtained by reacting a compound of Formula (I) with an acid. In some embodiments, the compound of Formula (I) (i.e. free base form) is basic and is reacted with an organic acid or an inorganic acid. Inorganic acids include, but are not limited to, hydrochloric acid, hydrobromic acid, sulfuric acid, phosphoric acid, nitric acid, and metaphosphoric acid. Organic acids include, but are not limited to, 1-hydroxy-2-naphthoic acid; 2,2-dichloroacetic acid; 2-hydroxyethanesulfonic acid; 2-oxoglutaric acid; 4-acetamidobenzoic acid; 4-aminosalicylic acid; acetic acid; adipic acid; ascorbic acid (L); aspartic acid (L); benzenesulfonic acid; benzoic acid; camphoric acid (+); camphor-10-sulfonic acid (+); capric acid (decanoic acid); caproic acid (hexanoic acid); caprylic acid (octanoic acid); carbonic acid; cinnamic acid; citric acid; cyclamic acid; dodecylsulfuric acid; ethane-1,2-disulfonic acid; ethanesulfonic acid; formic acid; fumaric acid; galactaric acid; gentisic acid; glucoheptonic acid (D); gluconic acid (D); glucuronic acid (D); glutamic acid; glutaric acid; glycerophosphoric acid; glycolic acid; hippuric acid; isobutyric acid; lactic acid (DL); lactobionic acid; lauric acid; maleic acid; malic acid (−L); malonic acid; mandelic acid (DL); methanesulfonic acid; naphthalene-1,5-disulfonic acid; naphthalene-2-sulfonic acid; nicotinic acid; oleic acid; oxalic acid; palmitic acid; pamoic acid; phosphoric acid; proprionic acid; pyroglutamic acid (−L); salicylic acid; sebacic acid; stearic acid; succinic acid; sulfuric acid; tartaric acid (+L); thiocyanic acid; toluenesulfonic acid (p); and undecylenic acid.

In some embodiments, a compound of Formula (I) is prepared as a chloride salt, sulfate salt, bromide salt, mesylate salt, maleate salt, citrate salt or phosphate salt.

In some embodiments, pharmaceutically acceptable salts are obtained by reacting a compound of Formula (I) with a base. In some embodiments, the compound of Formula (I) is acidic and is reacted with a base. In such situations, an acidic proton of the compound of Formula (I) is replaced by a metal ion, e.g., lithium, sodium, potassium, magnesium, calcium, or an aluminum ion. In some cases, compounds described herein coordinate with an organic base, such as, but not limited to, ethanolamine, diethanolamine, triethanolamine, tromethamine, meglumine, N-methylglucamine, dicyclohexylamine, tris(hydroxymethyl)methylamine. In other cases, compounds described herein form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, potassium carbonate, sodium hydroxide, lithium hydroxide, and the like. In some embodiments, the compounds provided herein are prepared as a sodium salt, calcium salt, potassium salt, magnesium salt, meglumine salt, N-methylglucamine salt or ammonium salt.

It should be understood that a reference to a pharmaceutically acceptable salt includes the solvent addition forms. In some embodiments, solvates contain either stoichiometric or non-stoichiometric amounts of a solvent, and are formed during the process of crystallization with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. In addition, the compounds provided herein optionally exist in unsolvated as well as solvated forms.

The methods and formulations described herein include the use of N-oxides (if appropriate), or pharmaceutically acceptable salts of compounds having the structure of Formula (I), as well as active metabolites of these compounds having the same type of activity.

In some embodiments, sites on the organic radicals (e.g. alkyl groups, aromatic rings) of compounds of Formula (I) are susceptible to various metabolic reactions. Incorporation of appropriate substituents on the organic radicals will reduce, minimize or eliminate this metabolic pathway. In specific embodiments, the appropriate substituent to decrease or eliminate the susceptibility of the aromatic ring to metabolic reactions is, by way of example only, a halogen, deuterium, an alkyl group, a haloalkyl group, or a deuteroalkyl group.

In another embodiment, the compounds described herein are labeled isotopically (e.g. with a radioisotope) or by another other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, sulfur, fluorine chlorine, iodine, phosphorus, such as, for example, ²H, ³H, ¹³C, ¹⁴C, ¹⁵N, ¹⁸O, ¹⁷O, ³⁵S, ¹⁸F, ³⁶Cl, ¹²³I, ¹²⁴I, ¹²⁵I, ¹³¹I, ³²P and ³³P. In one aspect, isotopically-labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements.

In some embodiments, the compounds of Formula (I) possess one or more stereocenters and each stereocenter exists independently in either the R or S configuration. In some embodiments, the compound of Formula (I) exists in the R configuration. In some embodiments, the compound of Formula (I) exists in the S configuration. The compounds presented herein include all diastereomeric, individual enantiomers, atropisomers, and epimeric forms as well as the appropriate mixtures thereof. The compounds and methods provided herein include all cis, trans, syn, anti, entgegen (E), and zusammen (Z) isomers as well as the appropriate mixtures thereof.

Individual stereoisomers are obtained, if desired, by methods such as, stereoselective synthesis and/or the separation of stereoisomers by chiral chromatographic columns or the separation of diastereomers by either non-chiral or chiral chromatographic columns or crystallization and recrystallization in a proper solvent or a mixture of solvents. In certain embodiments, compounds of Formula (I) are prepared as their individual stereoisomers by reacting a racemic mixture of the compound with an optically active resolving agent to form a pair of diastereoisomeric compounds/salts, separating the diastereomers and recovering the optically pure individual enantiomers. In some embodiments, resolution of individual enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley And Sons, Inc., 1981. In some embodiments, stereoisomers are obtained by stereoselective synthesis.

In some embodiments, compounds described herein are prepared as prodrugs. A “prodrug” refers to an agent that is converted into the parent drug in vivo. Prodrugs are often useful because, in some situations, they are easier to administer than the parent drug. They are, for instance, bioavailable by oral administration whereas the parent is not. Further or alternatively, the prodrug also has improved solubility in pharmaceutical compositions over the parent drug. In some embodiments, the design of a prodrug increases the effective water solubility. An example, without limitation, of a prodrug is a compound described herein, which is administered as an ester (the “prodrug”) but then is metabolically hydrolyzed to provide the active entity. A further example of a prodrug is a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound.

Prodrugs of the compounds described herein include, but are not limited to, esters, ethers, carbonates, thiocarbonates, N-acyl derivatives, N-acyloxyalkyl derivatives, N-alkyloxyacyl derivatives, quaternary derivatives of tertiary amines, N-Mannich bases, Schiff bases, amino acid conjugates, phosphate esters, and sulfonate esters. See for example Design of Prodrugs, Bundgaard, A. Ed., Elseview, 1985 and Method in Enzymology, Widder, K. et al., Ed.; Academic, 1985, vol. 42, p. 309-396; Bundgaard, H. “Design and Application of Prodrugs” in A Textbook of Drug Design and Development, Krosgaard-Larsen and H. Bundgaard, Ed., 1991, Chapter 5, p. 113-191; and Bundgaard, H., Advanced Drug Delivery Review, 1992, 8, 1-38, each of which is incorporated herein by reference. In some embodiments, a hydroxyl group in the compounds disclosed herein is used to form a prodrug, wherein the hydroxyl group is incorporated into an acyloxyalkyl ester, alkoxycarbonyloxyalkyl ester, alkyl ester, aryl ester, phosphate ester, sugar ester, ether, and the like. In some embodiments, a hydroxyl group in the compounds disclosed herein is a prodrug wherein the hydroxyl is then metabolized in vivo to provide a carboxylic acid group. In some embodiments, a carboxyl group is used to provide an ester or amide (i.e. the prodrug), which is then metabolized in vivo to provide a carboxylic acid group. In some embodiments, compounds described herein are prepared as alkyl ester prodrugs.

Prodrug forms of the herein described compounds, wherein the prodrug is metabolized in vivo to produce a compound of Formula (I) as set forth herein are included within the scope of the claims. In some cases, some of the herein-described compounds is a prodrug for another derivative or active compound.

In some embodiments, any one of the hydroxyl group(s), amino group(s) and/or carboxylic acid group(s) are functionalized in a suitable manner to provide a prodrug moiety. In some embodiments, the prodrug moiety is as described above.

In additional or further embodiments, the compounds described herein are metabolized upon administration to an organism in need to produce a metabolite that is then used to produce a desired effect, including a desired therapeutic effect.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulfhydryl groups. Metabolites of the compounds disclosed herein are optionally identified either by administration of compounds to a host and analysis of tissue samples from the host, or by incubation of compounds with hepatic cells in vitro and analysis of the resulting compounds.

In some instances, heterocyclic rings may exist in tautomeric forms. In such situations, it is understood that the structures of said compounds are illustrated or named in one tautomeric form but could be illustrated or named in the alternative tautomeric form. The alternative tautomeric forms are expressly included in this disclosure, such as, for example, the structures illustrated below. For example, benzimidazoles or imidazoles could exist in the following tautomeric forms:

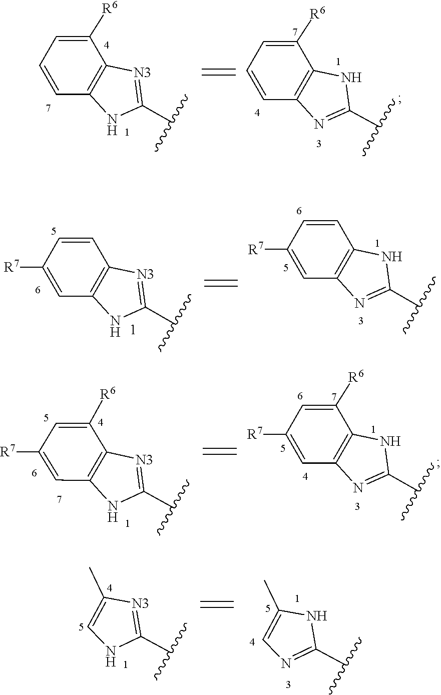

### Preparation of Compounds

Compounds of Formula (I) described herein are synthesized using standard synthetic techniques or using methods known in the art in combination with methods described herein.

Unless otherwise indicated, conventional methods of mass spectroscopy, NMR, HPLC are employed.

Compounds are prepared using standard organic chemistry techniques such as those described in, for example, March's Advanced Organic Chemistry, 6^{th} Edition, John Wiley and Sons, Inc. Alternative reaction conditions for the synthetic transformations described herein may be employed such as variation of solvent, reaction temperature, reaction time, as well as different chemical reagents and other reaction conditions.

In some embodiments, compounds described herein are prepared as described in Scheme A.

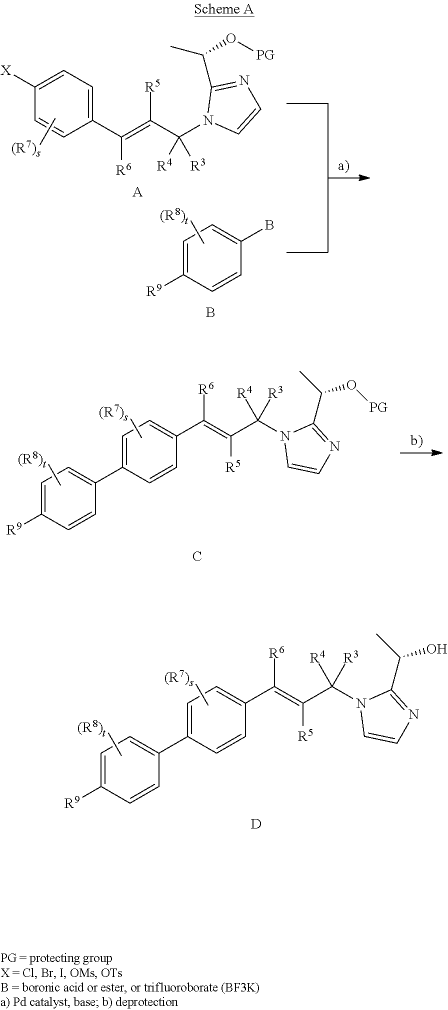

An organometallic coupling reaction such as Suzuki-Miyaura reaction between Intermediate A and the appropriate aryl boronic acid or its ester or an organotrifluoroborate (BF₃K) B provided Intermediate C. Removal of the protecting group using appropriate deprotection methods yielded final Compound D.

In some other embodiments, compounds described herein are prepared as described in Scheme B.

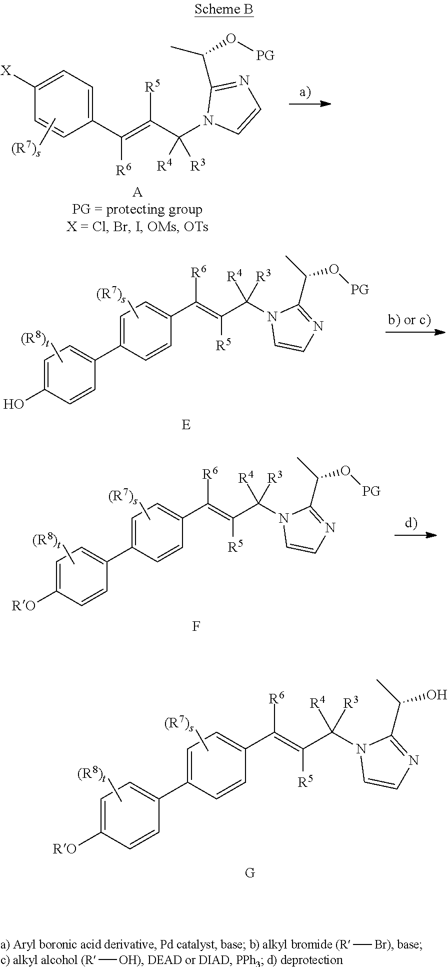

An organometallic coupling reaction such as Suzuki-Miyaura reaction between Intermediate A and the appropriate aryl boronic acid or its ester or an organotrifluoroborate (BF₃K) provided Intermediate E. Substitution reaction with an appropriate alkylbromide (R′—Br) provided Intermediate F. Alternatively, Intermediate E can be treated with a suitable alcohol (R′—OH) under Mitsunobu reaction conditions to provide Intermediate F. Removal of the protecting group using appropriate deprotection methods yielded final Compound G.

In some embodiments, further elaboration of intermediates is performed before or after the deprotection step.

For example, in some embodiments, compounds described herein are prepared as described in Scheme C.

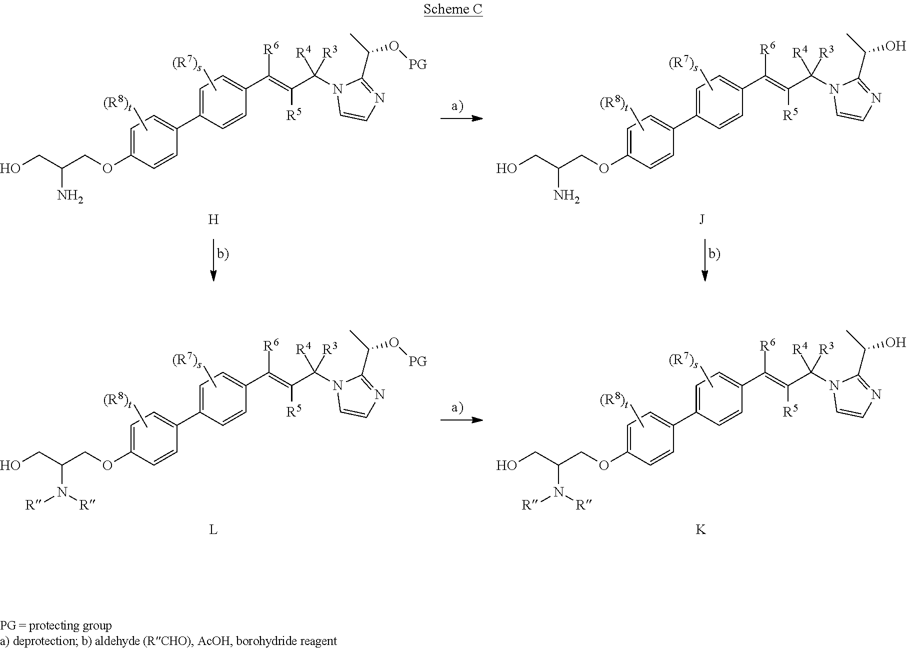

Free amine (NH₂)-containing Intermediate H is deprotected to final Compound J using appropriate deprotection conditions. Free amine (NH₂)-containing Compound J can be further reacted with an appropriate aldehyde (R″—CHO) under appropriate reductive amination conditions (such as treatment with a borohydride reagent: for example, NaBH₄, NaCNBH₃, or NaB(OAc)₃H) to provide final Compound K. Alternatively, Intermediate H can be reacted under the reductive amination conditions to provide Intermediate L, which is deprotected using appropriate deprotection methods yielded final Compound K.

In some embodiments, further elaboration of intermediates is performed before final deprotection.

In some other embodiments, compounds described herein are prepared as described in Scheme D.

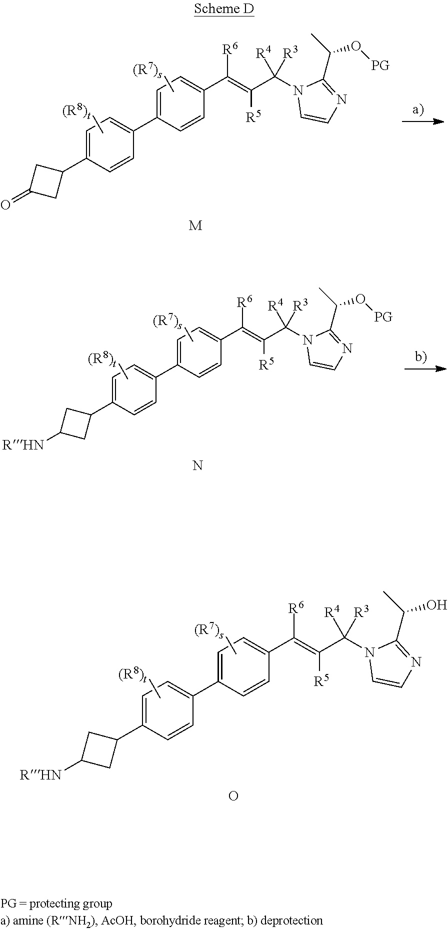

Ketone containing Intermediate M is reacted with an appropriate amine (R″′—NH₂) under appropriate reductive amination conditions (such as treatment with a borohydride reagent: for example, NaBH₄, NaCNBH₃, or NaB(OAc)₃H) to provide Intermediate N. Removal of the protecting group using appropriate deprotection methods yielded final Compound O.

In some embodiments, compounds are prepared as described in the Examples.

### Certain Terminology

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

Unless otherwise stated, the following terms used in this application have the definitions given below. The use of the term “including” as well as other forms, such as “include”, “includes,” and “included,” is not limiting. The section headings used herein are for organizational purposes only and are not to be construed as limiting the subject matter described.

As used herein, C₁-Cₓ includes C₁-C₂, C₁-C₃ . . . C₁-Cₓ. By way of example only, a group designated as “C₁-C₆” indicates that there are one to six carbon atoms in the moiety, i.e. groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl.

An “alkyl” group refers to an aliphatic hydrocarbon group. The alkyl group is branched or straight chain. In some embodiments, the “alkyl” group has 1 to 10 carbon atoms, i.e. a C₁-C₁₀alkyl. Whenever it appears herein, a numerical range such as “1 to 10” refers to each integer in the given range; e.g., “1 to 10 carbon atoms” means that the alkyl group consist of 1 carbon atom, 2 carbon atoms, 3 carbon atoms, etc., up to and including 10 carbon atoms, although the present definition also covers the occurrence of the term “alkyl” where no numerical range is designated. In some embodiments, an alkyl is a C₁-C₆alkyl. In one aspect the alkyl is methyl, ethyl, propyl, iso-propyl, n-butyl, iso-butyl, sec-butyl, or t-butyl. Typical alkyl groups include, but are in no way limited to, methyl, ethyl, propyl, isopropyl, butyl, isobutyl, sec-butyl, tertiary butyl, pentyl, neopentyl, or hexyl. In some embodiments, an alkyl is methyl.

An “alkylene” group refers to a divalent alkyl radical. Any of the above mentioned monovalent alkyl groups may be an alkylene by abstraction of a second hydrogen atom from the alkyl. In some embodiments, an alkylene is a C₁-C₆alkylene. In other embodiments, an alkylene is a C₁-C₄alkylene. Typical alkylene groups include, but are not limited to, —CH₂—, —CH₂CH₂—, —CH₂CH₂CH₂—, —CH₂CH₂CH₂CH₂—, and the like. In some embodiments, an alkylene is —CH₂—.

An “alkoxy” group refers to a —O(alkyl) group, where alkyl is as defined herein.

The term “alkylamine” refers to the —N(alkyl)ₓH_{y} group, where x is 0 and y is 2, or where x is 1 and y is 1, or where x is 2 and y is 0.

An “hydroxyalkyl” refers to an alkyl in which one hydrogen atom is replaced by a hydroxyl. In some embodiments, a hydroxyalkyl is a C₁-C₄hydroxyalkyl. Typical hydroxyalkyl groups include, but are not limited to, —CH₂OH, —CH₂CH₂OH, —CH₂CH₂CH₂OH, —CH₂CH₂CH₂CH₂OH, and the like. In some embodiments, a hydroxyalkyl is —CH₂OH or —CH₂CH₂OH. In some embodiments, a hydroxyalkyl is —CH₂OH. In some embodiments, a hydroxyalkyl is —CH₂CH₂OH.

An “aminoalkyl” refers to an alkyl in which one hydrogen atom is replaced by an amino. In some embodiments, aminoalkyl is a C₁-C₄aminoalkyl. Typical aminoalkyl groups include, but are not limited to, —CH₂NH₂, —CH₂CH₂NH₂, —CH₂CH₂CH₂NH₂, —CH₂CH₂CH₂CH₂NH₂, and the like.

The term “alkenyl” refers to a type of alkyl group in which at least one carbon-carbon double bond is present. In one embodiment, an alkenyl group has the formula —C(R)═CR₂, wherein R refers to the remaining portions of the alkenyl group, which may be the same or different. In some embodiments, R is H or an alkyl. In some embodiments, an alkenyl is selected from ethenyl (i.e., vinyl), propenyl (i.e., allyl), butenyl, pentenyl, pentadienyl, and the like. Non-limiting examples of an alkenyl group include —CH═CH₂, —C(CH₃)═CH₂, —CH═CHCH₃, —C(CH₃)═CHCH₃, and —CH₂CH═CH₂.

The term “alkynyl” refers to a type of alkyl group in which at least one carbon-carbon triple bond is present. In one embodiment, an alkynyl group has the formula —C≡C—R, wherein R refers to the remaining portions of the alkynyl group. In some embodiments, R is H or an alkyl.

In some embodiments, an alkynyl is selected from ethynyl, propynyl, butynyl, pentynyl, hexynyl, and the like. Non-limiting examples of an alkynyl group include —C≡CH, —C≡CCH₃—C≡CCH₂CH₃, —CH₂C≡CH.

The term “heteroalkyl” refers to an alkyl group in which one or more skeletal atoms of the alkyl are selected from an atom other than carbon, e.g., oxygen, nitrogen (e.g. —NH—, —N(alkyl)-, sulfur, or combinations thereof. A heteroalkyl is attached to the rest of the molecule at a carbon atom of the heteroalkyl. In one aspect, a heteroalkyl is a C₁-C₆heteroalkyl. In some embodiments, a heteroalkyl is a C₁-C₆heteroalkyl where one or two atoms are independently selected from O, NH and S.

The term “aromatic” refers to a planar ring having a delocalized 7r-electron system containing 4n+2π electrons, where n is an integer. The term “aromatic” includes both carbocyclic aryl (“aryl”, e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups.

The term “carbocyclic” or “carbocycle” refers to a ring or ring system where the atoms forming the backbone of the ring are all carbon atoms. The term thus distinguishes carbocyclic from “heterocyclic” rings or “heterocycles” in which the ring backbone contains at least one atom which is different from carbon. In some embodiments, at least one of the two rings of a bicyclic carbocycle is aromatic. In some embodiments, both rings of a bicyclic carbocycle are aromatic. Carbocycles include aryls and cycloalkyls.

As used herein, the term “aryl” refers to an aromatic ring wherein each of the atoms forming the ring is a carbon atom. In one aspect, aryl is phenyl or a naphthyl. In some embodiments, an aryl is a phenyl. In some embodiments, an aryl is a phenyl, naphthyl, indanyl, indenyl, or tetrahydronaphthyl. In some embodiments, an aryl is a phenyl. In some embodiments, an aryl is a C₆-C₁₀aryl. Depending on the structure, an aryl group is a monoradical or a diradical (i.e., an arylene group).

The term “cycloalkyl” refers to a monocyclic or polycyclic aliphatic, non-aromatic radical, wherein each of the atoms forming the ring (i.e. skeletal atoms) is a carbon atom. In some embodiments, cycloalkyls are spirocyclic or bridged compounds. In some embodiments, cycloalkyls are optionally fused with an aromatic ring, and the point of attachment is at a carbon that is not an aromatic ring carbon atom. Cycloalkyl groups include groups having from 3 to 10 ring atoms. In some embodiments, cycloalkyl groups are selected from among cyclopropyl, cyclobutyl, cyclopentyl, cyclopentenyl, cyclohexyl, cyclohexenyl, cycloheptyl, cyclooctyl, spiro[2.2]pentyl, norbornyl and bicyclo[1.1.1]pentyl. In some embodiments, a cycloalkyl is a C₃-C₆cycloalkyl. In some embodiments, a cycloalkyl is a C₃-C₄cycloalkyl. In some embodiments, a cycloalkyl is a cyclopropyl. In some embodiments, a cycloalkyl is a cyclobutyl.

The term “halo” or, alternatively, “halogen” or “halide” means fluoro, chloro, bromo or iodo. In some embodiments, halo is fluoro, chloro, or bromo.

The term “fluoroalkyl” refers to an alkyl in which one or more hydrogen atoms are replaced by a fluorine atom. In one aspect, a fluoroalkyl is a C₁-C₆fluoroalkyl. In some embodiments, a fluoroalkyl is —CF₃.

The term “heterocycle” or “heterocyclic” refers to heteroaromatic rings (also known as heteroaryls) and heterocycloalkyl rings containing one to four heteroatoms in the ring(s), where each heteroatom in the ring(s) is selected from O, S and N, wherein each heterocyclic group has from 3 to 10 atoms in its ring system, and with the proviso that any ring does not contain two adjacent O or S atoms. Non-aromatic heterocyclic groups (also known as heterocycloalkyls) include rings having 3 to 10 atoms in its ring system and aromatic heterocyclic groups include rings having 5 to 10 atoms in its ring system. The heterocyclic groups include benzo-fused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, indolin-2-onyl, isoindolin-1-onyl, isoindoline-1,3-dionyl, 3,4-dihydroisoquinolin-1(2H)-onyl, 3,4-dihydroquinolin-2(1H)-onyl, isoindoline-1,3-dithionyl, benzo[d]oxazol-2(3H)-onyl, 1H-benzo[d]imidazol-2(3H)-onyl, benzo[d]thiazol-2(3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic.

The terms “heteroaryl” or, alternatively, “heteroaromatic” refers to an aryl group that includes one or more ring heteroatoms selected from nitrogen, oxygen and sulfur. Illustrative examples of heteroaryl groups include monocyclic heteroaryls and bicyclic heteroaryls. Monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Monocyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, a heteroaryl contains 0-4 N atoms in the ring. In some embodiments, a heteroaryl contains 1-4 N atoms in the ring. In some embodiments, a heteroaryl contains 0-4 N atoms, 0-1 O atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉heteroaryl. In some embodiments, monocyclic heteroaryl is a C₁-C₅heteroaryl. In some embodiments, monocyclic heteroaryl is a 5-membered or 6-membered heteroaryl. In some embodiments, bicyclic heteroaryl is a C₆-C₉heteroaryl.

A “heterocycloalkyl” group refers to a cycloalkyl group that includes at least one heteroatom selected from nitrogen, oxygen and sulfur. In some embodiments, a heterocycloalkyl is fused with an aryl or heteroaryl. In some embodiments, the heterocycloalkyl is oxazolidinonyl, pyrrolidinyl, tetrahydrofuranyl, tetrahydrothienyl, tetrahydropyranyl, tetrahydrothiopyranyl, piperidinyl, morpholinyl, thiomorpholinyl, piperazinyl, piperidin-2-onyl, pyrrolidine-2,5-dithionyl, pyrrolidine-2,5-dionyl, pyrrolidinonyl, imidazolidinyl, imidazolidin-2-onyl, or thiazolidin-2-onyl. In one aspect, a heterocycloalkyl is a C₂-C₁₀heterocycloalkyl. In another aspect, a heterocycloalkyl is a C₄-C₁₀heterocycloalkyl. In some embodiments, a heterocycloalkyl is monocyclic or bicyclic. In some embodiments, a heterocycloalkyl is monocyclic and is a 3, 4, 5, 6, 7, or 8-membered ring. In some embodiments, a heterocycloalkyl is monocyclic and is a 3, 4, 5, or 6-membered ring. In some embodiments, a heterocycloalkyl is monocyclic and is a 3 or 4-membered ring. In some embodiments, a heterocycloalkyl contains 0-2 N atoms in the ring. In some embodiments, a heterocycloalkyl contains 0-2 N atoms, 0-2 O atoms and 0-1 S atoms in the ring.

The term “bond” or “single bond” refers to a chemical bond between two atoms, or two moieties when the atoms joined by the bond are considered to be part of larger substructure. In one aspect, when a group described herein is a bond, the referenced group is absent thereby allowing a bond to be formed between the remaining identified groups.

The term “moiety” refers to a specific segment or functional group of a molecule. Chemical moieties are often recognized chemical entities embedded in or appended to a molecule.

The term “optionally substituted” or “substituted” means that the referenced group is optionally substituted with one or more additional group(s) individually and independently selected from halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from halogen, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁—C₄alkyl, —S(═O)C₁-C₄alkyl, and —S(═O)₂C₁-C₄alkyl. In some embodiments, optional substituents are independently selected from halogen, —CN, —NH₂, —OH, —NH(CH₃), —N(CH₃)₂, —CH₃, —CH₂CH₃, —CHF₂, —CF₃, —OCH₃, —OCHF₂, and —OCF₃. In some embodiments, substituted groups are substituted with one or two of the preceding groups. In some embodiments, an optional substituent on an aliphatic carbon atom (acyclic or cyclic) includes oxo (═O).

In some embodiments, each substituted alkyl, substituted fluoroalkyl, substituted heteroalkyl, substituted carbocycle, and substituted heterocycle is substituted with one or more R⁵ groups independently selected from the group consisting of halogen, C₁-C₆alkyl, monocyclic carbocycle, monocyclic heterocycle, —CN, —OR²¹, —CO₂R²¹, —C(═O)N(R²¹)₂, —N(R²¹)₂, —NR²¹C(═O)R²², —SR²¹, —S(═O)R²², —SO₂R²², or —SO₂N(R²¹)₂; each R²¹ is independently selected from hydrogen, C₁-C₆alkyl, C₁-C₆fluoroalkyl, C₁-C₆heteroalkyl, C₃-C₆cycloalkyl, C₂-C₆heterocycloalkyl, phenyl, benzyl, 5-membered heteroaryl and 6-membered heteroaryl; or two R²¹ groups are taken together with the N atom to which they are attached to form a N-containing heterocycle; each R²² is independently selected from C₁-C₆alkyl, C₁-C₆fluoroalkyl, C₁-C₆heteroalkyl, C₃-C₆cycloalkyl, C₂-C₆heterocycloalkyl, phenyl, benzyl, 5-membered heteroaryl and 6-membered heteroaryl.

The term “acceptable” with respect to a formulation, composition or ingredient, as used herein, means having no persistent detrimental effect on the general health of the subject being treated.

The term “modulate” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator” as used herein, refers to a molecule that interacts with a target either directly or indirectly. The interactions include, but are not limited to, the interactions of an agonist, partial agonist, an inverse agonist, antagonist, degrader, or combinations thereof. In some embodiments, a modulator is an antagonist. In some embodiments, a modulator is an inhibitor.

The terms “administer,” “administering”, “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein. In some embodiments, the compounds and compositions described herein are administered orally.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered, which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound of Formula (I), or a pharmaceutically acceptable salt thereof, and a co-agent, are both administered to a patient simultaneously in the form of a single entity or dosage. The term “non-fixed combination” means that the active ingredients, e.g. a compound of Formula (I), or a pharmaceutically acceptable salt thereof, and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The terms “article of manufacture” and “kit” are used as synonyms.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development or progression of the disease or condition, relieving the disease or condition, causing regression of the disease or condition, relieving a secondary condition caused by the disease or condition, or stopping the symptoms of the disease or condition either prophylactically and/or therapeutically.

### Pharmaceutical Compositions

In certain embodiments, the heterocyclic LpxC inhibitory compound as described herein is administered as a pure chemical. In other embodiments, the heterocyclic LpxC inhibitory compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Provided herein is a pharmaceutical composition comprising at least one heterocyclic LpxC inhibitory compound as described herein, or a stereoisomer, pharmaceutically acceptable salt, or N-oxide thereof, together with one or more pharmaceutically acceptable carriers. The carrier(s) (or excipient(s)) is acceptable or suitable if the carrier is compatible with the other ingredients of the composition and not deleterious to the recipient (i.e., the subject or patient) of the composition.

Some embodiments provide a pharmaceutical composition comprising a compound of Formula (I), or a pharmaceutically acceptable salt thereof, and a pharmaceutically acceptable excipient.

In certain embodiments, the heterocyclic LpxC inhibitory compound as described by Formula (I) is substantially pure, in that it contains less than about 5%, or less than about 1%, or less than about 0.1%, of other organic small molecules, such as unreacted intermediates or synthesis by-products that are created, for example, in one or more of the steps of a synthesis method.

Suitable oral dosage forms include, for example, tablets, pills, sachets, or capsules of hard or soft gelatin, methylcellulose or of another suitable material easily dissolved in the digestive tract. In some embodiments, suitable nontoxic solid carriers are used which include, for example, pharmaceutical grades of mannitol, lactose, starch, magnesium stearate, sodium saccharin, talcum, cellulose, glucose, sucrose, magnesium carbonate, and the like. (See, e.g., Remington: The Science and Practice of Pharmacy (Gennaro, 21^{st} Ed. Mack Pub. Co., Easton, PA (2005)).

The dose of the composition comprising at least one heterocyclic LpxC inhibitory compound as described herein differ, depending upon the patient's condition, that is, stage of the disease, general health status, age, and other factors.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome), or a lessening of symptom severity. Optimal doses are generally determined using experimental models and/or clinical trials. The optimal dose depends upon the body mass, weight, or blood volume of the patient.

Oral doses typically range from about 1.0 mg to about 1000 mg, one to four times, or more, per day.

### Combination Treatments

In certain instances, it is appropriate to administer at least one compound of Formula (I), or a pharmaceutically acceptable salt thereof, in combination with one or more other therapeutic agents.

In one embodiment, the therapeutic effectiveness of one of the compounds described herein is enhanced by administration of an adjuvant (i.e., by itself the adjuvant has minimal therapeutic benefit, but in combination with another therapeutic agent, the overall therapeutic benefit to the patient is enhanced). Or, in some embodiments, the benefit experienced by a patient is increased by administering one of the compounds described herein with another agent (which also includes a therapeutic regimen) that also has therapeutic benefit.

In one specific embodiment, a compound of Formula (I), or a pharmaceutically acceptable salt thereof, is co-administered with a second therapeutic agent, wherein the compound of Formula (I), or a pharmaceutically acceptable salt thereof, and the second therapeutic agent modulate different aspects of the disease, disorder or condition being treated, thereby providing a greater overall benefit than administration of either therapeutic agent alone.

In any case, regardless of the disease, disorder or condition being treated, the overall benefit experienced by the patient is simply be additive of the two therapeutic agents or the patient experiences a synergistic benefit.

For combination therapies described herein, dosages of the co-administered compounds vary depending on the type of co-drug employed, on the specific drug employed, on the disease or condition being treated and so forth. In additional embodiments, when co-administered with one or more other therapeutic agents, the compound provided herein is administered either simultaneously with the one or more other therapeutic agents, or sequentially.

In combination therapies, the multiple therapeutic agents (one of which is one of the compounds described herein) are administered in any order or even simultaneously. If administration is simultaneous, the multiple therapeutic agents are, by way of example only, provided in a single, unified form, or in multiple forms (e.g., as a single pill or as two separate pills).

The compounds of Formula (I), or a pharmaceutically acceptable salt thereof, as well as combination therapies, are administered before, during or after the occurrence of a disease or condition, and the timing of administering the composition containing a compound varies. Thus, in one embodiment, the compounds described herein are used as a prophylactic and are administered continuously to subjects with a propensity to develop conditions or diseases in order to prevent the occurrence of the disease or condition. In another embodiment, the compounds and compositions are administered to a subject during or as soon as possible after the onset of the symptoms. In specific embodiments, a compound described herein is administered as soon as is practicable after the onset of a disease or condition is detected or suspected, and for a length of time necessary for the treatment of the disease. In some embodiments, the length required for treatment varies, and the treatment length is adjusted to suit the specific needs of each subject.

Other embodiments and uses will be apparent to one skilled in the art in light of the present disclosures. The following examples are provided merely as illustrative of various embodiments and shall not be construed to limit the invention in any way.

### EXAMPLES

As used above, and throughout the description of the invention, the following abbreviations, unless otherwise indicated, shall be understood to have the following meanings:

### Abbreviations:

ACN or MeCN: acetonitrile;
aq: aqueous;
Boc or BOC: tert-butoxycarbonyl;
DCM: dichloromethane;
DEAD: diethyl azodicarboxylate;
DIBAL(-H): diisobutylaluminum hydride;
DIPEA or DIEA: diisopropylethylamine;
DMAP: 4-dimethylaminopyridine;
DMF: dimethylformamide;
DMP: Dess-Martin periodinane
DPPA: diphenylphosphoryl azide;
Eq. or equiv: equivalents;
EtOAc: ethyl acetate;
g: grams
h or hr(s): hour(s);
HPLC: high-performance liquid chromatography;
LC-MS, LC MS, or LCMS: liquid chromatography-mass spectrometry;
LDA: lithium diisopropylamide;
M: molar;
MeOH: methanol;
mg: milligrams;
min: minute;
mL: milliliter;
mmol: millimole;
MsCl: methanesulfonyl (mesyl) chloride;
MTBE: methyl tert-butyl ether;
N: normal;
NMR: nuclear magnetic resonance;
Pet ether: petroleum ether;
p-TSA: para-toluenesuflonic acid
rt: room temperature;
SFC: supercritical fluid chromatography;
TFA: trifluoroacetic acid;
THF: tetrahydrofuran;
TLC: thin layer chromatography;
TsCl: para-toluenesulfonyl (tosyl) chloride.

The following examples are provided for illustrative purposes only and not to limit the scope of the claims provided herein.

### I. Chemical Synthesis

Unless otherwise noted, reagents and solvents were used as received from commercial suppliers. Anhydrous solvents and oven-dried glassware were used for synthetic transformations sensitive to moisture and/or oxygen. Yields were not optimized. Reaction times are approximate and were not optimized. Column chromatography and thin layer chromatography (TLC) were performed on silica gel unless otherwise noted. Spectra are given in ppm (D) and coupling constants, J are reported in Hertz. For proton spectra the solvent peak was used as the reference peak.

### Example 1: Preparation of 2-((1S)-1-((tetrahydro-2H-pyran-2-yl)oxy)ethyl)-1H-imidazole (Int-5)

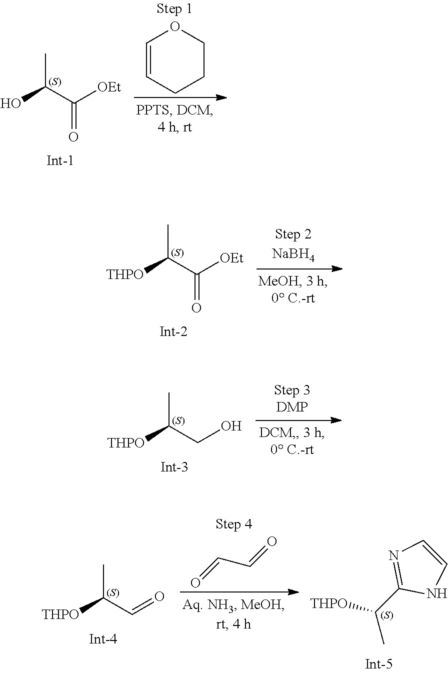

Step 1: Pyridinium p-toluenesulfonate (0.408 g, 1.62 mmol) was added to a solution of ethyl (s)-(−)-lactate (Int-1, 50 g, 0.4232 mol) and 3,4-dihydro-2H-pyran (46.2 g, 0.549 mol) in DCM (566 mL, 11 v/w). The reaction mixture was stirred 4 h at room temperature. After completion of the reaction, as monitored by TLC, the reaction mixture was diluted with DCM and washed with saturated NaHCO₃ solution. The layers were separated, the organic layer dried over sodium sulphate, filtered and concentrated under reduced pressure to get pure Int-2 as a colorless liquid as a mixture of diastereomers. The ¹H NMR data was in accordance with that reported in J. Org. Chem. 2009, 74, 8154. Yield: 80 g, 93%.

Step 2: To a 0° C. cooled solution of Int-2 (50 g, 0.2472 mol) in MeOH (750 mL, 15 v/w), was added NaBH₄ (37.40 g, 0.988 mol, 4 equiv.) in portions over 1 h. The reaction mixture was allowed to warm to room temperature over 3 h. After completion of the reaction, as monitored by TLC, the reaction mixture was quenched with sat. NH₄Cl solution and extracted with DCM (500 mL×3). The layers were separated, and the combined organic layer was dried over sodium sulphate, filtered and concentrated under reduced pressure. The crude product was purified by silica column chromatography (60-120 mesh, eluted with 30-50% EtOAc and pet ether) to get pure Int-3 as a colorless liquid. Yield: 25 g, 63%.

Step 3: To a 0° C. cooled solution of Int-3 (40 g, 0.2496 mol) in DCM (800 mL, 20 v/w) was added Dess-Martin periodinane (158.8 g, 0.3744 mol) in portions over 30 min. The reaction mixture was allowed to warm to room temperature over 3 h. After completion of the reaction, as monitored by TLC, the reaction mixture was filtered on Celite bed and the bed was further washed with DCM (1000 mL). The filtrate was washed with saturated NaHCO₃ solution followed by brine. The organic layer was dried over sodium sulphate, filtered and concentrated under reduced pressure at 30° C. Some white solid was observed in the crude product. To remove this impurity, the crude product was dissolved in diethyl ether and washed with 10% NaOH solution (500 mL×2). The organic layer was dried over sodium sulphate, filtered and concentrated under reduced pressure to get a crude mass. Since the formation of the white solid was observed yet again, the crude mass was dissolved in diethyl ether (500 mL), filtered on celite bed and washed with diethyl ether (750 mL) to completely remove the unwanted impurity. The filtrate was concentrated under reduced pressure at 30° C. to get Int-4 as a colorless liquid as a mixture of diastereomers, which was taken to the next step without further purification. The ¹H NMR showed all the characteristic resonances reported in Org. Lett. 2009, 11, 1103. Yield: 39 g, 98%.

Step 4: To a solution of Int-4 (39 g, 246.5 mol) in MeOH (390 mL, 10 v/w), was added glyoxal (40% in water, 97.5 mL, 2.5 v/w). To the above mixture cooled to 10° C., was added 28% aqueous ammonia (120 mL, 3 v/w). The ice bath was removed, and the reaction mixture temperature allowed to run at room temperature for 4 h. After completion of the reaction, as monitored by TLC and LC-MS, the reaction mixture was concentrated under reduced pressure. The crude product was purified by column chromatography (SiO2 60-120 mesh, eluted with 80-100% EtOAc in Pet ether) to get 2-((1S)-1-((tetrahydro-2H-pyran-2-yl)oxy)ethyl)-1H-imidazole (Int-5) as an off white solid as a mixture of diastereomers. The 1H NMR recorded in DMSO-d6 and LCMS were supportive of the structure. For ¹H NMR values recorded in CDCl₃, see WO 2018216822A1. Yield: 27 g, 56%. LC MS: Calculated for C₁₀H₁₆N₂O₂ is 196.25, Observed: 197.1 [M+H]⁺.

### Example 2: Preparation of 1-((E)-3-(4-iodophenyl)allyl)-2-((1S)-1-((tetrahydro-2H-pyran-2-yl)oxy)ethyl)-1H-imidazole (Int-8)

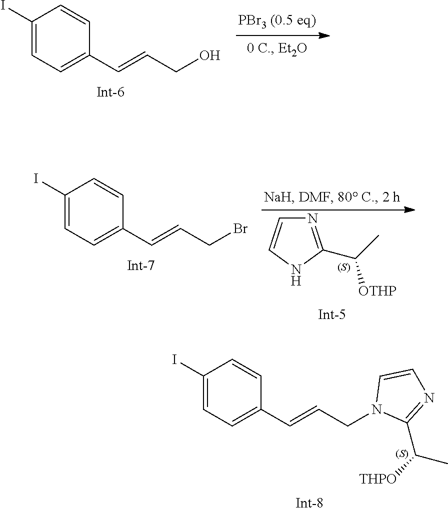

Step 1: A solution of 4-iodocinnmayl alcohol Int-6 (2 g, 7.6 mmol) in diethyl ether (30 mL) was cooled to 0° C. To this cooled solution, PBr₃ (1.24 g, 4.6 mmol) was added and stirred at 0° C. for 2 h under darkness. After consumption of starting material, the reaction mixture was diluted with diethyl ether and quenched by pouring to the cooled sat. NaHCO₃ solution and extracted twice with diethyl ether. The combined organic layers were dried over anhydrous Na₂SO₄, filtered and concentrated under reduced pressure to afford pure product Int-7. Yield: 2.01 g, 84%. LC MS: Calculated for C₉H₈BrI is 322.97, not ionized.

Step 2: A solution of Int-7 (1 g, 5.1 mmol, see part II) in DMF (25 mL) was added to the slurry of NaH (5.1 mmol) in DMF (5 mL) and the reaction mixture heated to 50° C. for 30 min. To the preheated reaction mixture, a solution of 7 (1.64 g, 5.1 mmol) in DMF (5 mL) was added and the heating continued at 80° C. for 3 h. After consumption of the starting materials, the reaction mixture was cooled to rt and quenched with sat. NH₄Cl. The reaction mixture was diluted with water and extracted twice with EtOAc. The combined organic layers were dried over Na₂SO₄, filtered and concentrated under reduced pressure. The crude product was purified by column chromatography using silica gel (230-400 mesh) by eluting with 1-2% methanol in DCM to afford pure product Int-8. Yield: 1.05 g, 48%. LC MS: Calculated for C₁₉H₂₃IN₂O₂ is 438.31, Observed: 439.2 [M+H]⁺.

### Example 3: Preparation of (2S,E)-4-(4-iodophenyl)-2-(2-((1S)-1-((tetrahydro-2H-pyran-2-yl)oxy)ethyl)-1H-imidazol-1-yl)but-3-en-1-ol (Int-16a) and (3R,E)-5-(4-iodophenyl)-3-(2-((1S)-1-((tetrahydro-2H-pyran-2-yl)oxy)ethyl)-1H-imidazol-1-yl)pent-4-en-1-ol (Int-16b)

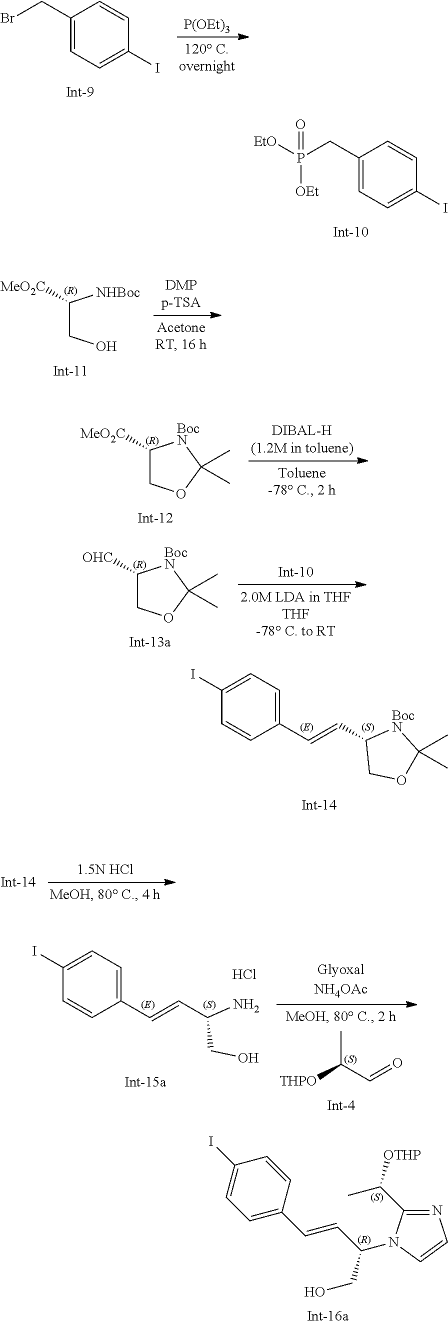

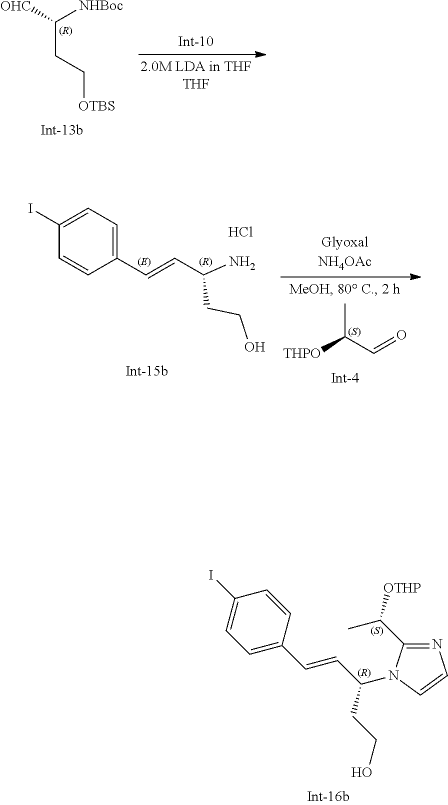

Step 1: A mixture of 4-Iodobenzyl bromide (Int-9) (130 g, 438 mmol) and triethyl phosphite (116 g, 700 mmol) was heated at 120° C. for overnight. The excess triethyl phosphite was removed by fractional distillation. The resulting mixture was diluted with water (200 mL) and extracted with EtOAc (500 mL×2). The combined organic layer was washed with brine (500 mL), dried over anhydrous sodium sulfate, filtered and concentrated under reduced pressure. The resulting crude material was purified by column chromatography (SiO₂ 100-200 mesh size; 30% EtOAc in hexane) to afford phosphonate Int-10 as a yellow oil. Yield: 148 g (95%). LC MS: Calculated for C₁₁H₁₄IO₃P is 354.12, Observed: 354.8 [M+H]⁺

Step 2: To a stirred solution of methyl (tert-butoxycarbonyl)-D-serinate (Int-11) (100 g, 456 mmol) in acetone (1000 ml), were added 2,2-Dimethoxypropane (337 ml, 2737 mmol) and p-toluenesulfonic acid monohydrate (2.60 g, 13.68 mmol). The resulting reaction mixture was stirred at room temperature for 16 h. The reaction was followed by TLC. The volatiles were evaporated under reduced pressure. To the residue was added of water (500 mL). This was extracted with EtOAc (800 ml×2). Combined organic layers were washed with brine (800 mL), dried over anhydrous sodium sulfate, filtered and concentrated under reduced pressure The crude product was purified by column chromatography (SiO₂ 100-200 mesh size; 20-30% EtOAc in hexane) to obtain Int-12 as a pale yellow liquid. Yield: 100 g (84%). LC-MS: Calculated for C₁₂H₂₁NO₅ is 259.30, Observed: No ionization observed.

Step 3: To a stirred solution of Int-12 (50 g, 193 mmol) in dry Toluene (500 ml) was added DIBAL-H (1.2 M in Toluene; 241 mL, 289 mmol) at −78° C. The reaction mixture was stirred at −78° C. for 2 h. The progress of the reaction was monitored by TLC. The reaction was quenched with MeOH (250 mL) at −78° C. and the resulting emulsion was slowly poured into ice cold 1 N HCl (500 mL) solution and extracted with EtOAc (500 mL×3). The combined organic layers were dried over anhydrous Na₂SO₄ and concentrated under reduced pressure to afford aldehyde Int-13a as colorless oil. Crude product was taken up for next step. Yield: 44 g.

Step 4: To a stirred solution of phosphonate Int-10 (45.9 g, 130 mmol) dry THE (350 mL), was added 2.0 M LDA in THE (88 mL, 177 mmol) at −78° C. The reaction mixture was stirred at −78° C. for 1 h. To this reaction mixture, was added a solution of aldehyde Int-13a (27 g, 118 mmol) in THE (150 mL) at −78° C. The reaction mixture was slowly allowed to attain room temperature and stirred for 5 h. After completion of the reaction, the reaction was quenched with sat. NH₄Cl solution (300 mL) and extracted with EtOAc (400 mL×3). The combined organic layers were dried over anhydrous Na₂SO₄ and concentrated under reduced pressure to afford the crude product. The crude product was purified by flash column chromatography (SiO2 230-400 mesh; 5-6% EtOAc in pet ether) to obtain the mixture of epimers. Further the epimers were separated by SFC purification to obtain Int-14 as an off-white solid. Yield: 21 g (41%). LC-MS: Calculated for C₁₈H₂₄INO₃ is 429.30, Observed: 373.9 [M−56+H]⁺

Steps 5: To a stirred solution of Int-14 (15 g, 34.9 mmol) in MeOH (150 mL), was added HCl (1.5 M HCl in water; 58.2 mL, 87 mmol) at room temperature. The reaction mixture was stirred at 80° C. for 4 h. The reaction was followed by TLC. The volatiles were evaporated under reduced pressure. The resulting product was azeotroped with toluene. The resulting crude product was triturated with EtOAc (20 mL). The resulting suspension was filtered through Buchner funnel and dried under reduced pressure to obtain Int-15a as a white solid. Yield: 10.4 g (91%). LC-MS: Calculated for C₁₀H₁₃C₁₁NO is 325.57; C₁₀H₁₂INO (for parent compound) is 289.12, Observed: 273.2 [M—NH₂+H]⁺.

Step 6: To a stirred solution of Int-15a (10.5 g, 32.3 mmol) in MeOH (100 mL), was added ammonium acetate (4.97 g, 64.5 mmol) and stirred at room temperature for 10 min. To this reaction mixture, was added aldehyde Int-4 (20.41 g, 129 mmol) and continued the stirring for 10 min. Finally, glyoxal (40% in water; 5.55 ml, 48.4 mmol) was added and stirred at room temperature for additional 10 min. The reaction mixture was stirred at 80° C. for 2 h. The reaction was monitored by LCMS. Excess solvent was distilled out, diluted with water (500 ml) and extracted with 5% MeOH/DCM (500 mL×3). The combined organic layers were dried over Na₂SO₄, filtered and concentrated under reduced pressure. The crude compound was purified by silica-gel column chromatography (SiO₂ 230-400 mesh; 10-100% EtOAc in pet ether followed by 0-5% methanol in DCM) to afford Int-16a as a brown gummy solid. Yield: 21 g (60%) (Yield for combined batches). LC MS: Calculated for C₂₀H₂₅1N₃O₃ is 468.34, Observed: 469.2 [M+H]⁺.

A similar method was adopted for the synthesis of 16b, starting with Int-13b and Int-10.

### Example 4: Preparation of 4-((4-bromophenoxy)methyl)-2,2-dimethyl-1,3-dioxolane (Int-20a)

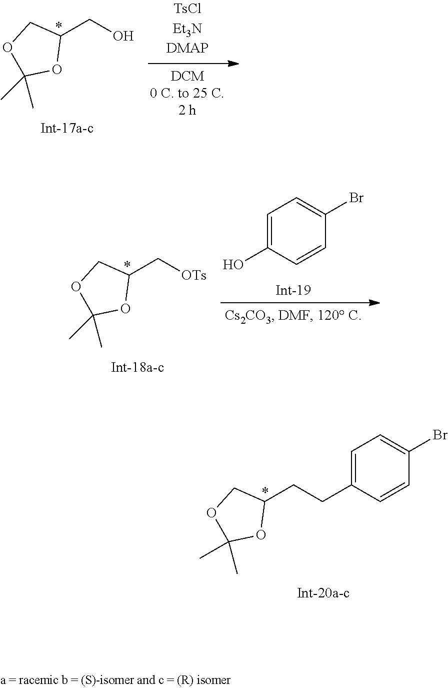

Step 1: To a solution of (1,2—O-Isopropylideneglycerol (Int-17a, 1.0 g, 7.57 mmol) in DCM (10 mL), were added triethylamine (3.16 mL, 22.70 mmol), tosyl chloride (1.875 g, 9.84 mmol) and DMAP (92 mg, 0.757 mmol) at 0° C. The reaction mixture was then stirred at 25° C. for 2 h. The reaction was monitored by TLC; TLC showed complete consumption of starting material. The reaction was quenched by the addition of saturated sodium bicarbonate solution, extracted with DCM (50 mL×2). The combined organic layers were washed with brine (50 mL), dried over anhydrous sodium sulphate, filtered and concentrated under reduced pressure.

The resulting crude product was purified by flash column chromatography (SiO₂ 230-400 mesh; 15% EtOAc in pet ether) to obtain Int-18a as a white solid. Yield: 1.5 g (68%). LC-MS: Calculated for C₁₃H₁₈O₅S is 286.34, Observed: 287.1 [M+1]⁺.

Step 2: To a solution of Int-18a (384 mg, 1.75 mmol) in DMF (5 mL), was added cesium carbonate (569 mg, 1.75 mmol) at 25° C. under nitrogen atmosphere and stirred for 20 min. To this reaction mixture was added Int-19 (500 mg, 1.75 mmol) and heated at 90° C. for 4 h. The reaction was monitored by TLC, showed complete consumption of starting material. The reaction mixture was cooled to room temperature, quenched by the addition of ice-cold water (30 mL), extracted with DCM (50 mL×3). The combined organic layers were washed with brine (50 mL), dried over anhydrous sodium sulphate, filtered and concentrated under reduced pressure. The resulting crude product (600 mg) was purified by flash column chromatography (SiO₂ 230-400 mesh; 10% EtOAc in pet ether) to afford Int-20a. Yield: 250 mg (29%). LC-MS: Calculated for C₁₈H₂₇BO₅ is 334.21, Observed: 335.2 [M+1]+(64% by LCMS).

A similar strategy was carried out to synthesize (R) and (S) isomer derivatives from their appropriate chiral starting materials to afford Int-20b and Int-20c.

A similar strategy was carried out to synthesize Int-20d using the 4-bromo-2-fluorophenol and the (S)-chiral isomer as the starting materials.

### Example 5: Preparation of 5-((4-bromophenoxy)methyl)-2,2-dimethyl-1,3-dioxane (Int-23)

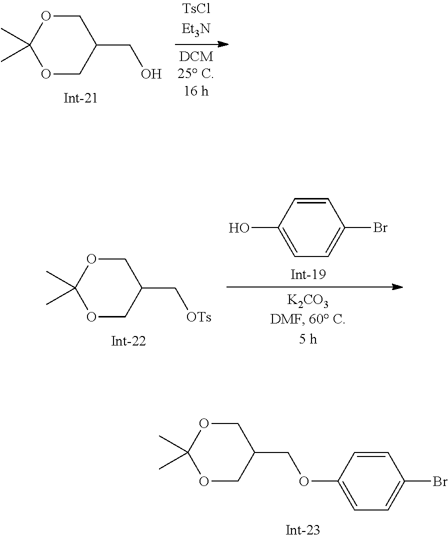

Step 1: To a stirred solution of (2,2-dimethyl-1,3-dioxan-5-yl)methanol (Int-21) (5.0 g, 34.2 mmol) in DCM (50 mL) were added triethylamine (9.53 mL, 68.4 mmol) and p-toluenesulfonyl chloride (7.17 g, 37.6 mmol) at 25° C. After stirring for 16 h, the reaction mixture followed by TLC showed complete consumption of starting material. The reaction was quenched by the addition of water (50 mL) and extracted with DCM (50 mL×2). The combined organic layers were washed with brine (100 mL), dried over anhydrous sodium sulphate, filtered and concentrated under reduced pressure. The resulting crude product was purified by flash column chromatography (SiO₂; 230-400 mesh; 8-10% EtOAc in hexane) to afford Int-22 as a pale-yellow liquid. Yield: 8.0 g (78%). LCMS: Calculated for C₁₄H₂₀O₅S is 300.37, Observed: 301.2 [M+1]⁺.

Step 2: To a stirred solution of Int-22 (8.0 g, 26.6 mmol) in DMF (80 mL), were added potassium carbonate (8.1 g, 58.6 mmol) and 4-bromophenol (Int-19) (5.07 g, 29.3 mmol) at 25° C. Then the reaction mixture was heated at 60° C. for 5 h. The reaction was followed by TLC, showed complete consumption of starting material. The reaction was quenched by the addition of water (100 mL) and extracted with EtOAc (100 mL×2). The combined organic layers were washed with brine (100 mL), dried over anhydrous sodium sulphate, filtered and concentrated under reduced pressure. The resulting crude residue was purified by flash column chromatography (SiO2; 230-400 mesh; 8% EtOAc in hexane) to afford Int-23. Yield: 5.0 g (62%). LC MS: Calculated for C₁₃H₁₇BrO₃ is 301.18, Observed: 301.2 [M]⁺ and 303.2 [M+2]⁺.

### Example 6: Preparation of (2S,Z)-4-(4-bromophenyl)-4-fluoro-2-(2-((1S)-1-((tetrahydro-2H-pyran-2-yl)oxy)ethyl)-1H-imidazol-1-yl)but-3-en-1-ol (Int-27)

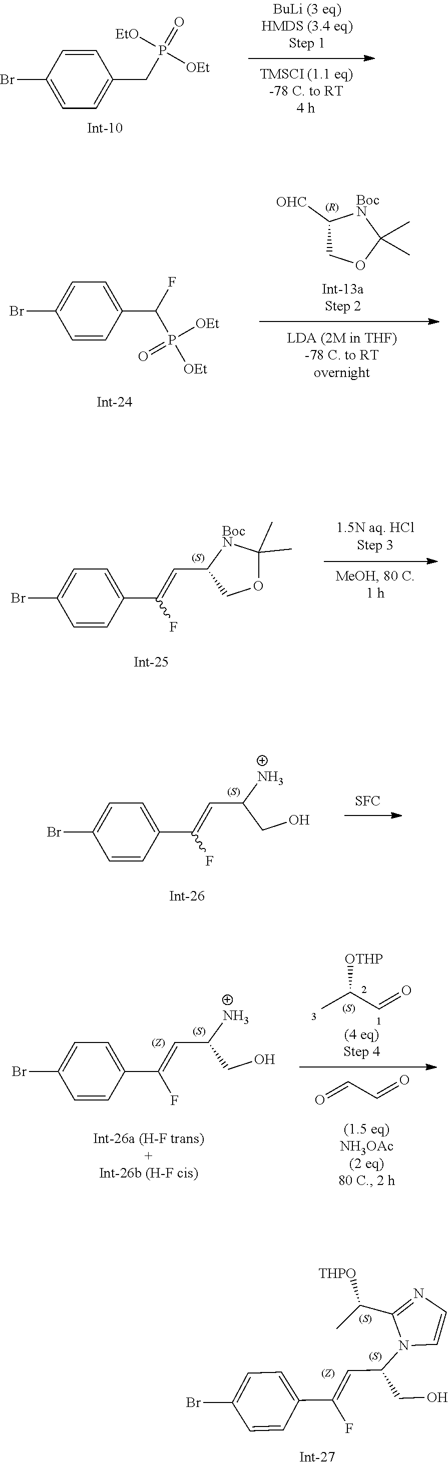

Step 1: To a stirred solution of n-BuLi (2.4 M in hexane, 20.35 mL, 48.84 mmol, 3 equiv) in THE (30 mL), were added a solution of diethyl (4-bromobenzyl)phosphonate (Int-10, 5 g, 16.28 mmol) in THE (20 mL) slowly followed by hexamethyldisilazane (8.93 g, 55.33 mmol) at −78° C. This mixture was stirred for 10 min at the same temperature and the dry ice-acetone batch was then exchanged for a water bath and stirred at RT for 1 h. TMS-C₁ (1.94 g, 17.85 mmol) dissolved in THE (20 mL) was added and stirred for 15 minutes at room temperature. The reaction mass was again cooled to −78° C. and NFSI (6.67 g, 21.16 mmol) dissolved in THE (20 mL) was added at −78° C. and stirred for 15 minutes and then stirred at 0° C. for 1 h. After completion of the reaction, the reaction mixture was quenched with 1 M LiOH (100 mL) and stirred at 0° C. for 15 minutes. The layers were separated, and the organic layer diluted with EtOAc (500 mL) and washed with 1.5 M HCl (500 mL×2). The organic layer was dried over anhydrous Na₂SO₄ and concentrated under reduced pressure to afford the crude product. The crude product was purified by column chromatography using silica gel (230-400 mesh) by eluting with 9-14% ethyl acetate in petroleum ether to afford (rac)-diethyl ((4-bromophenyl)fluoromethyl)phosphonate (Int-24, 50% purity) as a liquid. This was taken as such to the next step. Yield: 5 g (94%). LC MS: Calculated for C₁₁H₁₅BrFO₃P is 325.11, Observed: 325[M] and 327.1[M+2]⁺.

Step 2: To a stirred solution of Int-24 (1.7 g, 5.23 mmol) in THE (20 mL), LDA (2 M in THF, 3.27 mL, 6.54 mmol, 1.5 equiv.) was added at −78° C. and the reaction mixture stirred at −78° C. for 1 h. To the above reaction mass, Int-13a (1 g, 4.36 mmol) dissolved in THE (20 mL) was added at −78° C. and the reaction mixture stirred at −78° C. for 1 h and at room temperature overnight. After completion of the reaction, the reaction mixture was quenched with saturated NH4C₁ solution and extracted with EtOAc (200 mL×2). The combined organic layers were dried over anhydrous Na₂SO₄ and concentrated under reduced pressure to afford the crude product. The crude product was purified by column chromatography using silica gel (230-400 mesh) by eluting with 3-6% ethyl acetate in petroleum ether to afford Int-25. Analysis of the mixture by chiral SFC indicated a diastereomeric ratio of 49:2:47:1. This was carried forward as such. Yield: 378 mg (22%). LC MS: Calculated for C₁₈H₂₃BrFNO₃ is 400.29, Observed: 300[M-100]⁺. and 302[M-100]⁺.

Step 3: To a stirred solution of Int-25 (6 g, 0.015 mmol) in MeOH (60 mL), was added aq. HCl (1.5 N, 60 mL) and the reaction mixture stirred at 80° C. for 1 h. After completion of the reaction, the solvent was concentrated under reduced pressure to afford the crude product, which was triturated with pet-ether, decanted and dried under reduced pressure to get Int-26 as an off-white solid. Analysis of the mixture by chiral SFC indicated a ratio of 44:54. Separation of 2 g of Int-26 by SFC yielded 230 mg of Int-26a which was taken for the next step. Isomer Int-26a was identified to have (Z)-configuration by ¹H NMR (J_{HF}=37.6 Hz) and the chemical shift values of the fluorine in ¹⁹F NMR (−114 ppm for the (Z)-isomer vs. −91.47 ppm for the (E)-isomer in DMSO). Analysis of Int-26a by chiral SFC indicated that it was single, and this was used in the further steps. Yield: 4.6 g (Int-26). LC-MS: Calculated for C₁₀H₁₁BrFNOis 260.11, Observed: 243.1 [M-OH]⁺ and 245.1 [M—OH+2]⁺.

Step 4: To a stirred solution of Int-26a (230 mg, 0.884 mmol) in MeOH (5 mL), were added NH₄OAc (136.17 mg, 1.76 mmol), (2S)-2-((tetrahydro-2H-pyran-2-yl)oxy)propanal (559.1 mg, 3.53 mmol) and stirred for 10 min. Then Glyoxal (40% in H₂O, 0.2 mL, 1.32 mmol) was added at RT and stirred at 80° C. for 2 h. After completion of the reaction, the reaction mixture was concentrated under reduced pressure to get the crude product (Product formation by LCMS was only to an extent of 28%). The crude product was purified by RP-HPLC using 0.1% HCOOH in H₂O and Acetonitrile to afford product Int-27 as brown gum. Yield: 170 mg (43%). LC-MS: Calculated for C₂₀H₂₄BrFN₂O₃ is 439.33, Observed: 4 [M]⁺. and 441[M+2]⁺.

### Example 7: Preparation of tert-butyl 4-(((4-bromobenzyl)oxy)methyl)-2,2-dimethyloxazolidine-3-carboxylate (Int-30a)

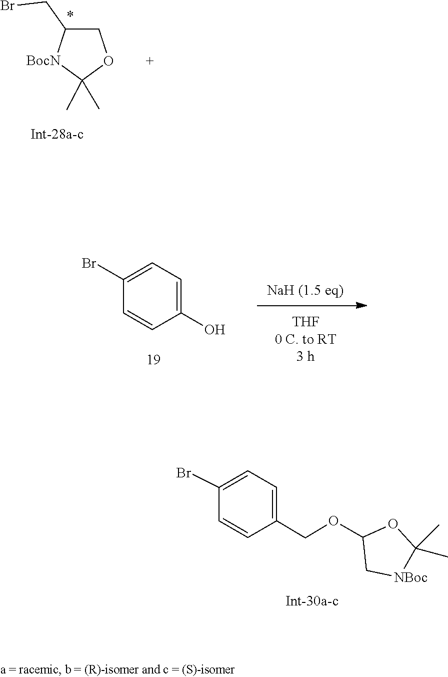 [SMILES: CC(C)(C)OC(=O)N1C(CBr)COC1(C)C ; Oc1ccc(Br)cc1 ; CC(C)(C)OC(=O)N1CC(OCc2ccc(Br)cc2)OC1(C)C]

Step 1: To a stirred solution of Int-28a (1.5 g, 0.0064 mol) in dry THE (15 mL) and NaH (60% dispersion in mineral oil) (0.38 g, 0.0097 mol) was added portion wise at 0° C. The reaction mixture was stirred at 0° C. for 30 minutes. Then 4-bromophenol (Int-19) (2.1 g, 0.0084 mol) was dissolved in THE and slowly added to the reaction mixture, stirred at 0° C. for 30 minutes. Then reaction mixture was stirred at RT for 2 h. After the completion of the reaction, the reaction mixture was quenched with sat. NH₄Cl solution, diluted with EtOAc (500 mL) and washed with ice cold water (200 mL), dried over anhydrous Na₂SO₄, filtered and concentrated under reduced pressure. The crude product was purified by column chromatography on silica gel (230-400 mesh) by eluting with 6-10% ethyl acetate in petrol ether to afford product Int-30a. Yield: 1.49 g (57.32%). LC MS: Calculated for C₁₈H₂₆BrNO₄ is 400.310, Observed: 344 [M−56] and 346[M-56]+2.

A similar strategy was carried out to synthesize (R) and (S) isomer derivatives from their appropriate chiral starting materials to afford Int-30b and Int-30c.

### Example 7a: Preparation of tert-butyl 4-(((4-bromobenzyl)oxy)methyl)-2,2-dimethyloxazolidine-3-carboxylate (Int-101a)

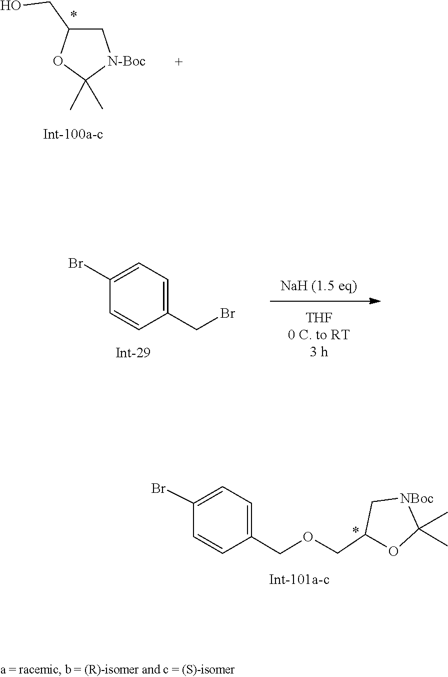

Step 1: To a stirred solution of Int-100a (1.5 g, 0.0064 mol) in dry THE (15 mL) and NaH (60% dispersion in mineral oil) (0.38 g, 0.0097 mol) was added portion wise at 0° C. The reaction mixture was stirred at 0° C. for 30 minutes. Then 4-Bromobenzyl bromide (Int-29) (2.1 g, 0.0084 mol) was dissolved in THE and slowly added to the reaction mixture, stirred at 0° C. for 30 minutes. Then reaction mixture was stirred at RT for 2 h. After the completion of the reaction, the reaction mixture was quenched with sat. NH₄Cl solution, diluted with EtOAc (500 mL) and washed with ice cold water (200 mL), dried over anhydrous Na₂SO₄, filtered and concentrated under reduced pressure. The crude product was purified by column chromatography on silica gel (230-400 mesh) by eluting with 6-10% ethyl acetate in petrol ether to afford product Int-101a. Yield: 1.49 g (57.32%). LC MS: Calculated for C₁₈H₂₆BrNO₄ is 400.310, Observed: 344 [M-56] and 346[M-56]+2.

A similar strategy was carried out to synthesize (R) and (S) isomer derivatives from their appropriate chiral starting materials to afford Int-101b and Int-101c.

### Example 8: Preparation of 3-(4-bromophenoxy)-1-((2,2-dimethyl-1,3-dioxolan-4-yl)methyl)azetidine (Int-34)

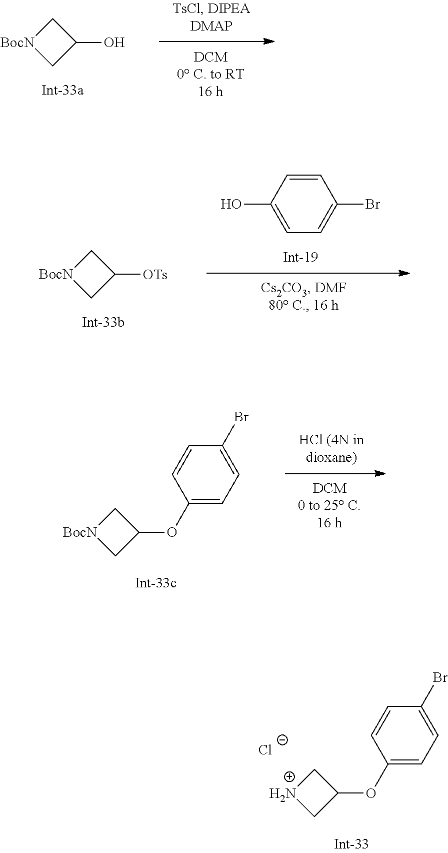 [SMILES: CC(C)(C)OC(=O)N1CC(O)C1 ; Oc1ccc(Br)cc1 ; Cc1ccc(S(=O)(=O)OC2CN(C(=O)OC(C)(C)C)C2)cc1 ; CC(C)(C)OC(=O)N1CC(Oc2ccc(Br)cc2)C1 ; [Cl-] ; Brc1ccc(OC2C[NH2+]C2)cc1]

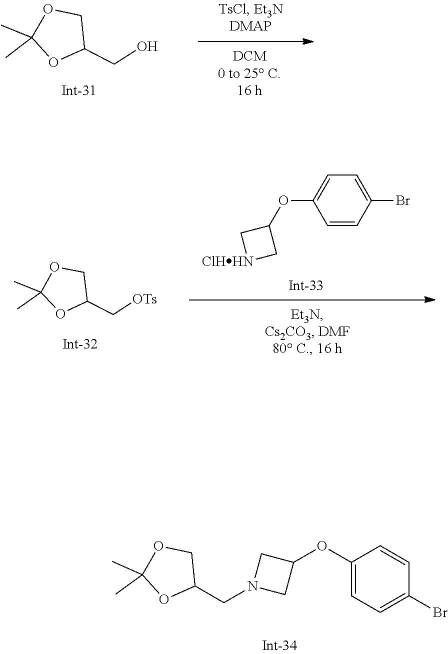

Step 1: To a solution of tert-butyl 3-hydroxyazetidine-1-carboxylate (Int-33a, 10.0 g, 57.7 mmol) in DCM (250 mL), were added triethylamine (12.09 mL, 87 mmol) and DMAP (0.705 g, 5.77 mmol) followed by p-toluenesulfonyl chloride (13.21 g, 69.3 mmol) at 0° C. The reaction mixture was stirred at room temperature for 16 h. The reaction, as monitored by TLC, showed complete consumption of starting material. The reaction was quenched by the addition of water (150 mL) and extracted with DCM (150 mL×2). The combined DCM layer was washed with cold water (50 mL), followed by brine solution (50 mL), dried over anhydrous sodium sulphate, filtered and concentrated under reduced pressure to give crude material. The resulting crude was purified by flash column chromatography (SiO₂ 100-200 mesh size; eluted with 5% EtOAc in Hexane) to afford Int-33b as pale-yellow liquid. Yield=17.05 g (90%). LC MS: Calculated for C₁₅H₂₁NO₅S is 327.39, Observed: Observed: 272 [M-tBu+1]⁺ and 228.4 [M−Boc+1]⁺.

Step 2: To a stirred solution of 4-bromophenol (Int-19, 7.61 g, 44.0 mmol) in DMF (100 mL), was added cesium carbonate (14.33 g, 44.0 mmol) at 25° C. and stirred for 30 min. Then added Int-33b (12.0 g, 36.7 mmol) in one portion and the resulting reaction mixture was heated at 80° C. for 16 h. The reaction was monitored by TLC. After completion, the reaction mixture was cooled to room temperature and quenched by the addition of cold water (100 mL) and extracted with MTBE (100 mL×2). The combined MTBE layer was washed with cold water (50 mL), followed by brine solution (50 mL), dried over anhydrous sodium sulphate, filtered and concentrated under reduced pressure to give crude material. The resulting crude was purified by flash column chromatography (SiO₂ 100-200 mesh size; 10% EtOAc in Hexane) to afford Int-33c as a white solid. Yield: 10.0 g (81%). LC MS: Calculated for C₁₄H₁₈BrNO₃ is 328.2, Observed: 228.0 [M-Boc]⁺ and 230.0 [M-Boc+2]⁺.

Step 3: To a solution of Int-33c (10.0 g, 30.5 mmol) in DCM (50 mL), was added HCl (4 N in dioxane, 22.85 mL, 91 mmol) in drop wise fashion at 0° C. The resulting reaction mixture was stirred at 25° C. for 16 h. The reaction was followed by TLC; TLC showed complete consumption of starting material. MTBE (50 mL) was added and stirring continued for 15 min. White solid precipitated, was filtered through a Buchner funnel. The solid was washed with hexane (50 mL) and dried under high vacuum to obtain Int-33 as a white solid. Yield: 6.5 g (80%). LC MS: Calculated for C₉H₁₁BrNO is 229.10 (for the ammonium ion); Observed: 228.8 [M]⁺ and 229.8 [M+2]⁺.

Step-4: To a stirred solution of 2,2-dimethyl-1,3-dioxolan-4-yl)methanol (Int-31, 2.0 g, 15.13 mmol) in DCM (20 mL), were added triethylamine (3.17 mL, 22.70 mmol) and DMAP (0.185 g, 1.513 mmol) at 0° C. followed by addition of p-toluenesulfonyl chloride (3.46 g, 18.16 mmol). The resulting reaction mixture was stirred at 25° C. for 16 h. The reaction was monitored by TLC. The reaction was quenched by the addition of water (50 mL) and extracted with DCM (50 mL×2). The combined DCM layer was washed with cold water (50 mL), followed by brine (25 mL), dried over anhydrous sodium sulphate, filtered and concentrated under reduced pressure to give crude material. The resulting crude was purified by flash column chromatography (SiO₂ 100-200 mesh size; 5% EtOAc in hexane) to obtain Int-32 as a white gum. Yield: 3.6 g (83%). LC MS: Calculated for C₁₃H₁₈O₅S is 286.34, Observed: 287.4 [M+1]⁺

Step-5: To a stirred solution of 3-(4-bromophenoxy)azetidine hydrochloride (Int-33, 0.8 g, 3.02 mmol) in DMF (10 mL), were added triethylamine (0.507 mL, 3.63 mmol) and Cs₂CO₃ (1.971 g, 6.05 mmol) at 0° C. and stirred for 10 min. To this reaction mixture, was added Int-32 (1.039 g, 3.63 mmol). The resulting reaction mixture was heated at 100° C. for 16 h. The reaction was monitored by TLC. The reaction was cooled to ambient temperature and quenched by the addition of water (50 mL) and extracted with EtOAc (50 mL×2). The combined EtOAc layer was washed with cold water (20 mL), followed by brine (10 mL), dried over anhydrous sodium sulphate, filtered and concentrated under reduced pressure to give crude material. The resulting crude was purified by flash column chromatography (SiO2 100-200 mesh size; 20% EtOAc in hexane) to obtain Int-34 as a colorless liquid. Yield: 0.6 g (52%). LC MS: Calculated for C₁₅H₂₀BrNO₃ is 342.23, Observed: 342[M]⁺ and 344.3 [M+2]⁺.

A similar strategy was carried out to synthesize 2-(3-(4-bromophenoxy)azetidin-1-yl)ethan-1-ol (Int-34a) from Int-33 and 2-bromoethanol: To a stirred solution of 3-(4-bromophenoxy)azetidine hydrochloride (Int-33, 3.02 mmol) in DMF (10 mL), were added triethylamine (3.63 mmol) and Cs₂CO₃ (7.05 mmol) at 0° C. and stirred for 10 min. To this reaction mixture, was added 2-bromoethanol (3.63 mmol). The resulting reaction mixture was heated at 100° C. for 16 h. The reaction was monitored by TLC. The reaction was cooled to ambient temperature and quenched by the addition of water (50 mL) and extracted with EtOAc (50 mL×2). The combined EtOAc layer was washed with cold water (20 mL), followed by brine (10 mL), dried over anhydrous sodium sulphate, filtered and concentrated under reduced pressure to give crude material. The resulting crude was purified by flash column chromatography (SiO2 100-200 mesh size; 20% EtOAc in hexane) to obtain Int-34a as a pale-yellow solid. Yield: 0.60 g (30%). LC-MS: Calculated for C₁₂H₁₆BrNO₃ is 302.16, Observed: 302.2 [M] and 304.2 [M+2]⁺.

### Example 9: Preparation of 2-(3-(4-bromophenoxy)azetidin-1-yl)propane-1,3-diol (Int-40)

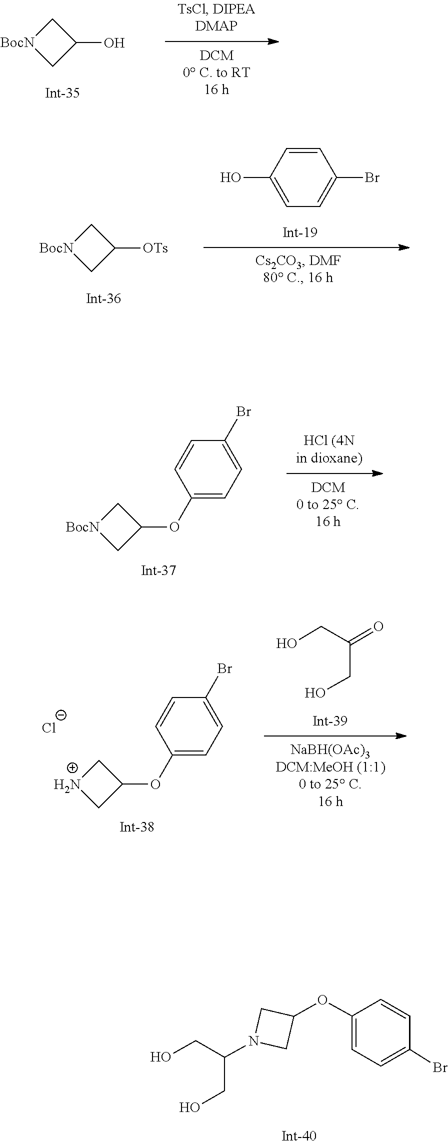

Step 1: To a solution of tert-butyl 3-hydroxyazetidine-1-carboxylate (Int-35, 10.0 g, 57.7 mmol) in DCM (250 mL), were added triethylamine (12.09 mL, 87 mmol) and DMAP (0.705 g, 5.77 mmol) followed by p-toluenesulfonyl chloride (13.21 g, 69.3 mmol) at 0° C. The reaction mixture was stirred at room temperature for 16 h. The reaction, as monitored by TLC, showed complete consumption of starting material. The reaction was quenched by the addition of water (150 mL) and extracted with DCM (150 mL×2). The combined DCM layer was washed with cold water (50 mL), followed by brine solution (50 mL), dried over anhydrous sodium sulphate, filtered and concentrated under reduced pressure to give crude material. The resulting crude was purified by flash column chromatography (SiO₂ 100-200 mesh size; eluted with 5% EtOAc in Hexane) to afford Int-36 as pale-yellow liquid. Yield=17.05 g (90%). LC MS: Calculated for C₁₅H₂₁NO₅S is 327.39, Observed: Observed: 272 [M-tBu+1]⁺ and 228.4 [M−Boc+1]⁺.

Step 2: To a stirred solution of 4-bromophenol (Int-19, 7.61 g, 44.0 mmol) in DMF (100 mL), was added cesium carbonate (14.33 g, 44.0 mmol) at 25° C. and stirred for 30 min. Then added Int-36 (12.0 g, 36.7 mmol) in one portion and the resulting reaction mixture was heated at 80° C. for 16 h. The reaction was monitored by TLC. After completion, the reaction mixture was cooled to room temperature and quenched by the addition of cold water (100 mL) and extracted with MTBE (100 mL×2). The combined MTBE layer was washed with cold water (50 mL), followed by brine solution (50 mL), dried over anhydrous sodium sulphate, filtered and concentrated under reduced pressure to give crude material. The resulting crude was purified by flash column chromatography (SiO₂ 100-200 mesh size; 10% EtOAc in Hexane) to afford Int-37 as a white solid. Yield: 10.0 g (81%). LC MS: Calculated for C₁₄H₁₈BrNO₃ is 328.2, Observed: 228.0 [M-Boc]⁺ and 230.0 [M-Boc+2]⁺.

Step 3: To a solution of Int-37 (10.0 g, 30.5 mmol) in DCM (50 mL), was added HCl (4 N in dioxane, 22.85 mL, 91 mmol) in drop wise fashion at 0° C. The resulting reaction mixture was stirred at 25° C. for 16 h. The reaction was followed by TLC; TLC showed complete consumption of starting material. MTBE (50 mL) was added and stirring continued for 15 min. White solid precipitated, was filtered through a Buchner funnel. The solid was washed with hexane (50 mL) and dried under high vacuum to obtain Int-38 as a white solid. Yield: 6.5 g (80%). LC MS: Calculated for C₉H₁₁BrNO is 229.10 (for the ammonium ion); Observed: 228.8 [M]⁺ and 229.8 [M+2]⁺.

Step 4: To a stirred solution of 3-(4-bromophenoxy)azetidine hydrochloride (Int-38, 1.50 g, 6.58 mmol) in DCM: MeOH (1:1, 30 mL), were added dihydroxyacetone (Int-39, 0.889 g, 9.86 mmol) followed by sodium triacetoxyborohydride (2.79 g, 13.15 mmol) at 0° C. The reaction mixture was stirred at 25° C. for 16 h. After completion of the reaction, the reaction mixture was basified with aqueous 10% sat. NaHCO₃ solution. The aqueous layer was extracted with DCM (50 mL×2). The combined organic layer was dried over anhydrous sodium sulphate, filtered and concentrated under reduced pressure. The resulting crude was purified by flash column chromatography (SiO₂ 100-200 mesh size, 0-5% methanol in DCM) to afford Int-40 as a pale-yellow solid. Yield: 0.60 g (30%). LC-MS: Calculated for C₁₂H₁₆BrNO₃ is 302.16, Observed: 302.2 [M] and 304.2 [M+2]⁺.

### Example 10: Preparation of 2-(4-(4-bromophenoxy)piperidin-1-yl)ethan-1-ol (Int-45a)

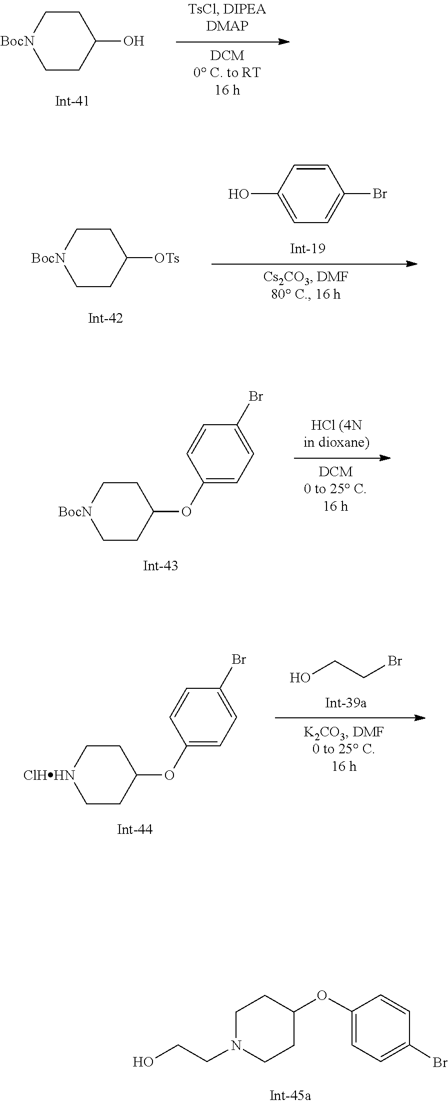

Step 1: To a solution of tert-butyl 4-hydroxypiperidine-1-carboxylate (Int-41, 57.7 mmol) in DCM (250 mL), were added triethylamine (87 mmol) and DMAP (5.77 mmol) followed by p-toluenesulfonyl chloride (69.3 mmol) at 0° C. The reaction mixture was stirred at room temperature for 16 h. The reaction, as monitored by TLC, showed complete consumption of starting material. The reaction was quenched by the addition of water (150 mL) and extracted with DCM (150 mL×2). The combined DCM layer was washed with cold water (50 mL), followed by brine solution (50 mL), dried over anhydrous sodium sulphate, filtered and concentrated under reduced pressure to give crude material. The resulting crude was purified by flash column chromatography (SiO₂ 100-200 mesh size; eluted with 5% EtOAc in Hexane) to afford Int-42 as pale-yellow liquid. Yield: 88%

Step 2: To a stirred solution of 4-bromophenol (Int-42, 44.0 mmol) in DMF (100 mL), was added cesium carbonate (44.0 mmol) at 25° C. and stirred for 30 min. Then added Int-19 (36.7 mmol) in one portion and the resulting reaction mixture was heated at 80° C. for 16 h. The reaction was monitored by TLC. After completion, the reaction mixture was cooled to room temperature and quenched by the addition of cold water (100 mL) and extracted with MTBE (100 mL×2). The combined MTBE layer was washed with cold water (50 mL), followed by brine solution (50 mL), dried over anhydrous sodium sulphate, filtered and concentrated under reduced pressure to give crude material. The resulting crude was purified by flash column chromatography (SiO₂ 100-200 mesh size; 10% EtOAc in Hexane) to afford Int-43 as a white solid. Yield: 76%

Step 3: To a solution of Int-43 (30.5 mmol) in DCM (50 mL), was added HCl (4 N in dioxane, 22.85 mL, 91 mmol) in drop wise fashion at 0° C. The resulting reaction mixture was stirred at 25° C. for 16 h. The reaction was followed by TLC, showed complete consumption of starting material. MTBE (50 mL) was added and stirring continued for 15 min. White solid precipitated, was filtered through a Buchner funnel. The solid was washed with hexane (50 mL) and dried under high vacuum to obtain 4-(4-bromophenoxy)piperidine hydrochloride (Int-44) as a white solid. Yield: 71%

Step 4: To a stirred solution of 4-(4-bromophenoxy)piperidine hydrochloride (Int-44, 1.50 g, 6.58 mmol) in DMF (7 mL), followed by the addition of K₂CO₃. The reaction mixture was stirred at 30 minutes. To this, a respective alkyl halide Int-39a (6.58 mmol) was added and heated at 80° C. for a period of 8 h. After completion of the reaction, the reaction mixture poured into ice cold water and extracted with Ethyl acetate. The aqueous layer was further extracted with Ethyl acetate (50 mL×2). The combined organic layer was dried over anhydrous sodium sulphate, filtered and concentrated under reduced pressure. The resulting crude was purified by flash column chromatography (SiO2 100-200 mesh size, 0-5% methanol in DCM) to afford Int-45a as a pale-yellow solid. Yield 30%.

A similar strategy was carried out to synthesize 3-(4-(4-bromophenoxy)piperidin-1-yl)propane-1,2-diol (Int-45b) from Int-44 and Int-32.

### Example 11: Preparation of tert-butyl ((1S,2S)-2-(4-iodophenoxy)cyclopropyl)carbamate (Int-51a)

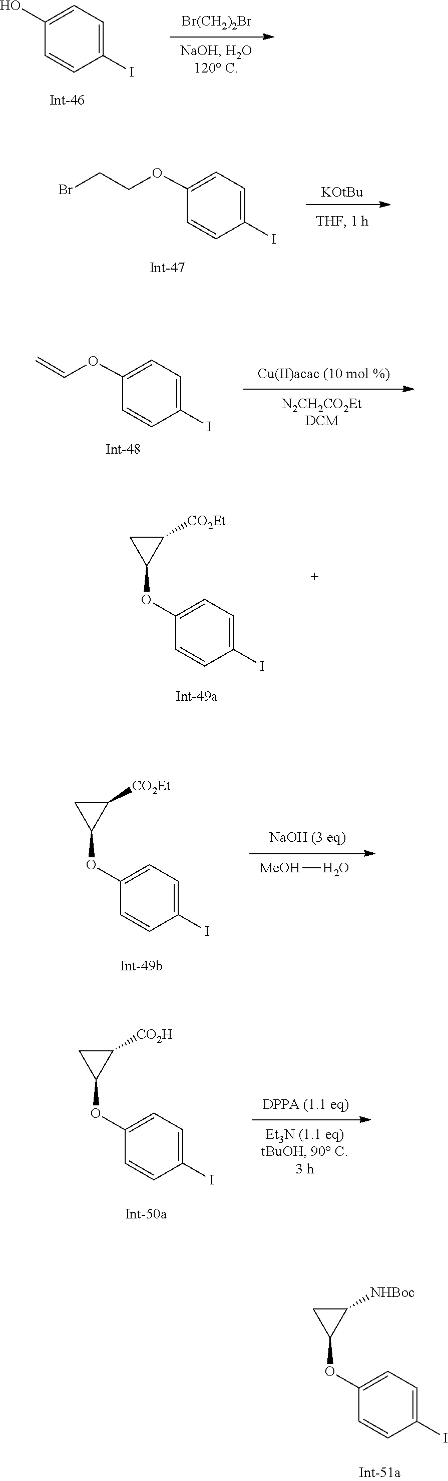

Step 1: To a stirred solution of 4-iodo phenol (Int-46, 38 g, 172 mmol) in water (200 mL), NaOH (14.5 g, 362 mmol) and dibromoethane (162.2 g, 863 mmol) were added and the mixture was heated to 120° C. overnight. After completion of the reaction, reaction mass was extracted with EtOAc (1000 mL×2). The combined organic layers were dried over anhydrous Na₂SO₄ and concentrated under reduced pressure. The crude product was purified by column chromatography using silica gel (60-120 mesh) by eluting with ethyl acetate in pet. ether to afford pure product Int-47 as off solid. Yield: 53 g, (94%)

Step 2: To a stirred solution of Int-47 (67 g, 204 mmol) in THF (670 mL) was added t-BuOK (34.4 g, 307 mmol) and stirred at room temperature for 1 h. After completion of the reaction, reaction mass was diluted with water and extracted with EtOAc (1000 mL×2). The combined organic layers were dried over anhydrous Na₂SO₄ and concentrated under reduced pressure. The crude product was purified by column chromatography using silica gel (60-120 mesh) by eluting with ethyl acetate in pet ether to afford pure product Int-48 as a colorless liquid. Yield: 49 g, (97%).

Step 3: To a stirred solution of Int-48 (20 g, 81.3 mmol) in DCM (200 mL) and copper (II) acetyl acetonate (2.12 g, 8.13 mmol), ethyl diazo acetate (50.27 g, 440 mmol) was added through syringe pump at a rate of 0.2 mL/min at 0° C. After completion of the addition, the reaction mixture was stirred at room temperature 18 h. After completion of the reaction the DCM was removed under reduced pressure. The residue was dissolved in EtOAc (500 mL) and washed with water and brine solution. The organic layer was dried over anhydrous Na₂SO₄, filtered and concentrated under reduced pressure. The crude product was purified by column chromatography using silica gel (230-400 mesh) by eluting with 2-5% of ethyl acetate in pet. ether to afford Int-49a (trans; non-polar) & Int-49b (cis; polar) as a liquid. Yield: Compound Int-49a—11.7 g, (22%) and Compound Int-49b—28 g (contaminated with ethyl diazoacetate).

Step 4: To a stirred solution of Int-49a (38 g, 114 mmol) in water (200 mL) and MeOH (200 mL), NaOH (22.8 g,570 mmol) was added and stirred at room temperature for 1 h. After the completion of the reaction, the reaction mixture was concentrated under reduced pressure, aqueous layer was washed with EtOAc (500 mL×2) and acidified the aqueous layer with dil HCl, extracted with EtOAc (500 mL×2). The combined organic layers were dried over anhydrous Na₂SO₄ and concentrated under reduced pressure. The crude product obtained Int-50a was used without further purification. Yield: 30 g, (88%). LC MS: m/z calculated for C₁₀H₉IO₃ is 304.08 Observed: 302.9 [M−H]⁺.

Step 5: To a stirred solution of Int-50a (30 g, 98.6 mmol) in dry t-BuOH (300 mL), were added triethylamine (15.1 mL,108 mmol) and DPPA (29.9 g,108 mmol) and stirred the reaction mixture at 90° C. for 3 h. After the completion of reaction, the reaction mixture was concentrated under reduced pressure, diluted with EtOAc (500 mL) and washed with NaHCO₃and water. The organic layer was dried over anhydrous Na₂SO₄ and concentrated under reduced pressure to afford the crude product. The crude product was purified by column chromatography using silica gel (60-120 mesh and 230-400 mesh of 1:1 ratio) by eluting with 5-6% ethyl acetate in petrol ether to afford pure product Int-51a as off white solid. Yield: 26.6 g, (70%). LC MS: m/z calculated for C₁₄H₁₈INO₃ is 375.21, Observed:276 [M-100+H]⁺.

A similar strategy was carried out to prepare the cis derivative Int-51b from Int-49b.

### Example 12: Preparation of (trans)-4-(4-bromophenoxy)tetrahydrofuran-3-ol (Int-53)

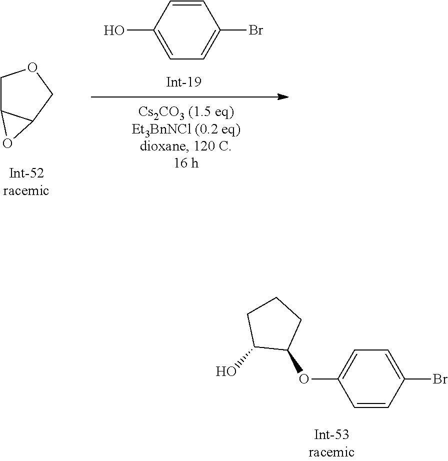 [SMILES: Oc1ccc(Br)cc1 ; C1OCC2OC12 ; O[C@@H]1CCC[C@H]1Oc1ccc(Br)cc1]

Step 1: To a stirred solution of 4-bromophenol (Int-19) (4 g, 23.12 mmol) in 1,4-Dioxane (40 mL), were added 3,6-dioxabicyclo[3.1.0]hexane (Int-52, 1.98 g, 23.12 mmol), Cs₂CO₃ (11.3 g, 34.68 mmol) and Benzyltriethylammonium chloride (1.05 g, 4.62 mmol) at RT and the reaction mixture heated for 16 h at 120° C. After completion of the reaction, the reaction mixture was diluted in EtOAc (200 mL) and washed with sat. NaHCO₃ solution (100 mL), water (100 mL). The organic layer was dried over Na₂SO₄ and concentrated under reduced pressure.

The crude product was triturated with petroleum ether and dried to afford Int-53. Yield: 5 g, (84%). LC-MS: Calculated for C₁₀H₁₁BrO₃ is 259.10, Observed: no ionization observed.

### Example 13: Preparation of tert-butyl ((trans)-4-(4-bromophenoxy)tetrahydrofuran-3-yl)carbamate (Int-59)

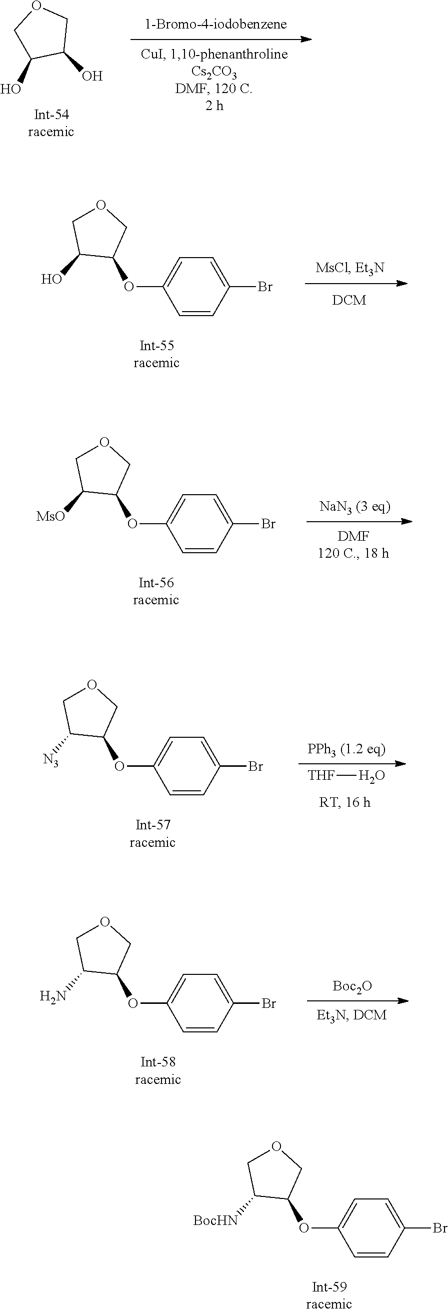

Step 1: To a stirred solution of 4-bromophenol (Int-19)(4 g, 23.12 mmol) in 1,4-Dioxane (40 mL), were added 3,6-dioxabicyclo[3.1.0]hexane (Int-54, 1.98 g, 23.12 mmol), Cs₂CO₃ (11.3 g, 34.68 mmol) and Benzyltriethylammonium chloride (1.05 g, 4.62 mmol) at RT and the reaction mixture heated for 16 h at 120° C. After completion of the reaction, the reaction mixture was diluted in EtOAc (200 mL) and washed with sat. NaHCO₃ solution (100 mL), water (100 mL). The organic layer was dried over Na₂SO₄ and concentrated under reduced pressure. The crude product was triturated with petroleum ether and dried to afford Int-55. Yield: 5 g, (84%). LC-MS: Calculated for C₁₀H₁₁BrO₃ is 259.10, Observed: no ionization observed.

Step-2: To a stirred solution of Int-55 (0.600 g, 2.31 mmol) in DCM (6 mL), were added Et₃N (0.5 mL, 3.47 mmol) and Mesyl Chloride (0.2 mL, 2.54 mmol) at 0° C. and the reaction mixture stirred at RT for 1 h. After completion of the reaction, the reaction mass was diluted with DCM (50 mL) and then washed with water (20 mL). The organic layer was dried over Na₂SO₄ and concentrated under reduced pressure. The crude mesylate Int-56 was used in the next step without any further purification. Yield: 0.75 g (Crude). LC-MS: Calculated for C₁₁H₁₃BrO₅S is 337.18, Observed: mass was not ionized.

Step-3: To a stirred solution of the mesylate (Int-56, 0.750 g, 2.22 mmol) in DMF (10 mL), was added sodium azide (0.433 g, 6.67 mmol) at RT and the reaction mixture heated for 18 h at 120° C. After completion of the reaction, the reaction mass was dissolved in water and extracted with EtOAc (2×50 mL). The combined organic layers were washed with brine (50 mL), the organic layer dried over Na₂SO₄ and concentrated under reduced pressure. The crude product Int-57 was used in the next step without any further purification. Yield: 0.600 g, (Crude).

Step-4: To a stirred solution of Int-57 (0.600 g, 2.11 mmol) in THE (9 mL) and water (3 mL), was added PPh₃ (0.663 g, 2.53 mmol) at 0° C. and the reaction mixture stirred at RT for 16 h. After completion of the reaction, the reaction mixture was concentrated under reduced pressure. The resulting residue was acidified with 1.5 N HCl and extracted with EtOAc. The aqueous layer was basified with 10% NaOH solution and extracted with EtOAc (2×50 mL). The combined organic layers were dried over Na₂SO₄ and concentrated under reduced pressure to afford crude product Int-58 which was used in the next step without any further purification. Yield: 0.22 g (40%). LC-MS: Calculated for C₁₀H₁₂BrNO₂ is 258.12, Observed: 258 [M] and 260.0 [M+2]⁺.

Step-5: To the stirred solution of Int-58 (0.220 g, 0.852 mmol) in dry DCM (3 mL) were added Et₃N (0.35 mL, 2.55 mmol) and (Boc)₂O (0.3 mL, 1.279 mmol) at 0° C. and the reaction mixture stirred at RT for 2 h. After completion of the reaction, the reaction mixture was diluted with dichloromethane (40 mL) and washed with water (20 mL). The organic layer was dried over anhydrous Na₂SO₄, filtered and concentrated under reduced pressure to afford crude product Int-59 which was used in the next step without any further purification. Yield: 0.250 g (Crude).

### Example 13a: Preparation of tert-butyl ((trans)-4-(4-bromophenoxy)tetrahydrofuran-3-yl)carbamate (Int-98)

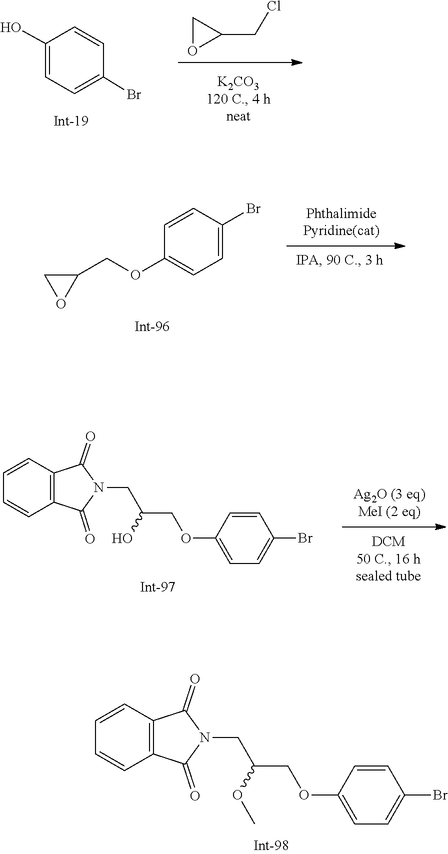 [SMILES: Oc1ccc(Br)cc1 ; ClCC1CO1 ; Brc1ccc(OCC2CO2)cc1 ; O=C1c2ccccc2C(=O)N1CC(O)COc1ccc(Br)cc1 ; COC(COc1ccc(Br)cc1)CN1C(=O)c2ccccc2C1=O]

Step-1: To a mixture of 4-bromophenol (Int-19, 3 g, 17.34 mmol) and K₂CO₃ (2.87 g, 20.80 mmol), was added epichlorohydrin (8.02 g, 86.70 mmol) at RT and the reaction mixture was heated for 4 h at 120° C. After completion of the reaction, the reaction mixture was concentrated under reduced pressure. The resulting residue was dissolved in water and extracted with EtOAc (2×200 mL). The combined organic layers were dried over Na₂SO₄ and concentrated under reduced pressure. The crude product was purified by column chromatography using silica gel (60-120 mesh) by eluting with 17% ethyl acetate in petrol ether to afford pure product Int-96. Yield: 3.0 g, (76%). LC-MS: Calculated for C₉H₉BrO₂ is 229.07, Observed: no ionization observed.

Step-2: To a stirred solution of Int-96 (1.5 g, 6.55 mmol) in Isopropyl alcohol (20 mL), were added phthalimide (1.15 g, 7.86 mmol) and pyridine (cat. 0.75 mL) at RT, and the reaction mixture heated for 3 h at 90° C. After completion of the reaction, the reaction mixture was concentrated under reduced pressure. The resulting residue was dissolved in water and extracted with DCM (2×200 mL). The combined organic layers were dried over Na₂SO₄ and concentrated under reduced pressure. The crude product was triturated with diethyl ether and dried to afford pure product Int-97. Yield: 1.4 g, (56%). LC-MS: Calculated for C₁₇H₁₄BrNO₄ is 376.21, Observed: 375.9 [M]⁺ and 378 [M+2]⁺.

Step-3: To a stirred solution of Int-97 (1.3 g, 3.45 mmol) in DCM (15 mL), were added Ag₂O (2.4 g, 10.36 mmol) and Methyl iodide (0.4 mL, 6.91 mmol), and the reaction mixture stirred for 16 h in darkness at 50° C. in a sealed pressure tube. After completion of the reaction, the reaction mixture was filtered. The filtrate was concentrated under reduced pressure to afford the crude product. The crude product was purified by column chromatography using silica gel (60-120 mesh) by eluting with 15-20% ethyl acetate in petrol ether to afford pure product Int-98. Yield: 1 g, (74%). LC MS: Calculated for C₁₈H₁₆BrNO₄ is 390.23, Observed: 390.2 [M]+, 392.2 [M+2]⁺.

### Example 14: Preparation of (1R,2S)-2-(4-bromophenyl)cyclopropyl acetate (Int-115)

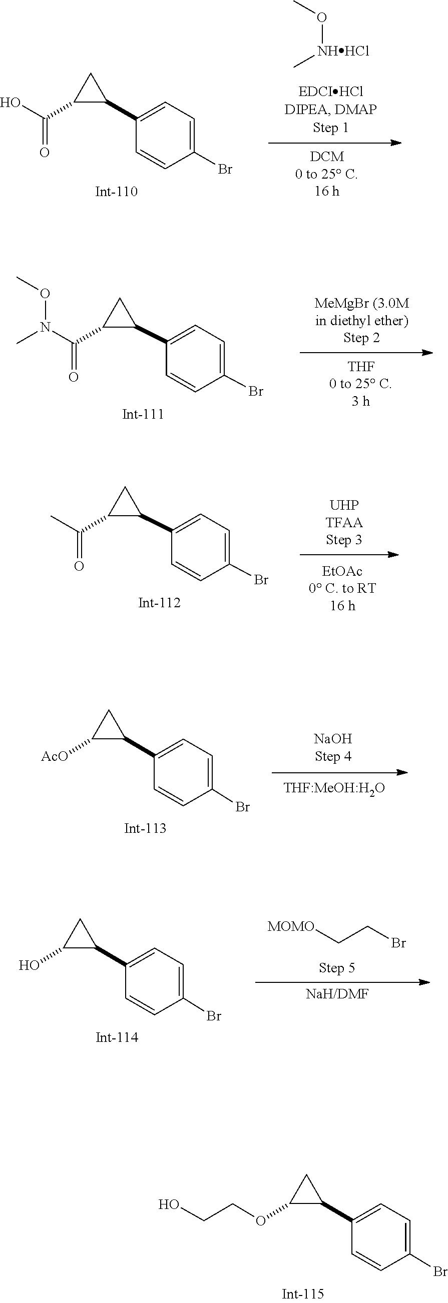

Step 1: To a stirred solution of Int-110 (4.0 g, 16.59 mmol) in DCM (80 mL), were added DMAP (0.020 g, 0.166 mmol), N,O-dimethylhydroxylamine hydrochloride (2.43 g, 24.89 mmol), EDCI.HCl (3.82 g, 19.91 mmol) and DIPEA (2.98 mL, 16.59 mmol) at 0° C. The reaction mixture was stirred at 25° C. for 16 h. The progress of the reaction was monitored by TLC. The reaction was quenched with water (100 mL) and extracted with DCM (30 mL×2). The combined organic layer was washed with brine, dried over Na₂SO₄, filtered, and concentration in vacuo. The obtained residue was purified by flash chromatography (SiO2 100-200 mesh size; 20% EtOAc in hexane) to give Int-111 as an off-white solid. Yield: 3.8 g (81%).

Step 2: To a solution of Int-111 (3.65 g, 12.85 mmol) in THE (50 mL), was added methyl magnesium bromide (3.0 M in Diethyl ether) (8.56 mL, 25.7 mmol) in drop wise fashion at 0° C. The reaction mixture was gradually warmed to room temperature and stirred for 3 h. The reaction was followed by TLC. After completion, the reaction mixture was cooled to 0° C., quenched with sat NH₄Cl solution (100 mL) and extracted with EtOAc (50 mL×2). The combined organic layers were washed with brine solution (50 mL), dried over anhydrous sodium sulphate, filtered and concentrated to get Int-112 as colorless liquid. Yield: 2.7 g (88%).

Step 3: To a stirred solution of Int-112 (2.7 g, 11.29 mmol) in EtOAc (27 mL), were added urea hydrogen peroxide (4.25 g, 45.2 mmol) and TFAA (6.28 mL, 45.2 mmol) at 0° C. The reaction mixture was stirred at room temperature for 16 h. The reaction was followed by TLC. To this reaction mixture again were added urea hydrogen peroxide (4.25 g, 45.2 mmol) and TFAA (9.49 g, 45.2 mmol) at 0° C. The reaction mixture was stirred at RT for 16 h. The reaction was followed by TLC. The reaction was quenched with water (100 mL) and extracted with EtOAc (20 mL×2). The organic layer was washed with brine, dried over anhydrous sodium sulphate, filtered, and concentrated. The crude compound was purified by flash chromatography (SiO2 230-400 mesh size; 5-10% EtOAc in hexane) to afford Int-113 as pale-yellow liquid. Yield: 0.5 g (17%).

Step 4: To a stirred solution Int-113 (0.600 g, 2.37 mmol) in a mixture of THF:MeOH:H₂O (6:2:3) (10 ml), were added NaOH (0.189 g, 4.74 mmol) and stirred for a period of 30 minutes. The reaction mixture was followed by TLC. To this reaction mixture was diluted with H₂O (10 ml) and acidified with 3M HCl. The reaction mixture was extracted with EtOAc (2×35 ml). The organic layer was washed with brine, dried over anhydrous sodium sulfate, filtered and concentrated. The crude compound (Int-114) (yield:0.490 g, 98%) was further taken up to the next step without further purification.

Step 5: To a stirred solution of NaH (0.112 g, 4.64 mmol, 1.2 eq) in DMF (3 mL) in 0° C., Int-114 was dissolved in DMF and added dropwise to the reaction mixture for a period of 20 minutes. To this 1-Bromo-2-(methoxymethoxy)ethane (1.1 eq) was added and stirred for a period of 1 h. The reaction mixture was quenched with satd.NH₄Cl (10 mL) and 3M HCl (1 ml) and extracted with EtOAc (2×25 ml). The organic layer was washed with brine, dried over anhydrous sodium sulfate, filtered and concentrated. The crude compound was purified by flash chromatography (SiO₂ 230-400 mesh size; 25-30% EtOAc in hexane) to afford Int-115 as a brown liquid. Yield (0.400 g, 68%).

### Example 15: Preparation of 2-((1R,2S)-2-(4-bromophenyl)cyclopropoxy)propane-1,3-diol (Int-118)

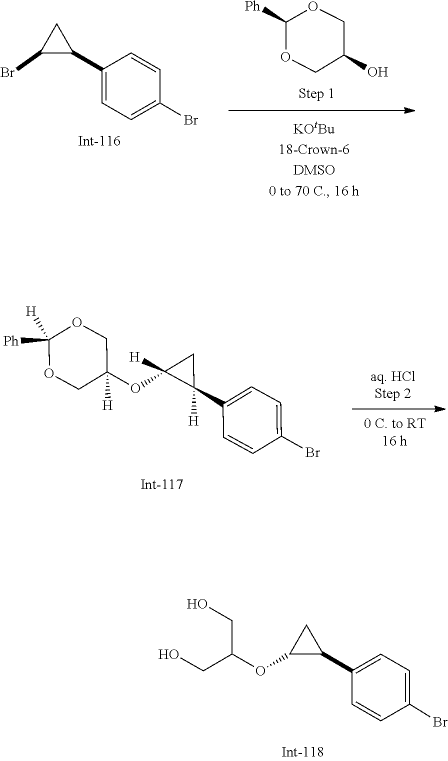

Step-1: To a solution of cis-1,3—O-Benzylideneglycerol (1 g, 5.55 mmol) in DMSO (15 mL), were added KO^{I}Bu (0.623 g, 5.55 mmol) and 18-crown-6 (0.293 g, 1.110 mmol) at 0° C. and the reaction mixture was stirred at the same temperature for 15 min and then allowed to warm to RT. Then, Int-116 (0.766 g, 2.77 mmol) was added and the reaction mixture was stirred at 70° C. for 16 h. The reaction mixture was then diluted with water (25 mL) and extracted with EtOAc (3×50 mL). The combined organic layers were washed with brine (20 mL), dried over anhydrous Na₂SO₄, filtered and concentrated in vacuo. The resulting crude residue was purified by flash column chromatography (SiO₂; 100-200 mesh; eluent: 0-20% EtOAc in n-hexane) to afford Int-117 as a brown gum. Yield: 500 mg (24.01%). UPLC: Calculated for C₁₉H₁₉BrO₃is 375.26, Observed: 375.0 [M]⁺, 377.0 [M+2]⁺.

Step-2: To a solution of Int-117 (0.5 g, 1.332 mmol) in THE (5 mL) and water (5 mL) at 0° C., was added 6N aq. HCl (1.1 mL, 6.66 mmol) and the reaction mixture was stirred at RT for 16 h. The reaction mixture was then concentrated in vacuo and co-distilled with toluene (2×10 mL). It was then triturated with 10% EtOAc in n-hexane (10 mL) and dried to afford Int-118 as a brown gum. Yield: 350 mg (91%). UPLC: Calculated for C₁₂H₁₅BrO₃is 287.15, Observed:284.9 [M-2]-, 286.9 [M].

### Example 16: Preparation of (1R,2R)-2-(4-bromophenoxy)cyclopentan-1-ol (Int-119)

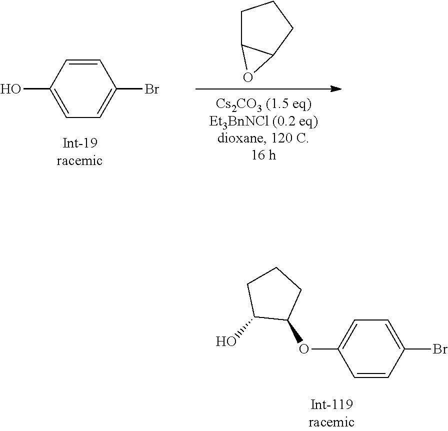 [SMILES: Oc1ccc(Br)cc1 ; C1CC2OC2C1 ; O[C@@H]1CCC[C@H]1Oc1ccc(Br)cc1]

Step 1: To a stirred solution of 4-bromophenol (Int-19) (4 g, 23.12 mmol) in 1,4-Dioxane (40 mL), were added 6—Oxabicyclo[3.1.0]Hexane (1.98 g, 23.12 mmol), Cs₂CO₃ (11.3 g, 34.68 mmol) and Benzyltriethylammonium chloride (1.05 g, 4.62 mmol) at RT and the reaction mixture heated for 16 h at 120° C. After completion of the reaction, the reaction mixture was diluted in EtOAc (200 mL) and washed with sat. NaHCO₃ solution (100 mL), water (100 mL). The organic layer was dried over Na₂SO₄ and concentrated under reduced pressure. The crude product was triturated with petroleum ether and dried to afford Int-119. Yield: 5 g, (84%). LC-MS: Calculated for C₁₁H₁₃BrO₂ is 256.10, Observed: no ionization observed.

### Example 17: Preparation of (3-((4-bromophenoxy)methyl)oxetan-3-yl)methanol (Int-120)

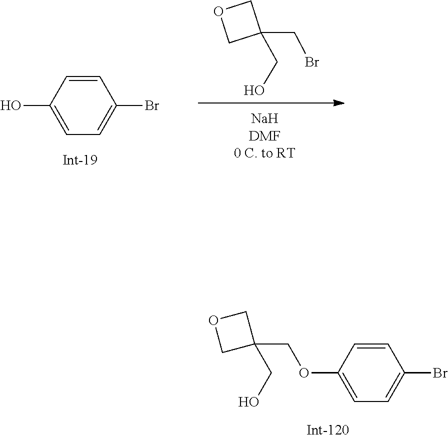 [SMILES: Oc1ccc(Br)cc1 ; OCC1(CBr)COC1 ; OCC1(COc2ccc(Br)cc2)COC1]

Step-1: 4-bromophenol (Int-19, 5 g, 28.9 mmol) was dissolved in DMF (75 ml) at 0° C. under nitrogen atmosphere. NaH (60% dispersion in mineral oil, 1.271 g, 31.8 mmol) was added portion-wise at the same temperature and the solution was stirred for 15 min. (3-(bromomethyl)oxetan-3-yl)methanol (3.27 ml, 28.9 mmol) dissolved in DMF (25 ml) was added dropwise to the above reaction mixture at 0° C., slowly warmed to RT over 4 h and stirred at RT for 16 h. After completion of the reaction as monitored by TLC, the reaction mixture was quenched with water (50 mL) and extracted with EtOAc (50 mL×2). The combined organic layers were washed with brine (100 mL), dried over anhydrous Na₂SO₄, filtered and concentrated under reduced pressure to get the crude product. The crude product was purified by flash column chromatography (SiO₂ 100-200 mesh; 70% EtOAc in Hexane) to get Int-120 as a colorless gummy compound. The isolated product contained DMF and was taken for subsequent step without any further purification. Yield: 3.6 g (46%)

### Example 18: Preparation of tert-butyl (1-((1R,2S)-2-(4-bromophenyl)cyclopropoxy)-3-hydroxypropan-2-yl)carbamate (Int-125)

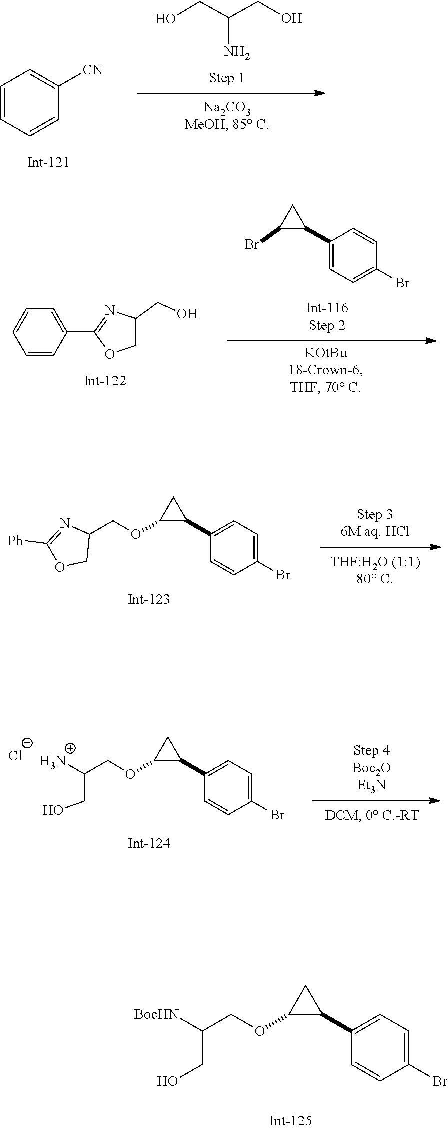

Step-1: To a stirred solution of benzonitrile (Int-121, 3 g, 29.1 mmol) in MeOH (30 mL) were added 2-aminopropane-1,3-diol (15.90 g, 175 mmol) and Na₂CO₃ (3.08 g, 29.1 mmol) at RT. The reaction mixture was stirred at 85° C. for 12 h. The reaction mixture was concentrated under reduced pressure to remove methanol. The reaction mixture was diluted with water (50 mL) and extracted with DCM (50 mL×2). The combined organic layers were washed with brine (50 mL), dried over anhydrous Na₂SO₄, filtered and concentrated under reduced pressure to get crude product. The crude product was purified by using flash column chromatography (SiO₂ 60-120 mesh; 60% EtOAc in Hexane) to afford 2-phenyl-4-hydroxymethyloxazoline (Int-122) as a white color solid. Yield: 2.8 g (54%). LC MS: Calculated for C₁₀H₁₁NO₂ is 177.2, Observed: 178.2 [M+1]⁺.

Step-2: To a stirred solution of Int-122 (1.5 g, 8.46 mmol) in THF (30 mL) were added KO^{I}Bu (0.950 g, 8.46 mmol) and 18-crown-6 (0.447 g, 1.693 mmol) at 0° C. under N₂ atmosphere. The reaction mixture was stirred at 0° C. for 15 min before the addition of 1-bromo-4-((1r,2r)-2-bromocyclopropyl)benzene (Int-116, 1.168 g, 4.23 mmol). The reaction mixture was then heated to 70° C. and stirred for 16 h. The reaction mixture was monitored by TLC: 20% EtOAc in Hexane, indicated some of the unreacted starting material. The reaction mixture was quenched with water (50 mL) and extracted with EtOAc (50 mL×2). The combined organic layers were washed with brine (50 mL), dried over anhydrous Na₂SO₄, filtered and concentrated under reduced pressure to get crude product. The crude product was purified by using flash column chromatography (SiO₂ 100-200 mesh; 20% EtOAc in Hexane) to afford Int-123 as a red color gummy compound in 52% purity as judged by UPLC. Yield: 420 mg (13%).

Step-3: To a solution of Int-123 (0.42 g, 1.128 mmol) in THE (4 mL) and water (4.00 mL), was added HCl (6 M Aqueous solution, 0.94 mL, 5.64 mmol) at 0° C. The reaction mixture was stirred at 80° C. for 48 h. The LCMS of the crude reaction mixture showed only 5% desired product along with unreacted Int-123. HCl (6M Aqueous solution, 0.94 mL, 5.64 mmol) was added again and continued the stirring at 80° C. for 16 h. The LCMS of crude reaction mixture indicated the formation of 46% desired product. Further, to improve the conversion, HCl (6M Aqueous solution, 0.94 mL, 5.64 mmol) was added second time and continued the stirring at 80° C. for additional 16 h. The reaction mixture was concentrated under reduced pressure, resulting residue was co-distilled with toluene, EtOAc and Hexane successively and concentrated under reduced pressure to get Int-124 as a red color gummy compound. Yield: 0.59 g (53% pure by LCMS, the product was taken as such to the next step without further purification). LC MS: Calculated for C₁₂H₁₆BrNO₂ is 286.17, Observed: 286.1 [M]⁺ and 288.1 [M+2]⁺.

Step-4: To a stirred solution of Int-124 (0.35 g, 1.085 mmol) in DCM (10 mL), were added Et₃N (0.756 mL, 5.42 mmol) followed by Boc₂O (0.504 mL, 2.170 mmol) at 0° C. The reaction mixture was stirred at RT for 16 h. The reaction mixture, as monitored by TLC, showed complete consumption of the starting material. The reaction mixture was quenched with water (30 mL) and extracted with DCM (30 mL×2). The combined organic layers were washed with brine (50 mL), dried over anhydrous Na₂SO₄, filtered and concentrated under reduced pressure. The crude product was purified by using flash column chromatography (SiO₂ 100-200 mesh; 30% EtOAc in Hexane) to afford Int-125 as a red color gummy compound. Yield: 170 mg (51% pure by LCMS, the product was taken as such to the next step without further purification). LC MS: Calculated for C₁₇H₂₄BrNO₄ is 386.29, Observed: 286.1 [M-Boc]⁺ and 288.1 [M-Boc+2].

### Example 19: Preparation of 4-(4,4,5,5-tetramethyl-1,3,2-dioxaborolan-2-yl)phenol (Int-61)

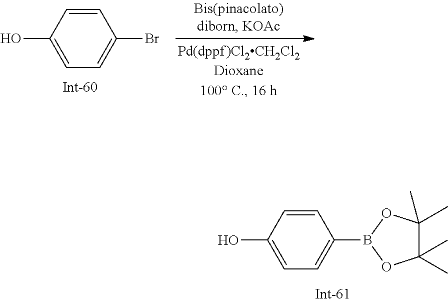 [SMILES: Oc1ccc(Br)cc1 ; CC1(C)OB(c2ccc(O)cc2)OC1(C)C]

Step 1: To a stirred solution of Int-60 (2.0 g, 9.09 mmol) in 1,4-dioxane (20 mL), were added bis(pinacolato)diboron (3.46 g, 13.64 mmol) and potassium acetate (2.2 g, 22.73 mmol) at room temperature. The reaction mixture was degassed using nitrogen gas for 10 min. To this mixture, was added Pd(dppf)Cl₂ (665 mg, 0.91 mmol) and degassing continued for 2 min. The reaction mixture was then heated at 90° C. for 4 h. The reaction was monitored by TLC, showed complete consumption of starting material. The reaction was filtered through Celite bed. The filtrate was diluted with EtOAc (100 mL), washed with water (50 mL), brine (50 mL), dried over sodium sulphate, filtered and concentrated under reduced pressure. The resulting crude product (2.5 g) was purified by flash column chromatography (SiO₂ 230-400 mesh; 500 EtOAc in pet ether) to obtain Int-61 as a white solid. Yield=1.8 g (82). LC-MS: Calculated for C₁₂H₁₇BO₃ is 220.08, Observed: 218.9 [M-1]⁺.

The following compounds were prepared according to Example 19 using the appropriate starting material. The 4-phenylcyclobutanone boronic acid derivative (Int-73). 4-phenyl-2-fluorocyclobutanone boronic acid (Int-73a) and 4-methyl 3-(4-(4,4,5,5-tetramethyl-1,3,2-dioxaborolan-2-yl)phenyl)cyclobutane-1I-carb oxyl ate (Int-74) were prepared from commercially available 4-bromophenylcyclobutanone, 4-bromo-2-fluorophenylcyclobutanone, and methyl 3-(4-bromophenyl)cyclobutane-1I-carb oxyl ate, respectively.

| Intermediate | Structure | Starting Material |
|---|---|---|
| Int-62a; Int-62b; Int-62c | 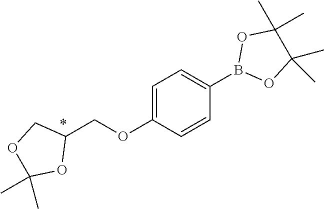 | Int-20a; Int-20b; Int-20c |
| Int-62d | 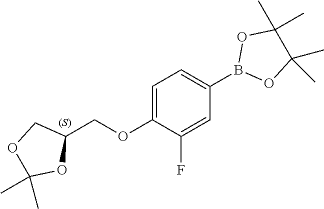 | Int-20d |
| Int-63 | 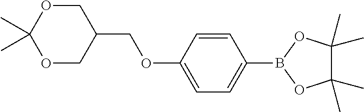 | Int-23 |
| Int-65a; Int-65b; Int-65c | 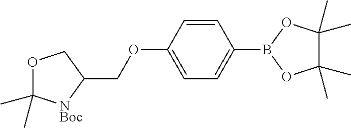 | Int-30a; Int-30b; Int-30c |
| Int-66 | 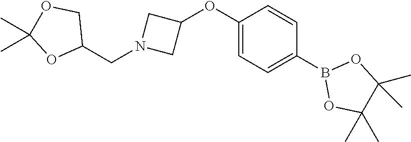 | Int-34 |
| Int-66a | 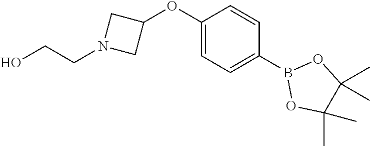 [SMILES: CC1(C)OB(c2ccc(OC3CN(CCO)C3)cc2)OC1(C)C] | Int-34a |
| Int-67 | 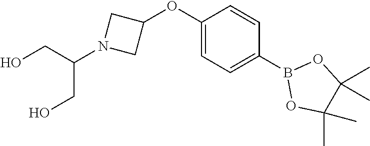 [SMILES: CC1(C)OB(c2ccc(OC3CN(C(CO)CO)C3)cc2)OC1(C)C] | Int-40 |
| Int-68 | 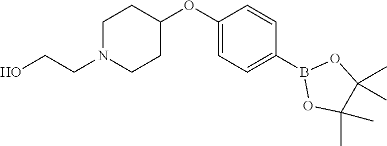 [SMILES: CC1(C)OB(c2ccc(OC3CCN(CCO)CC3)cc2)OC1(C)C] | Int-45a |
| Int-69 | 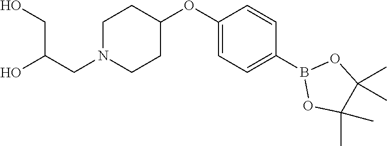 [SMILES: CC1(C)OB(c2ccc(OC3CCN(CC(O)CO)CC3)cc2)OC1(C)C] | Int-45b |
| Int-70a; Int-70b | 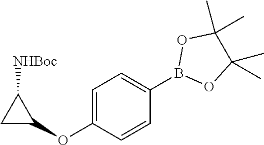 | Int-51a; Int-51b |
| Int-71 | 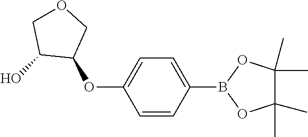 [SMILES: CC1(C)OB(c2ccc(O[C@@H]3COC[C@H]3O)cc2)OC1(C)C] | Int-53 |
| Int-72 | 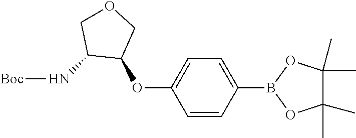 [SMILES: CC(C)(C)OC(=O)N[C@@H]1COC[C@H]1Oc1ccc(B2OC(C)(C)C(C)(C)O2)cc1] | Int-59 |
| Int-73 | 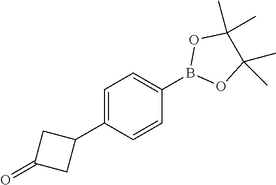 [SMILES: CC1(C)OB(c2ccc(C3CC(=O)C3)cc2)OC1(C)C] | 4-bromophenylcyclobutanone |
| Int-73a | 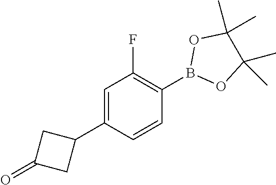 [SMILES: CC1(C)OB(c2ccc(C3CC(=O)C3)cc2F)OC1(C)C] | 4-bromo-2- fluorophenylcyclobutanone |
| Int-74 | 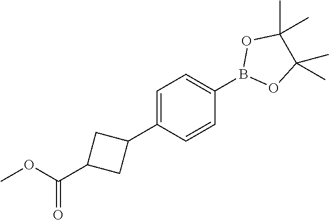 | 4- bromophenylcyclobutylmethylester |
| Int-99 | 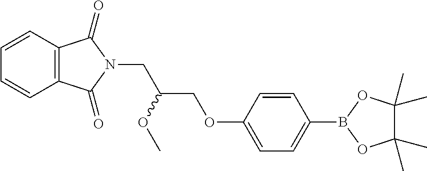 [SMILES: COC(COc1ccc(B2OC(C)(C)C(C)(C)O2)cc1)CN1C(=O)c2ccccc2C1=O] | Int-98 |
| Int-102a; Int-102b; Int-102c | 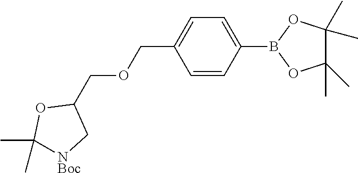 | Int-101a; Int-101b; Int-101c |
| Int-126 | 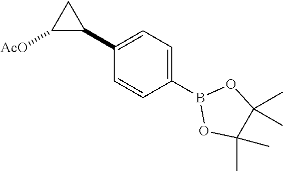 | Int-113 |
| Int-127 | 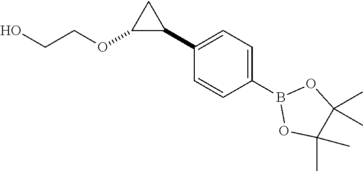 | Int-115 |
| Int-128 | 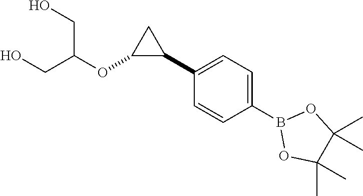 | Int-118 |
| Int-129 | 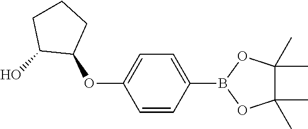 [SMILES: CC1(C)OB(c2ccc(O[C@@H]3CCC[C@H]3O)cc2)OC1(C)C] | Int-119 |
| Int-130 | 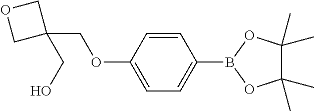 [SMILES: CC1(C)OB(c2ccc(OCC3(CO)COC3)cc2)OC1(C)C] | Int-120 |
| Int-131 | 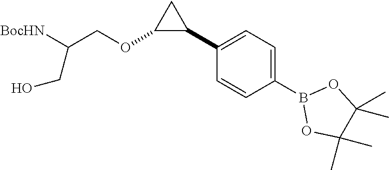 | Int-125 |

### Example 20: Preparation of Certain Compounds of Formula (I) via Suzuki Reaction

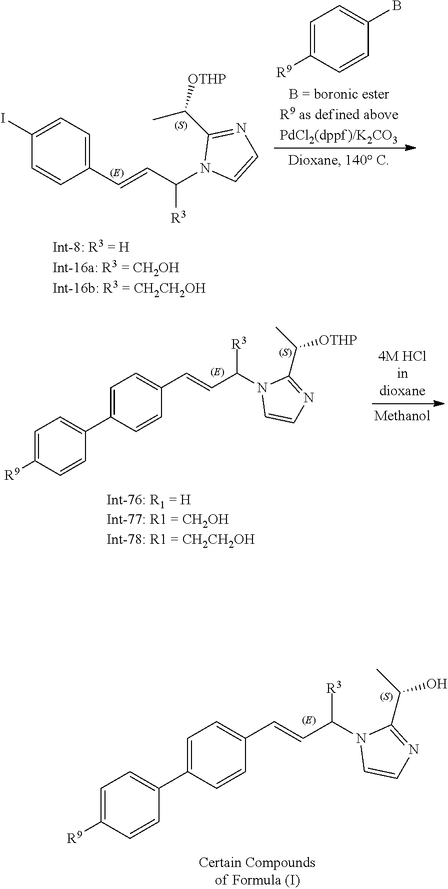

Step 1: To a stirred solution of appropriate aryl iodide (Int-8, Int-16a, or Int-16b) (0.45 mmol) in DMF (1 mL), were added an appropriate aryl boronic ester intermediate (see Example 14) (0.32 mmol), potassium phosphate tribasic (0.96 mmol) and water (0.5 mL) at room temperature. The reaction mixture was degassed with nitrogen gas for 5 min. To this reaction mixture was added Pd(dppf)Cl₂ (11.7 mg, 0.02 mmol) and degassing continued for 2 min. The reaction mixture was then subjected to microwave irradiation at 100° C., for 3 h. After completion of the reaction, the inorganic solids were filtered through Celite pad. The filtrate was diluted in EtOAc (50 mL) and washed with water (30 mL), brine (30 mL), dried over anhydrous sodium sulphate, filtered and concentrated under reduced pressure. The resulting crude product was purified by flash column chromatography (SiO₂ 230-400 mesh; 300 MeGH in DCM) to afford intermediates Int-76, Int-77, and Int-78 compounds.

Some of the Int-76, Int-77, and Int-78 compounds were deprotected to give the desired final compounds:

Step 2: To a stirred solution of the Int-76, Int-77, or Int-7S compound (0.219 mmol) in DCM (2 mL), was added 4.0 M HCl in dioxane (1 mL) at 0° C. The reaction mixture was then stirred for 2 h at 25° C. The reaction was monitored by TLC; TLC showed complete consumption of starting material. The volatiles were evaporated under reduced pressure. The resulting crude product was purified by reversed phase preparative HPLC (10 mM ammonium bicarbonate in water and acetonitrile) to afford Certain Compounds of Formula (I) as a white solid. Yields ranged between 28-45%.

The following compounds were prepared according to Example 20:

| Compound No. | Aryl Iodide | Boronic Ester | Mass [M + H]+ |
|---|---|---|---|
| 1 | Int-8 | Int-68 | 448.5 |
| 2 | Int-8 | Int-69 | 478.5 |
| 3 | Int-8 | Int-65b | 394.4 |
| 4 | Int-8 | Int-70a | 376.4 |
| 8 | Int-16a | Int-65a | 424.505 |
| 9 | Int-16a | Int-65b | 424.505 |
| 10 | Int-16a | Int-65c | 424.505 |
| 11 | Int-27 | Int-65a | 442.3 |
| 13 | Int-16a | Int-70a | 406.49 |
| 14 | Int-16a | Int-70a* | 406.54 |
| 15 | Int-16a | Int-70a* | 406.5 |
| 16 | Int-16a | Int-70b | 420.516 |
| 17 | Int-16a | Int-68 | 478.595 |
| 18 | Int-16b | Int-68 | 492.622 |
| 19 | Int-16b | Int-65a | 438.531 |
| 20 | Int-16a | Int-66a | 450.5 |
| 22 | Int-16a | Int-102a | 424.5 |
| 23 | Int-16a | Int-62b | 425.49 |
| 24 | Int-16a | Int-62c | 425.49 |
| 25 | Int-16a | Int-65a | 438.531 |
| 29 | Int-16a | Int-102b | 424.5 |
| 30 | Int-27 | Int-102a | 442.5 |
| 32 | Int-16a | Int-62d | 443.5 |
| 33 | Int-16a | Int-63 | 439.52 |
| 34 | Int-16a | Int-72 | 436.52 |
| 35 | Int-16a | Int-71 | 437.20 |
| 68 | Int-16a | Int-130 | 451.4 |
| 69 | Int-16a | Int-126 | 433.5 |
| 70 | Int-16a | Int-128 | 465.4 |
| 71 | Int-16a | Int-127 | 435.4 |
| 72 | Int-16a | Int-131 | 501.0 |
| 73 | Int-16a | Int-129 | 435.5 |

*isomers separated on purification

For some compounds, final derivatizations were carried out before deprotection to give the desired final targets.

### Example 21: Preparation of Certain Compounds of Formula (I) via O-Alkylation

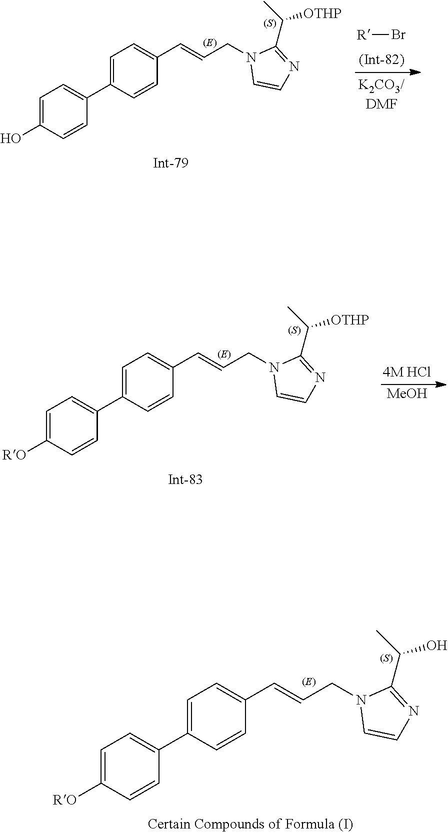

Step 1: To a stirred solution of Int-79 (prepared via the procedures in Example 22) (1.50 g, 6.58 mmol) in DMF (7 mL) was added K₂CO₃. The reaction mixture was stirred at 30 minutes. To this, corresponding alkyl halide Int-82 (6.58 mmol) was added and heated at 80° C. for a period of 8 h. After completion of the reaction, the reaction mixture poured into ice cold water and extracted with Ethyl acetate. The aqueous layer was further extracted with Ethyl acetate (50 mL×2). The combined organic layer was dried over anhydrous sodium sulphate, filtered and concentrated under reduced pressure. The resulting crude was purified by flash column chromatography (SiO₂ 100-200 mesh size, 0-5% methanol in DCM) to afford the intermediate compounds Int-83 as solids. Yield 30-45%.

Step 2: To a stirred solution of Int-83 (0.2476 mmol) in dry DCM (2 mL), was added HCl (4 M in 1,4-Dioxane, 2 mL) at 0° C. The reaction mixture was stirred at 0° C. to RT for 1 h. After the completion of the reaction, the reaction mixture was concentrated under reduced pressure to afford the crude product. The crude product was purified by using 0.1% HCOOH in H₂O and ACN to afford desired Certain Compounds of Formula (I) as off white semi-solids. Yields ranged between 23.5-50%.

The following compounds were prepared according to Example 21:

| Compound | | Mass |
|---|---|---|
| No. | Alkyl Halide | [M + H]⁺ |
| 5 | 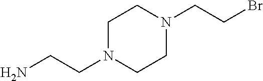 [SMILES: NCCN1CCN(CCBr)CC1] | 476.626 |
| 6 | 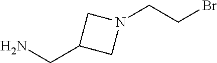 | 477.61 |
| 7 | 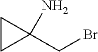 | 390.49 |

### Example 22: Preparation of 2,2′-((2-((4′—((S,E)-4-hydroxy-3-(2-((S)-1-hydroxyethyl)-1H-imidazol-1-yl)but-1-en-1-yl)-[1,1′-biphenyl]-4-yl)oxy)ethyl)azanediyl)bis(ethan-1-ol) (Compound 28)

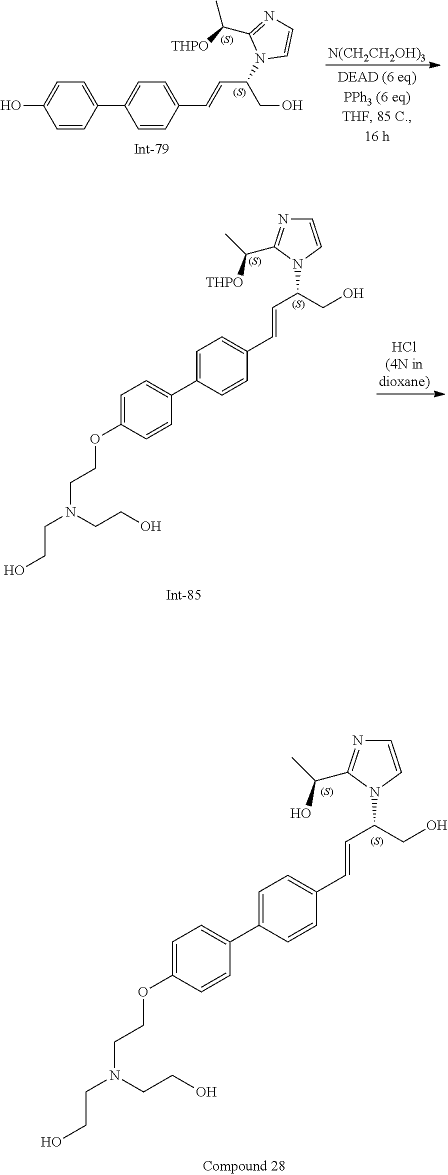

Step 1: To a stirred solution of triethanolamine (309 mg,2.070 mmol) in THF (5 mL), was added PPh₃ (543 mg, 2.070 mmol) and cooled to 0° C. To this cooled solution, DEAD (361 mg, 2.070 mmol) and Int-79 (prepared via the procedures in Example 22) (150 mg, 0.3451 mmol) were added slowly and stirred at 0° C. for 30 minutes. Then, temperature raised gradually up to 85° C. and stirred for 16 h. After completion of the reaction, the reaction mixture was diluted with EtOAc (20 mL) and washed with water (10 mL). The organic layer was dried over anhydrous Na₂SO₄ and concentrated under reduced pressure to afford the crude product. The crude product was purified by column chromatography on silica gel (60-120 mesh) by eluting with 7%-10% MeOH in DCM to afford Int-85. Yield: 72%. LC MS: Calculated for C₃₂H₄₂N₃O₆ is 565.32, Observed: 566.22[M+H]⁺.

Step 2: To a stirred solution of Int-85 (120 mg, 0.219 mmol) in DCM (2 mL), was added 4.0 M HCl in dioxane (1 mL) at 0° C. The reaction mixture was then stirred for 2 h at 25° C. The reaction was monitored by TLC; TLC showed complete consumption of starting material. The volatiles were evaporated under reduced pressure. The resulting crude product was purified by reversed phase preparative HPLC (10 mM ammonium bicarbonate in water and acetonitrile) to afford Compound 28 as a white solid. Yield: 12 mg (28%). LC MS: Calculated for C₂₄H₂₈N₂O₅ is 424.49, Observed: 425.3 [M+1]⁺.

### Example 23: Preparation of (2S,E)-4-(4′-(2-(dimethylamino)-3-hydroxypropoxy)-[1,1′-biphenyl]-4-yl)-2-(2-((S)-1-hydroxyethyl)-1H-imidazol-1-yl)but-3-en-1-ol (Compound 12)

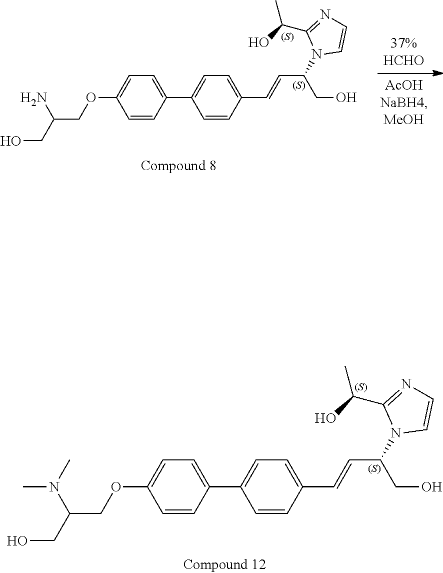

Step 1: To a stirred solution of Compound 8 (25 mg, 0.0591 mmol) in dry MeOH (1 mL), were added Formaldehyde (37 wt. % in H₂O, 0.03 mL, 0.2922 mmol) and Acetic acid (3.5 mg, 0.0591 mmol) at RT. The reaction mixture was stirred at RT for 15 minutes. Then MP-Cyanoborohydride (2.22 mmol/g, 53.2 mg, 0.118 mmol) was added at RT. The reaction mixture was stirred at RT for 16 h. After the completion of the reaction, the reaction mixture was filtered and concentrated under reduced pressure to afford the crude product. The crude product was purified by reverse phase prep HPLC purification using 0.1% HCOOH in H₂O and ACN to afford Compound 12 as off white solid. Yield: 6 mg (22.5%). LC MS: Calculated for C₂₆H₃₃N₃O₄ is 451.25, Observed:452.1.

### Example 24: Preparation of Certain Compounds of Formula (I) via Reductive Amination of Cyclobutanone Intermediate (Int-80)

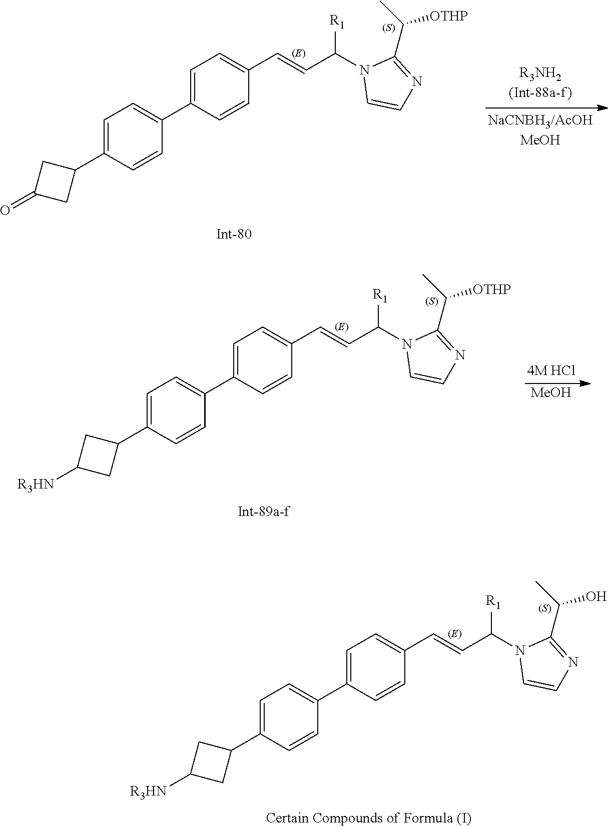

Step:1 To a stirred solution of Int-80 (prepared via the procedures in Example 15 from Int-73) (60 mmol) in methanol (10 mL), appropriate respective amine Int-88 was added (78 mmol, 1.3 eq) followed by a drop of acetic acid. The reaction was stirred for a period of 45 minutes. To this sodium cyanoborohydride (66 mol, 1.1 eq) was added and the reaction was stirred for a period of 4 h. After 4 h reaction mixture was concentrated, diluted with water and extracted with ethyl acetate (2×25 mL). The organic layer was washed with satd. NaCl (15 mL), dried MgSO₄ and concentrated to give the crude product of Int-89. Yields ranged between 75-90%. The reaction mixture is taken up to the next step without further purification.

Step 2: To a stirred solution of Int-89 (50 mmol) in methanol (5 mL), was added 4 M HCl in dioxane (1.0 mL) at 0° for 30 minutes. The reaction was monitored by TLC, showed complete consumption of starting material. After completion of reaction, the volatiles were evaporated under reduced pressure. The resulting crude product was purified by reversed phase preparative HPLC (10 mM ammonium bicarbonate buffer and acetonitrile) to afford desired Certain Compounds of Formula (I). Yields ranged between 57-89%.

The following compounds were prepared according to Example 24:

| Compound | | Mass |
|---|---|---|
| No. | Amine | [M + H]⁺ |
| 37 | 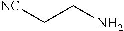 | 456.25 |
| 38 | 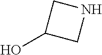 | 459.25 |
| 39 | no amine* | 404.21 |
| 40 | 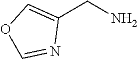 | 484.25 |
| 41 | 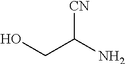 | 472.24 |
| 42 | 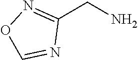 | 485.35 |
| 44 | 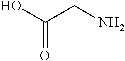 | 462.5 |
| 45 | 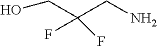 | 498.5 |
| 46 | 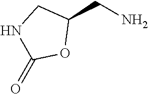 | 503.6 |
| 47 | 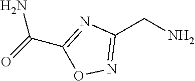 | 529.6 |
| 48 | NH₂ | 404.5 |
| 75** | 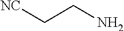 | 460.5 |

*ketone reduced to alcohol directly
**starting ketone was made from Int-73a

### Example 25: Preparation of Certain Compounds of Formula (I) via Amide Formation from Acid Intermediate (Int-91)

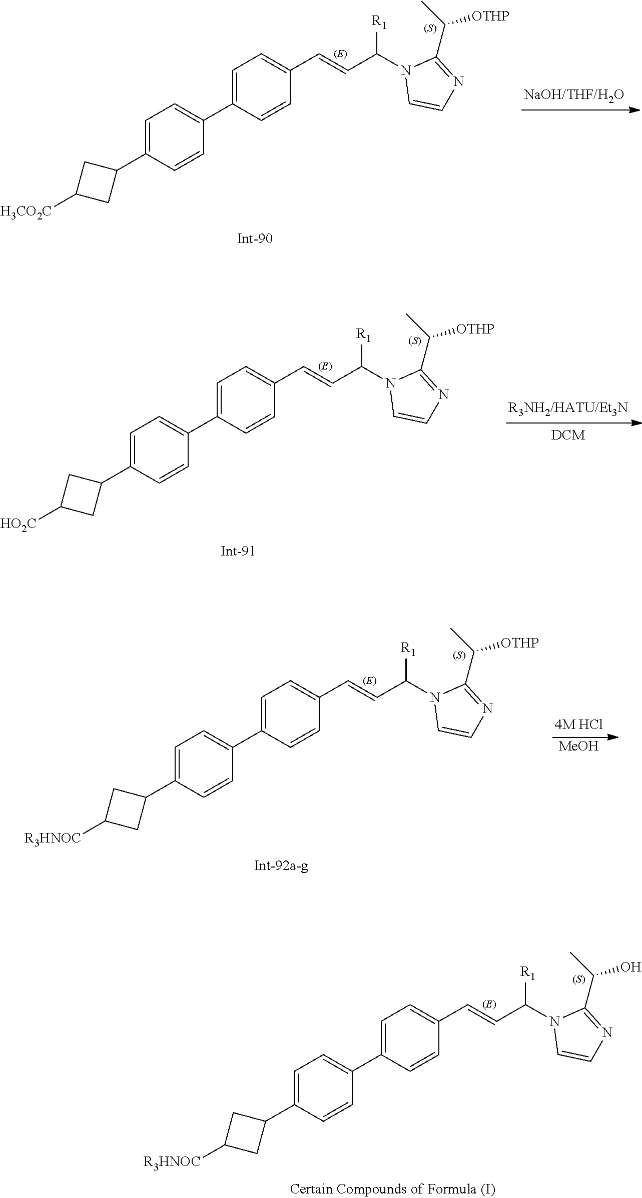

Step:1 To a stirred solution of Int-90 (prepared via the procedures in Example 22 from Int-74) (60 mmol) in THE (10 mL), NaOH (1.2 eq) dissolved in water was added and stirred for a period of 30 minutes. LCMS indicated complete conversion. The reaction mixture was acidified using 6 M HCL to pH=2 and extracted with ethyl acetate. The organic layer is washed with brine, dried (Na₂SO₄) and concentrated to give us the desired acid Int-91. Yields ranged between 87-90%. The reaction mixture is taken up to the next step without further purification.

Step2: To a stirred solution of Int-91 in DCM (10 mL), amines (1.2 eq) was added along with HATU (1.1 eq) and triethyl amine (1.5 eq) and stirred for period of 90 minutes. The reaction mixture was poured into ice cold water and extracted with DCM (2×25 ml). The organic layers were washed with satd. NaCl (10 ml), dried (Na₂SO₄) and concentrated under vacuum. The crude mixture Int-92a-g was taken up to the next step without further purification with yields ranging from 58-72%.

Step 3: To a stirred solution of Int-92a-g (50 mmol) in methanol (5 mL), was added 4 M HCl in dioxane (1.0 mL) at 0° for 30 minutes. The reaction was monitored by TLC, showed complete consumption of starting material. After completion of reaction, the volatiles were evaporated under reduced pressure. The resulting crude product was purified by reversed phase preparative HPLC (10 mM ammonium bicarbonate buffer and acetonitrile) to afford desired Certain Compounds of Formula (I). Yields ranged between 57-89%. Compounds 51 (m/z: 433.25) and 67 (m/z: 447.50) were prepared from similar reactions on Int-91 directly.

The following compounds were prepared according to Example 25:

| Compound | | Mass |
|---|---|---|
| No. | Amide | [M + H]⁺ |
| 49 | 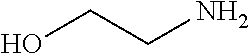 | 476.5 |
| 50 | 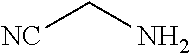 | 471.5 |
| 52 | 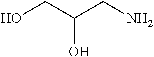 | 506.5 |
| 53 | 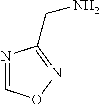 | 514.5 |
| 54 | 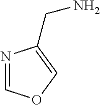 | 513.5 |
| 55 | 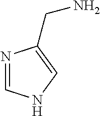 | 512.5 |

### Example 26: Preparation of Certain Compounds of Formula (I) via Reductive Amination from Aldehyde Intermediate (Int-93)

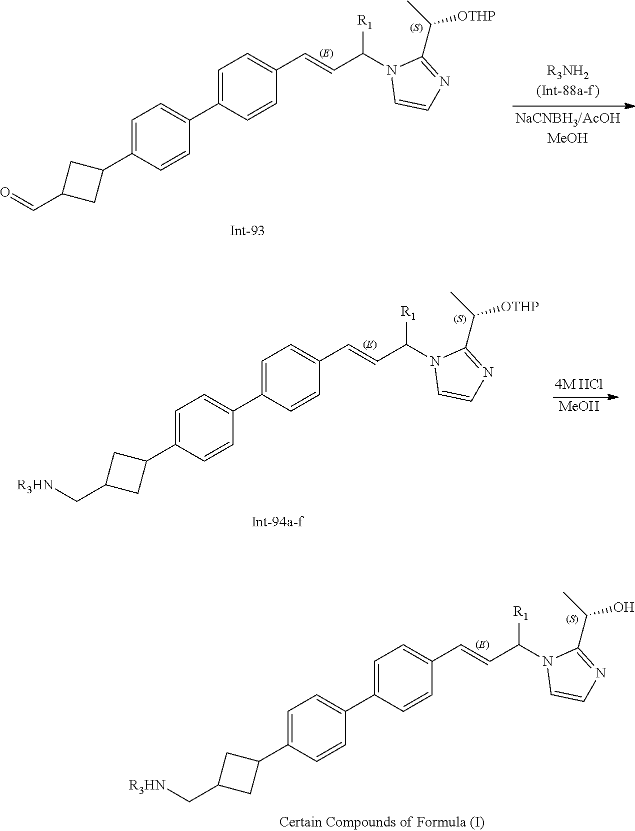

Step:1 To a stirred solution of Int-93 (prepared via the procedures in Examples 14 and 15 from 3-(4-bromophenyl)cyclobutane-1-carbaldehyde) (60 mmol) in methanol (10 mL), appropriate respective amine Int-88 was added (78 mmol, 1.3 eq) followed by a drop of acetic acid. The reaction was stirred for a period of 45 minutes. To this sodium cyanoborohydride (66 mol, 1.1 eq) was added and the reaction was stirred for a period of 4 h. After 4 h reaction mixture was concentrated, diluted with water and extracted with ethyl acetate (2×25 mL). The organic layer was washed with satd. NaCl (15 mL), dried MgSO₄ and concentrated to give the crude product of Int-94. Yields ranged between 75-90%. The reaction mixture is taken up to the next step without further purification.

Step 2: To a stirred solution of Int-94 (50 mmol) in methanol (5 mL), was added 4 M HCl in dioxane (1.0 mL) at 0° for 30 minutes. The reaction was monitored by TLC, showed complete consumption of starting material. After completion of reaction, the volatiles were evaporated under reduced pressure. The resulting crude product was purified by reversed phase preparative HPLC (10 mM ammonium bicarbonate buffer and acetonitrile) to afford desired Certain Compounds of Formula (I). Yields ranged between 65-79%.

The following compounds were prepared according to Example 26:

| Compound | | Mass |
|---|---|---|
| No. | Amide | [M + H]⁺ |
| 56 | 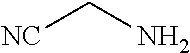 | 457.5 |
| 57 | 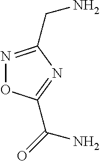 | 543.5 |
| 58 | 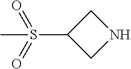 | 536.5 |
| 59 | 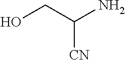 | 487.5 |
| 60 | 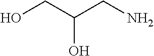 | 492.6 |
| 61 | 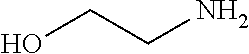 | 462.5 |

### Example 27: Preparation of Certain Compounds of Formula (I) via Amide Formation from Amine Intermediate (Int-60)

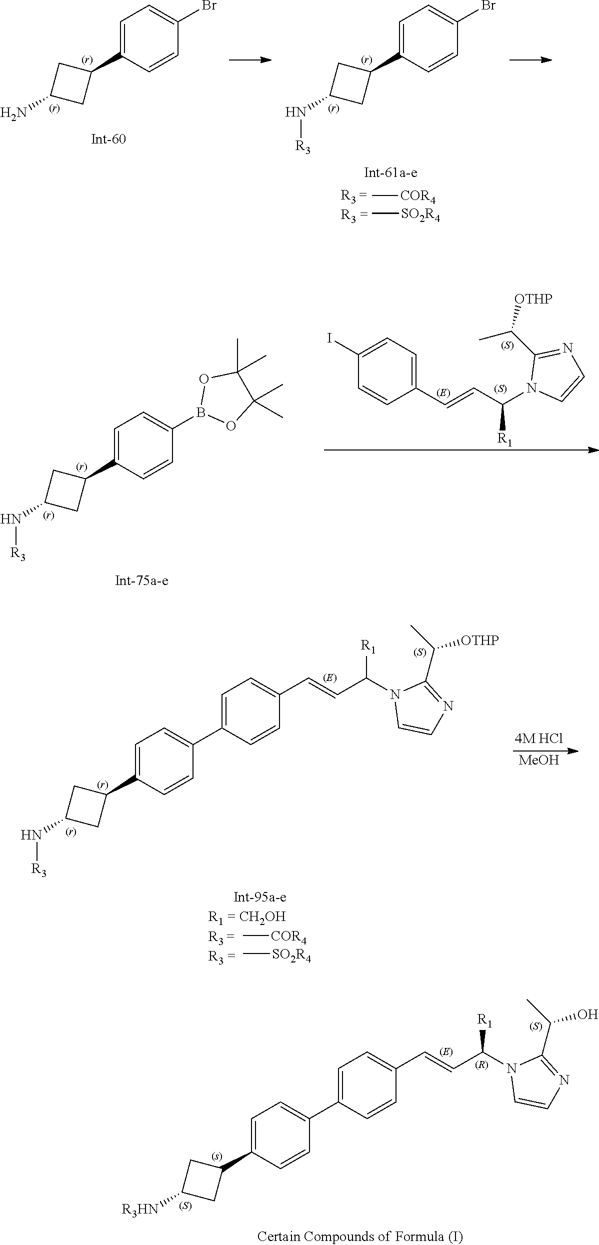

Step 1: To a stirred solution of the Int-60 (300 mg) in DCM (10 ml), triethylamine (2.5 eq) was added followed by the addition of acid chloride/sulfonyl chloride (1.2 eq) and stirred for a period of 45 minutes. TLC indicated completion of the reaction. The reaction mixture was poured into water (15 ml) and extracted with DCM (2×15 mL). The combined organic layer was washed with satd. NaCl solution (15 mL), dried (Na₂SO₄) and concentrated to dryness. The crude Int-61a-e was taken up to the next without further purification. Yields range between 90-95%.

Step 2: To a stirred solution of Int-61a-e (0.300 g) in 1,4-dioxane (8 mL), were added bis(pinacolato)diboron (1.2 eq) and potassium acetate (2 eq) at room temperature. The reaction mixture was degassed using nitrogen gas for 10 min. To this mixture, was added Pd(dppf)Cl₂ (20 mg) and degassing continued for 2 min. The reaction mixture was then heated at 120° C. for 4 h. The reaction was monitored by TLC, showed complete consumption of starting material. The reaction was filtered through Celite bed. The filtrate was diluted with EtOAc (100 mL), washed with water (50 mL), brine (50 mL), dried over sodium sulphate, filtered and concentrated under reduced pressure. The resulting crude products Int-75a-e were taken on to the next step without further purifications.

Step 3: To a stirred solution of appropriate aryl iodide Int-75a-e (0.45 mmol) in DMF (1 mL), were added an appropriate aryl boronic ester intermediate (see Example 14) (0.32 mmol), potassium carbonate (0.96 mmol) and water (0.5 mL) at room temperature. The reaction mixture was degassed with nitrogen gas for 5 min. To this reaction mixture was added Pd(dppf)Cl₂ (11.7 mg, 0.02 mmol) and degassing continued for 2 min. The reaction mixture was then subjected to microwave irradiation at 100° C., for 3 h. After completion of the reaction, the inorganic solids were filtered through Celite pad. The filtrate was diluted in EtOAc (50 mL) and washed with water (30 mL), brine (30 mL), dried over anhydrous sodium sulphate, filtered and concentrated under reduced pressure. The resulting crude product was purified by flash column chromatography (SiO₂ 230-400 mesh; 3% MeOH in DCM) to afford intermediates Int-95a-e.

Step 4: To a stirred solution of Int-95a-e (50 mmol) in methanol (5 mL), was added 4 M HCl in dioxane (1.0 mL) at 0° for 30 minutes. The reaction was monitored by TLC, showed complete consumption of starting material. After completion of reaction, the volatiles were evaporated under reduced pressure. The resulting crude product was purified by reversed phase preparative HPLC (10 mM ammonium bicarbonate buffer and acetonitrile) to afford desired Certain Compounds of Formula (I). Yields ranged between 65-79%.

The following compounds were prepared according to Example 27:

| Compound | Acid Chloride/ | Mass |
|---|---|---|
| No. | Sulfonyl Chloride | [M + H]⁺ |
| 62 | 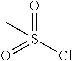 | 482.6 |
| 63 | 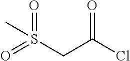 | 524.5 |
| 64 | 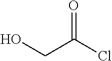 | 462.5 |
| 65 | 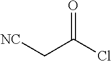 | 471.5 |
| 66 | 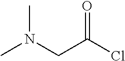 | 489.5 |

### Example 28: Preparation of tert-butyl 4-(((4′—((S,Z)-1-fluoro-4-hydroxy-3-(2-((S)-1-hydroxyethyl)-1H-imidazol-1-yl)but-1-en-1-yl)-[1,1′-biphenyl]-4-yl)oxy)methyl)-2,2-dimethyloxazolidine-3-carboxylate (Compound 11)

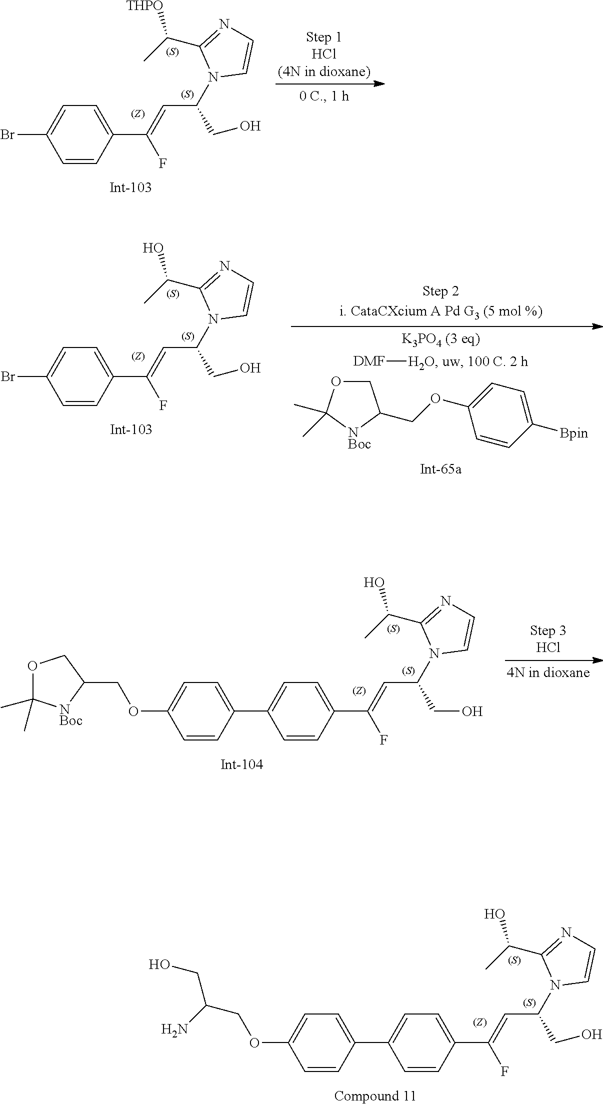

Step 1: To a stirred solution of Int-27 (170 mg, 0.386 mmol) in DCM (5 mL) was added 4 N HCl in dioxane (3 mL) and stirred at 0° C. for 2 h. After completion of the reaction, the solvent was concentrated under reduced pressure to afford the crude product, which was triturated with petroleum ether, decanted and dried under reduced pressure to get Int-103. Analysis of Int-103 by chiral SFC indicated purity of 95% which was used in the further steps. Yield: 140 mg. LC MS: Calculated for C₁₅H₁₆BrFN₂O₂ is 355.21, Observed: 355 [M]+ and 357[M+2]⁺.

Step 2: To a stirred solution of Int-103 (140 mg, 0.394 mmol) in DMF (4 mL), were added Int-65a (187.98 mg, 0.433 mmol) and K₃PO₄ (251 mg, 1.183 mmol) and water (0.5 mL), and the reaction mixture was degassed with nitrogen for 5 minutes. Then CataCXium A® Pd G3 (14.36 mg, 0.0197 mmol, 5 mol %) was added, again degassed under nitrogen for 5 minutes. The reaction mixture was then subjected to microwave irradiation at 100° C., 15 psi for 2 h. After completion of the reaction, the reaction mass was diluted with EtOAc (100 mL×2) and washed with water (50 mL). The organic layer was dried over anhydrous Na₂SO₄ and concentrated under reduced pressure to afford the crude product. The crude product was purified by RP-HPLC using 0.1% formic acid in H₂O and Acetonitrile to afford product Int-104 (20 mg tail+50 mg main peak). Analysis of Int-104 main peak by chiral SFC indicated purity of 48: 49% and the tail peak a purity of 51:45. The tail fraction was taken for the next step. Yield: 70 mg. LC MS: Calculated for C₃₂H₄₀FN₃O is 581.69, Observed: 582.4[M+H]⁺.

Step-3: To a stirred solution of Int-104 (20 mg, 34.38 mmol) in dry DCM (5 mL), was added HCl (4 N in 1,4-dioxane, 1 mL) at 0° C. and the reaction mixture stirred at 0° C. to RT for 1 h. After completion of the reaction, the solvent was concentrated under reduced pressure to afford the crude product, which purified by RP-HPLC using 0.1% formic acid in H₂O and Acetonitrile to get Compound 11. Yield: 4.1 mg (27%). LC MS: Calculated for C₂₄H₂₈FN₃O is 441.5, Observed: 442.4 [M+H]⁺.

### Example 29: Preparation of (2S,E)-4-(4′-(3-(dimethylamino)-2-hydroxypropoxy)-[1,1′-biphenyl]-4-yl)-2-(2-((S)-1-hydroxyethyl)-1H-imidazol-1-yl)but-3-en-1-ol (Compound 21)

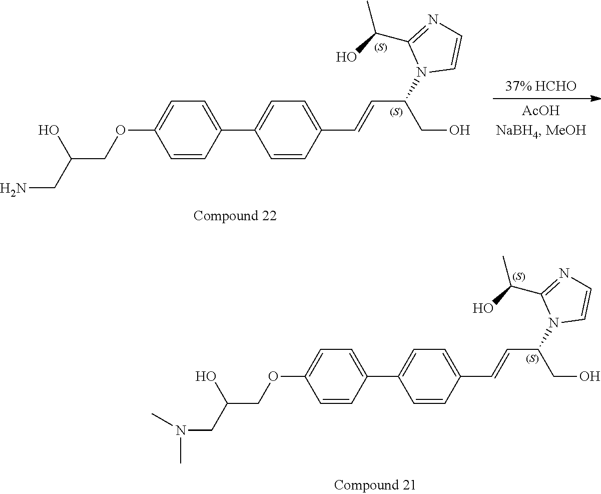

Step 1: To a stirred solution of Compound 22 (25 mg, 0.0591 mmol) in dry MeOH (1 mL), were added Formaldehyde (37 wt. % in H₂O, 0.03 mL, 0.2922 mmol) and Acetic acid (3.5 mg, 0.0591 mmol) at RT. The reaction mixture was stirred at RT for 15 minutes. Then MP-Cyanoborohydride (2.22 mmol/g, 53.2 mg, 0.118 mmol) was added at RT. The reaction mixture was stirred at RT for 16 h. After the completion of the reaction, the reaction mixture was filtered and concentrated under reduced pressure to afford the crude product. The crude product was purified by reverse phase prep HPLC purification using 0.1% HCOOH in H₂O and ACN to afford Compound 21 as off white solid. Yield: 6 mg (22.5%). LC MS: Calculated for C₂₆H₃₃N₃O₄ is 451.25, Observed:452.1. The same procedure was repeated to using the chiral (S) isomer of Compound 22 derivative to produce Compound 26.

### Example 30: Preparation of (2S,E)-4-(4′-(3-amino-2-methoxypropoxy)-[1,1′-biphenyl]-4-yl)-2-(2-((S)-1-hydroxyethyl)-1H-imidazol-1-yl)but-3-en-1-ol (Compound 27)

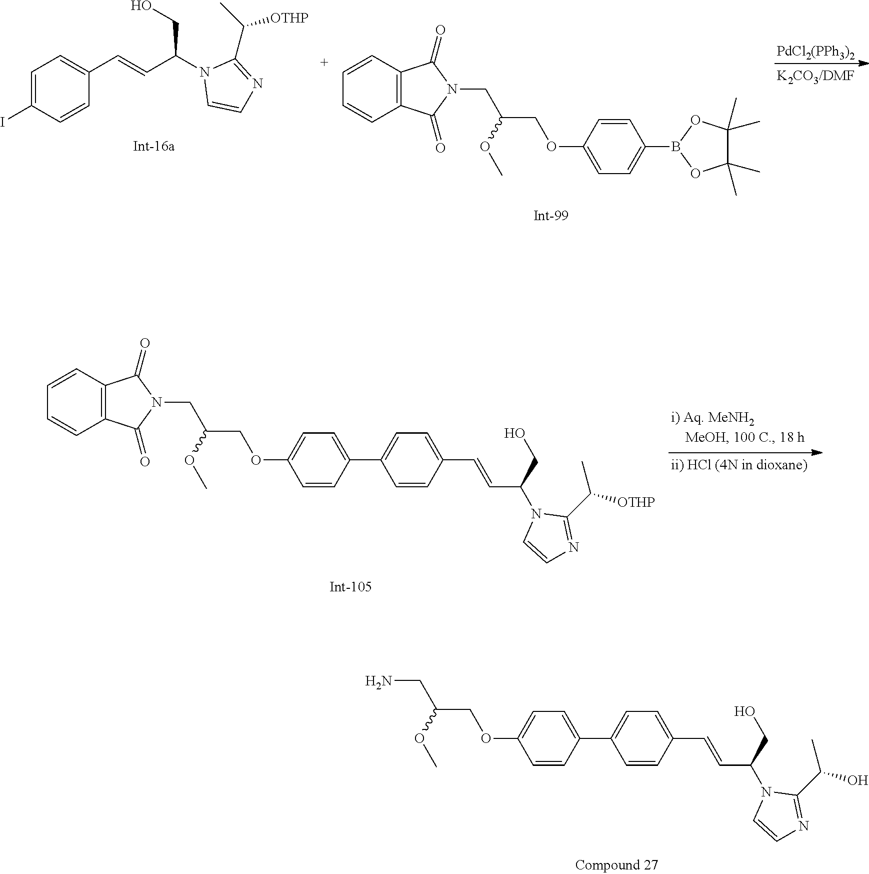

Step 1: To a stirred solution of Int-16a (2.31 mmol) in DMF (10 mL) were added Int-99 (3.46 mmol) and potassium carbonate (6.92 mmol) at RT, the reaction mixture was degassed with nitrogen for 5 minutes. To this was added Pd(dppf)Cl₂ (0.188 g, 0.03 mmol), again degassed with nitrogen for 2 minutes. The reaction mixture was then subjected to microwave irradiation at 100° C. for 4 h. After completion of the reaction, the inorganic solids were filtered through celite pad and washed with EtOAc. The filtrate was dissolved in EtOAc (100 mL) and washed with water (50 mL), dried over anhydrous Na₂SO₄, filtered and concentrated under reduced pressure to afford crude product. The crude product was purified by column chromatography using silica gel (60-120 mesh) by eluting with 10% Methanol in DCM to afford Int-105. Yield: 76%.

Steps-2 & 3: To a stirred solution of Int-105 (0.600 g, 0.92 mmol) in methanol (10 mL), was added 40% aqueous methyl amine solution (0.7 mL, 9.20 mmol) at RT. The reaction mixture was heated at 100° C. for 18 h. After completion of the reaction, the reaction mixture was concentrated under reduced pressure to afford a crude residue. The crude residue was diluted with 10% MeOH in DCM (100 mL) and washed with water (50 mL). The organic layer was dried over anhydrous Na₂SO₄ and concentrated under reduced pressure to afford the crude product. The crude product was dissolved in dry DCM (5 mL), HCl (4 N in 1,4-Dioxane; 10 mL) was added at 0° C. and the reaction mixture stirred at RT for 2 h. After completion of the reaction, the reaction mixture was concentrated under reduced pressure and triturated with diethyl ether to afford the crude product. The crude product was purified by using 0.1% ammonium acetate in H₂O and ACN to afford Compound 27 as an off white solid. Yield: 0.020 g (10%).

### Example 31: Preparation of (2S,E)-4-(4′-(2-amino-3-methoxypropoxy)-[1,1′-biphenyl]-4-yl)-2-(2-((S)-1-hydroxyethyl)-1H-imidazol-1-yl)but-3-en-1-ol (Compound 31)

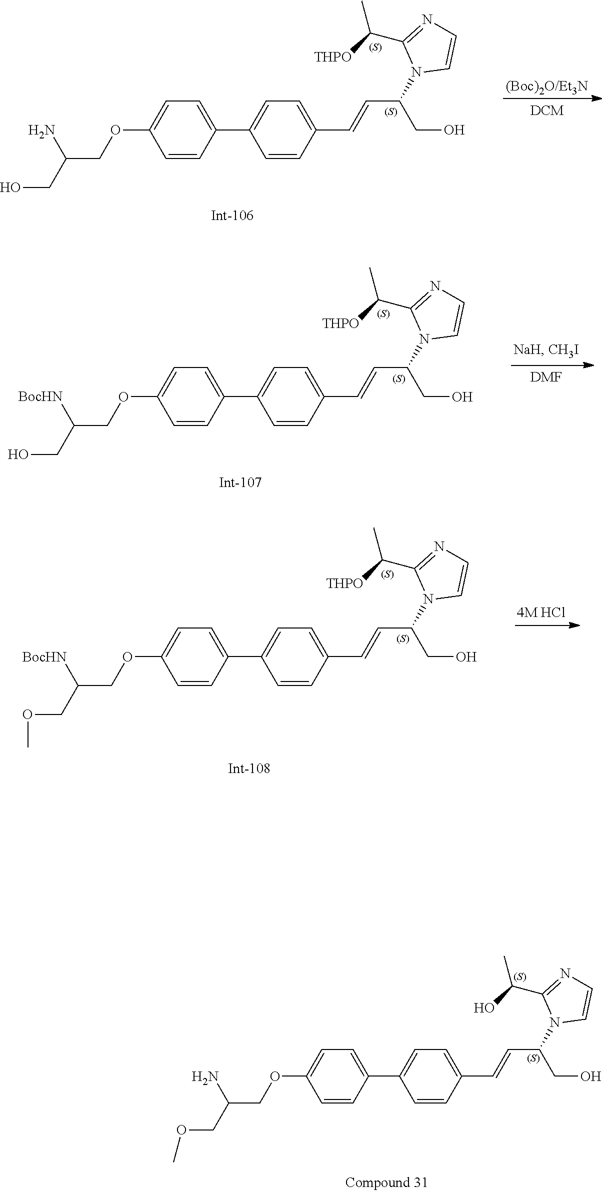

Step 1: To a stirred solution of Int-106 (prepared as described in Example 15 from Int-16a and Int-65a) (2 mmol) in DCM (10 mL), (Boc)₂O (3 mmol) was added followed by triethyl amine (4 mmol) and stirred for a period of 25 minutes. TLC indicated complete conversion. The reaction mixture is diluted with DCM (25 mL) washed with brine (10 mL), dried (Na₂SO₄) and concentrated under vacuum to give a crude Int-107 which yielded close to 99% which was taken to next step without further purification.

Step 2: To a stirred solution of NaH (12 mmol) in DMF (3 mL), Int-107 (10 mmol) was added dropwise in DMF (5 mL) over a period of 5 minutes. The reaction mixture was stirred at room temperature for 10 minutes followed by addition of methyl iodide (15 mmol). The reaction mixture was quenched with satd. NH₄Cl (10 mL) and extracted with ethyl acetate (2×15 mL). The organic layer was washed with satd. NaCl (10 mL), dried (Na₂SO₄) and concentrated under vacuum. The crude mixture Int-108 which yield around 56% was taken to the next step without further purification.

Step 3: To a stirred solution of Int-108 (0.219 mmol) in DCM (2 mL), was added 4.0 M HCl in dioxane (1 mL) at 0° C. The reaction mixture was then stirred for 2 h at 25° C. The reaction was monitored by TLC; TLC showed complete consumption of starting material. The volatiles were evaporated under reduced pressure. The resulting crude product was purified by reversed phase preparative HPLC (10 mM ammonium bicarbonate in water and acetonitrile) to afford Compound 31 as a white solid. Yield: 12 mg (28%). LC MS: Calculated for C₂₅H₃₁N₃O₄ is 437.5, Observed: 438.5 [M+1]⁺.

### Example 32: (2S,E)-4-(4′-(3-ethoxy-2-hydroxypropoxy)-[1,1′-biphenyl]-4-yl)-2-(2-((S)-1-hydroxyethyl)-1H-imidazol-1-yl)but-3-en-1-ol (Compound 36)

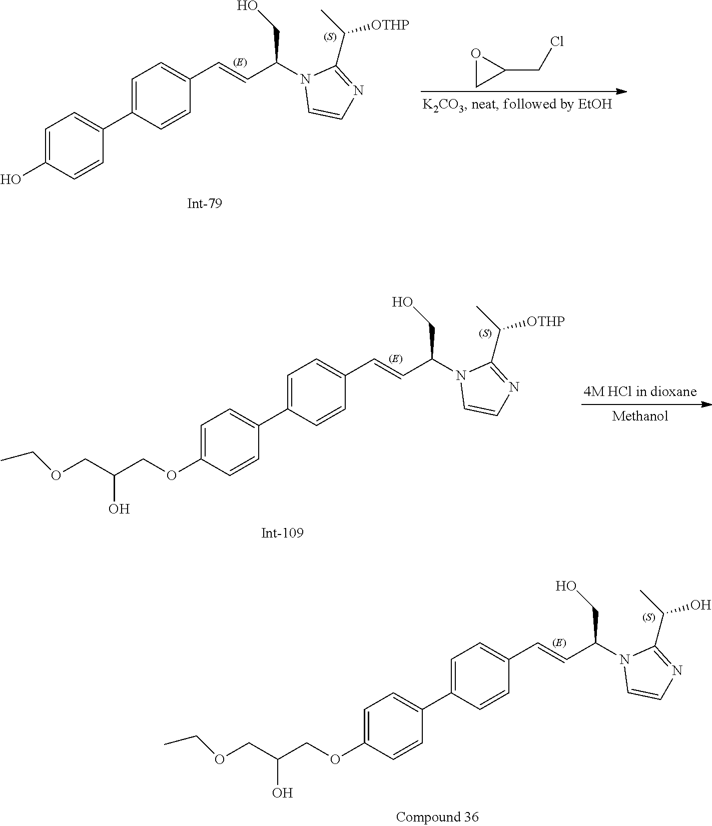

Step-1: To a mixture of Int-79 (5.0 mmol) and K₂CO₃ (10.00 mmol), was added epichlorohydrin (35 mmol) at RT and the reaction mixture was heated for 4 h at 120° C. After completion of the reaction, the reaction mixture was concentrated under reduced pressure. The resulting residue was dissolved in water and extracted with EtOAc (2×75 mL). The combined organic layers were dried over Na₂SO₄ and concentrated under reduced pressure. The crude product was purified by column chromatography using silica gel (60-120 mesh) by eluting with 10% Methanol in DCM to afford pure product Int-109. Yield: 76%. LC-MS: Calculated for C₃₁H₄₀N₂O₆ is 536.67, Observed: 537.55 [M+1]⁺.

Step 2: To a stirred solution of Int-109 (0.219 mmol) in DCM (2 mL), was added 4.0 M HCl in dioxane (1 mL) at 0° C. The reaction mixture was then stirred for 2 h at 25° C. The reaction was monitored by TLC; TLC showed complete consumption of starting material. The volatiles were evaporated under reduced pressure. The resulting crude product was purified by reversed phase preparative HPLC (10 mM ammonium bicarbonate in water and acetonitrile) to afford Compound 36 as a white solid. Yield: 12 mg (28%). LC MS: Calculated for C₂₆H₃₂N₂O₅ is 452.55, Observed: 453.5 [M+1]⁺.

Compound 43 was prepared with the same procedure using Int-79 and 4-(2-bromoethyl)-2,2-dimethyl-1,3-dioxolane as starting materials.

### Example A-1: Parenteral Pharmaceutical Composition

To prepare a parenteral pharmaceutical composition suitable for administration by injection (subcutaneous, intravenous), 1-100 mg of a water-soluble salt of a compound Formula (I), or a pharmaceutically acceptable salt or solvate thereof, is dissolved in sterile water and then mixed with 10 mL of 0.9% sterile saline. A suitable buffer is optionally added as well as optional acid or base to adjust the pH. The mixture is incorporated into a dosage unit form suitable for administration by injection.

### Example A-2: Oral Solution

To prepare a pharmaceutical composition for oral delivery, a sufficient amount of a compound of Formula (I), or a pharmaceutically acceptable salt thereof, is added to water (with optional solubilizer(s),optional buffer(s) and taste masking excipients) to provide a 20 mg/mL solution.

### Example A-3: Oral Tablet

A tablet is prepared by mixing 20-50% by weight of a compound of Formula (I), or a pharmaceutically acceptable salt thereof, 20-50% by weight of microcrystalline cellulose, 1-10% by weight of low-substituted hydroxypropyl cellulose, and 1-10% by weight of magnesium stearate or other appropriate excipients. Tablets are prepared by direct compression. The total weight of the compressed tablets is maintained at 100 −500 mg.

### Example A-4: Oral Capsule

To prepare a pharmaceutical composition for oral delivery, 10-500 mg of a compound of Formula (I), or a pharmaceutically acceptable salt thereof, is mixed with starch or other suitable powder blend. The mixture is incorporated into an oral dosage unit such as a hard gelatin capsule, which is suitable for oral administration.

In another embodiment, 10-500 mg of a compound of Formula (I), or a pharmaceutically acceptable salt thereof, is placed into Size 4 capsule, or size 1 capsule (hypromellose or hard gelatin) and the capsule is closed.

### II. Biological Evaluation

### Example B-1: In vitro Assays to Screen Compounds and Metalloprotein Modulators Bacterial Susceptibility Testing

Minimal inhibitory concentrations (MIC) were determined by the broth microdilution method in accordance with the Clinical and Laboratory Standards Institute (CLSI) guidelines. In brief, organism suspensions were adjusted to a final inoculum between 3×10⁵ and 7×10⁵ colony-forming units (CFU)/mL. Drug dilutions and inocula were made in sterile, cation adjusted Mueller-Hinton Broth (Beckton Dickinson). In wells, an inoculum volume of 100 μL was mixed to 2 μL of DMSO with 2-fold serial dilutions of drug. All inoculated microdilution trays were incubated in ambient air at 35° C. for 18-24 h. Following incubation, the lowest concentration of the drug that prevented visible growth (OD600 nm<0.05) was recorded as the MIC. Performance of the assay was monitored by the use of laboratory quality-control strains and compounds with defined MIC spectrum, in accordance with CLSI guidelines.

Exemplary in vitro assay data against select bacteria for compounds in embodiments of the disclosure is provided in Table A. Compounds of the disclosure do not inhibit S. aureus.

TABLE A

| | MIC (μg/mL) | | | |
|---|---|---|---|---|
| Compound | E. coli | K. pneumoniae | P. aeruginosa | P. aeruginosa |
| No. | ATCC25922 | ATCC13883 | 209 PAO1 | ATCC27835 |
| 1 | 1 | 1 | 4 | 4 |
| 2 | 1 | 2 | 4 | 4 |
| 3 | 2 | 8 | 4 | 4 |
| 4 | 0.12 | 0.12 | 2 | 4 |
| 5 | 2 | 4 | 4 | 4 |
| 6 | 4 | 16 | 4 | 4 |
| 7 | 0.25 | 0.25 | 2 | 4 |
| 8 | 16 | 64 | 4 | 4 |
| 9 | — | — | 2 | 2 |
| 10 | 8 | 16 | 1 | 2 |
| 11 | 4 | 16 | 1 | 2 |
| 12 | 4 | 16 | 4 | 4 |
| 13 | 4 | 4 | 2 | 16 |
| 14 | 1 | 2 | 1 | 8 |
| 15 | 0.5 | — | 0.5 | 2 |
| 16 | 2 | 8 | 2 | 4 |
| 17 | 4 | 16 | 4 | 4 |
| 18 | 8 | 16 | 4 | 4 |
| 19 | 8 | 64 | 4 | 4 |
| 20 | 4 | — | 2 | 2 |
| 21 | 16 | 64 | 8 | 4 |
| 22 | 16 | 64 | 4 | 2 |
| 23 | 8 | 16 | 4 | 4 |
| 24 | 2 | 8 | 2 | 2 |
| 25 | 2 | 4 | 2 | 2 |
| 26 | — | 2 | 4 | 8 |
| 27 | 8 | 32 | 4 | 4 |
| 28 | 4 | 16 | 4 | 4 |
| 29 | 32 | 64 | 8 | 4 |
| 30 | 16 | 32 | 8 | 8 |
| 31 | 2 | 4 | 1 | 4 |
| 32 | 2 | 8 | 4 | 4 |
| 33 | — | 8 | 2 | 4 |
| 34 | — | 2 | 0.5 | 4 |
| 35 | — | 2 | 1 | 4 |
| 36 | — | 1 | 2 | 4 |
| 37 | — | 0.5 | 2 | 4 |
| 38 | — | 2 | 2 | 2 |
| 39 | — | 0.5 | 1 | 4 |
| 40 | — | 1 | 2 | 2 |
| 41 | — | 2 | 2 | 2 |
| 42 | — | 0.5 | 4 | 4 |
| 43 | — | 2 | 2 | 2 |
| 44 | 4 | 8 | 4 | 4 |
| 45 | 0.25 | 0.5 | 4 | 4 |
| 46 | 0.5 | 2 | 4 | 4 |
| 47 | 0.5 | 1 | 4 | 4 |
| 48 | 2 | 16 | 2 | 2 |
| 49 | 4 | 4 | 2 | 2 |
| 50 | 0.5 | 1 | 2 | 2 |
| 51 | 4 | 8 | 4 | 4 |
| 52 | 4 | 16 | 4 | 4 |
| 53 | 0.5 | 1 | 2 | 4 |
| 54 | 0.5 | 1 | 2 | 4 |
| 55 | 0.5 | 2 | 2 | 2 |
| 56 | 0.25 | 0.5 | 2 | 4 |
| 57 | 0.25 | 1 | 4 | 4 |
| 58 | 1 | 2 | 4 | 8 |
| 59 | 2 | 4 | 2 | 4 |
| 60 | 8 | 16 | 4 | 4 |
| 61 | 2 | 8 | 4 | 4 |
| 62 | 1 | 0.5 | 1 | 2 |
| 63 | 2 | 4 | 2 | 4 |
| 64 | 1 | 2 | 1 | 2 |
| 65 | 0.5 | 1 | 2 | 2 |
| 66 | 4 | 8 | 4 | 4 |
| 67 | 0.5 | 1 | 2 | 2 |
| 68 | 1 | 2 | 4 | 4 |
| 69 | 0.25 | 0.50 | 2 | 4 |
| 70 | 2 | 8 | 4 | 4 |
| 71 | 0.5 | 0.5 | 2 | 4 |
| 72 | 2 | 8 | 2 | 2 |
| 73 | 0.5 | 1 | 2 | 4 |
| 74 | 8 | >32 | 2 | 1 |
| 75 | 0.25 | 0.50 | 2 | 4 |

### LpxC Binding Assay

IC₅₀ values against E. coli and P. aeruginosa LpxC are determined using a Rapid Fire MS assay as previously described J. Med. Chem. 2012, 55, 1662-1670.

Exemplary in vitro assay data against select bacteria for compounds in embodiments of the disclosure is provided in Table B.

TABLE B

| | E. coli | K. pneumoniae | P. aeruginosa |
|---|---|---|---|
| Compound | LpxC | LpxC | LpxC |
| No. | IC₅₀ (nM) | IC₅₀ (nM) | IC₅₀ (nM) |
| 1 | 25.8 | — | 81 |
| 2 | 17 | — | 15.5 |
| 3 | 11.4 | — | 132 |
| 4 | 9.25 | — | 23.2 |
| 11 | 2.7 | 4.0 | 31.3 |
| 12 | 82.5 | — | 69.2 |
| 14 | 9.51 | — | 22.3 |
| 17 | 4.2 | 6.3 | 66.4 |
| 18 | 3.6 | 5.4 | 50.1 |
| 19 | 5.1 | 8.0 | 62.3 |
| 24 | 0.5 | 1.1 | 17.5 |
| 25 | 44.6 | — | 70.6 |
| 26 | 0.6 | 1.4 | 17.4 |
| 27 | 2.9 | 6.2 | 41.2 |
| 28 | 2.6 | 4.3 | 36.9 |
| 29 | 5.9 | 9.1 | 120 |
| 30 | 5.0 | 7.6 | 79.5 |
| 32 | 1.0 | 1.8 | 13.4 |
| 33 | 1.0 | 1.6 | 11.9 |
| 34 | 1.1 | 1.9 | 18.9 |
| 35 | <0.5 | 0.9 | 8.2 |
| 36 | <0.5 | 0.8 | 21.3 |
| 37 | <0.5 | 0.7 | 15.2 |
| 38 | 0.9 | 1.6 | 27.5 |
| 39 | <0.5 | 0.6 | 12.6 |

The examples and embodiments described herein are for illustrative purposes only and various modifications or changes suggested to persons skilled in the art are to be included within the spirit and purview of this application and scope of the appended claims.

What is claimed is:

1. A compound of Formula (I): 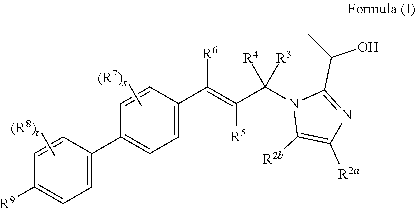
or a pharmaceutically acceptable salt, or solvate thereof, wherein:
R¹ is C₁-C₄ alkyl;
R^{2a} and R^{2b} are each independently hydrogen, halogen, or C₁-C₄ alkyl;
R³ is hydrogen or —(C₁-C₄ alkylene)-OH;
R⁴ is hydrogen or C₁-C₄ alkyl;
R⁵ is hydrogen or halogen;
R⁶ is hydrogen or halogen;
each R⁷ and R⁸ is independently hydrogen, halogen, or C₁-C₄ alkyl;
R⁹ is C₁-C₆ alkoxy, C₃-C₆ cycloalkyl, 4- to 6-membered heterocycloalkyl, —O—(C₃-C₆ cycloalkyl), —O—(4- to 6-membered heterocycloalkyl), —(C₁-C₄ alkylene)-(C₃-C₆ cycloalkyl), —(C₁-C₄ alkylene)-(4- to 6-membered heterocycloalkyl), —O—(C₁-C₄ alkylene)-(C₃-C₆ cycloalkyl), —O—(C₁-C₄ alkylene)-(4- to 6-membered heterocycloalkyl), —(C₁-C₄ alkylene)-O—(C₃-C₆ cycloalkyl), or —(C₁-C₄ alkylene)-O—(4- to 6-membered heterocycloalkyl); wherein the alkoxy, cycloalkyl, or heterocycloalkyl is unsubstituted or substituted by 1, 2, or 3 groups independently selected from —OR¹⁰, —N(R¹⁰)₂, —CO₂R¹⁰, —CON(R¹⁰)₂, —CH₂N(R¹⁰)₂, —NHCOR¹⁰, —NHSO₂R¹⁰, —OCOR¹⁰, phenyl, monocyclic heteroaryl, C₁-C₄ alkyl, C₁-C₄ hydroxyalkyl, C₁-C₄ aminoalkyl, C₃-C₆ cycloalkyl, and 4- to 6-membered heterocycloalkyl which is unsubstituted or substituted by 1, 2, or 3 —OH groups;
each R¹⁰ is independently hydrogen, 4- to 6-membered heterocycloalkyl, or C₁-C₄ alkyl which is unsubstituted or substituted by 1, 2, or 3 groups independently selected from —F, —CN, —OH, —NH₂, —OMe, —N(CH₃)₂, —CO₂H, —CONH₂, —SO₂CH₃, phenyl, oxazolidinone, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃;
or two R¹⁰ attached to the same nitrogen are taken together to form a 4- to 6-membered heterocycloalkyl which is unsubstituted or substituted by 1, 2, or 3 groups independently selected from —F, —CN, —OH, —NH₂, —OMe, —CO₂H, —CONH₂, and —SO₂CH₃;
s is 1 or 2; and
t is 1 or 2.

2. The compound of claim 1, or a pharmaceutically acceptable salt, or solvate thereof, wherein:
R¹ is —CH₃.

3. The compound of claim 1 or claim 2, or a pharmaceutically acceptable salt, or solvate thereof, wherein:
R^{2a} is hydrogen; and
R^{2b} is hydrogen.

4. The compound of any one of claims 1-3, or a pharmaceutically acceptable salt, or solvate thereof, wherein:
R⁴ is hydrogen.

5. The compound of any one of claims 1-4, or a pharmaceutically acceptable salt, or solvate thereof, wherein:
R⁵ is hydrogen.

6. The compound of any one of claims 1-5, or a pharmaceutically acceptable salt, or solvate thereof, wherein:
R⁶ is hydrogen or fluoro.

7. The compound of any one of claims 1-6, or a pharmaceutically acceptable salt, or solvate thereof, wherein:
each R⁷ is independently hydrogen, fluoro, chloro, or —CH₃.

8. The compound of any one of claims 1-7, or a pharmaceutically acceptable salt, or solvate thereof, wherein:
s is 1.

9. The compound of any one of claims 1-8, or a pharmaceutically acceptable salt, or solvate thereof, wherein:
each R⁸ is independently hydrogen, fluoro, chloro, or —CH₃.

10. The compound of any one of claims 1-9, or a pharmaceutically acceptable salt, or solvate thereof, wherein:
t is 1.

11. The compound of claim 1, wherein the compound is a compound of Formula (IIa): 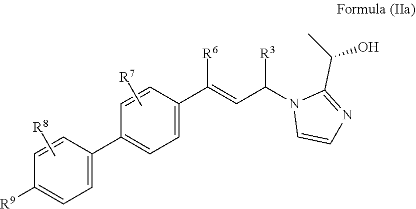
or a pharmaceutically acceptable salt, or solvate thereof, wherein:
R⁶ is hydrogen or fluoro;
R⁷ is hydrogen or fluoro; and
R⁸ is hydrogen or fluoro.

12. The compound of any one of claims 1-11, or a pharmaceutically acceptable salt, or solvate thereof, wherein:
R³ is hydrogen.

13. The compound of any one of claims 1-11, or a pharmaceutically acceptable salt, or solvate thereof, wherein:
R³ is —(C₁-C₄ alkylene)-OH.

14. The compound of claim 1, wherein the compound is a compound of Formula (IIIa): 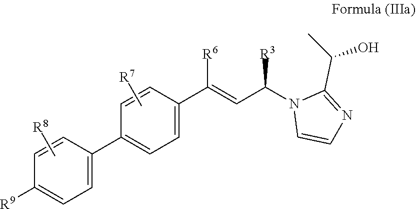
or a pharmaceutically acceptable salt, or solvate thereof, wherein:
R³ is —(C₁-C₄ alkylene)-OH;
R⁶ is hydrogen or fluoro;
R⁷ is hydrogen or fluoro; and
R⁸ is hydrogen or fluoro.

15. The compound of any one of claims 1-11, or a pharmaceutically acceptable salt, or solvate thereof, wherein:
R³ is hydrogen, —CH₂OH, or —CH₂CH₂OH.

16. The compound of any one of claims 1-11 or 13-15, or a pharmaceutically acceptable salt, or solvate thereof, wherein:
R³ is —CH₂OH.

17. The compound of claim 1, wherein the compound is a compound of Formula (IVa): 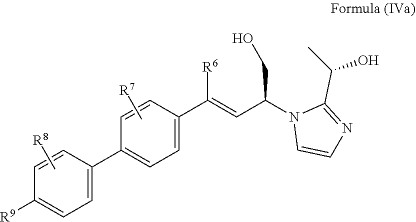
or a pharmaceutically acceptable salt, or solvate thereof, wherein:
R⁶ is hydrogen or fluoro;
R⁷ is hydrogen or fluoro; and
R⁸ is hydrogen or fluoro.

18. The compound of any one of claims 1-17, or a pharmaceutically acceptable salt, or solvate thereof, wherein:
R⁹ is C₁-C₆ alkoxy, C₃-C₆ cycloalkyl, —O—(C₃-C₆ cycloalkyl), —O—(4- to 6-membered heterocycloalkyl), —O—(C₁-C₄ alkylene)-(C₃-C₆ cycloalkyl), —O—(C₁-C₄ alkylene)-(4- to 6-membered heterocycloalkyl), or —(C₁-C₄ alkylene)-O—(C₃-C₆ cycloalkyl); wherein the alkoxy, cycloalkyl, or heterocycloalkyl is unsubstituted or substituted by 1 or 2 groups independently selected from —OR¹⁰, —N(R¹⁰)₂, —CO₂R¹⁰, —CON(R¹⁰)₂, —CH₂N(R¹⁰)₂, —NHCOR¹⁰, —NHSO₂R¹⁰, —OCOR¹⁰, C₁-C₄ hydroxyalkyl, C₁-C₄ aminoalkyl, and 4- to 6-membered heterocycloalkyl which is unsubstituted or substituted by 1 —OH group; and
each R¹⁰ is independently hydrogen, 4- to 6-membered heterocycloalkyl, or C₁-C₄ alkyl which is unsubstituted or substituted by 1, 2, or 3 groups independently selected from —F, —CN, —OH, —N(CH₃)₂, —CO₂H, —SO₂CH₃, oxazolidinone, and monocyclic heteroaryl which is unsubstituted or substituted by 1 —CONH₂ group;
or two R¹⁰ attached to the same nitrogen are taken together to form an azetidinyl which is unsubstituted or substituted by 1 —SO₂CH₃ group.

19. The compound of any one of claims 1-18, or a pharmaceutically acceptable salt, or solvate thereof, wherein:
R⁹ is C₁-C₄ alkoxy, C₃-C₄ cycloalkyl, —O—(C₃-C₄ cycloalkyl), —O—(4- to 6-membered heterocycloalkyl), —O—(C₁-C₃ alkylene)-(C₃-C₄ cycloalkyl), —O—(C₁-C₃ alkylene)-(4- to 6-membered heterocycloalkyl), or —(C₁-C₃ alkylene)-O—(C₃-C₄ cycloalkyl); wherein the alkoxy, cycloalkyl, or heterocycloalkyl is substituted by 1 or 2 groups independently selected from —OR¹⁰, —N(R¹⁰)₂, —CO₂R¹⁰, —CON(R¹⁰)₂, —CH₂N(R¹⁰)₂, —NHCOR¹⁰, —NHSO₂R¹⁰, —OCOR¹⁰, C₁-C₃ hydroxyalkyl, C₁-C₃ aminoalkyl, and 4- to 6-membered heterocycloalkyl which is unsubstituted or substituted by 1 —OH group; and
each R¹⁰ is independently hydrogen or C₁-C₂ alkyl which is unsubstituted or substituted by 1, 2, or 3 groups independently selected from —F, —CN, —OH, —N(CH₃)₂, —CO₂H, —SO₂CH₃, oxazolidinone, and monocyclic 5-membered heteroaryl which is unsubstituted or substituted by 1 —CONH₂ group.

20. The compound of any one of claims 1-19, or a pharmaceutically acceptable salt, or solvate thereof, wherein:
R⁹ is C₁-C₄ alkoxy, C₃-C₄ cycloalkyl, —O—(C₃-C₄ cycloalkyl), or —O—(4- to 6-membered heterocycloalkyl); wherein the alkoxy, cycloalkyl, or heterocycloalkyl is substituted by 1 or 2 groups independently selected from —OH, —N(R¹⁰)₂, —CON(R¹⁰)₂, —NHCOR¹⁰, —NHSO₂R¹⁰, —CH₂OH, —CH₂CH₂OH, and azetidinyl which is substituted by 1 —OH group; and
each R¹⁰ is independently hydrogen or C₁-C₂ alkyl which is unsubstituted or substituted by a —CN, —OH, oxazolyl, or imidazolyl group.

21. The compound of any one of claims 1-17, or a pharmaceutically acceptable salt, or solvate thereof, wherein:
R⁹ is C₁-C₆ alkoxy; wherein the alkoxy is substituted by 1, 2, or 3 groups independently selected from —OH, —OCH₃, —NH₂, —N(CH₃)₂, and —CH₂OH.

22. The compound of claim 21, or a pharmaceutically acceptable salt, or solvate thereof, wherein:
R⁹ is C₁-C₄ alkoxy; wherein the alkoxy is substituted by 2 —OH groups.

23. The compound of any one of claims 1-17, or a pharmaceutically acceptable salt, or solvate thereof, wherein:
R⁹ is C₃-C₄ cycloalkyl, —O—(C₃-C₄ cycloalkyl), —O—(4- to 6-membered heterocycloalkyl), —O—(C₁-C₃ alkylene)-(C₃-C₄ cycloalkyl), —O—(C₁-C₃ alkylene)-(4- to 6-membered heterocycloalkyl), or —(C₁-C₃ alkylene)-O—(C₃-C₄ cycloalkyl); wherein the cycloalkyl or heterocycloalkyl is substituted by 1 or 2 groups independently selected from —OR¹⁰, —N(R¹⁰)₂, —CO₂R¹⁰, —CON(R¹⁰)₂, —CH₂N(R¹⁰)₂, —NHCOR¹⁰, —NHSO₂R¹⁰, —OCOR¹⁰, C₁-C₃ hydroxyalkyl, C₁-C₃ aminoalkyl, and 4- to 6-membered heterocycloalkyl, which is unsubstituted or substituted by 1 —OH group; and
each R¹⁰ is independently hydrogen or C₁-C₂ alkyl which is unsubstituted or substituted by 1, 2, or 3 groups independently selected from —F, —CN, —OH, —N(CH₃)₂, —CO₂H, —SO₂CH₃, oxazolidinone, and monocyclic 5-membered heteroaryl which is unsubstituted or substituted by 1 —CONH₂ group;
or two R¹⁰ attached to the same nitrogen are taken together to form an azetidinyl which is unsubstituted or substituted by 1 —SO₂CH₃ group.

24. The compound of claim 23, or a pharmaceutically acceptable salt, or solvate thereof, wherein:
R⁹ is —O—(C₃-C₄ cycloalkyl); wherein the cycloalkyl is substituted by 1 or 2 groups independently selected from —OH, —OCH₃, —NH₂, and —N(CH₃)₂.

25. The compound of claim 24, or a pharmaceutically acceptable salt, or solvate thereof, wherein:
R⁹ is —O-(cyclopropyl); wherein the cyclopropyl is substituted by 1 —NH₂ group.

26. The compound of claim 23, or a pharmaceutically acceptable salt, or solvate thereof, wherein:
R⁹ is —O—(4- to 6-membered heterocycloalkyl); wherein the heterocycloalkyl is substituted by 1 or 2 groups independently selected from —OH, —OCH₃, —NH₂, —N(CH₃)₂, —CH₂OH, and —CH₂CH₂OH.

27. The compound of claim 26, or a pharmaceutically acceptable salt, or solvate thereof, wherein:
R⁹ is —O-(azetidinyl); wherein the azetidinyl is substituted by 1 —CH₂CH₂OH group.

28. The compound of claim 23, or a pharmaceutically acceptable salt, or solvate thereof, wherein:
R⁹ is C₃-C₄ cycloalkyl; wherein the cycloalkyl is substituted by 1 or 2 groups independently selected from —N(R¹⁰)₂, —CO₂R¹⁰, —CON(R¹⁰)₂, —CH₂N(R¹⁰)₂, —NHCOR¹⁰, —NHSO₂R¹⁰, —OCOR¹⁰, and 4- to 6-membered heterocycloalkyl which is unsubstituted or substituted by 1 —OH group.

29. The compound of claim 28, or a pharmaceutically acceptable salt, or solvate thereof, wherein:
R⁹ is cyclobutyl; wherein the cyclobutyl is substituted by 1 group selected from —N(R¹⁰)₂, —CO₂R¹⁰, —CON(R¹⁰)₂, —CH₂N(R¹⁰)₂, —NHCOR¹⁰, —NHSO₂R¹⁰, and azetidinyl which is substituted by 1 —OH group.

30. The compound of any one of claims 1-17, or a pharmaceutically acceptable salt, or solvate thereof, wherein:
R⁹ is,

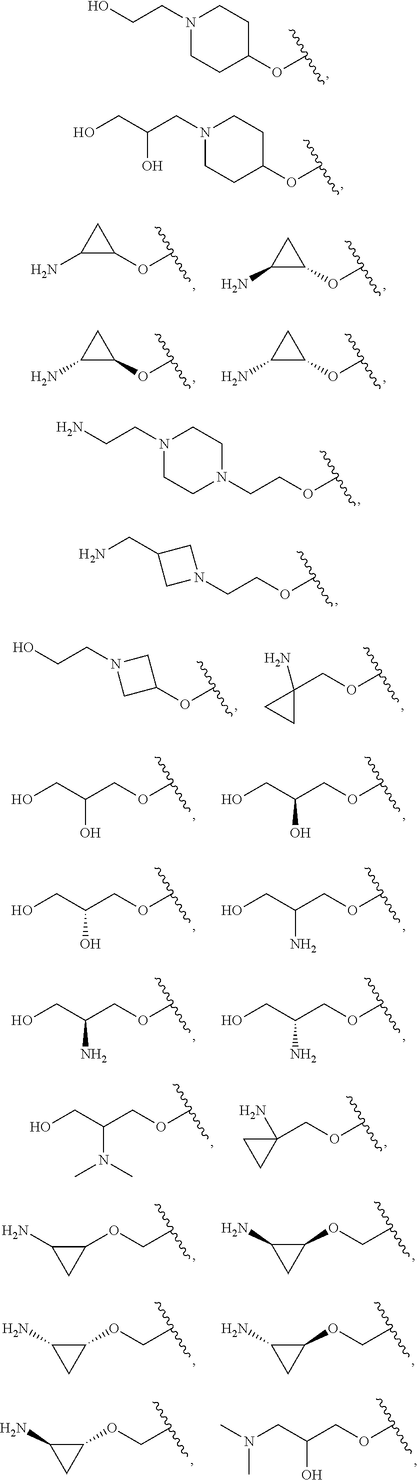

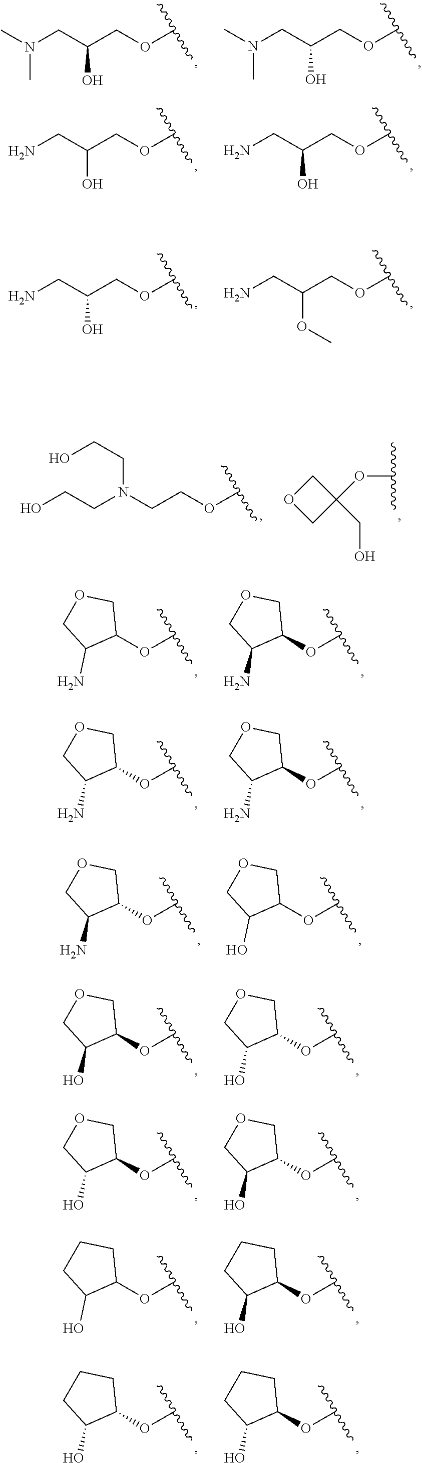

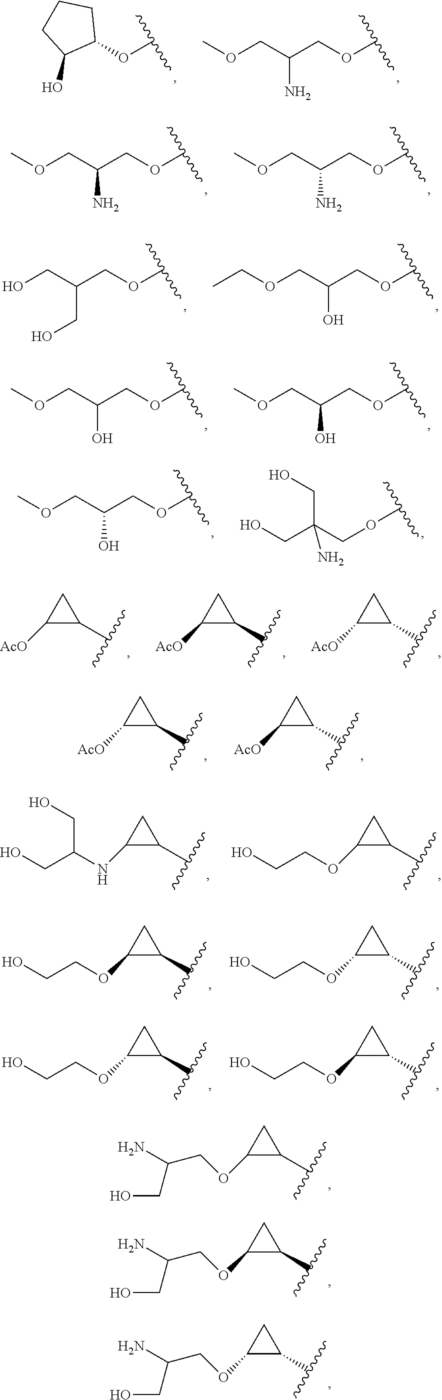

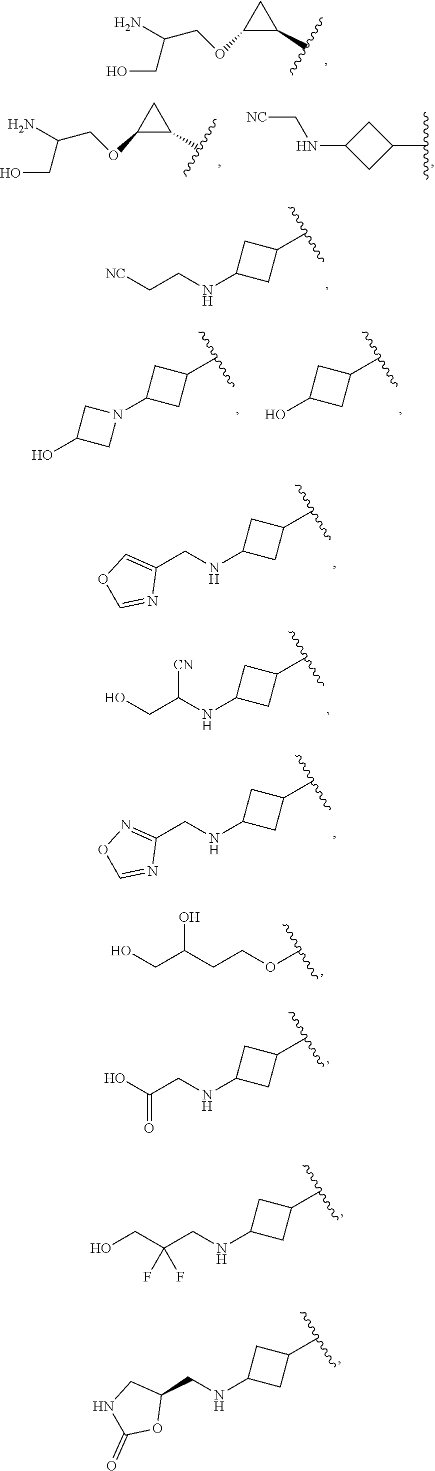

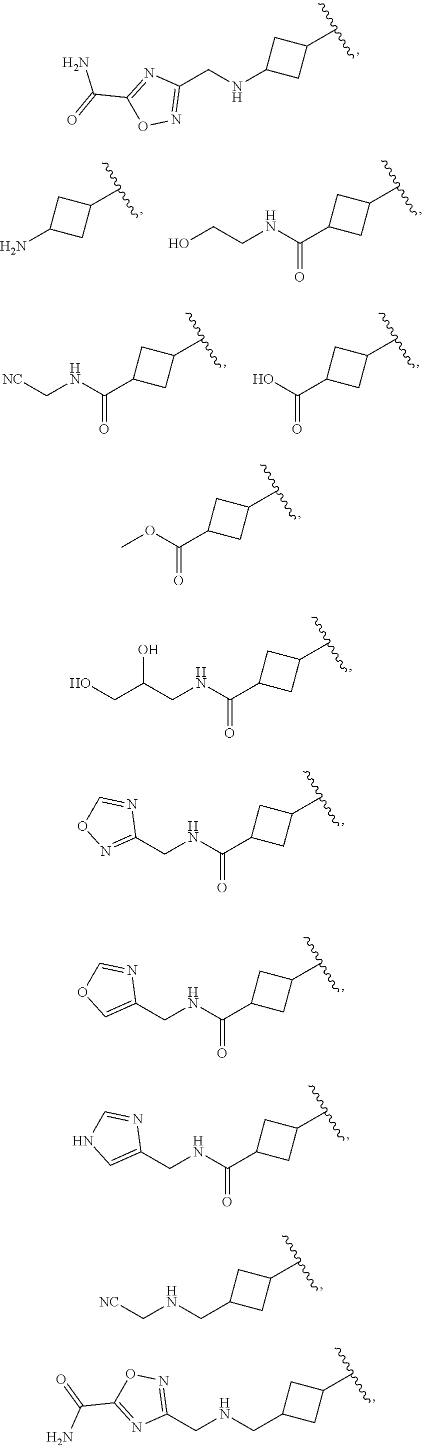

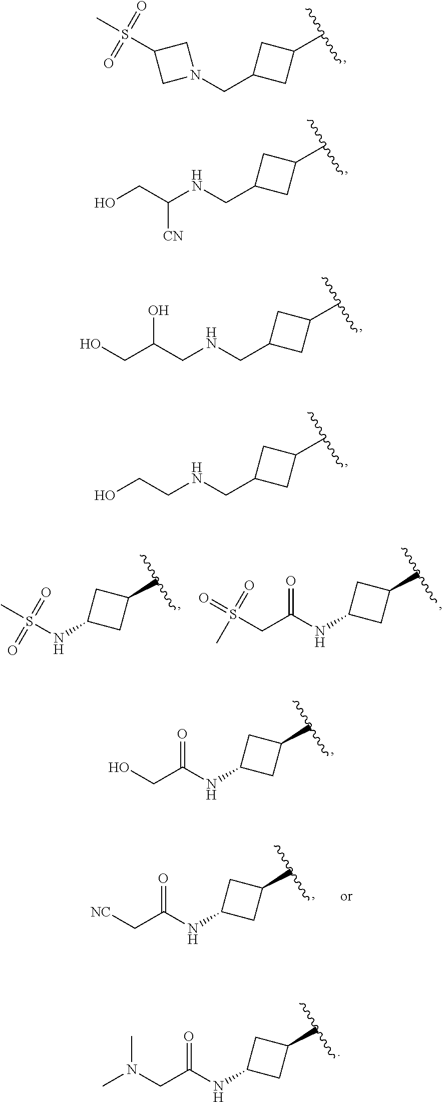

31. The compound of any one of claims 1-17, or a pharmaceutically acceptable salt, or solvate thereof, wherein:
R⁹ is

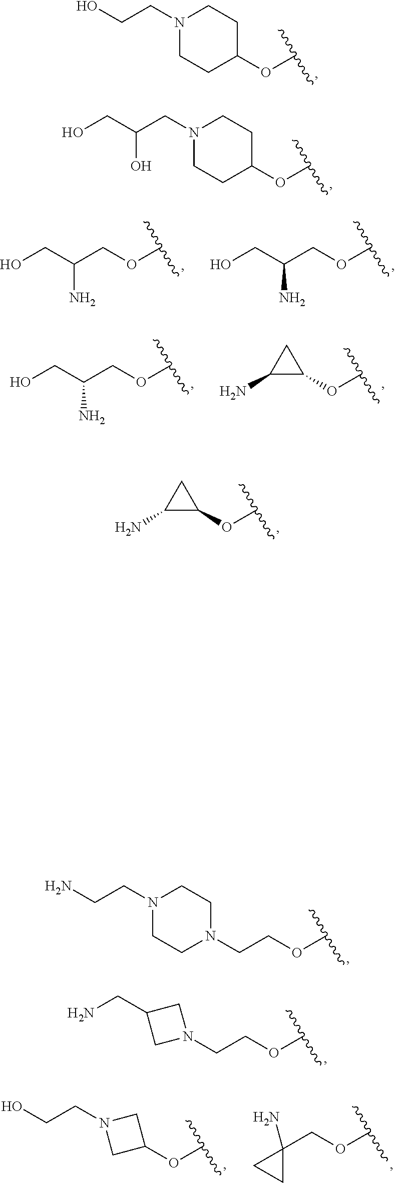

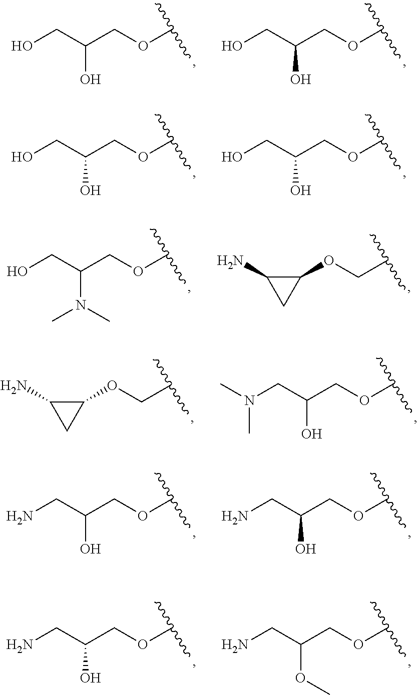

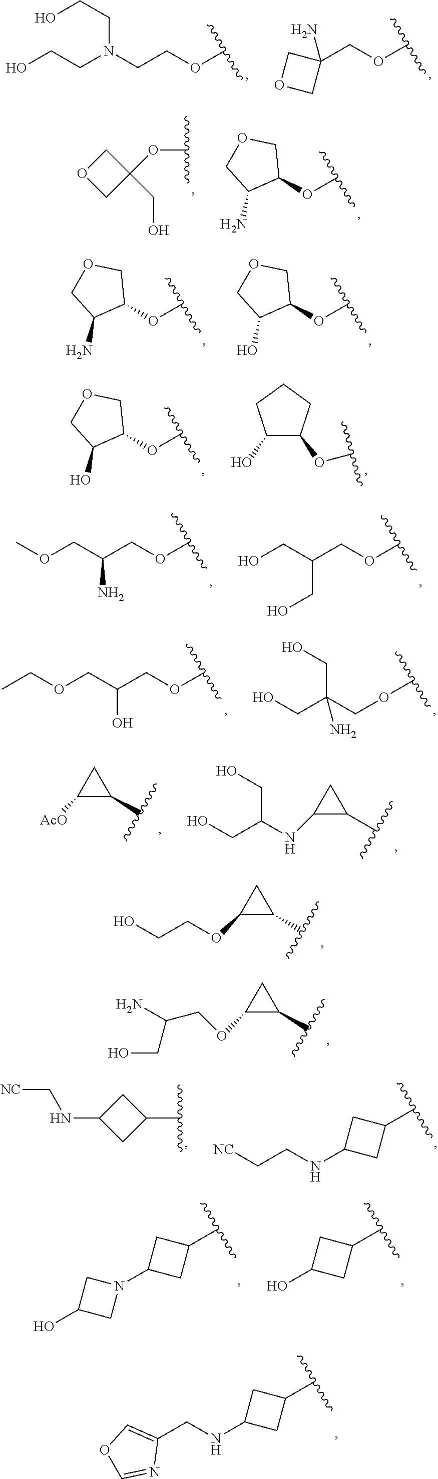

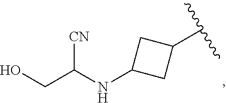 [SMILES: *C1CC(NC(C#N)CO)C1]

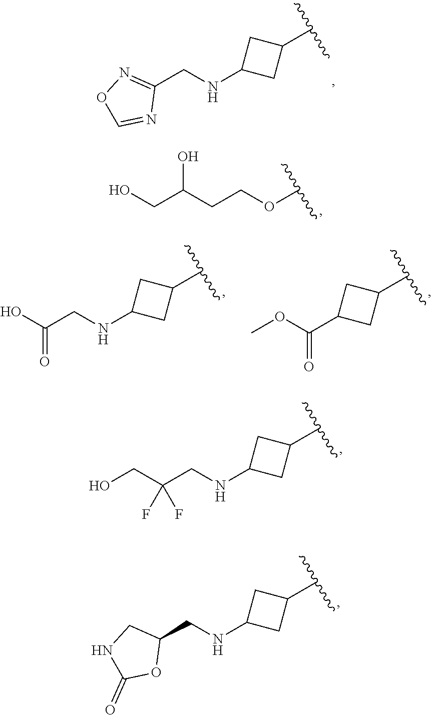

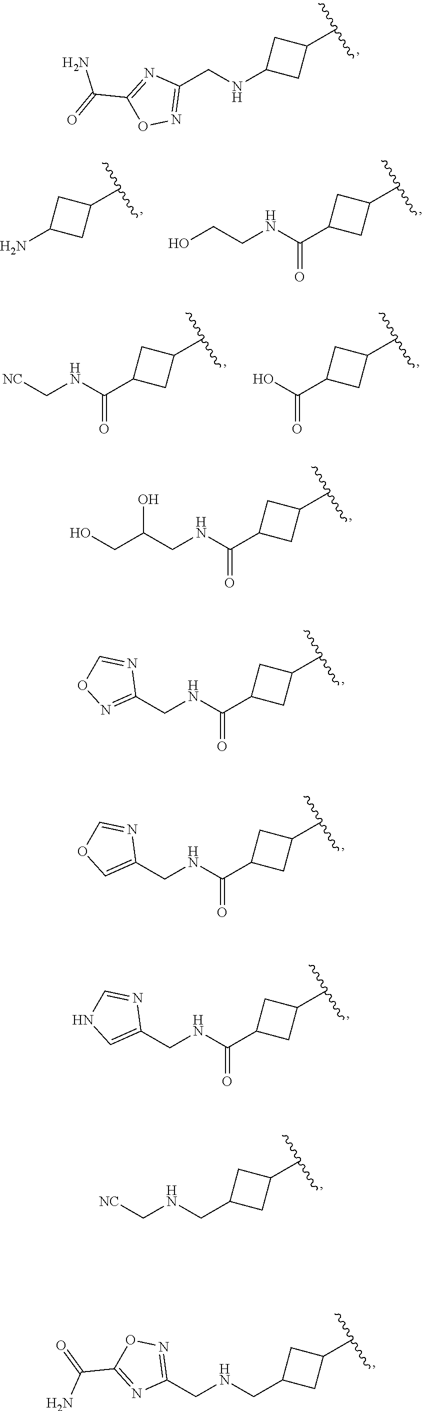

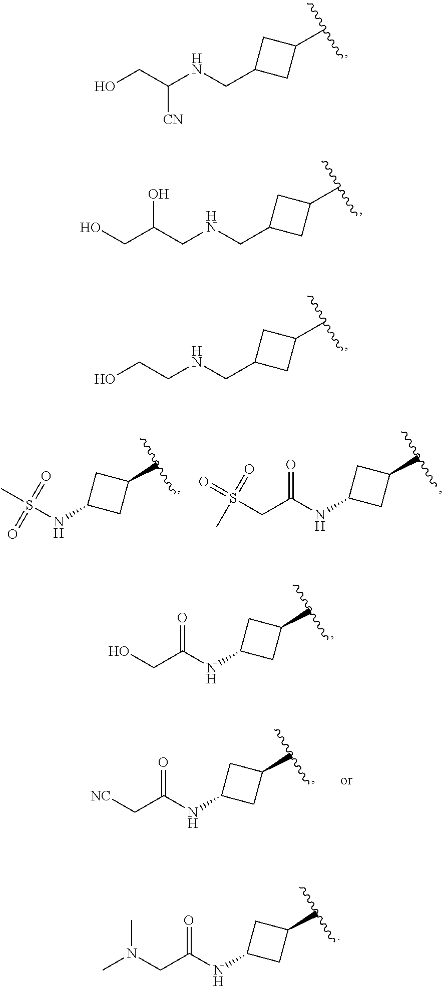

32. The compound of any one of claims 1-17, or a pharmaceutically acceptable salt, or solvate thereof, wherein:
R⁹ is

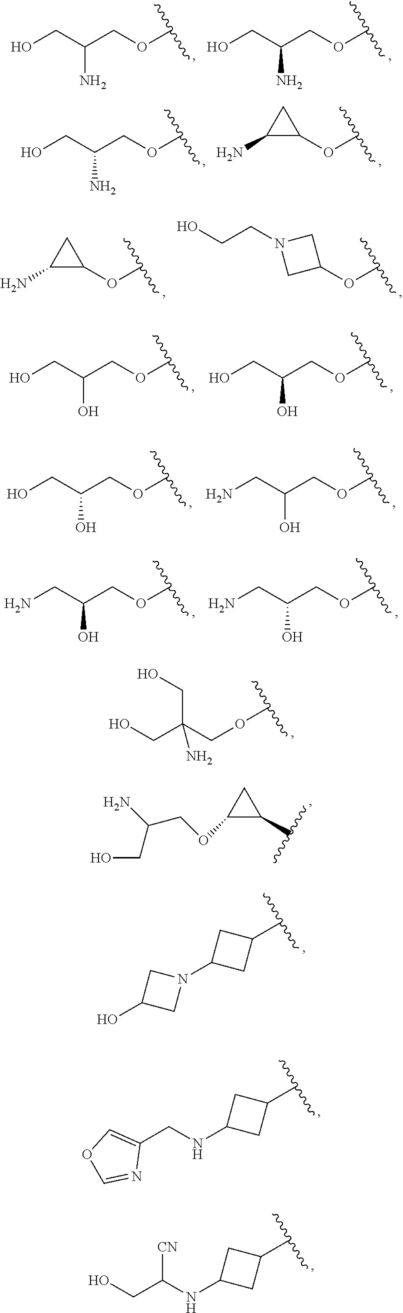

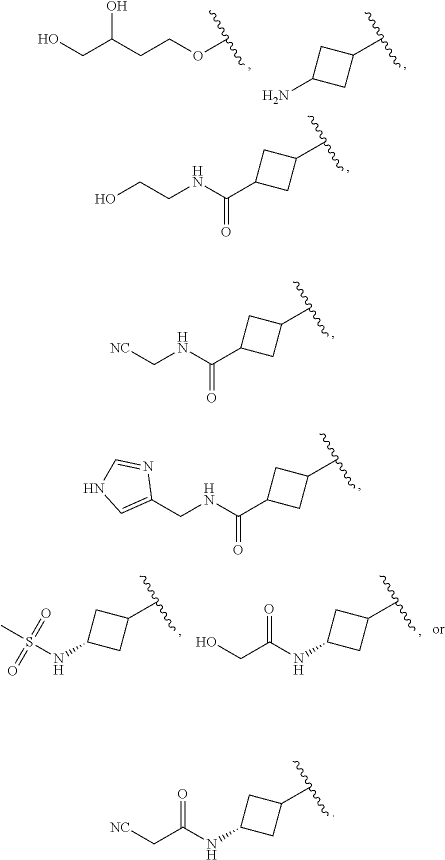

33. The compound of claim 1, or a pharmaceutically acceptable salt, or solvate thereof, wherein:
R¹ is —CH₃;
R^{2a} and R^{2b} are each hydrogen;
R³ is hydrogen or —(C₁-C₄ alkylene)-OH;
R⁴ is hydrogen;
R⁵ is hydrogen;
R⁶ is hydrogen or fluoro;
each R⁷ and R⁸ is independently hydrogen or fluoro;
R⁹ is C₁-C₆ alkoxy, C₃-C₆ cycloalkyl, —O—(C₃-C₆ cycloalkyl), —O—(4- to 6-membered heterocycloalkyl), —O—(C₁-C₄ alkylene)-(C₃-C₆ cycloalkyl), —O—(C₁-C₄ alkylene)-(4- to 6-membered heterocycloalkyl), or —(C₁-C₄ alkylene)-O—(C₃-C₆ cycloalkyl); wherein the alkoxy, cycloalkyl, or heterocycloalkyl is unsubstituted or substituted by 1 or 2 groups independently selected from —OR¹⁰, —N(R¹⁰)₂, —CO₂R¹⁰, —CON(R¹⁰)₂, —CH₂N(R¹⁰)₂, —NHCOR¹⁰, —NHSO₂R¹⁰, —OCOR¹⁰, C₁-C₄ hydroxyalkyl, C₁-C₄ aminoalkyl, and 4- to 6-membered heterocycloalkyl which is unsubstituted or substituted by 1 —OH group;
each R¹⁰ is independently hydrogen, 4- to 6-membered heterocycloalkyl, or C₁-C₄ alkyl which is unsubstituted or substituted by 1, 2, or 3 groups independently selected from —F, —CN, —OH, —N(CH₃)₂, —CO₂H, —SO₂CH₃, oxazolidinone, and monocyclic heteroaryl which is unsubstituted or substituted by 1 —CONH₂ group;
or two R¹⁰ attached to the same nitrogen are taken together to form an azetidinyl which is unsubstituted or substituted by 1 —SO₂CH₃ group;
s is 1; and
t is 1.

34. The compound of claim 33, or a pharmaceutically acceptable salt, or solvate thereof, wherein:
R³ is hydrogen or —(C₁-C₄ alkylene)-OH;
R⁹ is C₁-C₄ alkoxy, C₃-C₄ cycloalkyl, —O—(C₃-C₄ cycloalkyl), —O—(4- to 6-membered heterocycloalkyl), —O—(C₁-C₃ alkylene)-(C₃-C₄ cycloalkyl), —O—(C₁-C₃ alkylene)-(4- to 6-membered heterocycloalkyl), or —(C₁-C₃ alkylene)-O—(C₃-C₄ cycloalkyl); wherein the alkoxy, cycloalkyl, or heterocycloalkyl is substituted by 1 or 2 groups independently selected from —OR¹⁰, —N(R¹⁰)₂, —CO₂R¹⁰, —CON(R¹⁰)₂, —CH₂N(R¹⁰)₂, —NHCOR¹⁰, —NHSO₂R¹⁰, —OCOR¹⁰, C₁-C₃ hydroxyalkyl, C₁-C₃ aminoalkyl, and 4- to 6-membered heterocycloalkyl which is unsubstituted or substituted by 1 —OH group; and
each R¹⁰ is independently hydrogen or C₁-C₂ alkyl which is unsubstituted or substituted by 1, 2, or 3 groups independently selected from —F, —CN, —OH, —N(CH₃)₂, —CO₂H, —SO₂CH₃, oxazolidinone, and monocyclic 5-membered heteroaryl which is unsubstituted or substituted by 1 —CONH₂ group.

35. The compound of claim 33 or claim 34, or a pharmaceutically acceptable salt, or solvate thereof, wherein:
R³ is —CH₂OH;
R⁹ is C₁-C₄ alkoxy, C₃-C₄ cycloalkyl, —O—(C₃-C₄ cycloalkyl), or —O—(4- to 6-membered heterocycloalkyl); wherein the alkoxy, cycloalkyl, or heterocycloalkyl is substituted by 1 or 2 groups independently selected from —OH, —N(R¹⁰)₂, —CON(R¹⁰)₂, —NHCOR¹⁰, —NHSO₂R¹⁰, —CH₂OH, —CH₂CH₂OH, and azetidinyl which is substituted by 1 —OH group; and
each R¹⁰ is independently hydrogen or C₁-C₂ alkyl which is unsubstituted or substituted by a —CN, —OH, oxazolyl, or imidazolyl group.

36. The compound of claim 1, wherein the compound is a compound of Formula (V): 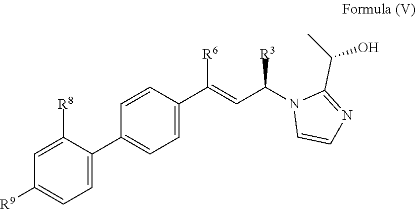
or a pharmaceutically acceptable salt, or solvate thereof, wherein:
R³ is hydrogen or —(C₁-C₄ alkylene)-OH;
R⁶ is hydrogen or fluoro;
R⁸ is hydrogen or fluoro;
R⁹ is C₁-C₆ alkoxy, C₃-C₆ cycloalkyl, —O—(C₃-C₆ cycloalkyl), —O—(4- to 6-membered heterocycloalkyl), —O—(C₁-C₄ alkylene)-(C₃-C₆ cycloalkyl), —O—(C₁-C₄ alkylene)-(4- to 6-membered heterocycloalkyl), or —(C₁-C₄ alkylene)-O—(C₃-C₆ cycloalkyl); wherein the alkoxy, cycloalkyl, or heterocycloalkyl is unsubstituted or substituted by 1 or 2 groups independently selected from —OR¹⁰, —N(R¹⁰)₂, —CO₂R¹⁰, —CON(R¹⁰)₂, —CH₂N(R¹⁰)₂, —NHCOR¹⁰, —NHSO₂R¹⁰, —OCOR¹⁰, C₁-C₄ hydroxyalkyl, C₁-C₄ aminoalkyl, and 4- to 6-membered heterocycloalkyl which is unsubstituted or substituted by 1 —OH group; and
each R¹⁰ is independently hydrogen, 4- to 6-membered heterocycloalkyl, or C₁-C₄ alkyl which is unsubstituted or substituted by 1, 2, or 3 groups independently selected from —F, —CN, —OH, —N(CH₃)₂, —CO₂H, —SO₂CH₃, oxazolidinone, and monocyclic heteroaryl which is unsubstituted or substituted by 1 —CONH₂ group;
or two R¹⁰ attached to the same nitrogen are taken together to form an azetidinyl which is unsubstituted or substituted by 1 —SO₂CH₃ group.

37. The compound of claim 36, or a pharmaceutically acceptable salt, or solvate thereof, wherein:
R⁶ is hydrogen or fluoro;
R⁸ is hydrogen or fluoro;
R⁹ is C₁-C₄ alkoxy, C₃-C₄ cycloalkyl, —O—(C₃-C₄ cycloalkyl), —O—(4- to 6-membered heterocycloalkyl), —O—(C₁-C₃ alkylene)-(C₃-C₄ cycloalkyl), —O—(C₁-C₃ alkylene)-(4- to 6-membered heterocycloalkyl), or —(C₁-C₃ alkylene)-O—(C₃-C₄ cycloalkyl); wherein the alkoxy, cycloalkyl, or heterocycloalkyl is substituted by 1 or 2 groups independently selected from —OR¹⁰, —N(R¹⁰)₂, —CO₂R¹⁰, —CON(R¹⁰)₂, —CH₂N(R¹⁰)₂, —NHCOR¹⁰, —NHSO₂R¹⁰, —OCOR¹⁰, C₁-C₃ hydroxyalkyl, C₁-C₃ aminoalkyl, and 4- to 6-membered heterocycloalkyl which is unsubstituted or substituted by 1 —OH group; and
each R¹⁰ is independently hydrogen or C₁-C₂ alkyl which is unsubstituted or substituted by 1, 2, or 3 groups independently selected from —F, —CN, —OH, —N(CH₃)₂, —CO₂H, —SO₂CH₃, oxazolidinone, and monocyclic 5-membered heteroaryl which is unsubstituted or substituted by 1 —CONH₂ group.

38. The compound of claim 36 or claim 37, or a pharmaceutically acceptable salt, or solvate thereof, wherein:
R³ is —CH₂OH;
R⁶ is hydrogen or fluoro;
R⁸ is hydrogen;
R⁹ is C₁-C₄ alkoxy, C₃-C₄ cycloalkyl, —O—(C₃-C₄ cycloalkyl), or —O—(4- to 6-membered heterocycloalkyl); wherein the alkoxy, cycloalkyl, or heterocycloalkyl is substituted by 1 or 2 groups independently selected from —OH, —N(R¹⁰)₂, —CON(R¹⁰)₂, —NHCOR¹⁰, —NHSO₂R¹⁰, —CH₂OH, —CH₂CH₂OH, and azetidinyl which is substituted by 1 —OH group; and
each R¹⁰ is independently hydrogen or C₁-C₂ alkyl which is unsubstituted or substituted by a —CN, —OH, oxazolyl, or imidazolyl group.

39. The compound of claim 1, wherein the compound is a compound of Formula (VI): 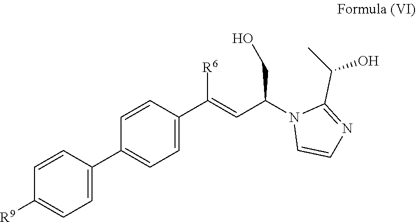
or a pharmaceutically acceptable salt, or solvate thereof, wherein:
R⁶ is hydrogen or fluoro;
R⁹ is C₁-C₆ alkoxy, C₃-C₆ cycloalkyl, —O—(C₃-C₆ cycloalkyl), —O—(4- to 6-membered heterocycloalkyl), —O—(C₁-C₄ alkylene)-(C₃-C₆ cycloalkyl), —O—(C₁-C₄ alkylene)-(4- to 6-membered heterocycloalkyl), or —(C₁-C₄ alkylene)-O—(C₃-C₆ cycloalkyl); wherein the alkoxy, cycloalkyl, or heterocycloalkyl is unsubstituted or substituted by 1 or 2 groups independently selected from —OR¹⁰, —N(R¹⁰)₂, —CO₂R¹⁰, —CON(R¹⁰)₂, —CH₂N(R¹⁰)₂, —NHCOR¹⁰, —NHSO₂R¹⁰, —OCOR¹⁰, C₁-C₄ hydroxyalkyl, C₁-C₄ aminoalkyl, and 4- to 6-membered heterocycloalkyl which is unsubstituted or substituted by 1 —OH group; and
each R¹⁰ is independently hydrogen, 4- to 6-membered heterocycloalkyl, or C₁-C₄ alkyl which is unsubstituted or substituted by 1, 2, or 3 groups independently selected from —F, —CN, —OH, —N(CH₃)₂, —CO₂H, —SO₂CH₃, oxazolidinone, and monocyclic heteroaryl which is unsubstituted or substituted by 1 —CONH₂ group;
or two R¹⁰ attached to the same nitrogen are taken together to form an azetidinyl which is unsubstituted or substituted by 1 —SO₂CH₃ group.

40. The compound of claim 39, or a pharmaceutically acceptable salt, or solvate thereof, wherein:
R⁹ is C₁-C₄ alkoxy, C₃-C₄ cycloalkyl, —O—(C₃-C₄ cycloalkyl), —O—(4- to 6-membered heterocycloalkyl), —O—(C₁-C₃ alkylene)-(C₃-C₄ cycloalkyl), —O—(C₁-C₃ alkylene)-(4- to 6-membered heterocycloalkyl), or —(C₁-C₃ alkylene)-O—(C₃-C₄ cycloalkyl); wherein the alkoxy, cycloalkyl, or heterocycloalkyl is substituted by 1 or 2 groups independently selected from —OR¹⁰, —N(R¹⁰)₂, —CO₂R¹⁰, —CON(R¹⁰)₂, —CH₂N(R¹⁰)₂, —NHCOR¹⁰, —NHSO₂R¹⁰, —OCOR¹⁰, C₁-C₃ hydroxyalkyl, C₁-C₃ aminoalkyl, and 4- to 6-membered heterocycloalkyl which is unsubstituted or substituted by 1 —OH group; and
each R¹⁰ is independently hydrogen or C₁-C₂ alkyl which is unsubstituted or substituted by 1, 2, or 3 groups independently selected from —F, —CN, —OH, —N(CH₃)₂, —CO₂H, —SO₂CH₃, oxazolidinone, and monocyclic 5-membered heteroaryl which is unsubstituted or substituted by 1 —CONH₂ group.

41. The compound of claim 39 or claim 40, or a pharmaceutically acceptable salt, or solvate thereof, wherein:
R⁹ is C₁-C₄ alkoxy, C₃-C₄ cycloalkyl, —O—(C₃-C₄ cycloalkyl), or —O—(4- to 6-membered heterocycloalkyl); wherein the alkoxy, cycloalkyl, or heterocycloalkyl is substituted by 1 or 2 groups independently selected from —OH, —N(R¹⁰)₂, —CON(R¹⁰)₂, —NHCOR¹⁰, —NHSO₂R¹⁰, —CH₂OH, —CH₂CH₂OH, and azetidinyl which is substituted by 1 —OH group; and
each R¹⁰ is independently hydrogen or C₁-C₂ alkyl which is unsubstituted or substituted by a —CN, —OH, oxazolyl, or imidazolyl group.

42. The compound of claim 36 or 39, or a pharmaceutically acceptable salt, or solvate thereof, wherein:
R⁹ is

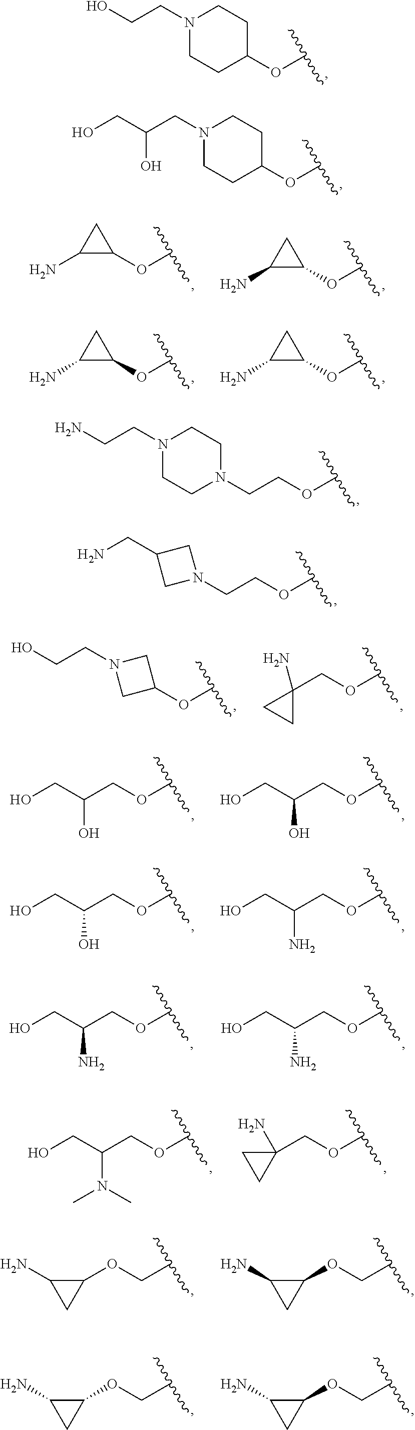

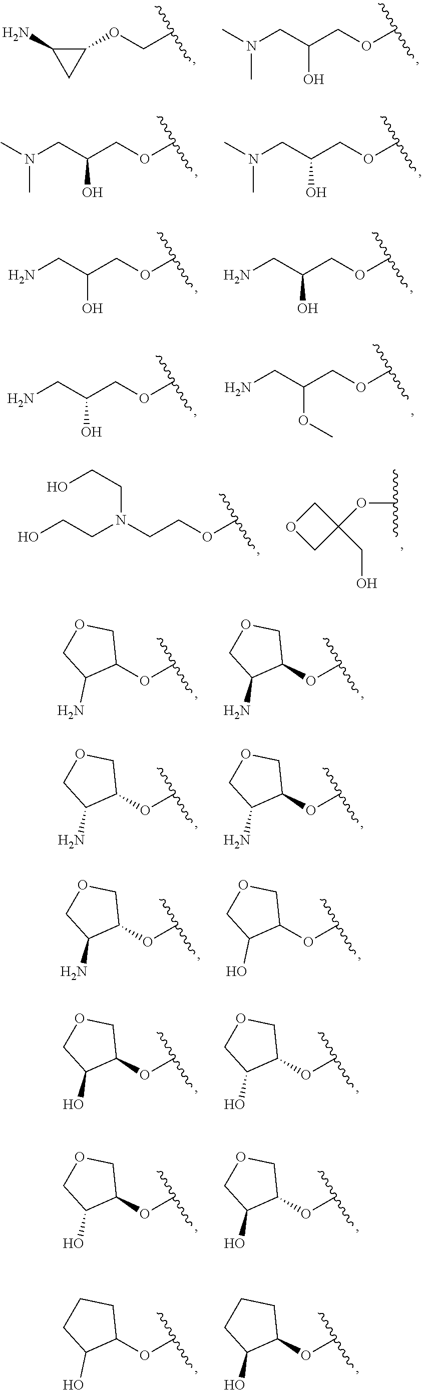

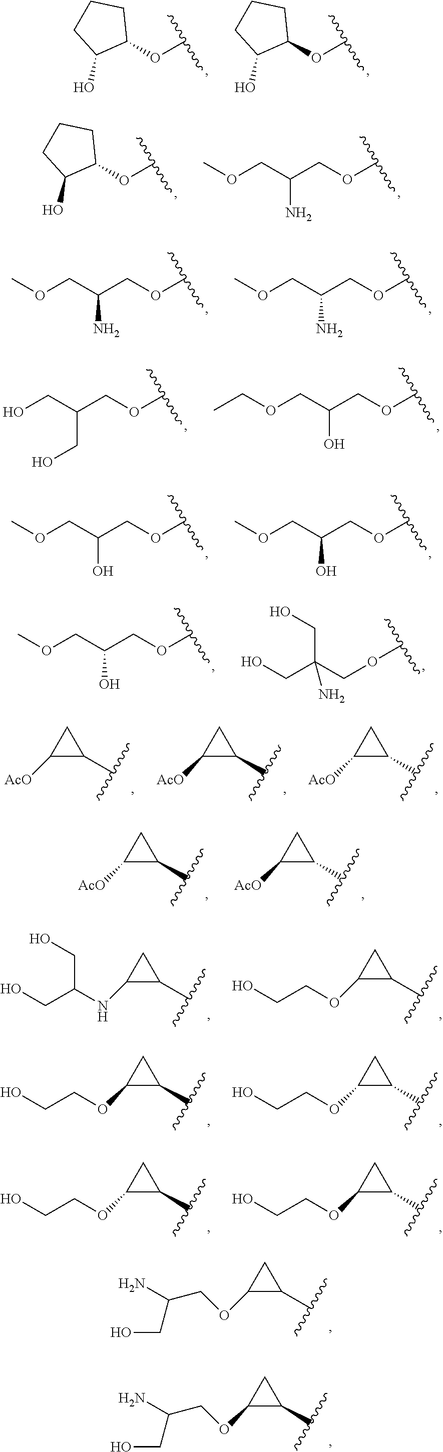

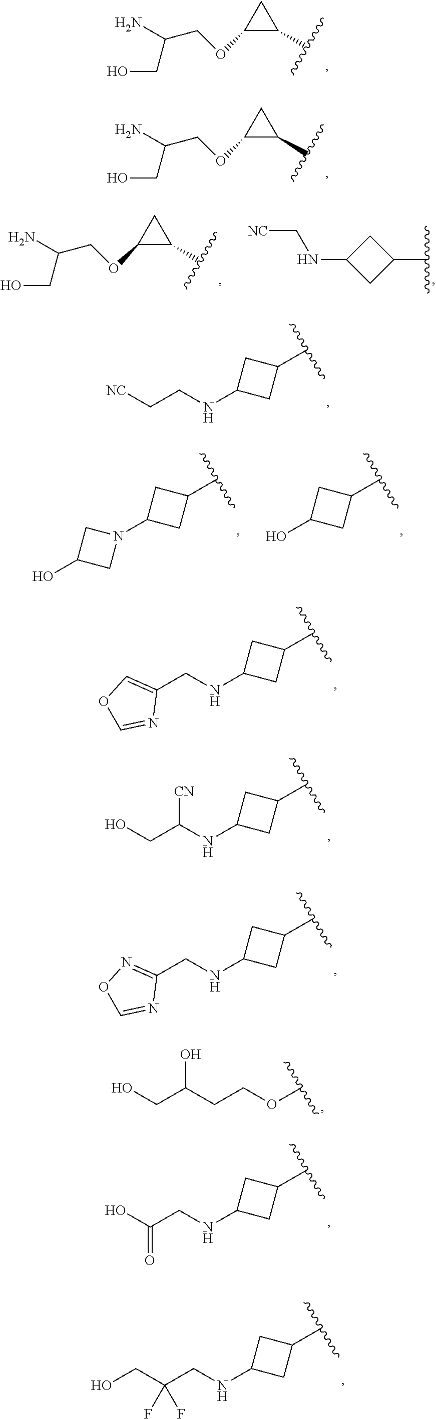

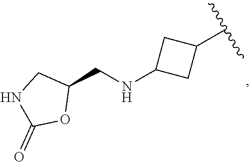 [SMILES: *C1CC(NC[C@@H]2CNC(=O)O2)C1]

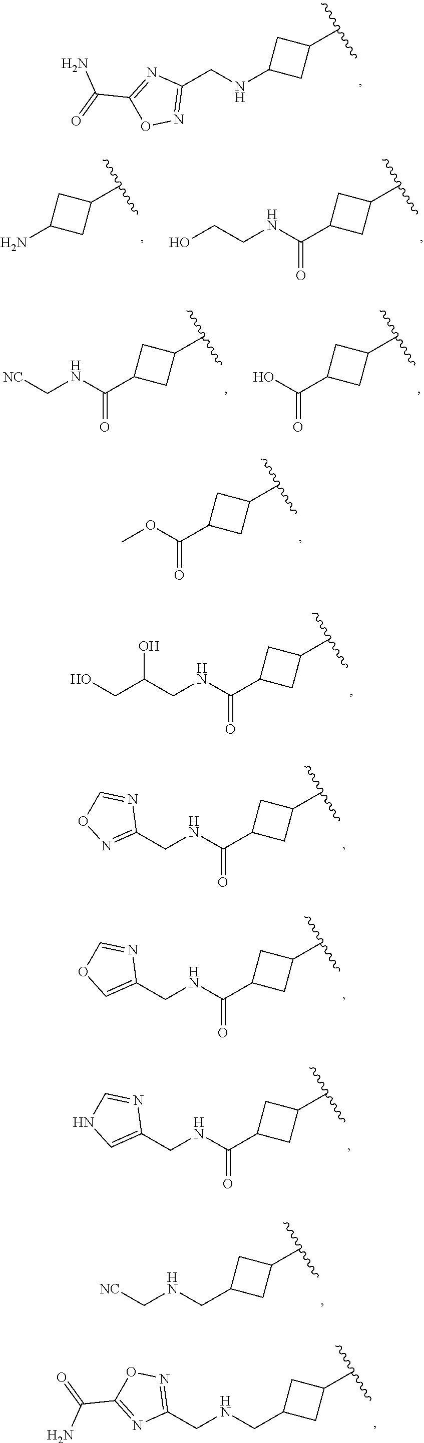

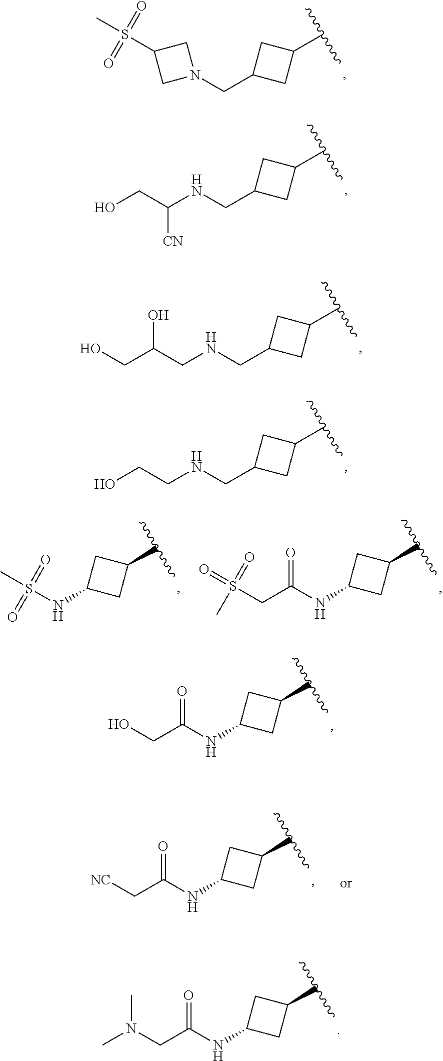

43. The compound of claim 1, selected from:
1: (S,E)-2-(4-((4′-(3-(2-(1-hydroxyethyl)-1H-imidazol-1-yl)prop-1-en-1-yl)-[1,1′-biphenyl]-4-yl)oxy)piperidin-1-yl)ethan-1-ol;
2: 3-(4-((4′-((E)-3-(2-((S)-1-hydroxyethyl)-1H-imidazol-1-yl)prop-1-en-1-yl)-[1,1′-biphenyl]-4-yl)oxy)piperidin-1-yl)propane-1,2-diol;
3: (R)—2-amino-3-((4′-((E)-3-(2-((S)-1-hydroxyethyl)-1H-imidazol-1-yl)prop-1-en-1-yl)-[1,1′-biphenyl]-4-yl)oxy)propan-1-ol;
4: (S)-1-(1-((E)-3-(4′-((trans)-2-aminocyclopropoxy)-[1,1′-biphenyl]-4-yl)allyl)-1H-imidazol-2-yl)ethan-1-ol;
5: (S,E)-1-(1-(3-(4′-(2-(4-(2-aminoethyl)piperazin-1-yl)ethoxy)-[1,1′-biphenyl]-4-yl)allyl)-1H-imidazol-2-yl)ethan-1-ol;
6: (S,E)-1-(1-(3-(4′-(2-(3-(aminomethyl)azetidin-1-yl)ethoxy)-[1,1′-biphenyl]-4-yl)allyl)-1H-imidazol-2-yl)ethan-1-ol;
7: (S,E)-1-(1-(3-(4′-((1-aminocyclopropyl)methoxy)-[1,1′-biphenyl]-4-yl)allyl)-1H-imidazol-2-yl)ethan-1-ol;
8: (2S,E)-4-(4′-(2-amino-3-hydroxypropoxy)-[1,1′-biphenyl]-4-yl)-2-(2-((S)-1-hydroxyethyl)-1H-imidazol-1-yl)but-3-en-1-ol;
9: (S,E)-4-(4′-((R)—2-amino-3-hydroxypropoxy)-[1,1′-biphenyl]-4-yl)-2-(2-((S)-1-hydroxyethyl)-1H-imidazol-1-yl)but-3-en-1-ol;
10: (S,E)-4-(4′-((S)-2-amino-3-hydroxypropoxy)-[1,1′-biphenyl]-4-yl)-2-(2-((S)-1-hydroxyethyl)-1H-imidazol-1-yl)but-3-en-1-ol;
11: (2S,Z)-4-(4′-(2-amino-3-hydroxypropoxy)-[1,1′-biphenyl]-4-yl)-4-fluoro-2-(2-((S)-1-hydroxyethyl)-1H-imidazol-1-yl)but-3-en-1-ol;
12: (2S,E)-4-(4′-(2-(dimethylamino)-3-hydroxypropoxy)-[1,1′-biphenyl]-4-yl)-2-(2-((S)-1-hydroxyethyl)-1H-imidazol-1-yl)but-3-en-1-ol;
13: (S,E)-4-(4′-((trans)-2-aminocyclopropoxy)-[1,1′-biphenyl]-4-yl)-2-(2-((S)-1-hydroxyethyl)-1H-imidazol-1-yl)but-3-en-1-ol;
14: (S,E)-4-(4′-((1S,2S)-2-aminocyclopropoxy)-[1,1′-biphenyl]-4-yl)-2-(2-((S)-1-hydroxyethyl)-1H-imidazol-1-yl)but-3-en-1-ol;
15: (S,E)-4-(4′-((1R,2R)-2-aminocyclopropoxy)-[1,1′-biphenyl]-4-yl)-2-(2-((S)-1-hydroxyethyl)-1H-imidazol-1-yl)but-3-en-1-ol;
16: (S,E)-4-(4′-(((cis)-2-aminocyclopropoxy)methyl)-[1,1′-biphenyl]-4-yl)-2-(2-((S)-1-hydroxyethyl)-1H-imidazol-1-yl)but-3-en-1-ol;
17: (S,E)-2-(2-((S)-1-hydroxyethyl)-1H-imidazol-1-yl)-4-(4′-((1-(2-hydroxyethyl)piperidin-4-yl)oxy)-[1,1′-biphenyl]-4-yl)but-3-en-1-ol;
18: (R,E)-3-(2-((S)-1-hydroxyethyl)-1H-imidazol-1-yl)-5-(4′-((1-(2-hydroxyethyl)piperidin-4-yl)oxy)-[1,1′-biphenyl]-4-yl)pent-4-en-1-ol;
19: (3R,E)-5-(4′-(2-amino-3-hydroxypropoxy)-[1,1′-biphenyl]-4-yl)-3-(2-((S)-1-hydroxyethyl)-1H-imidazol-1-yl)pent-4-en-1-ol;
20: (S,E)-2-(2-((S)-1-hydroxyethyl)-1H-imidazol-1-yl)-4-(4′-((1-(2-hydroxyethyl)azetidin-3-yl)oxy)-[1,1′-biphenyl]-4-yl)but-3-en-1-ol;
21: (2S,E)-4-(4′-(3-(dimethylamino)-2-hydroxypropoxy)-[1,1′-biphenyl]-4-yl)-2-(2-((S)-1-hydroxyethyl)-1H-imidazol-1-yl)but-3-en-1-ol;
22: (2S,E)-4-(4′-(3-amino-2-hydroxypropoxy)-[1,1′-biphenyl]-4-yl)-2-(2-((S)-1-hydroxyethyl)-1H-imidazol-1-yl)but-3-en-1-ol;
23: (R)—3-((4′-((S,E)-4-hydroxy-3-(2-((S)-1-hydroxyethyl)-1H-imidazol-1-yl)but-1-en-1-yl)-[1,1′-biphenyl]-4-yl)oxy)propane-1,2-diol;
24: (S)-3-((4′-((S,E)-4-hydroxy-3-(2-((S)-1-hydroxyethyl)-1H-imidazol-1-yl)but-1-en-1-yl)-[1,1′-biphenyl]-4-yl)oxy)propane-1,2-diol;
25: (2S,E)-4-(4′-(2-amino-3-hydroxypropoxy)-[1,1′-biphenyl]-4-yl)-2-(2-((S)-1-hydroxyethyl)-1H-imidazol-1-yl)but-3-en-1-ol;
26: (S,E)-4-(4′-((S)-3-(dimethylamino)-2-hydroxypropoxy)-[1,1′-biphenyl]-4-yl)-2-(2-((S)-1-hydroxyethyl)-1H-imidazol-1-yl)but-3-en-1-ol;
27: (2S,E)-4-(4′-(3-amino-2-methoxypropoxy)-[1,1′-biphenyl]-4-yl)-2-(2-((S)-1-hydroxyethyl)-1H-imidazol-1-yl)but-3-en-1-ol;
28: 2,2′-((2-((4′-((S,E)-4-hydroxy-3-(2-((S)-1-hydroxyethyl)-1H-imidazol-1-yl)but-1-en-1-yl)-[1,1′-biphenyl]-4-yl)oxy)ethyl)azanediyl)bis(ethan-1-ol);
29: (S,E)-4-(4′-((R)—3-amino-2-hydroxypropoxy)-[1,1′-biphenyl]-4-yl)-2-(2-((S)-1-hydroxyethyl)-1H-imidazol-1-yl)but-3-en-1-ol;
30: (S,Z)-4-(4′-((R)—3-amino-2-hydroxypropoxy)-[1,1′-biphenyl]-4-yl)-4-fluoro-2-(2-((S)-1-hydroxyethyl)-1H-imidazol-1-yl)but-3-en-1-ol; (S,Z)-4-(4′-((S)-3-amino-2-hydroxypropoxy)-[1,1′-biphenyl]-4-yl)-4-fluoro-2-(2-((S)-1-hydroxyethyl)-1H-imidazol-1-yl)but-3-en-1-ol;
31: (S,E)-4-(4′-((S)-2-amino-3-methoxypropoxy)-[1,1′-biphenyl]-4-yl)-2-(2-((S)-1-hydroxyethyl)-1H-imidazol-1-yl)but-3-en-1-ol;
32: (S)-3-((2-fluoro-4′-((S,E)-4-hydroxy-3-(2-((S)-1-hydroxyethyl)-1H-imidazol-1-yl)but-1-en-1-yl)-[1,1′-biphenyl]-4-yl)oxy)propane-1,2-diol;
33: 2-(((4′-((S,E)-4-hydroxy-3-(2-((S)-1-hydroxyethyl)-1H-imidazol-1-yl)but-1-en-1-yl)-[1,1′-biphenyl]-4-yl)oxy)methyl)propane-1,3-diol;
34: (S,E)-4-(4′-(((trans)-4-aminotetrahydrofuran-3-yl)oxy)-[1,1′-biphenyl]-4-yl)-2-(2-((S)-1-hydroxyethyl)-1H-imidazol-1-yl)but-3-en-1-ol;
35: (trans)-4-((4′-((S,E)-4-hydroxy-3-(2-((S)-1-hydroxyethyl)-1H-imidazol-1-yl)but-1-en-1-yl)-[1,1′-biphenyl]-4-yl)oxy)tetrahydrofuran-3-ol;
36: (2S,E)-4-(4′-(3-ethoxy-2-hydroxypropoxy)-[1,1′-biphenyl]-4-yl)-2-(2-((S)-1-hydroxyethyl)-1H-imidazol-1-yl)but-3-en-1-ol;
37: 3-((3-(4′-((S,E)-4-hydroxy-3-(2-((S)-1-hydroxyethyl)-1H-imidazol-1-yl)but-1-en-1-yl)-[1,1′-biphenyl]-4-yl)cyclobutyl)amino)propanenitrile;
38: 1-(3-(4′-((S,E)-4-hydroxy-3-(2-((S)-1-hydroxyethyl)-1H-imidazol-1-yl)but-1-en-1-yl)-[1,1′-biphenyl]-4-yl)cyclobutyl)azetidin-3-ol;
39: 3-(4′-((S,E)-4-hydroxy-3-(2-((S)-1-hydroxyethyl)-1H-imidazol-1-yl)but-1-en-1-yl)-[1,1′-biphenyl]-4-yl)cyclobutan-1-ol;
40: (S,E)-2-(2-((S)-1-hydroxyethyl)-1H-imidazol-1-yl)-4-(4′-(3-((oxazol-4-ylmethyl)amino)cyclobutyl)-[1,1′-biphenyl]-4-yl)but-3-en-1-ol;
41: 3-hydroxy-2-((3-(4′-((S,E)-4-hydroxy-3-(2-((S)-1-hydroxyethyl)-1H-imidazol-1-yl)but-1-en-1-yl)-[1,1′-biphenyl]-4-y)cyclobutyl)amino)propanenitrile;
42: (S,E)-4-(4′-(3-(((1,2,4-oxadiazol-3-yl)methyl)amino)cyclobutyl)-[1,1′-biphenyl]-4-yl)-2-(2-((S)-1-hydroxyethyl)-1H-imidazol-1-yl)but-3-en-1-ol;
43: 4-((4′-((S,E)-4-hydroxy-3-(2-((S)-1-hydroxyethyl)-1H-imidazol-1-yl)but-1-en-1-yl)-[1,1′-biphenyl]-4-yl)oxy)butane-1,2-diol;
44: (3-(4′-((S,E)-4-hydroxy-3-(2-((S)-1-hydroxyethyl)-1H-imidazol-1-yl)but-1-en-1-yl)-[1,1′-biphenyl]-4-yl)cyclobutyl)glycine;
45: (S,E)-4-(4′-(3-((2,2-difluoro-3-hydroxypropyl)amino)cyclobutyl)-[1,1′-biphenyl]-4-yl)-2-(2-((S)-1-hydroxyethyl)-1H-imidazol-1-yl)but-3-en-1-ol;
46: (R)—5-(((3-(4′-((S,E)-4-hydroxy-3-(2-((S)-1-hydroxyethyl)-1H-imidazol-1-yl)but-1-en-1-yl)-[1,1′-biphenyl]-4-yl)cyclobutyl)amino)methyl)oxazolidin-2-one;
47: 3-(((3-(4′-((S,E)-4-hydroxy-3-(2-((S)-1-hydroxyethyl)-1H-imidazol-1-yl)but-1-en-1-yl)-[1,1′-biphenyl]-4-yl)cyclobutyl)amino)methyl)-1,2,4-oxadiazole-5-carboxamide;
48: (S,E)-4-(4′-(3-aminocyclobutyl)-[1,1′-biphenyl]-4-yl)-2-(2-((S)-1-hydroxyethyl)-1H-imidazol-1-yl)but-3-en-1-ol;
49: 3-(4′-((S,E)-4-hydroxy-3-(2-((S)-1-hydroxyethyl)-1H-imidazol-1-yl)but-1-en-1-yl)-[1,1′-biphenyl]-4-yl)-N-(2-hydroxyethyl)cyclobutane-1-carboxamide;
50: N-(cyanomethyl)-3-(4′-((S,E)-4-hydroxy-3-(2-((S)-1-hydroxyethyl)-1H-imidazol-1-yl)but-1-en-1-yl)-[1,1′-biphenyl]-4-y)cyclobutane-1-carboxamide;
51: 3-(4′-((S,E)-4-hydroxy-3-(2-((S)-1-hydroxyethyl)-1H-imidazol-1-yl)but-1-en-1-yl)-[1,1′-biphenyl]-4-yl)cyclobutane-1-carboxylic acid;
52: N-(2,3-dihydroxypropyl)-3-(4′-((S,E)-4-hydroxy-3-(2-((S)-1-hydroxyethyl)-1H-imidazol-1-yl)but-1-en-1-yl)-[1,1′-biphenyl]-4-yl)cyclobutane-1-carboxamide;
53: N-((1,2,4-oxadiazol-3-yl)methyl)-3-(4′-((S,E)-4-hydroxy-3-(2-((S)-1-hydroxyethyl)-1H-imidazol-1-yl)but-1-en-1-yl)-[1,1′-biphenyl]-4-yl)cyclobutane-1-carboxamide;
54: 3-(4′-((S,E)-4-hydroxy-3-(2-((S)-1-hydroxyethyl)-1H-imidazol-1-yl)but-1-en-1-yl)-[1,1′-biphenyl]-4-yl)-N-(oxazol-4-ylmethyl)cyclobutane-1-carboxamide;
55: N-((1H-imidazol-4-yl)methyl)-3-(4′-((S,E)-4-hydroxy-3-(2-((S)-1-hydroxyethyl)-1H-imidazol-1-yl)but-1-en-1-yl)-[1,1′-biphenyl]-4-yl)cyclobutane-1-carboxamide;
56: 2-(((3-(4′-((S,E)-4-hydroxy-3-(2-((S)-1-hydroxyethyl)-1H-imidazol-1-yl)but-1-en-1-yl)-[1,1′-biphenyl]-4-yl)cyclobutyl)methyl)amino)acetonitrile;
57: 3-((((3-(4′-((S,E)-4-hydroxy-3-(2-((S)-1-hydroxyethyl)-1H-imidazol-1-yl)but-1-en-1-yl)-[1,1′-biphenyl]-4-yl)cyclobutyl)methyl)amino)methyl)-1,2,4-oxadiazole-5-carboxamide;
58: (S,E)-2-(2-((S)-1-hydroxyethyl)-1H-imidazol-1-yl)-4-(4′-(3-((3-(methylsulfonyl)azetidin-1-yl)methyl)cyclobutyl)-[1,1′-biphenyl]-4-yl)but-3-en-1-ol;
59: 3-hydroxy-2-(((3-(4′-((S,E)-4-hydroxy-3-(2-((S)-1-hydroxyethyl)-1H-imidazol-1-yl)but-1-en-1-yl)-[1,1′-biphenyl]-4-yl)cyclobutyl)methyl)amino)propanenitrile;
60: 3-(((3-(4′-((S,E)-4-hydroxy-3-(2-((S)-1-hydroxyethyl)-1H-imidazol-1-yl)but-1-en-1-yl)-[1,1′-biphenyl]-4-yl)cyclobutyl)methyl)amino)propane-1,2-diol;
61: (S,E)-2-(2-((S)-1-hydroxyethyl)-1H-imidazol-1-yl)-4-(4′-(3-(((2-hydroxyethyl)amino)methyl)cyclobutyl)-[1,1′-biphenyl]-4-yl)but-3-en-1-ol;
62: N-((1S,3r)-3-(4′-((S,E)-4-hydroxy-3-(2-((S)-1-hydroxyethyl)-1H-imidazol-1-yl)but-1-en-1-yl)-[1,1′-biphenyl]-4-yl)cyclobutyl)methanesulfonamide;
63: N-((1S,3r)-3-(4′-((S,E)-4-hydroxy-3-(2-((S)-1-hydroxyethyl)-1H-imidazol-1-yl)but-1-en-1-yl)-[1,1′-biphenyl]-4-yl)cyclobutyl)-2-(methylsulfonyl)acetamide;
64: 2-hydroxy-N-((1S,3r)-3-(4′-((S,E)-4-hydroxy-3-(2-((S)-1-hydroxyethyl)-1H-imidazol-1-yl)but-1-en-1-yl)-[1,1′-biphenyl]-4-yl)cyclobutyl)acetamide;
65: 2-cyano-N-((1S,3r)-3-(4′-((S,E)-4-hydroxy-3-(2-((S)-1-hydroxyethyl)-1H-imidazol-1-yl)but-1-en-1-yl)-[1,1′-biphenyl]-4-yl)cyclobutyl)acetamide;
66: 2-(dimethylamino)-N-((1S,3r)-3-(4′-((S,E)-4-hydroxy-3-(2-((S)-1-hydroxyethyl)-1H-imidazol-1-yl)but-1-en-1-yl)-[1,1′-biphenyl]-4-yl)cyclobutyl)acetamide; and
67: methyl 3-(4′-((S,E)-4-hydroxy-3-(2-((S)-1-hydroxyethyl)-1H-imidazol-1-yl)but-1-en-1-yl)-[1,1′-biphenyl]-4-yl)cyclobutane-1-carboxylate;
68: (S,E)-2-(2-((S)-1-hydroxyethyl)-1H-imidazol-1-yl)-4-(4′-((3-(hydroxymethyl)oxetan-3-yl)methoxy)-[1,1′-biphenyl]-4-yl)but-3-en-1-ol;
69: (1R,2S)-2-(4′-((S,E)-4-hydroxy-3-(2-((S)-1-hydroxyethyl)-1H-imidazol-1-yl)but-1-en-1-yl)-[1,1′-biphenyl]-4-yl)cyclopropyl acetate;
70: 2-(2-(4′-((S,E)-4-hydroxy-3-(2-((S)-1-hydroxyethyl)-1H-imidazol-1-yl)but-1-en-1-yl)-[1,1′-biphenyl]-4-yl)cyclopropoxy)propane-1,3-diol;
71: (S,E)-4-(4′-((1S,2R)-2-(2-hydroxyethoxy)cyclopropyl)-[1,1′-biphenyl]-4-yl)-2-(2-((S)-1-hydroxyethyl)-1H-imidazol-1-yl)but-3-en-1-ol;
72: (2S,E)-4-(4′-((1S,2R)-2-(2-amino-3-hydroxypropoxy)cyclopropyl)-[1,1′-biphenyl]-4-yl)-2-(2-((S)-1-hydroxyethyl)-1H-imidazol-1-yl)but-3-en-1-ol;
73: (1R,2R)-2-((4′-((S,E)-4-hydroxy-3-(2-((S)-1-hydroxyethyl)-1H-imidazol-1-yl)but-1-en-1-yl)-[1,1′-biphenyl]-4-yl)oxy)cyclopentan-1-ol;
74: 2-amino-2-(((4′-((S,E)-4-hydroxy-3-(2-((S)-1-hydroxyethyl)-1H-imidazol-1-yl)but-1-en-1-yl)-[1,1′-biphenyl]-4-yl)oxy)methyl)propane-1,3-diol;
75: 2-((3-(2-fluoro-4′-((S,E)-4-hydroxy-3-(2-((S)-1-hydroxyethyl)-1H-imidazol-1-yl)but-1-en-1-yl)-[1,1′-biphenyl]-4-yl)cyclobutyl)amino)acetonitrile;
or a pharmaceutically acceptable salt, or solvate thereof.

44. A pharmaceutical composition comprising the compound of any one of claims 1-43, or a pharmaceutically acceptable salt, or solvate thereof, and a pharmaceutically acceptable excipient.

45. A method of treating or preventing a gram-negative bacterial infection in a patient in need thereof comprising administering to the patient the compound of any one of claims 1-43, or a pharmaceutically acceptable salt, or solvate thereof, or the pharmaceutical composition of claim 44.

46. The method of claim 45, wherein the gram-negative bacterial infection is associated with Pseudomonas aeruginosa.

47. The method of claim 45, wherein the gram-negative bacterial infection is a respiratory infection.

48. The method of claim 47, wherein the respiratory infection is pneumonia.

49. The method of claim 48, wherein the pneumonia is community-acquired pneumonia (CAP), health care-associated pneumonia (HCAP), hospital-acquired pneumonia (HAP), ventilator-associate pneumonia (VAP), or a combination thereof.

50. A method of treating or preventing a P. aeruginosa infection in a patient in need thereof comprising administering to the patient the compound of any one of claims 1-43, or a pharmaceutically acceptable salt, or solvate thereof, or the pharmaceutical composition of claim 44.

51. The method of any one of claims 45-50, wherein the patient has been identified as having a lung disease.

52. The method of claim 51, wherein the lung disease is a structural lung disease.

53. The method of claim 51 or claim 52, wherein the lung disease is cystic fibrosis, bronchiectasis, emphysema, chronic obstructive pulmonary disease (COPD), chronic destroyed lung disease, or a combination thereof.

54. The method of any one of claims 45-53, wherein the administration is to treat an existing infection.

55. The method of any one of claims 45-53, wherein the administration is provided as prophylaxis.

56. The method of any one of claims 45-55, wherein the compound of any one of claims 1-43, or a pharmaceutically acceptable salt, or solvate thereof, or the pharmaceutical composition of claim 44, is administered in a solution by inhalation, intravenous injection, or intraperitoneal injection.

57. A compound of any one of claims 1-43 for use as therapeutically active substance.

58. A compound of any one of claims 1-43 for use in treating or preventing a gram-negative bacterial infection.

59. The compound for use of claim 58, wherein the gram-negative bacterial infection is associated with Pseudomonas aeruginosa.

60. The compound for use of claim 58, wherein the gram-negative bacterial infection is a respiratory infection.

61. The compound for use of claim 60, wherein the respiratory infection is pneumonia.

62. The compound for use of claim 60, wherein the pneumonia is community-acquired pneumonia (CAP), health care-associated pneumonia (HCAP), hospital-acquired pneumonia (HAP), ventilator-associate pneumonia (VAP), or a combination thereof.

63. A compound of any one of claims 1-43 for use in treating or preventing a P. aeruginosa infection.

64. The compound for use of any one of claims 58-63, wherein the patient has been identified as having a lung disease.

65. The compound for use of claim 64, wherein the lung disease is a structural lung disease.

66. The compound for use of claim 64 or claim 65, wherein the lung disease is cystic fibrosis, bronchiectasis, emphysema, chronic obstructive pulmonary disease (COPD), chronic destroyed lung disease, or a combination thereof.

67. The use of a compound of any one of claims 1-43 for the preparation of a medicament for treating or preventing a gram-negative bacterial infection.
